(19) United States
(12) Patent Application Publication
(10) Pub. No.: US 2024/0327353 A1
(43) Pub. Date: Oct. 3, 2024

(54) Serotonin 5-HT2A, 5-HT2B, and 5-HT2C Receptor Inverse Agonists

(71) Applicant: Northeastern University, Boston, MA (US)

(72) Inventor: Raymond BOOTH, Arlington, MA (US)

(21) Appl. No.: 18/578,066

(22) PCT Filed: Jul. 14, 2022
(86) PCT No.: PCT/US2022/037220

§ 371 Date: Jan. 10, 2024

Related U.S. Application Data
(60) Provisional application No. 63/221,920, filed on Jul. 14, 2021.

(51) Int. Cl. C07D 213/38; A61K 31/135; A61K 31/341; A61K 31/381; A61K 31/4409; C07C 211/42; C07C 211/49; C07D 307/52; C07D 333/20

(52) U.S. Cl. CPC C07D 213/38; A61K 31/135; A61K 31/341; A61K 31/381; A61K 31/4409; C07C 211/42; C07C 211/49; C07D 307/52; C07D 333/20; C07C 2602/10

(57) ABSTRACT

4-phenyl-2-dimethylaminotetralin compounds, formulations, and methods are provided for selective modulation of serotonin 5-HT2A and 5-HT2C receptors without causing sedation at doses that are antipsychotic. Mechanisms for selective modulation are shown to involve inverse agonism at one or more of the 5-HT2A-2C receptors based on stereochemistry and substituents. The technology can be targeted to receptors inside or outside the central nervous system.

### CROSS-REFERENCE TO RELATED APPLICATIONS

This application claims priority to U.S. Provisional Application No. 63/221,920, filed 14 Jul. 2021, which is hereby incorporated by reference in its entirety.

### STATEMENT REGARDING FEDERALLY SPONSORED RESEARCH OR DEVELOPMENT

This invention was made with government support under Grant No. RO1 DA030989, RO1 DA047130, and RO1 MH081193 awarded by the NIH National Institutes of Health. The government has certain rights in the invention.

### BACKGROUND

G protein-coupled receptors (GPCRs) are targeted by about 34% of FDA approved drugs, many of which mediate aminergic neurotransmission (Hauser, et al., 2017). Among at least thirteen serotonin (5-hydroxytryptamine, 5-HT) GPCRs are 5-HT_{2A}, 5-HT_{2B}, and 5-HT_{2C} receptors (Rs), which represent promising neurotherapeutic targets. However, extensive structural homology complicates the design of selective therapeutic agents. For example, 5-HT₂-type receptors share 60-70% amino acid identity within structurally conserved regions, and 27-31% identity with histamine receptors (H₁Rs), (Pandy-Szekeres, et al., 2018).

Antagonism of 5-HT_{2A}Rs is associated with the improved efficacy of so-called atypical antipsychotics toward schizophrenia, and hallucinations and delusions in psychosis (Meltzer, 1999; Weiner, et al., 2001; Hacksell, et al., 2014). Additionally, inverse agonism of 5-HT_{2C}Rs, a mainstay of atypical antipsychotic polypharmacology, might be pharmacotherapeutic for generalized anxiety, major depression, and schizophrenia (Chagraoui, et al., 2016; Demireva, et al., 2018).

The selective 5-HT_{2A}/5-HT_{2C}R inverse agonist pimavanserin (PIMA) is approved to treat hallucinations and delusions associated with Parkinson's disease psychosis (Meltzer, 1999; Cummings, et al., 2014), though the contribution of 5-HT_{2C}Rs to PIMA's efficacy is unclear (Stahl, 2016). Meanwhile, activation of 5-HT_{2B}Rs is linked to valvular heart disease (Rothman, et al., 2000; Ayme-Dietrich, et al., 2017), and deficiency or antagonism of 5-HT_{2B}Rs is linked to psychotic-like and impulsive behaviors in laboratory animals and humans (Bevilacqua, et al., 2010; Pitychoutis, et al., 2015). Thus, engagement of 5-HT_{2B}Rs may be undesirable for antipsychotic medications. Likewise, H₁Rs represent a common ‘off-target’ for CNS-penetrating drugs (Weiner, et al., 2001) and H₁R antagonism is associated with sedative-hypnotic effects (Nicholson, et al., 1991; Stahl, 2008). Notably, PIMA has nil affinity for H₁Rs and does not appear to cause daytime sleepiness in humans (Cummings, et al., 2014; Meltzer, et al., 2010; Ancoli-Israel, et al., 2011; Fava, et al., 2019). Structural homology of receptors, broad distribution of 5-HT receptors and various side effects such as QT interval prolongation, locomotion problems, and nonspecific receptor binding prevent accurate targeting of indications in subjects. Modulators of 5-HT_{2A}, 5-HT_{2B} and 5-HT_{2C} receptors with improved selectivity are needed.

Through receptors in the central nervous system (CNS) and the periphery, serotonin can modulate many organ systems in the body, including cardiac functions, the cardiovascular system, the gastrointestinal (GI) system, genitourinary systems, the endocrine system, metabolism, reproductive function and pregnancy as well as the CNS. Targeting 5-HT receptors in the periphery can affect multiple systems in the body.

### SUMMARY

The present technology provides novel 4-phenyl-2-dimethylaminotetralin (4-PAT) compounds that are shown to provide inverse agonism at one or more 5-HT_{2A-C} receptors. The compounds do not cause sedation at doses that are antipsychotic. The technology demonstrates mechanisms that can be predictive of the selective efficacy of substituents and stereochemistry of 4-PAT compounds. The technology also provides novel serotonin receptor-modulating compounds that do not substantially accumulate in the brain (or CNS) and therefore are useful in treating diseases or disorders of the periphery.

The technology can be further summarized by the following list of features.

1. A compound for selective modulation of one or more of serotonin 5-HT2A and 5-HT2C receptors, the compound having a structure according to Formula I:

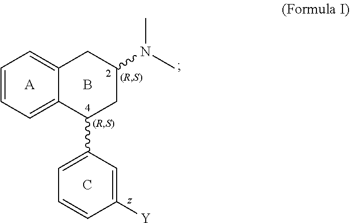

wherein Y is selected from the group consisting of

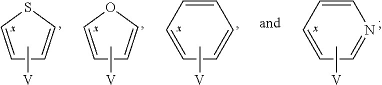

wherein covalent bond z is attached at any carbon atom of Y;
wherein Y is unsubstituted or is substituted with one or more moieties V, each of the one or more moieties V independently selected from the group consisting —F, —Cl, —Br, —I, —NH₂, —NH(CH₃), —N(CH₃)₂, —NH(CH₂CH₃), —N(CH₂CH₃)₂, —C═NH, —C═NNH₂, —C═ONH₂, —NO₂, —NO, —CN, —N₃, —N═C═O, —CH₃, —CH₂CH₃, —CH(CH₃)₂, —C═OOH, —CH₂C═OOH, —S═OCH₃, —S(═O)₂CH₃, —S(═O)₂OH, —S(═O)₂NH₂, —S(═O)₂N(CH₃)₂, —OH, —OCN, —OCH₃, —OCH₂CH₃, —CH₂OH, —CH₂CH₂OH, —CHOHCH₂OH, —CHOHCH₃, —SH, —SCN, —SCH₃, —SCH₂CH₃, —CH₂SH, —CH₂CH₂SH, —CHSHCH₂SH, —CHSHCH₃, and substituted or unsubstituted thiophene, furanyl, phenyl and pyridyl; and
wherein the compound comprises at least 50% of a single stereoisomer selected from the group of stereoisomers consisting of 2R4R, 2S4S, 2R4S, and 2S4R;
or a pharmaceutically acceptable salt, hydrate, or solvate thereof.
   2. The compound of feature 1, wherein one or more moieties V are independently selected from the group consisting of

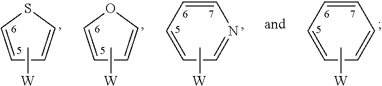

wherein V is attached to Y via a covalent bond to any one of carbons 5-7 of V; and
wherein V is substituted with one or more substituents W, each of the one or more substituents W independently selected from the group consisting of —F, —C, —Br, —I, —NH₂, —NH(CH₃), —N(CH₃)₂, —NH(CH₂CH₃), —N(CH₂CH₃)₂, —C═NH, —C═NNH₂, —C═ONH₂, —NO₂, —NO, —CN, —N₃, —N═C═O, —CH₃, —CH₂CH₃, —CH(CH₃)₂, —C═OOH, —CH₂C═OOH, —S═OCH₃, —S(═O)₂CH₃, —S(═O)₂OH, —S(═O)₂NH₂, —S(═O)₂N(CH₃)₂, —OH, —OCN, —OCH₃, —OCH₂CH₃, —CH₂OH, —CH₂CH₂OH, —CHOHCH₂OH, —CHOHCH₃, —SH, —SCN, —SCH₃, —SCH₂CH₃, —CH₂SH, —CH₂CH₂SH, —CHSHCH₂SH and —CHSHCH₃.
   3. The compound of any of the preceding features, wherein the compound comprises at least 60%, at least 70%, at least 80%, at least 90%, at least 95%, or at least 99% of said single stereoisomer.
   4. The compound of any of the preceding features, wherein Y is bound to C through the bond z attached at carbon atom x of Y.
   5. The compound of any of the preceding features, wherein the compound is selected from the group consisting of the following compounds:

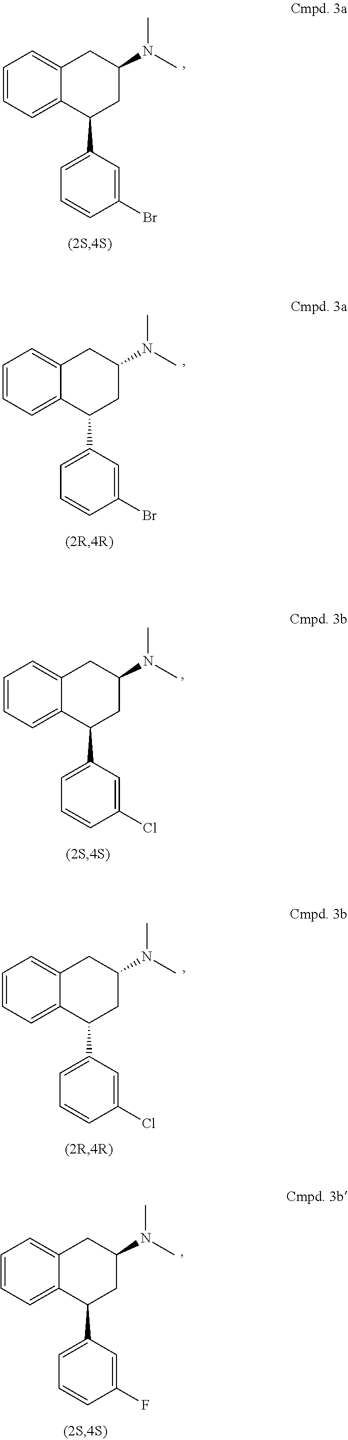 [SMILES: CN(C)[C@@H]1Cc2ccccc2[C@H](c2cccc(Br)c2)C1 ; CN(C)[C@H]1Cc2ccccc2[C@@H](c2cccc(Br)c2)C1 ; CN(C)[C@@H]1Cc2ccccc2[C@H](c2cccc(Cl)c2)C1 ; CN(C)[C@H]1Cc2ccccc2[C@@H](c2cccc(Cl)c2)C1 ; CN(C)[C@@H]1Cc2ccccc2[C@H](c2cccc(F)c2)C1]

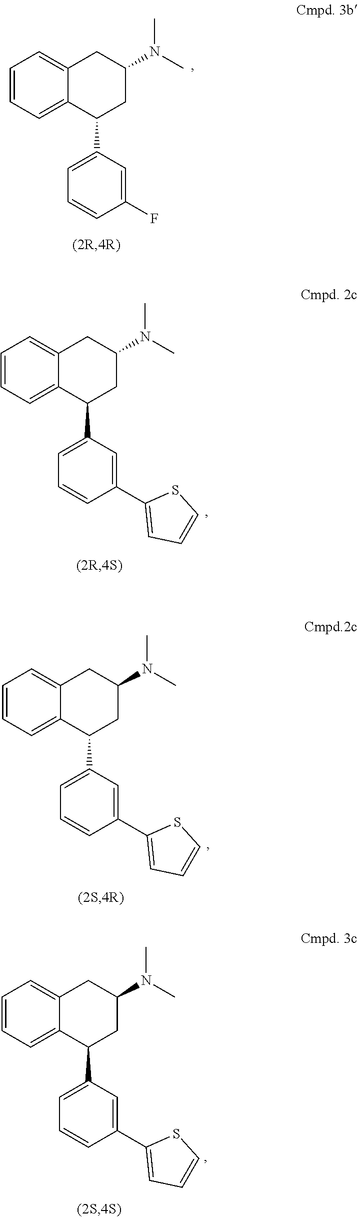 [SMILES: CN(C)[C@H]1Cc2ccccc2[C@@H](c2cccc(F)c2)C1 ; CN(C)[C@H]1Cc2ccccc2[C@H](c2cccc(-c3cccs3)c2)C1 ; CN(C)[C@@H]1Cc2ccccc2[C@@H](c2cccc(-c3cccs3)c2)C1 ; CN(C)[C@@H]1Cc2ccccc2[C@H](c2cccc(-c3cccs3)c2)C1]

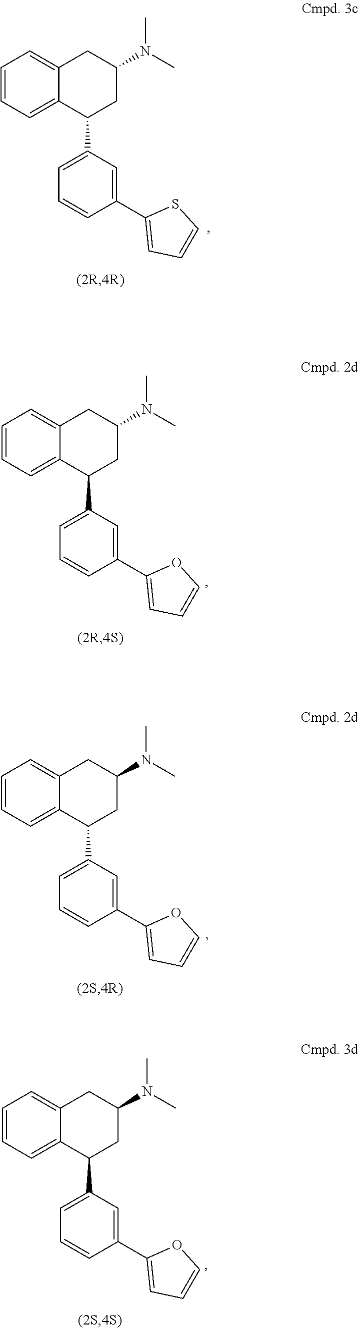 [SMILES: CN(C)[C@H]1Cc2ccccc2[C@@H](c2cccc(-c3cccs3)c2)C1 ; CN(C)[C@H]1Cc2ccccc2[C@H](c2cccc(-c3ccco3)c2)C1 ; CN(C)[C@@H]1Cc2ccccc2[C@@H](c2cccc(-c3ccco3)c2)C1 ; CN(C)[C@@H]1Cc2ccccc2[C@H](c2cccc(-c3ccco3)c2)C1]

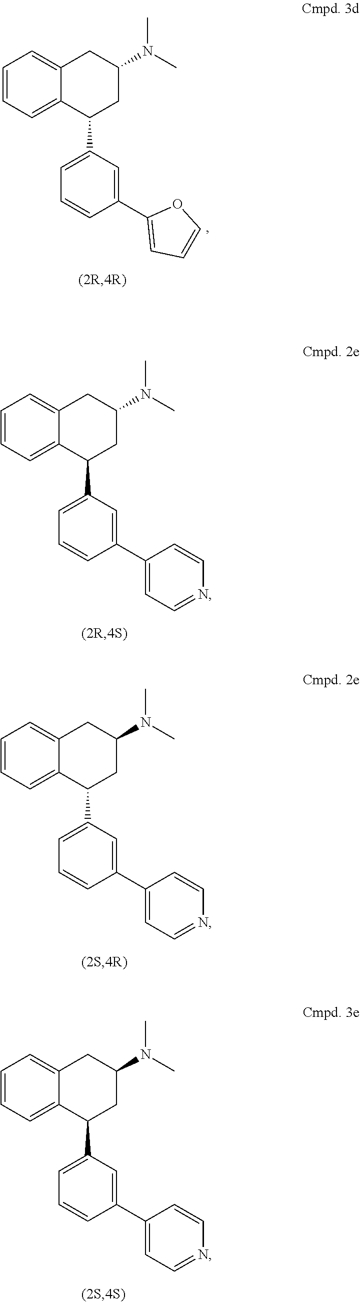 [SMILES: CN(C)[C@H]1Cc2ccccc2[C@@H](c2cccc(-c3ccco3)c2)C1 ; CN(C)[C@H]1Cc2ccccc2[C@H](c2cccc(-c3ccncc3)c2)C1 ; CN(C)[C@@H]1Cc2ccccc2[C@@H](c2cccc(-c3ccncc3)c2)C1 ; CN(C)[C@@H]1Cc2ccccc2[C@H](c2cccc(-c3ccncc3)c2)C1]

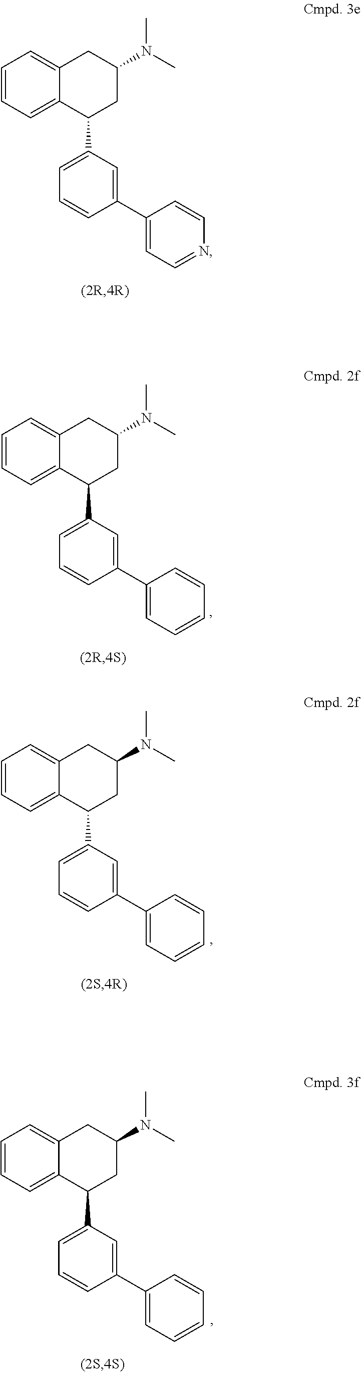 [SMILES: CN(C)[C@H]1Cc2ccccc2[C@@H](c2cccc(-c3ccncc3)c2)C1 ; CN(C)[C@H]1Cc2ccccc2[C@H](c2cccc(-c3ccccc3)c2)C1 ; CN(C)[C@@H]1Cc2ccccc2[C@@H](c2cccc(-c3ccccc3)c2)C1 ; CN(C)[C@@H]1Cc2ccccc2[C@H](c2cccc(-c3ccccc3)c2)C1]

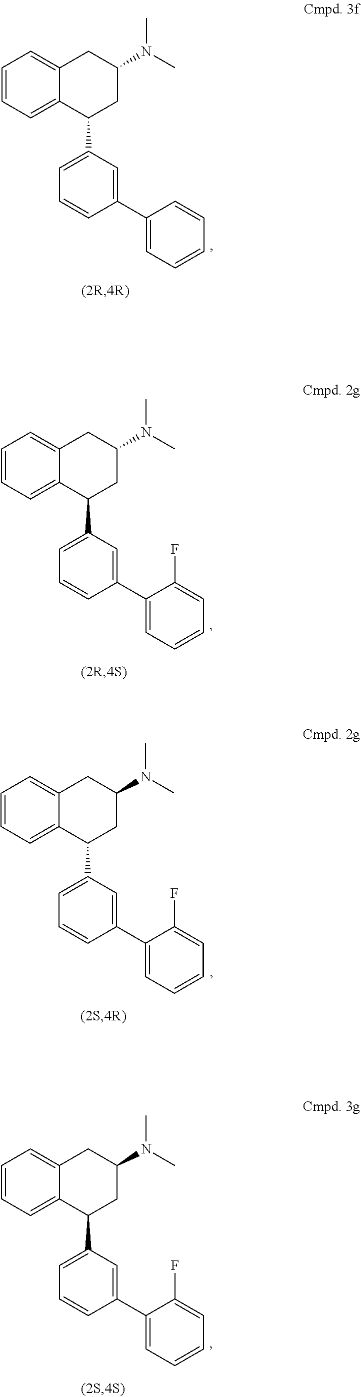 [SMILES: CN(C)[C@H]1Cc2ccccc2[C@@H](c2cccc(-c3ccccc3)c2)C1 ; CN(C)[C@H]1Cc2ccccc2[C@H](c2cccc(-c3ccccc3F)c2)C1 ; CN(C)[C@@H]1Cc2ccccc2[C@@H](c2cccc(-c3ccccc3F)c2)C1 ; CN(C)[C@@H]1Cc2ccccc2[C@H](c2cccc(-c3ccccc3F)c2)C1]

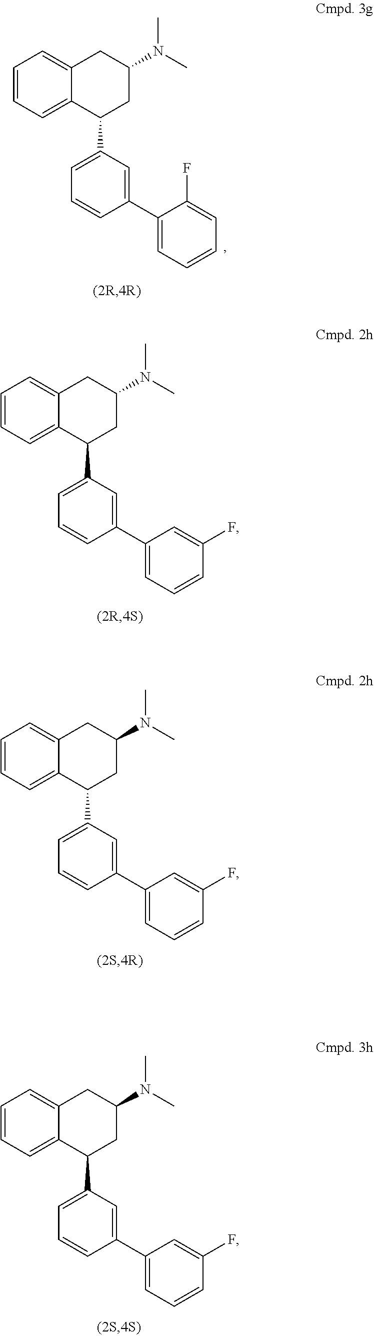 [SMILES: CN(C)[C@H]1Cc2ccccc2[C@@H](c2cccc(-c3ccccc3F)c2)C1 ; CN(C)[C@H]1Cc2ccccc2[C@H](c2cccc(-c3cccc(F)c3)c2)C1 ; CN(C)[C@@H]1Cc2ccccc2[C@@H](c2cccc(-c3cccc(F)c3)c2)C1 ; CN(C)[C@@H]1Cc2ccccc2[C@H](c2cccc(-c3cccc(F)c3)c2)C1]

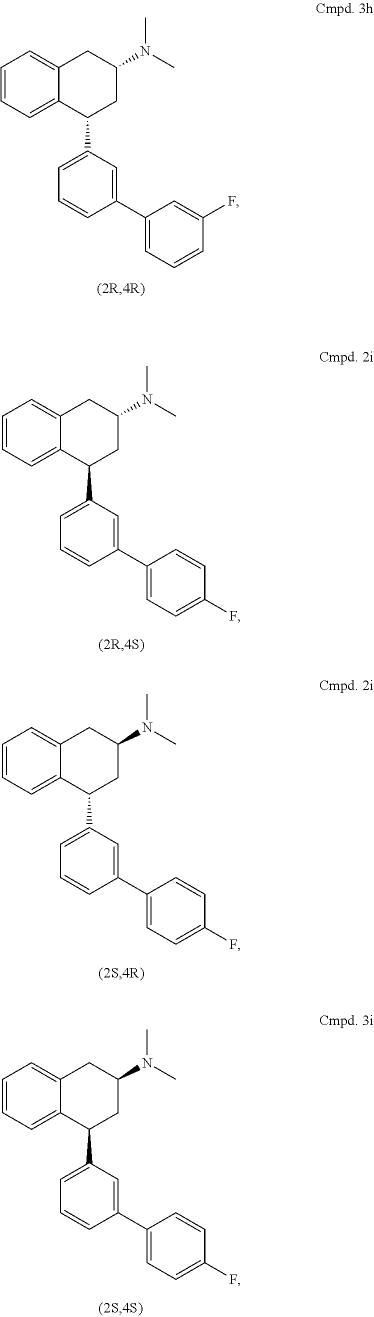 [SMILES: CN(C)[C@H]1Cc2ccccc2[C@@H](c2cccc(-c3cccc(F)c3)c2)C1 ; CN(C)[C@H]1Cc2ccccc2[C@H](c2cccc(-c3ccc(F)cc3)c2)C1 ; CN(C)[C@@H]1Cc2ccccc2[C@@H](c2cccc(-c3ccc(F)cc3)c2)C1 ; CN(C)[C@@H]1Cc2ccccc2[C@H](c2cccc(-c3ccc(F)cc3)c2)C1]

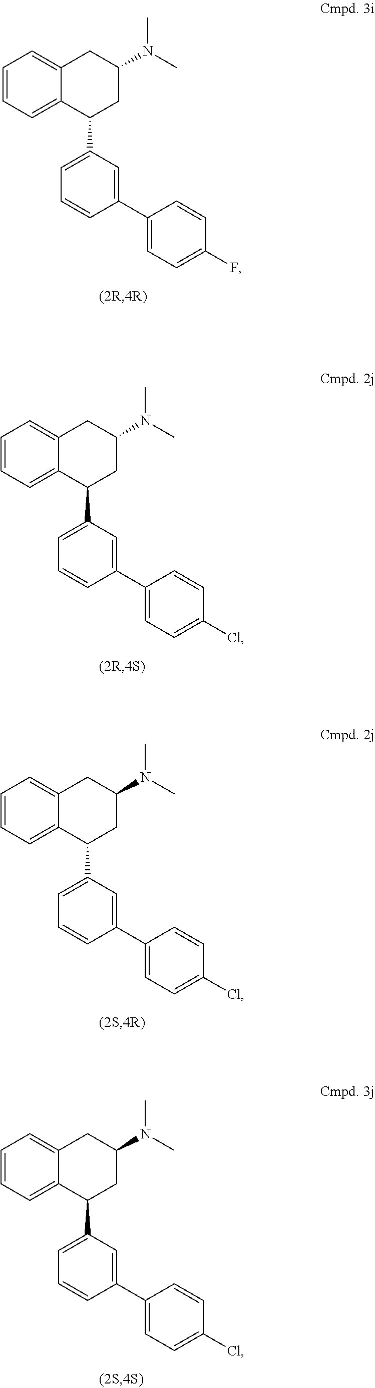 [SMILES: CN(C)[C@H]1Cc2ccccc2[C@@H](c2cccc(-c3ccc(F)cc3)c2)C1 ; CN(C)[C@H]1Cc2ccccc2[C@H](c2cccc(-c3ccc(Cl)cc3)c2)C1 ; CN(C)[C@@H]1Cc2ccccc2[C@@H](c2cccc(-c3ccc(Cl)cc3)c2)C1 ; CN(C)[C@@H]1Cc2ccccc2[C@H](c2cccc(-c3ccc(Cl)cc3)c2)C1]

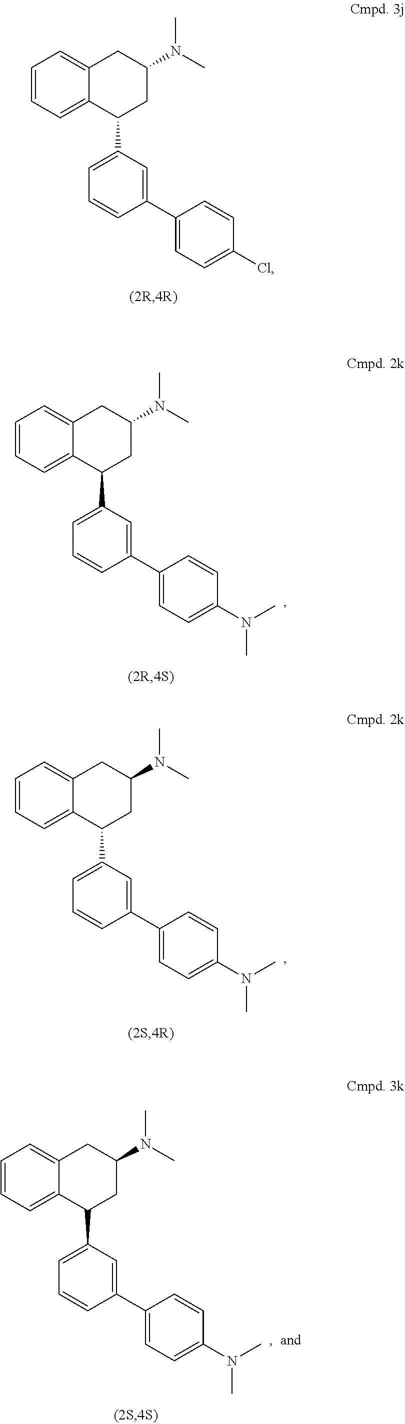

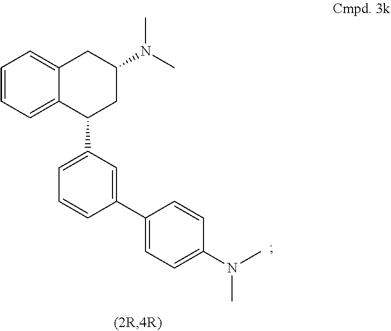 [SMILES: CN(C)c1ccc(-c2cccc([C@H]3C[C@@H](N(C)C)Cc4ccccc43)c2)cc1]

or a pharmaceutically acceptable salt, hydrate, or solvate thereof.
   6. The compound of any of the preceding features, wherein the compound is a neutral antagonist or an inverse agonist at one or more of the 5-HT2A and 5-HT2C receptors.
   7. The compound of any of the preceding features, wherein the compound does not cause sedation when administered to a subject at physiologically relevant levels.
   8. The compound of any of the preceding features, wherein the compound comprises a greater binding affinity at 5-HT2A receptor and/or 5-HT2C receptor than at 5-HT2B receptor.
   9. The compound of any of the preceding features, wherein the compound has a greater binding affinity for 5-HT2A receptor and 5-HT2C receptor than for 5-HT1A, 5-HT2B, 5-HT7, D2, D3, alpha1A, and/or alpha1 B receptors.
   10. The compound of any of the preceding features, wherein the compound is a neutral antagonist or an inverse agonist at a histamine (H1) receptor at physiologically relevant levels.
   11. The compound of any of the preceding features, wherein the compound comprises a greater binding affinity at 5-HT2A receptors and/or 5-HT2C receptors than at the H1 receptor.
   12. The compound of any of the preceding features, wherein the one or more moieties V and/or W comprise a positive and/or a negative charge at a physiological pH.
   13. The compound of feature 12, comprising a pharmaceutically acceptable anion comprising acetate, adipate, aspartate, benzenesulfonate, benzoate, besylate, bicarbonate, bitartrate, bromide, camsylate, caprate, caproate, caprylate, carbonate, chloride, citrate, decanoate, dodecylsulfate, edetate, esylate, formate, fumarate, gluceptate, gluconate, glutamate, glycolate, hexanoate, hydroxynaphthoate, iodide, isethionate, lactate, lactobionate, laurate, malate, maleate, mandelate, mesylate, methylsulfate, mucate, napsylate, nitrate, octanoate, oleate, oxalate, palmitate, pamoate, pantothenate, phosphate, dihydrogen phosphate dodecahydrate, dihydrogen phosphate dihydrate, polygalacturonate, propionate, sabacate, salicylate, stearate, acetate, succinate, sulfate, tartrate, teoclate, thiocyanate, tosylate, or undecylenate.
   14. The compound of feature 12, comprising a pharmaceutically acceptable cation comprising aluminum, arginine, benzathine, calcium, chloroprocaine, choline, diethanolamine, ethanolamine, ethylenediamine, lysine, magnesium, histidine, lithium, meglumine, potassium, procaine, sodium, triethylamine, or zinc.
   15. The compound of any of the preceding features, wherein the compound comprises a hydrate or a solvate comprising one or more water molecules and/or one or more solvent molecules associated via hydrogen bonding and/or ionic bonding to the compound and/or to an anion or cation associated with the compound.
   16. The compound of any of the preceding features, wherein the compound comprises one or more of ¹⁸F, ¹⁹F, ⁷⁵Br, ⁷⁶Br, ¹²³I, ¹²⁴I, ¹²⁵I, ¹³¹I, ¹¹C, ¹³C, ¹³N, ¹⁵O, or ³H.
   17. The compound of any of the preceding features, wherein the compound selectively modulates a physiological activity of 5-HT2A and/or 5-HT2C receptors over a physiological activity of one or more of 5-HT1A, 5-HT2B, 5HT7, D2, D3, α1A, and α1B receptors.
   18. The compound of feature 17, wherein said selective modulation is associated with a difference in binding affinity, inverse agonism, agonism, partial agonism, allosteric agonism, antagonism, partial antagonism, or allosteric antagonism.
   19. A pharmaceutical composition comprising a therapeutically effective amount of a compound of any of the preceding features and an excipient.
   20. The pharmaceutical composition of feature 19 comprising an amount of said compound that aids in treating psychosis, fragile X syndrome, autism, substance use disorder, or an impulsive behavior.
   21. The pharmaceutical composition of feature 19 comprising an amount of said compound that aids in treating hypertension, migraine, obesity, irritable bowel syndrome, Parkinson's disease, attention deficit hyperactivity disorder, anxiety or generalized anxiety, depression, schizophrenia, binge eating, opioid use disorder, amphetamine use disorder, panic disorder, social anxiety disorder, obsessive-compulsive disorder, pain, Alzheimer's disease, or Huntington's disease.
   22. The pharmaceutical composition of feature 19, wherein the compound comprises (2R,4S)-(trans)-4-(3-(thiophen-2-yl)phenyl)-N,N-dimethyl-1,2,3,4-tetrahydronaphthalen-2-amine, (2R,4S)-(trans)-4-(3-(furan-2-yl)phenyl)-N,N-dimethyl-1,2,3,4-tetrahydronaphthalen-2-amine, or (2S,4S)-(cis)-4-([1,1′-biphenyl]-3-yl)-N,N-dimethyl-1,2,3,4-tetrahydronaphthalen-2-amine.
   23. A method to aid in treating a disease or disorder, the method comprising administering an effective amount of a compound of any of features 1-18 to a mammalian subject in need thereof.
   24. The method of feature 23, wherein the compound is administered as the pharmaceutical composition of any of features 19-22.
   25. The method of feature 23 or feature 24, wherein said administering does not cause sedation, dizziness, and/or orthostatic hypotension.
   26. The method of feature 23, wherein the disease or disorder is a neuropsychiatric disorder is selected from the group consisting of psychosis, fragile X syndrome, autism, substance use disorder, and impulsive behaviors.
   27. The method of feature 23, wherein the disease or disorder is selected from the group consisting of hypertension, migraine, obesity, irritable bowel syndrome, Parkinson's disease, attention deficit hyperactivity disorder, anxiety or generalized anxiety, depression, schizophrenia, binge eating, opioid use disorder, amphetamine use disorder, panic disorder, social anxiety disorder, obsessive-compulsive disorder, pain, Alzheimer's disease, or Huntington's disease.
   28. The method of any of features 23-27, wherein said administering results in selective modulation of a serotonin 5-HT2A or 5-HT2C receptor in the subject.
   29. The method of feature 18, wherein the selective modulation comprises inverse agonism, agonism, partial agonism, allosteric agonism, antagonism, partial antagonism, allosteric antagonism, or a difference in binding affinity compared to a different receptor type.
   30. Use of the compound of any of features 1-18 or the composition of any of features 19-22 to treat or prevent psychosis, fragile X syndrome, autism, substance use disorder, impulsive behaviors, hypertension, migraine, obesity, irritable bowel syndrome, Parkinson's disease, attention deficit hyperactivity disorder, anxiety or generalized anxiety, depression, schizophrenia, binge eating, opioid use disorder, amphetamine use disorder, panic disorder, social anxiety disorder, obsessive-compulsive disorder, pain, Alzheimer's disease, and/or Huntington's disease in a mammalian subject.
   31. The use of feature 30, wherein said use does not cause sedation in the subject.
   32. The use of feature 30, wherein said use does not agonize 5-HT2B receptors and/or does not antagonize H1 receptors in the subject.
   33. A compound for selective modulation of one or more of peripheral serotonin 5-HT2A, 5-HT2B, and 5-HT2C receptors, the compound having a structure according to Formula I:

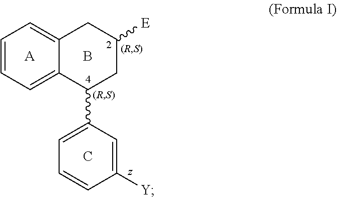

wherein E is a quaternary amine selected from the group consisting of —N⁺(CH₃)₃, —N⁺(CH₃)₂(CH₂CH₃), —N⁺(CH₃)(CH₂CH₃)₂, and —N⁺(CH₂CH₃)₃;
wherein Y is selected from the group consisting of:

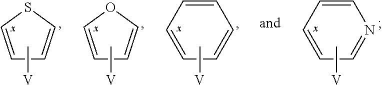

wherein covalent bond z is attached at any carbon atom of Y;
wherein Y is unsubstituted or is substituted with one or more moieties V, each of the one or more moieties V independently selected from the group consisting —F, —Cl, —Br, —I, —NH₂, —NH(CH₃), —N(CH₃)₂, —NH(CH₂CH₃), —N(CH₂CH₃)₂, —C═NH, —C═NNH₂, —C═ONH₂, —NO₂, —NO, —CN, —N₃, —N═C═O, —CH₃, —CH₂CH₃, —CH(CH₃)₂, —C═OOH, —CH₂C═OOH, —S═OCH₃, —S(═O)₂CH₃, —S(═O)₂OH, —S(═O)₂NH₂, —S(═O)₂N(CH₃)₂, —OH, —OCN, —OCH₃, —OCH₂CH₃, —CH₂OH, —CH₂CH₂OH, —CHOHCH₂OH, —CHOHCH₃, —SH, —SCN, —SCH₃, —SCH₂CH₃, —CH₂SH, —CH₂CH₂SH, —CHSHCH₂SH, —CHSHCH₃, and substituted or unsubstituted thiophene, furanyl, phenyl and pyridyl; and
wherein the compound comprises at least 50% of a single stereoisomer selected from the group of stereoisomers consisting of 2R4R, 2S4S, 2R4S, and 2S4R;
or a pharmaceutically acceptable salt, hydrate, or solvate thereof.
   34. The compound of feature 33, wherein one or more moieties V are independently selected from the group consisting of:

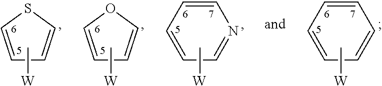

wherein V is attached to Y via a covalent bond to any one of carbons 5-7 of V; and
wherein V is substituted with one or more substituents W, each of the one or more substituents W independently selected from the group consisting of —F, —Cl, —Br, —I, —NH₂, —NH(CH₃), —N(CH₃)₂, —NH(CH₂CH₃), —N(CH₂CH₃)₂, —C═NH, —C═NNH₂, —C═ONH₂, —NO₂, —NO, —CN, —N₃, —N═C═O, —CH₃, —CH₂CH₃, —CH(CH₃)₂, —C═OOH, —CH₂C═OOH, —S═OCH₃, —S(═O)₂CH₃, —S(═O)₂OH, —S(═O)₂NH₂, —S(═O)₂N(CH₃)₂, —OH, —OCN, —OCH₃, —OCH₂CH₃, —CH₂OH, —CH₂CH₂OH, —CHOHCH₂OH, —CHOHCH₃, —SH, —SCN, —SCH₃, —SCH₂CH₃, —CH₂SH, —CH₂CH₂SH, —CHSHCH₂SH and —CHSHCH₃.
   35. The compound of feature 33 or 34, wherein the compound comprises at least 60%, at least 70%, at least 80%, at least 90%, at least 95%, or at least 99% of said single stereoisomer.
   36. The compound of any of features 33-35, wherein Y is bound to C through the bond z attached at the carbon atom x of Y.
   37. The compound of any of features 33-36, wherein the compound is selected from the group consisting of:

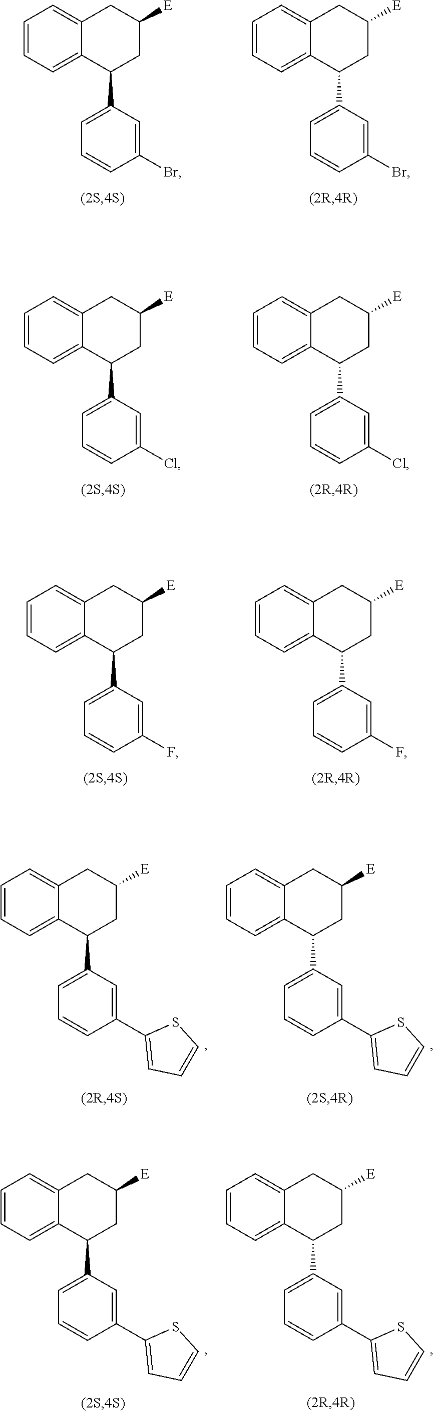

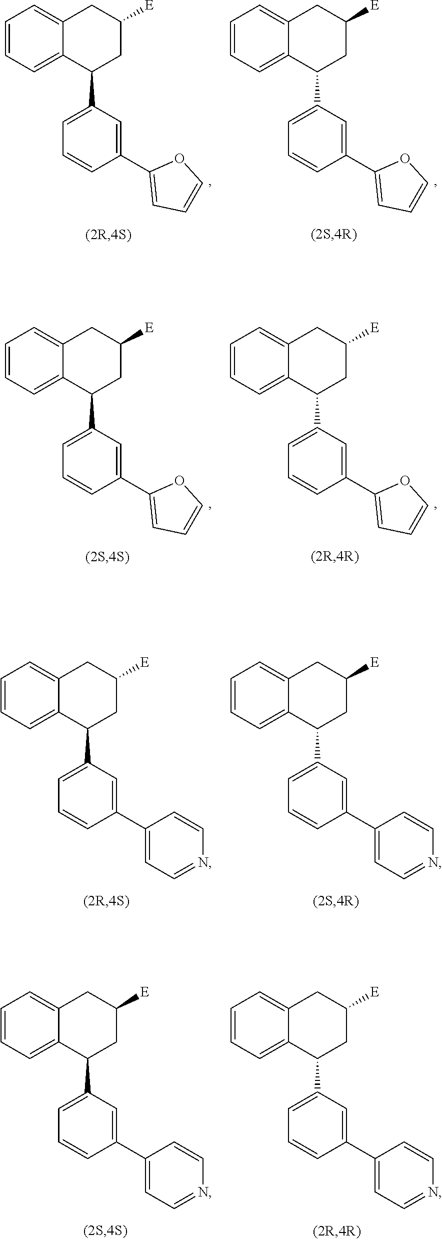

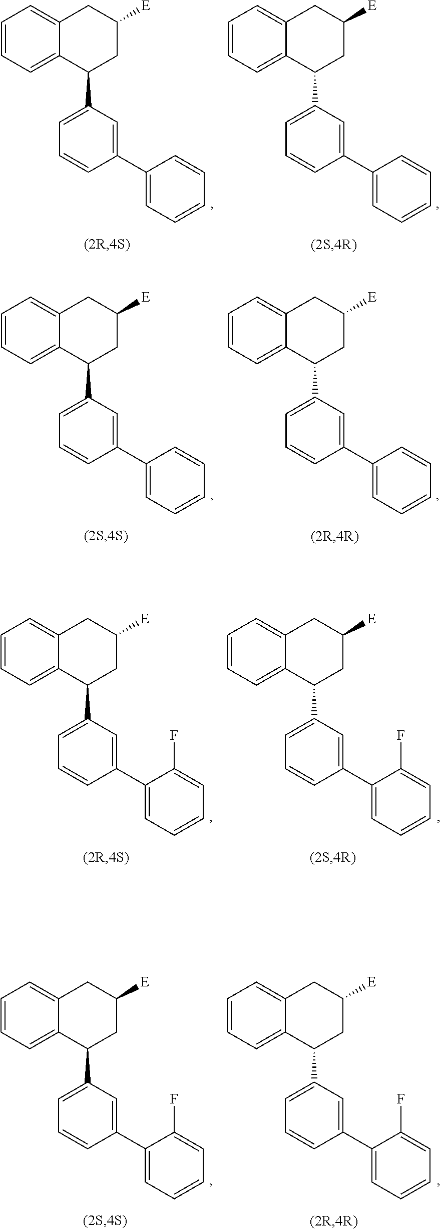

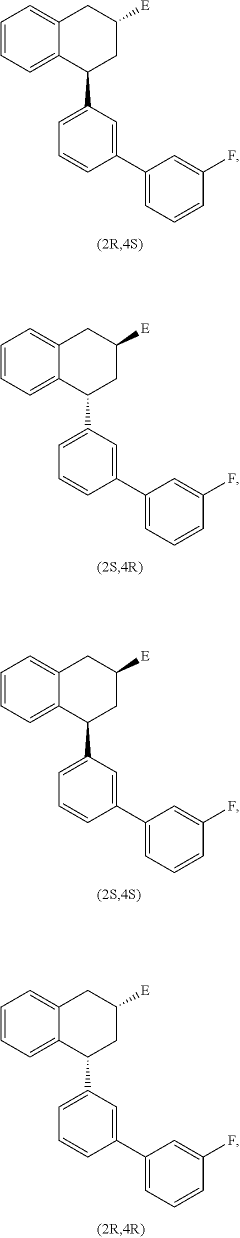

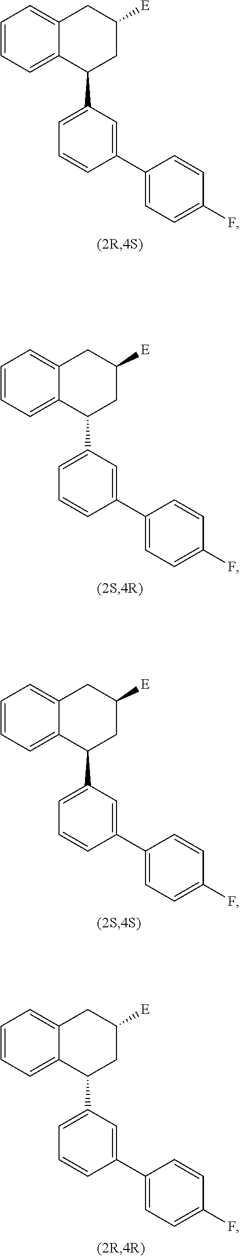

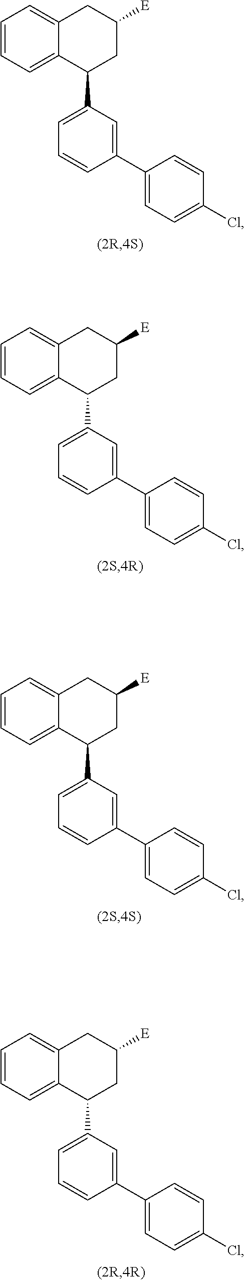

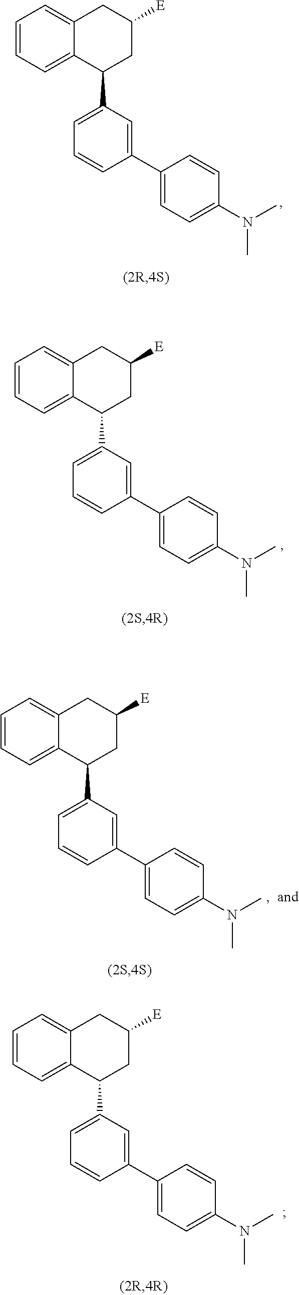

or a pharmaceutically acceptable salt, hydrate, or solvate thereof.
   38. The compound of any of features 33-37, wherein the compound is an antagonist, a neutral antagonist or an inverse agonist at one or more of the 5-HT2A, 5-HT2B, and 5-HT2C receptors.
   39. The compound of any of features 33-38, wherein the compound has a greater binding affinity for 5-HT2A, 5-HT2B, and/or 5-HT2C receptors than for 5-HT1A, 5-HT7, D2, D3, alpha1A, and/or alpha1 B receptors.
   40. The compound of any of features 33-39, comprising a pharmaceutically acceptable anion selected from the group consisting of acetate, adipate, aspartate, benzenesulfonate, benzoate, besylate, bicarbonate, bitartrate, bromide, camsylate, caprate, caproate, caprylate, carbonate, chloride, citrate, decanoate, dodecylsulfate, edetate, esylate, formate, fumarate, gluceptate, gluconate, glutamate, glycolate, hexanoate, hydroxynaphthoate, iodide, isethionate, lactate, lactobionate, laurate, malate, maleate, mandelate, mesylate, methylsulfate, mucate, napsylate, nitrate, octanoate, oleate, oxalate, palmitate, pamoate, pantothenate, phosphate, dihydrogen phosphate dodecahydrate, dihydrogen phosphate dihydrate, polygalacturonate, propionate, sabacate, salicylate, stearate, acetate, succinate, sulfate, tartrate, teoclate, thiocyanate, tosylate, and undecylenate.
   41. The compound of any of features 33-40, wherein the compound comprises one or more of ¹⁸F, ¹⁹F, ⁷⁵Br, ⁷⁶Br, ¹²³I, ¹²⁴I, ¹²⁵I, ¹³¹I, ¹¹C, ¹³C, ¹³N, ¹⁵O, or ³H.
   42. The compound of any of features 33-41, wherein the compound selectively modulates a physiological activity of 5-HT2A, 5-HT2B, and/or 5-HT2C receptors over a physiological activity of one or more of 5-HT1A, 5HT7, D2, D3, α1A, and α1B receptors.
   43. The compound of feature 42, wherein said selective modulation is associated with a difference in binding affinity, inverse agonism, agonism, partial agonism, allosteric agonism, antagonism, partial antagonism, or allosteric antagonism.
   44. A pharmaceutical composition comprising a compound of any of features 33-43 and an excipient.
   45. The pharmaceutical composition of feature 44, wherein the compound comprises (2R,4S)-(trans)-4-(3-(thiophen-2-yl)phenyl)-N,N,N-trimethyl-1,2,3,4-tetrahydronaphthalen-2-amine, (2R,4S)-(trans)-4-(3-(furan-2-yl)phenyl)-N,N,N-trimethyl-1,2,3,4-tetrahydronaphthalen-2-amine, or (2S,4S)-(cis)-4-([1,1-biphenyl]-3-yl)-N,N,N-trimethyl-1,2,3,4-tetrahydronaphthalen-2-amine.
   46. A method to aid in treating a disease or disorder, the method comprising administering an effective amount of a compound of any of features 33-43 or the pharmaceutical composition of any of features 44-45, to a mammalian subject in need thereof.
   47. The method of feature 46, wherein the disease or disorder is selected from the group consisting of hypertension, thrombosis, deep vein thrombosis, pulmonary embolus, atrial fibrillation, atherosclerosis, valvular atherosclerosis, cardiac fibrosis, obesity, irritable bowel syndrome, and lack of bladder control.
   48. The method of feature 47, wherein the subject further suffers from a neuropsychiatric disease or disorder, such as depression.
   49. The method of any of features 46-48, wherein the method results in inverse agonism, antagonism, partial antagonism, or allosteric antagonism at a peripheral 5-HT-2A, 5-HT2B, and/or 5-HT2C receptor.

As used herein, the term room temperature refers to a temperature within the range of about 15-30° C.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

### BRIEF DESCRIPTION OF THE DRAWINGS

FIG. 1A shows structures of the 4-phenyl-2-dimethylaminotetralin (4-PAT, 1) chemotype and derivatives with halogen (2a-2b′, 3a-3b′) or aryl substituents (2c-k and 3c-k) at the C(4)-phenyl meta position (Y).

FIG. 1B shows structures of the 4-phenyl-2-dimethylaminotetralin (4-PAT, 1) chemotype and derivatives with halogen (2a-2b′, 3a-3b′) or aryl substituents (2c-k and 3c-k) at the C(4)-phenyl meta position (Y). Examples of charged substituent E or quaternary amine substituent E (top right) are —N⁺(CH₃)₃, —N⁺(CH₃)₂(CH₂CH₃), —N⁺(CH₃)(CH₂CH₃)₂, and —N⁺(CH₂CH₃)₃;

FIG. 2A shows exploratory functional screening results for reference ligands and 4-PAT analogs at 5-HT₂-type receptors (Rs) 5-HT_{2A}, 5-HT_{2B}, 5-HT_{2C} and H₁Rs. The percent change from basal accumulation of inositol monophosphate (IP1) in clonal cells transiently expressing human wild-type 5-HT_{2A}, 5-HT_{2B}, 5-HT_{2C}, or H₁Rs following incubation with 10 μM of the indicated ligand is displayed in FIG. 2A. Reference ligands (top) include 5-HT (5-hydroxytryptamine), HIS (histamine), DOX (doxepine), RIT (ritanserin), and pimavanserin (PIMA). The mean percent change in basal signaling for each compound is normalized to the change for the reference agonist (i.e., 5-HT or histamine), and is shown numerically in each cell as the mean of at least two independent experiments performed using three technical replicates. Crossed spaces indicate no data. FIGS. 2B-2E show comparative functional assessments of PIMA and aryl substituted 4-PATs at 5-HT_{2A}, 5-HT_{2C}, 5-HT_{2B} and H₁Rs expressed in clonal cells. In FIGS. 2B-2C, PIMA, (2S,4R)-2k, and (2R,4R)-3h demonstrate inverse agonist activity at (FIG. 2B) constitutively activated C322K^{6.34} 5-HT_{2A}Rs and (FIG. 2C) WT 5-HT_{2C}Rs by attenuating basal (dotted line) IP1 accumulation. At FIG. 2D, 5-HT_{2B}Rs, PIMA, (2S,4R)-2k, and (2R,4R)-3h competitively antagonize 5-HT-stimulated IP1 accumulation. At FIG. 2E H₁Rs, (2S,4R)-2k and (2R,4R)-3h competitively antagonize histamine-stimulated IP1 accumulation, with minimal competition by PIMA. Concentration-response curves represent the mean±SD of n=5 independent experiments performed using three (FIG. 2B, FIG. 2C) or two (FIG. 2D, FIG. 2E) technical replicates.

FIGS. 3A-3C show comparative assessments of PIMA, (2S,4R)-2k and (2R,4R)-3h in male C57BL/6J mice. FIG. 3A shows head-twitch response elicited by 1 mg*kg⁻¹ (±)-DOI (s.c.) following pretreatment with vehicle, PIMA, (2S,4R)-2k, or (2R,4R)-3h. FIG. 3B shows locomotor activity of mice administered 1 mg*kg⁻¹ (±)-DOI (s.c.) following pretreatment with vehicle, PIMA, (2S,4R)-2k, or (2R,4R)-3h. FIG. 3C shows, after a 6-week washout period, the locomotor activity of the same mice from (3A) and (3B) is reassessed following administration (s.c.) of vehicle or 3 mg*kg⁻¹ PIMA, (2S,4R)-2k, or (2R,4R)-3h. Data represent the mean±SD of n=6 to 7 treatments, individual values are shown for each condition. Significance is determined using one-way ANOVA, with Tukey's correction for multiple comparisons, *p<0.05.

FIGS. 4A-4C show proposed binding modes of PIMA, (2S,4R)-2k, and (2R,4R)-3h at a model of the 5-HT₂AR. FIG. 4A shows the structure of PIMA at top and the proposed binding mode at the 5-HT₂AR at bottom. FIG. 4B shows the structure of (2S,4R)-2k at top and the proposed binding at the 5-HT₂AR mode at bottom. FIG. 4C shows the structure of (2R,4R)-3h at top and the proposed binding mode at the 5-HT₂AR at bottom. Side chains within 4.0 Å of each ligand are shown, as well as F213^{4.63} and D231^{5.35} which are experimentally point mutated herein. For clarity, only the anchoring side chain of D155^{3.32} is shown for residues in TM3.

FIGS. 5A-5B show molecular dynamics studies demonstrating the structural basis of 4-PAT stereoselectivity at histamine H₁Rs. FIG. 5A shows the proposed binding mode of (2S,4R)-2k at the H₁R resembles that observed at the 5-HT₂AR (see FIG. 4B), where the aminotetralin moiety could form aromatic T-stacking interactions with the side chain of W428^{6.48}, while the aryl substituent extends in a cavity between TM4 and TM5 where it could participate in aromatic T-stacking interactions with the side chain of W158^{4.56}. FIG. 5B shows the proposed binding mode of (2R,4R)-3h at the H₁R indicates that a stereochemical restriction at the C(2)-position causes the aryl substituent to position between TM5 and TM6, thus disfavoring productive aromatic interactions between the ligand and the side chains of W158^{4.56} and W428^{6.48}.

FIGS. 6A and 6B show X-ray crystal structures of (2R,4R)-3b (FIG. 6A) and (2S,4S)-3b′ (FIG. 6B). Coordinates are provided in the Examples section.

FIGS. 7A-7B show follow up in vitro assessment of potential (2S,4R)-2k and (2R,4S)-2c agonist activity at 5-HT_{2B} and 5-HT_{2C}Rs, respectively. FIG. 7A shows analog (2S,4R)-2k compared to 5-HT at 5-HT_{2B}Rs. FIG. 7B shows analogs (2S,4R)-2c and (2R,4S)-2c compared to 5-HT at 5-HT_{2C}Rs. Data are represented as the individual mean values from 5-8 independent experiments performed in triplicate, and the mean±SD of all experiments for a given concentration of ligand. An asterisk * indicates statistically significant effects (p<0.05) of ligand concentration on the accumulation of IP1, as determined by either an ordinary one-way ANOVA or Kruskal-Wallis test; ns, not significant.

FIG. 8 shows superimposed 5-HT_{2A}Rs stabilized in an inactive state by PIMA, (2S,4R)-2k, or (2R,4R)-3h (light gray, dark gray, and medium gray receptor, respectively). Insets highlight features of an inactive GPCR, including the orientation of the W336^{6.48} toggle switch being perpendicular to the lipid bilayer, as well as the state of the PIF motif, and the distance between R173^{3.50} and E318^{6.30} of the E/DRY domain being close enough such that an ionic bond could form.

FIG. 9 shows activity of (2S,4R)-2a at constitutively activated C322K^{6.34} 5-HT_{2A}Rs, showing analog (2S,4R)-2a behaves as an inverse agonist at C322K^{6.34} 5-HT_{2A}Rs, eliciting a reduction in basal IP accumulation by ˜50%. Data are shown as the mean±SD of n=5 independent experiments performed using three technical replicates.

FIG. 10A shows a top view (from extracellular to cytosol view) of (2S,4R)-2k after a 100 ns molecular dynamics simulation at the 5-HT₂AR shows the steric tolerance afforded by the side chain of G238^{5.42} between TM4 and TM5. FIG. 10B shows a plot of the minimum distance between (2S,4R)-2k and G238^{5.42} showing the interaction is stable over 100 ns (X-axis).

FIGS. 11A-11G show visualizations of the change in potency of 5-HT and various antagonists at point mutated 5-HT_{2A}Rs. FIG. 11A shows change in functional potency of 5-HT at point-mutated 5-HT_{2A}Rs. FIG. 11B shows concentration response for 5-HT at WT and point mutated 5-HT_{2A}Rs, normalized to the percent change from the basal concentration inositol monophosphates (IP1). FIGS. 11C-11G show antagonism of 1 μM 5-HT-mediated IP1 accumulation by five 5-HT₂AR antagonists (spanning three medicinal chemical chemotypes) at WT and point-mutated 5-HT_{2A}Rs. Data in (11A) are presented as the mean ΔpEC₅₀±SD, data in (11C-11F) are presented as the mean ΔpK_{b}±SD, where ΔpEC₅₀=ΔpEC₅₀₍ₘᵤₜₐₙₜ₎−ΔpEC_{50(WT)} and ΔpK_{b}=ΔpK_{b(mutant)}−ΔpK_{b(WT)}. For clarity, the concentration response curves of 5-HT in FIG. 11B are shown as the mean±SEM. The number of independent experiments (n) performed for each condition is 5-19, with exact n shown in Table 2. The asterisk * indicates statistical significance (p<0.05) between the wild-type and mutant receptor parameters, determined by an unpaired t-test or Mann-Whitney U-test where appropriate.

FIGS. 12A-12B show superimpositions of a zotepine bound 5-HT_{2A}R (PDB: 6A94), LSD bound 5-HT_{2B}R (PDB: 5TVN), and ritanserin bound 5-HT_{2C}R (PDB: 6BQH). The structures indicate that a unique rotamer of F^{5.38} exists in the 5-HT₂AR (black ellipse, FIG. 12A), where it is raised toward the extracellular end of TM4 through hydrophobic interactions with the non-conserved residue F^{4.63} (K^{4.63} and I^{4.63} in 5-HT_{2B} and 5-HT_{2C}Rs, respectively).

FIG. 13 shows comparative dynamics of F^{5.38} in 5-HT₂A and 5-HT_{2B}Rs. The side chain of F^{5.38} is more dynamic (measured by RMSD) in WT 5-HT_{2A}Rs (black trace) than it is in WT 5-HT_{2B}Rs (gray trace). When residue D5.35 in the 5-HT_{2A}R is mutated in silico to the equivalent residue (F5.35) in 5-HT_{2B}Rs (gray trace), the dynamics of F⁵³⁸ in the D5.35F 5-HT_{2A}R bear greater resemblance to that of F^{5.38} in WT 5-HT_{2B}Rs.

FIGS. 14A-14D show exploratory saturation binding results indicating that specific binding of [³H]mesulergine (FIG. 14A, FIG. 14B), [³H]ketanserin (FIG. 14C), or [³H]spiperone (FIG. 14D) cannot be detected for HEK293 cells transfected with cDNA encoding human D231F^{5.35} 5-HT_{2A}Rs. Data are presented as single experiments with three technical replicates for total (black circles) and nonspecific binding (gray squares, determined using 30 μM mianserin and 30 μM risperidone).

### DETAILED DESCRIPTION

The present technology provides novel 4-phenyl-2-dimethylaminotetralin (4-PAT) compounds that can be utilized in treating or preventing neuropsychiatric disorders. Similar to other 5HT_{2C} agonists with 5HT_{2A/2B} antagonist/inverse agonist compounds of the 2-aminotetralin chemotype, which are active in rodent and monkey models of psychosis, the present 4-PAT compounds can be used to treat, for example, fragile X syndrome, autism, impulsive behaviors such as occur with attention deficit hyperactivity disorder and binge eating, and substance use disorder (particularly, opioid use disorder and amphetamines use disorder). There are no drugs currently approved to treat psychosis associated with the neurodevelopmental disorder fragile X syndrome (orphan therapeutic indication) or autism. Current approved antipsychotic medications cause sedation (in non-fragile X patients) or other neurological side effects. The present 4-PAT compounds do not cause sedation.

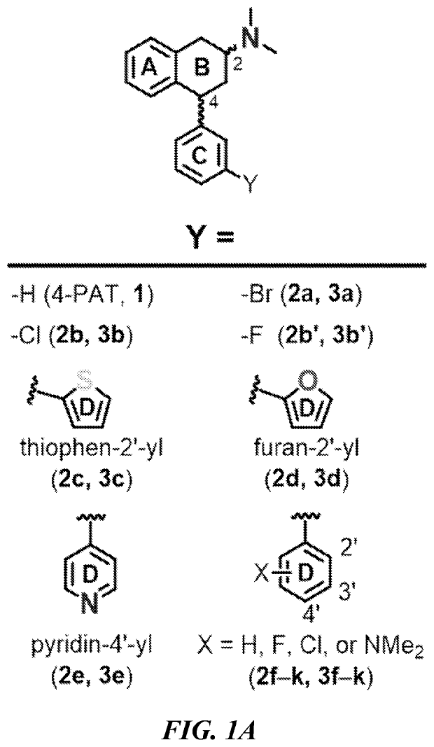

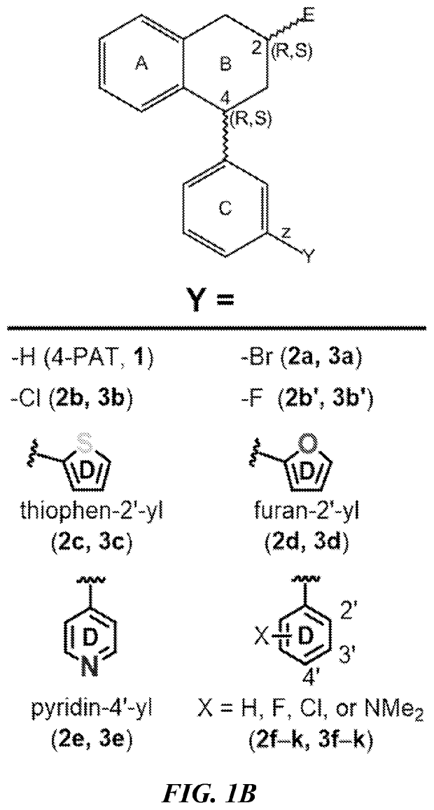

The present technology provides examples of at least 42 new chemical entities that are single enantiomer drug compounds. The examples of at least 42 new chemical entities that are single enantiomer drug compounds can optionally be configured so that the compound or composition does not substantially accumulate in the human brain (e.g., Scheme 11, FIG. 1B), thereby increasing the targeting efficacy of the technology. Mechanisms of single enantiomer specificity are elucidated. Synthesis and purification are described in detail. The technology can provide stereochemically-pure compounds having cis-2R4R, cis-2S,4S, trans-2R4S, or trans-2S4R stereochemistry. In the examples, there are compounds for use disclosed herein that are (2R,4S)-trans-4-(3-(thiophen-2-yl)phenyl)-N,N-dimethyl-1,2,3,4-tetrahydronaphthalen-2-amine (2c) and (2R,4S)-trans-4-(3-(furan-2-yl)phenyl)-N,N-dimethyl-1,2,3,4-tetrahydronaphthalen-2-amine (2d), and (2S,4S)-cis-4-([1,1′-biphenyl]-3-yl)-N,N-dimethyl-1,2,3,4-tetrahydronaphthalen-2-amine, (3f) (FIG. 1A, Table 1).

The present technology provides novel 4-phenyl-2-dimethylaminotetralin (4-PAT) compounds and compositions that can be utilized in treating or preventing a variety of disorders of the periphery, in particular because their distribution within the body of the subject is restricted to the periphery, i.e., the compound does not substantially accumulate in the human brain. The ‘E’ group shown in FIG. 1B is charged and can be a quaternary amine or other charged moiety; for example, E can be —N⁺(CH₃)₃, —N⁺(CH₃)₂(CH₂CH₃), —N⁺(CH₃)(CH₂CH₃)₂, or —N⁺(CH₂CH₃)₃. U.S. Ser. No. 10/548,856B2, which is hereby incorporated by reference in its entirety, describes charged 5-PAT compounds and methods for modulating serotonin receptors in the periphery. Using compounds of the present technology, the peripherally restricted, charged 4-PAT compounds can preventing side effects caused by binding to 5-HT receptor sites in the CNS. In another example, 5HT_{2B} is not expressed in the brain for a desired treatment, and the technology may treat a peripheral disease.

The compound or composition is not rapidly metabolized in the periphery. In various examples, the compounds and compositions, or formulations thereof, deliver a physiological amount of the present compound or composition to the periphery for at least about 6 hours, or at least about 9 hours, or at least about 12 hours, or at least about 15 hours, or at least about 18 hours, or at least about 21 hours, or at least about 24 hours. In various examples, the compounds and compositions, or formulations thereof, deliver a physiological amount of the present compound or composition to the periphery for about 6 hours, or about 9 hours, or about 12 hours, or about 15 hours, or about 18 hours, or about 21 hours, or about 24 hours.

The centrally-acting 4-PAT compounds of the present technology can be used to aid in treating or preventing, for example, migraine, Parkinson's disease, attention deficit hyperactivity disorder, anxiety or generalized anxiety, depression, schizophrenia, binge eating, opioid use disorder, fragile X syndrome, amphetamine use disorder, panic disorder, social anxiety disorder, obsessive-compulsive disorder, pain, Alzheimer's disease, or Huntington's disease.

The peripherally acting 4-PAT compounds of the present technology can be used to aid in treating or preventing, for example, hypertension, thrombosis, deep vein thrombosis, pulmonary embolus, atrial fibrillation, atherosclerosis, valvular atherosclerosis, cardiac fibrosis, obesity, irritable bowel syndrome, and lack of bladder control.

5HT_{2C} agonists with 5HT_{2A/2B} antagonist/inverse agonist activity of the 2-aminotetralin chemotype demonstrate high efficacy and safety in rodent and monkey animal models of psychoses and substance use disorders (amphetamines and opioids), as described in U.S. Pat. No. 8,586,634B2, U.S. Pat. No. 9,024,071B2, U.S. Pat. No. 9,862,674B2 and U.S. Ser. No. 10/017,458B2, each of which is hereby incorporated by reference in its entirety. There are no drugs approved to treat psychosis associated with the neurodevelopmental disorder fragile X syndrome (an orphan therapeutic indication) or autism, and the 4-PAT chemotypes disclosed herein demonstrate outstanding potential for these therapeutic indications.

Synthetic methods described herein include Friedel-Crafts cycli-acyl/alkylation of the commercially available 3-bromostyrene and phenylacetyl chloride to give the intermediate tetralone. The tetralone is subjected to reductive amination to afford a separable mixture of 3′-Br-4-phenyl-2-aminotetralin diastereomers. Reductive amination produces racemic cis or trans-4-phenyl-2-aminotetralins that are separated via silica gel column chromatography. Substituents are introduced at the 3′-position via Suzuki-Miyaura coupling of the 3′-Br-4-PAT diastereomers with the corresponding boronic acids. The ‘bench-top stable’ MIDA ester is used successfully to introduce thiophen-2′-yl and furan-2′-yl fragments into the 3′-Br-4-PATs. The racemic mixtures of trans-analogs are separated by a semi-preparative chiral HPLC column using conditions and solvents specific to each analog to elute the trans-(2R,4S) and trans-(2S,4R) enantiomers at representative retention times t1 and t2, respectively, with absolute stereochemistry assigned according to retention time of the previously published trans-3′CI-4-PAT analog. Chiral HPLC separation yields the (2R,4S)-trans-4-(3-(thiophen-2-yl)phenyl)-N,N-dimethyl-1,2,3,4-tetrahydronaphthalen-2-amine and (2R,4S)-trans-4-(3-(furan-2-yl)phenyl)-N,N-dimethyl-1,2,3,4-tetrahydronaphthalen-2-amine.

The compounds presented herein are water soluble and can be administered as oral formulations. The technology includes pharmaceutical compositions and formulations including the compounds. Examples of formulations can include drug in capsule with or without excipients additives or buffers, subcutaneous and IV formulations, mixtures with citric acid, lactic acid, solvents, propylene glycol, osmolality adjusting salts or sugars, purified water, or other excipients.

The centrally acting (e.g., uncharged) compounds presented herein cross into the brain when administered to rodents and engage serotonin 5HT2 receptors to produce antipsychotic effects. The compounds do not cause neurological side effects at doses that are antipsychotic. The 5-HT receptor (5-HTR) subtypes 5-HT_{2A} and 5-HT_{2C} are important neurotherapeutic targets, though, obtaining selectivity over 5-HT_{2B} and closely related histamine H₁Rs is challenging. Herein are delineated molecular determinants of selective binding to 5-HT₂A and 5-HT_{2C}Rs using novel 4-PATs.

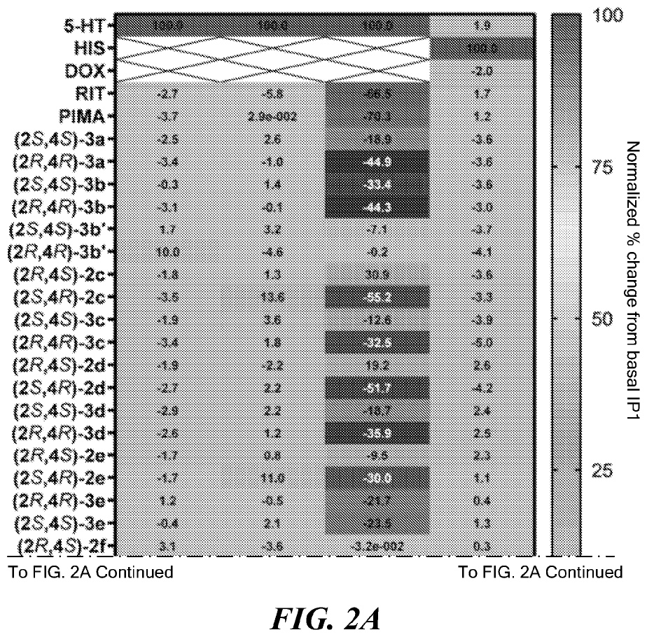

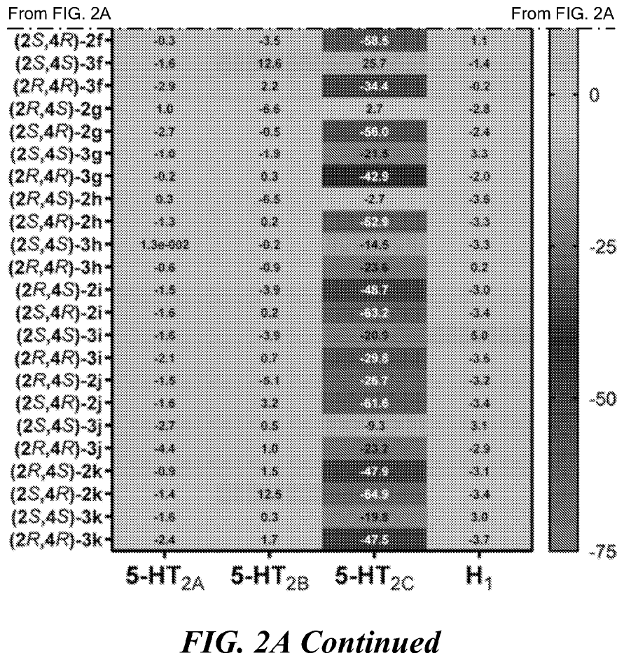

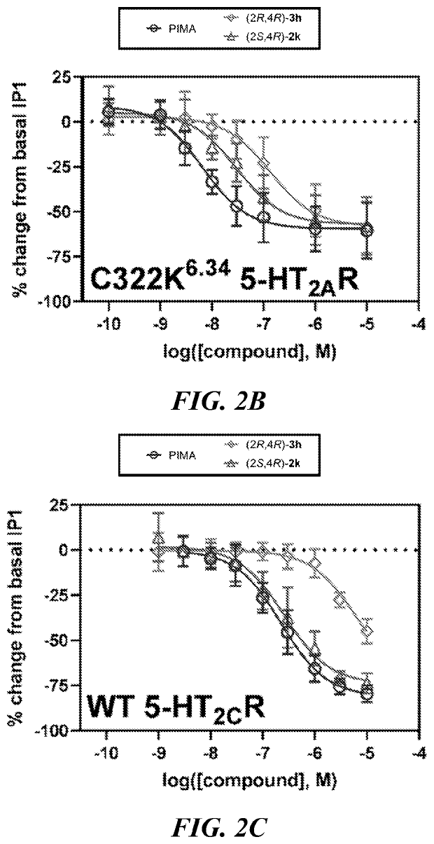

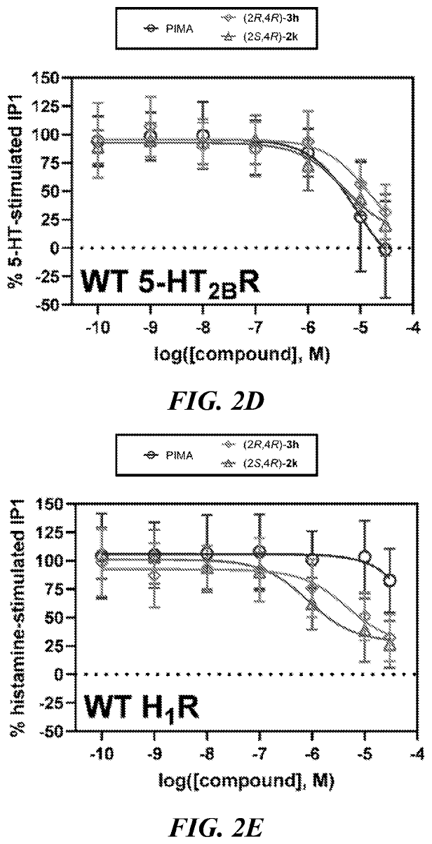

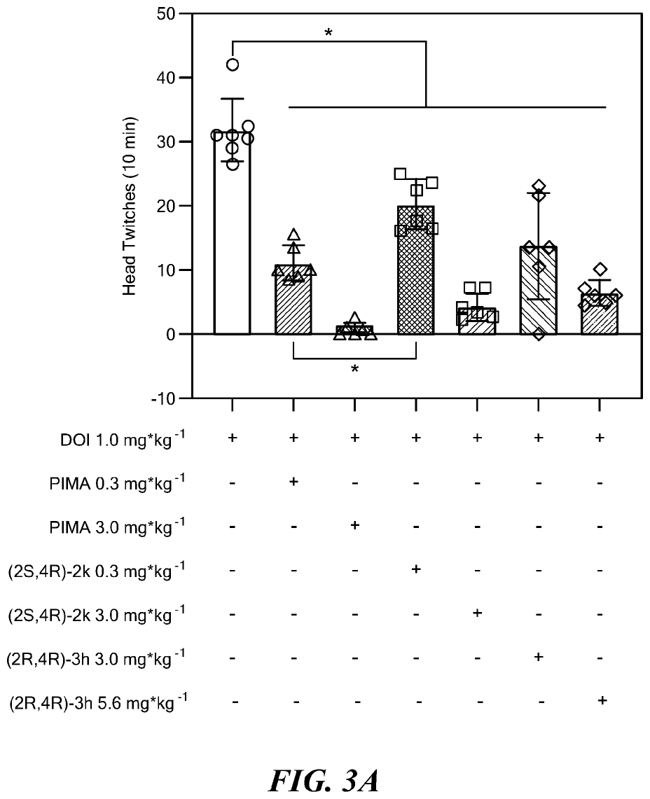

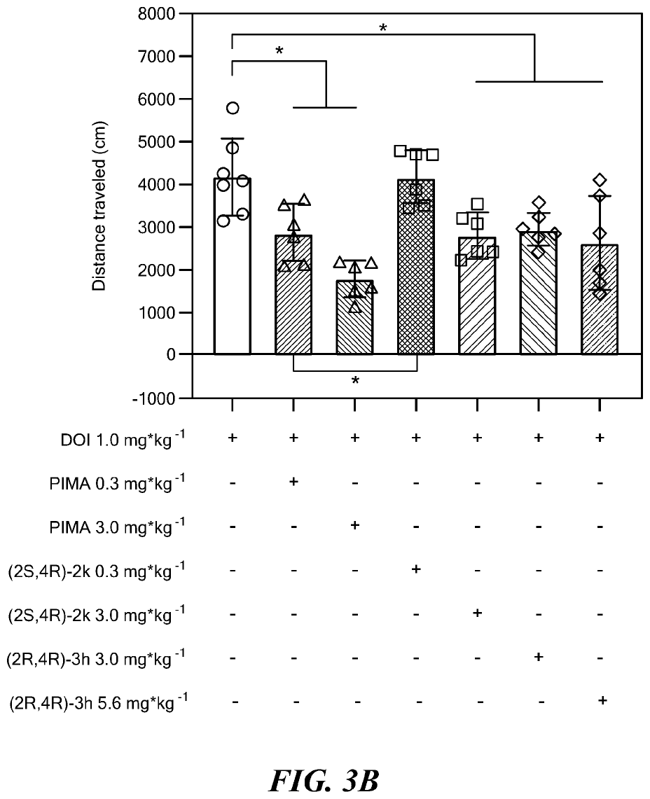

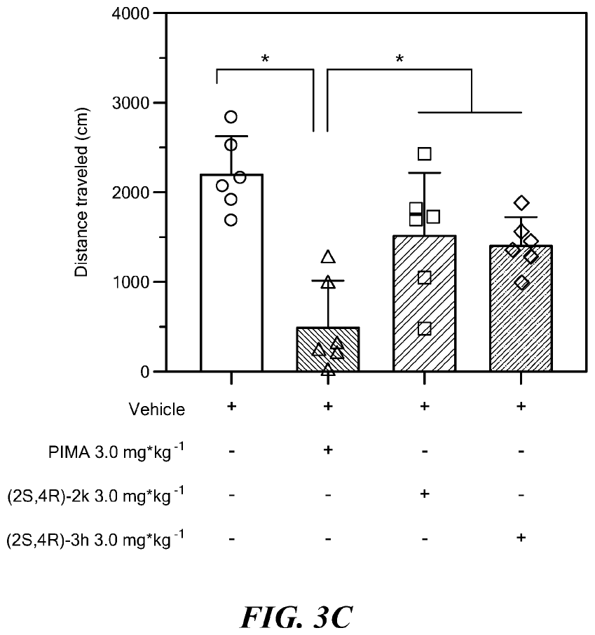

Example compounds are compared to 5-HT, HIS, DOX, RIT and PIMA in exploratory functional screening at 5-HT₂-type receptors (Rs) 5-HT₂A, 5-HT_{2B}, 5-HT_{2C} and H₁Rs in FIG. 2A. The percent change from basal accumulation of inositol monophosphate (IP1) in clonal cells transiently expressing human wild-type 5-HT_{2A}, 5-HT_{2B}, 5-HT_{2C}, or H₁Rs following incubation with 10 μM is shown in the form of a heat map (FIG. 2A). Further comparisons are made in FIGS. 2B-2C, wherein PIMA, (2S,4R)-2k, and (2R,4R)-3h (Table 1) show inverse agonist activity at (FIG. 2B) constitutively activated C322K^{6.34} 5-HT_{2A}Rs and (FIG. 2C) WT 5-HT_{2C}Rs by attenuating basal (dotted line) IP1 accumulation. In FIG. 2D, 5-HT_{2B}Rs, PIMA, (2S,4R)-2k, and (2R,4R)-3h competitively antagonize 5-HT-stimulated IP1 accumulation. In FIG. 2E H₁Rs, (2S,4R)-2k and (2R,4R)-3h competitively antagonize histamine-stimulated IP1 accumulation, with minimal competition by PIMA. Affinity, function, molecular modeling, and 5-HT_{2A}R mutagenesis studies are undertaken to understand structure-activity relationships at 5-HT₂-type and H₁Rs. Lead 4-PAT selective 5-HT_{2A}/5-HT_{2C}R inverse agonists are compared to PIMA, a selective 5-HT_{2A}/5-HT_{2C}R inverse agonist approved to treat psychoses, in the mouse head twitch response (FIG. 3A) and locomotor activity assays (FIG. 3B, FIG. 3C), as models relevant to antipsychotic drug development.

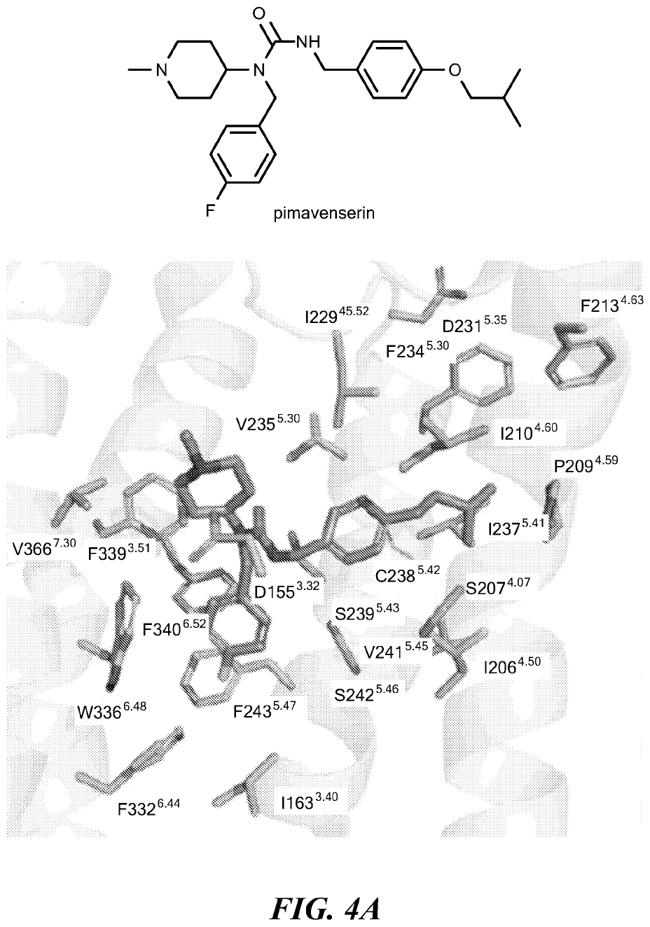

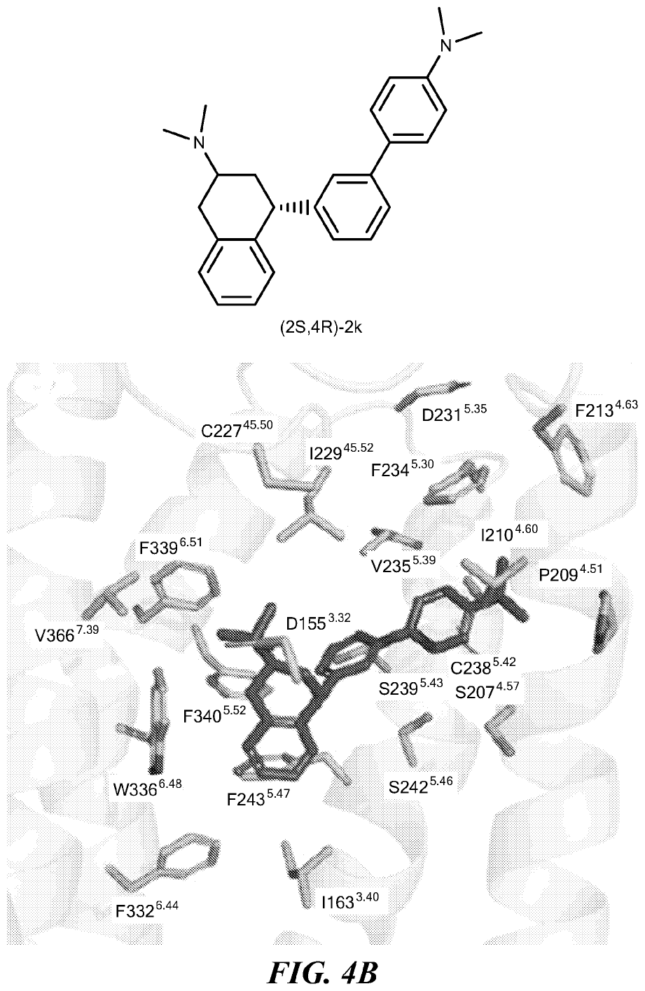

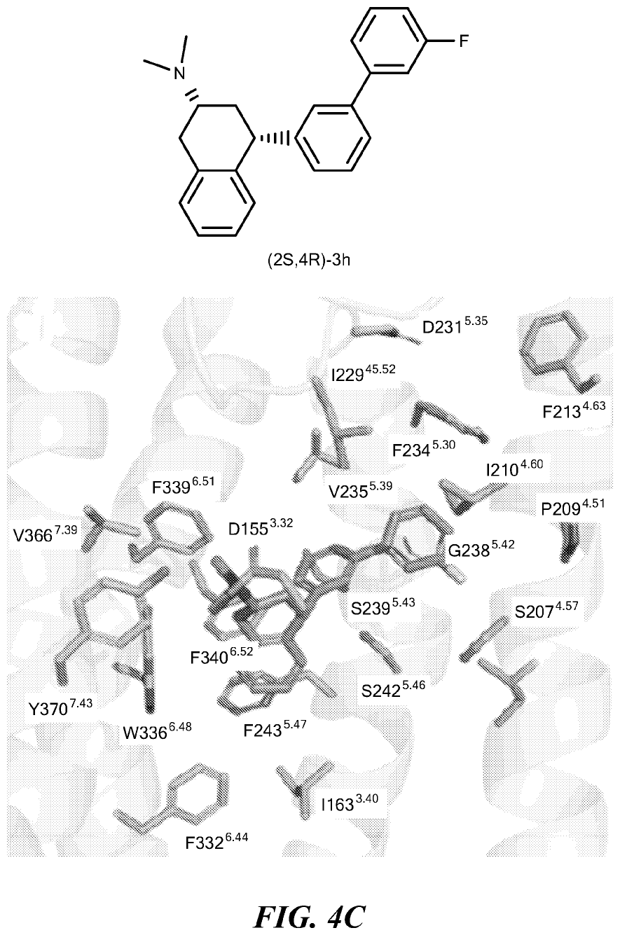

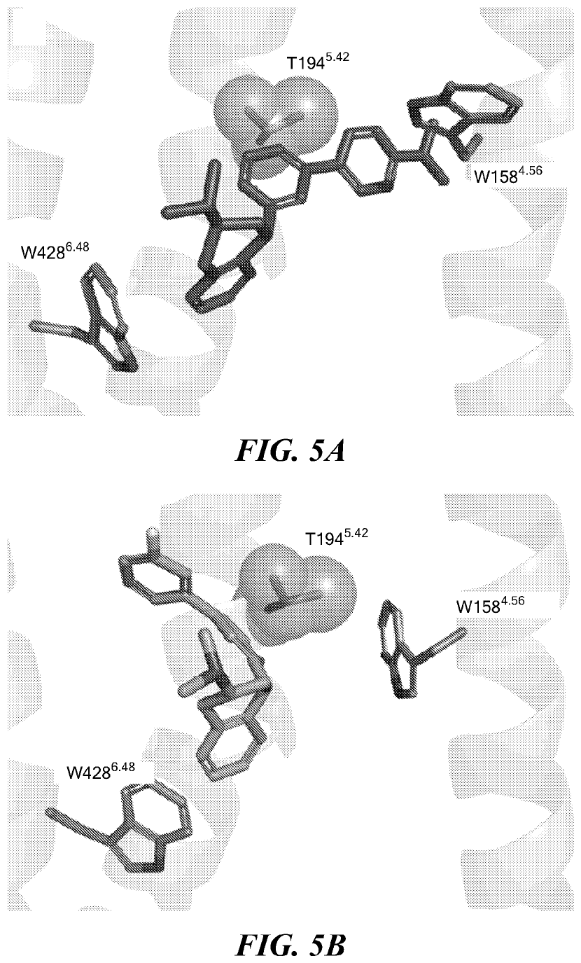

Most 4-PAT diastereomers in the (2S,4R)-configuration bind non-selectively to 5-HT_{2A}, 5-HT_{2C}, and H₁Rs, with >100-fold selectivity over 5-HT_{2B}Rs, whereas diastereomers in the (2R,4R)-configuration bind preferentially to 5-HT_{2A} over 5-HT_{2C}Rs and have >100-fold selectivity over 5-HT_{2B} and H₁Rs. Results suggest that G238^{5.42} and V235^{5.39} (FIGS. 4A-4C) in 5-HT_{2A}Rs (conserved in 5-HT_{2C}Rs) are important for high affinity binding, whereas interactions with T194^{5.42} and W158^{4.56} (FIGS. 5A-5B) are important for H₁Rs. The 4-PAT (2S,4R)-2k, a potent and selective 5-HT_{2A}/5-HT_{2C}R inverse agonist, has activity like PIMA in the mouse head-twitch response assay but is distinct in not suppressing locomotor activity. The 4′-NMe₂—C₆H₄ substituent on ring C (2k, 3k; Table 1), can be used as an example substituent to explore electronic and steric effects.

The novel 4-PAT chemotype can yield selective 5-HT_{2A}/5-HT_{2C}R inverse agonists for antipsychotic drug development by optimizing ligand-receptor interactions in transmembrane domain 5. It is shown that chirality can be exploited to attain selectivity over H₁Rs to help circumvent sedative effects. High homology between 5-HT₂-type and histamine H₁Rs could prevent development of antipsychotics without sedative effects.

The 4-phenyl-2-dimethylaminotetralin (4-PAT, FIG. 1A) configured in a (2S,4R)-1 chemotype can yield 5-HT_{2C}R agonists with antagonist/inverse agonist activity at 5-HT_{2A}, 5-HT_{2B}, and H₁Rs (Moniri, et al., 2004; Booth, et al., 2009). However, (2S,4R)-1 configurations can bind to H₁Rs with high affinity, and to 5-HT₂-type receptors with only moderate affinity. Bromine substitution at the meta position of 4-PAT ring C yields (2S,4R)-2a (FIG. 1A, Table 1) a ligand with lower affinity at H₁Rs, higher affinity at 5-HT₂-type receptors (Canal, et al., 2014; Sakhuja, et al., 2015), yet nil subtype-selectivity. Notably, (2S,4R)-2a demonstrates antipsychotic-like activity in several mouse models (Canal, et al., 2014). The current work can attain 4-PATs with selectivity for 5-HT_{2A} and/or 5-HT_{2C}Rs over 5-HT_{2B} and H₁Rs (Canal, et al., 2014; Sakhuja, et al., 2015). Herein, 42 novel 4-PAT analogs are synthesized, including diastereomers of meta-halo substituted 4-PATs (3a-b′, FIG. 1A), as well as aryl substituted analogs (2c-k, 3c-k, FIG. 1A).

TABLE 1
Affinity (pKᵢ) of reference ligands and novel 2-aminotetralins at 5-HT_{2A}, 5-HT _{2B}, 5-HT_{2C}, and H₁Rs^{a}.

| | | | pKᵢ⁽ⁿ⁾ | | | |
|---|---|---|---|---|---|---|
| | | Stereo- | Range | | | |
| Cmpd | Y | chemistry | 5-HT _{2A}R | 5-HT_{2B}R | 5-HT_{2C}R | H₁R |
| PIMA | | — | 9.62⁽²⁾ | 6.36 ± 0.50⁽⁵⁾ | 8.56⁽²⁾ | 5.50⁽²⁾ |
| | | | 9.64-9.60 | 6.86-5.68 | 8.67-8.46 | 5.76-5.25 |
| Risperidone | | — | 9.53⁽²⁾ | 7.81⁽²⁾ | 7.72⁽²⁾ | 7.74⁽²⁾ |
| | | | 9.54-9.52 | 7.9-7.71 | 7.76-7.68 | 7.8-7.68 |
| 3a | 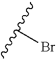 [SMILES: *Br] | (2S,4S) (2R,4R) | 6.40⁽²⁾ 6.41-6.40 7.50⁽³⁾ 7.74-7.33 | 6.41 ⁽²⁾ 6.45-6.37 7.07⁽²⁾ 7.14-7.00 | 6.71⁽³⁾ 6.93-6.47 7.24⁽⁴⁾ 7.44-7.06 | 8.55⁽³⁾ 8.58-8.53 7.39⁽²⁾ 7.46-7.31 |
| 3b | 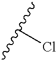 [SMILES: *Cl] | (2S,4S) (2R,4R) | 6.03⁽²⁾ 6.18-5.88 7.36⁽²⁾ 7.41-7.30 | 6.41⁽³⁾ 6.46-6.38 6.94⁽³⁾ 6.99-6.91 | 6.51⁽⁴⁾ 6.86-6.09 7.00⁽²⁾ 7.13-6.88 | 8.47⁽⁴⁾ 8.65-8.31 6.94⁽⁴⁾ 7.12-6.85 |
| 3b′ | 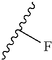 [SMILES: *F] | (2S,4S) (2R,4R) | 5.73⁽⁴⁾ 6.02-5.25 6.63⁽³⁾ 6.80-6.41 | 6.04⁽²⁾ 6.12-5.96 6.35⁽³⁾ 6.54-6.17 | 6.36⁽²⁾ 6.54-6.34 6.60⁽⁴⁾ 6.97-6.25 | 8.06⁽²⁾ 8.1-8.01 7.08⁽²⁾ 7.16-7.00 |
| 2c 3c | 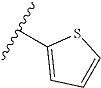 [SMILES: *c1cccs1] | (2R,4S) (2S,4R) (2S,4S) (2R,4R) | 7.71⁽²⁾ 7.72-7.70 8.67⁽²⁾ 8.77-8.56 6.82⁽³⁾ 6.98-6.54 8.45 ± 0.15⁽⁵⁾ 8.63-8.27 | 6.67 ± 0.33⁽⁵⁾ 7.13-6.26 7.10⁽³⁾ 7.28-6.93 7.05⁽³⁾ 7.15-6.89 6.98⁽⁴⁾ 7.19-6.69 | 7.48⁽³⁾ 7.76-7.27 8.42⁽²⁾ 8.54-8.30 6.65⁽²⁾ 6.75-6.55 7.62⁽²⁾ 7.89-7.36 | 6.90⁽²⁾ 7.01-6.79 8.94⁽⁴⁾ 9.11-8.79 6.36⁽²⁾ 6.39-6.33 6.63⁽²⁾ 6.70-6.56 |
| 2d 3d | 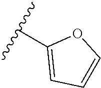 [SMILES: *c1ccco1] | (2R,4S) (2S,4R) (2S,4S) (2R,4R) | 7.14⁽²⁾ 7.16-7.13 8.75⁽³⁾ 9.06-8.52 6.97⁽³⁾ 7.18-6.77 7.72⁽³⁾ 8.27-7.34 | 6.64⁽³⁾ 6.92-6.35 7.2⁽³⁾ 7.41-7.05 6.45⁽³⁾ 6.89-6.22 6.89⁽³⁾ 7.30-6.61 | 7.10⁽⁴⁾ 8.04-6.50 8.60 ± 0.19⁽⁵⁾ 8.83-8.29 6.50⁽²⁾ 6.53-6.46 7.25⁽²⁾ 7.35-7.16 | ND^{b} 8.47⁽²⁾ 8.49-8.45 ND 7.30⁽²⁾ 7.34-7.27 |
| 2e 3e | 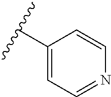 [SMILES: *c1ccncc1] | (2R,4S) (2S,4R) (2S,4S) (2R,4R) | 6.06⁽²⁾ 6.14-5.98 8.13⁽³⁾ 8.51-7.82 5.81⁽²⁾ 6.01-5.62 7.36⁽²⁾ 7.48-7.24 | 5.38⁽²⁾ 5.58-5.19 6.20⁽²⁾ 6.199-6.201 5.95⁽²⁾ 6.03-5.86 5.58⁽²⁾ 5.58-5.58 | 5.90⁽²⁾ 5.99-5.80 7.37⁽³⁾ 7.44-7.27 5.61⁽²⁾ 5.69-5.54 6.26⁽²⁾ 6.42-6.10 | ND 8.86⁽²⁾ 8.97-8.74 ND 7.65⁽³⁾ 7.79-7.47 |
| 2f 3f | 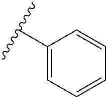 [SMILES: *c1ccccc1] | (2R,4S) (2S,4R) (2S,4S) (2R,4R) | 7.13⁽²⁾ 7.28-6.98 9.27⁽³⁾ 9.82-8.89 7.29⁽³⁾ 7.63-6.99 9.11⁽⁴⁾ 9.37-8.71 | 6.95⁽²⁾ 7.00-6.91 7.28⁽²⁾ 7.37-7.19 6.39⁽³⁾ 6.72-6.09 6.93 ± 0.25⁽⁵⁾ 7.21-6.70 | 7.16⁽²⁾ 7.19-7.12 8.48⁽²⁾ 8.51-8.46 6.82⁽²⁾ 6.98-6.67 7.57⁽²⁾ 7.69-7.46 | 6.37⁽²⁾ 6.57-6.18 9.3⁽³⁾ 9.42-9.20 7.41 ⁽²⁾ 7.41-7.41 6.94⁽²⁾ 6.96-6.91 |
| 2g 3g | 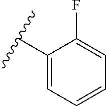 [SMILES: *c1ccccc1F] | (2R,4S) (2S,4R) (2S,4S) (2R,4R) | 7.51 ⁽²⁾ 7.58-7.44 9.09⁽²⁾ 9.17-9.01 7.39⁽³⁾ 7.62-7.25 8.69⁽⁴⁾ 9.13-8.19 | 7.36⁽²⁾ 7.36-7.35 7.09⁽³⁾ 7.23-6.90 6.53⁽³⁾ 6.71-6.30 6.33⁽²⁾ 6.49-6.17 | 7.54⁽³⁾ 7.76-7.41 8.41⁽³⁾ 8.66-8.12 6.73⁽²⁾ 6.74-6.72 7.70⁽²⁾ 7.78-7.62 | 6.93⁽²⁾ 7.02-6.84 8.75⁽⁴⁾ 9.1-8.62 ND 6.54⁽²⁾ 6.67-6.40 |
| 2h 3h | 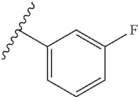 [SMILES: *c1cccc(F)c1] | (2R,4S) (2S,4R) (2S,4S) (2R,4R) | 8.03⁽⁴⁾ 8.18-7.91 9.22⁽³⁾ 9.37-8.96 7.69⁽³⁾ 7.82-7.53 9.46 ± 0.34⁽⁵⁾ 9.77-9.02 | 6.94⁽²⁾ 7.03-6.84 7.21 ± 0.21⁽⁵⁾ 7.49-6.99 6.58⁽²⁾ 6.60-6.56 6.77 ± 0.21⁽⁵⁾ 7.08-6.52 | 7.46⁽²⁾ 7.52-7.40 8.51⁽³⁾ 8.89-8.28 6.58⁽²⁾ 6.65-6.52 7.85 ± 0.12⁽⁵⁾ 7.95-7.66 | 7.31 ⁽²⁾ 7.39-7.23 8.96⁽³⁾ 9.11-8.88 6.20⁽²⁾ 6.23-6.18 6.30 ± 0.10⁽⁵⁾ 6.42-6.14 |
| 2i 3i | 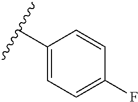 [SMILES: *c1ccc(F)cc1] | (2R,4S) (2S,4R) (2S,4S) (2R,4R) | 7.53⁽²⁾ 7.58-7.47 9.40⁽²⁾ 9.4-9.39 7.69⁽²⁾ 7.8-7.59 8.79⁽³⁾ 8.96-8.7 | 6.14⁽³⁾ 6.35-5.85 7.39⁽²⁾ 7.43-7.34 6.40⁽²⁾ 6.52-6.28 7.00⁽²⁾ 7.09-6.90 | 7.74⁽²⁾ 7.88-7.59 8.98⁽³⁾ 9.16-8.76 7.22⁽³⁾ 7.68-6.97 7.52⁽³⁾ 8.01-6.77 | 7.21 ⁽²⁾ 7.27-7.15 8.86⁽³⁾ 8.97-8.70 ND 6.69⁽³⁾ 6.71-6.66 |
| 2j 3j | 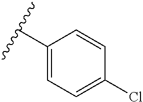 [SMILES: *c1ccc(Cl)cc1] | (2R,4S) (2S,4R) (2S,4S) (2R,4R) | 8.27⁽⁴⁾ 8.77-7.76 9.54 ± 0.62⁽⁶⁾ 10.03-8.46 7.20⁽⁴⁾ 7.87-6.71 8.77⁽²⁾ 8.85-8.68 | 6.55⁽⁴⁾ 6.73-6.44 6.98⁽⁴⁾ 7.18-6.75 5.91 ⁽²⁾ 5.98-5.84 6.77⁽²⁾ 6.80-6.73 | 8.15⁽²⁾ 8.39-7.92 8.31⁽⁴⁾ 8.56-8.15 7.01⁽³⁾ 7.4-6.59 8.03⁽²⁾ 8.11-7.94 | 7.84⁽²⁾ 7.89-7.78 8.84⁽³⁾ 8.86-8.82 ND 6.40⁽³⁾ 6.41-6.40 |
| 2k 3k | 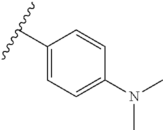 [SMILES: *c1ccc(N(C)C)cc1] | (2R,4S) (2S,4R) (2S,4S) (2R,4R) | 7.84⁽²⁾ 7.88-7.81 9.33 ± 0.14⁽⁵⁾ 9.54-9.17 6.91 ⁽²⁾ 7.02-6.80 8.67 ± 0.39⁽⁵⁾ 9.09-8.04 | 5.52⁽²⁾ 5.68-5.37 7.10 ± 0.21⁽⁵⁾ 7.40-6.82 5.97⁽²⁾ 6.08-5.87 5.97⁽²⁾ 6.09-5.86 | 7.73⁽²⁾ 7.79-7.67 9.37 ± 0.45⁽⁵⁾ 9.92-8.79 6.43⁽²⁾ 6.49-6.37 7.73⁽⁴⁾ 7.93-7.42 | 6.19⁽²⁾ 6.27-6.10 7.75 ± 0.10⁽⁵⁾ 7.88-7.64 ND 5.94⁽²⁾ 6.01-5.86 |

^{a}Data are presented as the mean pKᵢ and range for n independent experiments, shown in superscript. Where n = 5 independent experiments, the standard deviation is provided.
^{b}ND = not determined

Structure-activity relationships (SAR) for 4-PATs acting at 5-HT₂-type and H₁Rs are developed using competitive radioligand displacement and functional assays. The data reveal that aryl substituted 4-PATs are potent 5-HT_{2A}-preferring 5-HT_{2A}/5-HT_{2C}R inverse agonists with selectivity over 5-HT_{2B} and H₁Rs. To understand the molecular determinants for selectivity and inverse agonism at 5-HT_{2A}Rs, in silico molecular modeling is performed and used to guide site-directed mutagenesis of residues in receptor transmembrane (TM) domains 4 and 5. The potency and selectivity of certain aryl substituted 4-PATs at 5-HT_{2A} and 5-HT_{2C}Rs (e.g., [2S,4R]-2k, and [2R,4R]-3h) resemble PIMA, thus, a comparison is made of 4-PATs to PIMA in vitro and in silico. Comparative in vivo assessments in mice are also performed, utilizing the (±)-2,5-dimethoxy-4-iodoamphetamine (DOI)-elicited head twitch response as a model to screen for central 5-HT_{2A}R engagement and antipsychotic-like activity, and locomotor activity assays to assess behavioral disruption and untoward motor effects.

The optimized synthetic methods demonstrated herein generate novel cis- and trans-4-PAT enantiomers separable by chiral-HPLC. Competitive radioligand binding studies at 5-HT₂-type and H₁Rs indicate that small meta-halo substituents (e.g., —F, —Cl, —Br) on ring C (FIG. 1A) impart selectivity to bind H₁Rs over 5-HT₂-type receptors in the cis-(2S,4S)-configuration (˜40-280-fold). In contrast, aryl substituted 4-PATs in the cis-(2R,4R)-configuration selectively bind 5-HT_{2A}Rs over 5-HT_{2B}Rs (˜6-415-fold), 5-HT_{2C}Rs (˜2-40-fold), and H₁Rs (˜2-1,300-fold), with the greatest selectivity over all receptors observed for the 3′-F—C₆H4 substituted analog (2R,4R)-3h (Table 1). Interestingly, aryl substituted 4-PATs in the trans-(2S,4R)-configuration selectively bind to 5-HT_{2A} and 5-HT_{2C}Rs over 5-HT_{2B}Rs (˜15-180-fold), although only minimal selectivity is achieved over H₁Rs (≤5-fold). An exception is the attainment of 38-fold selectivity for 5-HT_{2A}Rs over H₁Rs with the 4′-NMe₂—C₆H₄ substituted analog (2S,4R)-2k, potentially due to the 4′-NMe₂ moiety forming unique van der Waals interactions within the binding pocket of 5-HT_{2A} and 5-HT_{2C}Rs. Notably, the —C₆H₄ substituted analog (2S,4R)-2f, a nonselective 5-HT_{2A}/H₁R antagonist with ˜70-fold selectivity over 5-HT_{2B}Rs and ˜5-fold selectivity over 5-HT_{2C}Rs, yields results here distinct from a prior report where it displayed high affinity and selectivity for 5-HT_{2C}Rs (Sakhuja, et al., 2015). The reason for this discontinuity is unclear, although variability in the assay conditions (radioligand K_{d}, buffer, incubation time, and temperature) may be contributing factors. Whereas a previous report explored the trans-enantiomers of 2f as the sole aryl substituted 4-PAT, here are explored all four stereoisomers of 9 distinct aryl substituted 4-PATs, which yield stereochemically consistent SAR across all aryl substituted 4-PATs in the (2S,4R)-configuration.

The affinity (pKᵢ) of the FDA-approved antipsychotic drugs PIMA and risperidone under that same assay conditions used for 4-PATs are assessed in Table 1. Notably, while the 4-PAT leads (2S,4R)-2k and (2R,4R)-3h, as well as PIMA and risperidone, have moderate to high selectivity over 5-HT_{2B} (˜50-3,000-fold) and H₁Rs (˜40-15,000-fold), only risperidone displays high affinity at all receptors tested. The 4-PAT leads (2S,4R)-2k and (2R,4R)-3h have high affinity and inverse agonist activity at 5-HT_{2A} and 5-HT_{2C}Rs comparable to PIMA and risperidone. While (2S,4R)-2k trends toward agonist activity at 5-HT_{2B}Rs, this ligand may not pose cardiovascular concerns given its low potency and efficacy (Unett, et al., 2013).

At 5-HT_{2A}Rs, no rank order in the radioligand-derived affinity (pKi) is observed. However, when comparing the functionally derived affinity (pKb), a clear rank order in potency appears (i.e., PIMA >[2S,4R]-2k>[2R,4R]-3h). The observed discontinuities are not investigated further but may involve differences in the biological milieu between experimental formats using a membrane preparation (radioligand binding assays) and whole live cells (functional assays). Biological milieu, including the membrane environment and effector expression, may vary between cell lines (Symons, et al., 2021; Zhang, et al., 2017) and transfections (Lee, et al., 2019) to impact functional signaling (Gutierrez, et al., 2016; Lefkowitz, et al., 2002). The results highlight the necessity of implementing orthogonal assays and reference ligands (e.g., PIMA and risperidone) to characterize ligand affinity and function (Tran, et al., 2019).

Comparing the affinity of 4-PATs at 5-HT₂-type and H₁Rs in Table 1, previous lab work shows that meta-halo substituted trans-4-PATs, including (2S,4R)-2a, display higher affinity at 5-HT₂-type receptors than the parent unsubstituted analog (2S,4R)-1 (Sakhuja, et al., 2015). However, the impact of meta-halo substitution on the affinity of cis-4-PATs at 5-HT₂-type receptors has not been reported. Here, it is shown that cis-diastereomers of the previously reported meta-Br substituted 4-PAT, 2a, and its meta-CI and meta-F congeners (3a, 3b and 3b′, respectively) demonstrate moderate (pKᵢ=6.5-7.5) to low (pKᵢ<6.5) affinity at 5-HT₂-type receptors in the (2R,4R)-configuration. These values resemble those for the unsubstituted cis-4-PATs previously reported (Booth, Fang et al., 2009), though larger, more polarizable substituents have higher affinity (pKᵢ 3a>3b>3b′). The corresponding (2S,4S)-enantiomers, on the other hand, exhibit high affinity (pKᵢ>7.5) at H₁Rs, with robust selectivity (40-280-fold) over 5-HT₂-type receptors. Due to their unimpressive affinity at, and selectivity for, 5-HT₂-type receptors, analogs 3a, 3b or 3b′ are not explored further (Table 1).

It is hypothesized that the chiral nature of the 4-PAT chemotype combined with steric bulk at the meta-position of ring C (FIG. 1A), reveals important SAR information about ligand binding at 5-HT₂-type and H₁Rs. Therefore, a variety of aromatic moieties are substituted at the meta-position of ring C, yielding 36 aryl substituted 4-PAT analogs (2c-k, 3c-k), and their affinity at 5-HT₂-type and H₁Rs is determined (Table 1).

Heteroaromatic substitution on ring C (i.e., 2c-e and 3c-e) yields five-membered heterocycles (2c-d, 3c-d) which bind to 5-HT₂A, 5-HT_{2C}, and H₁Rs in the (2S,4R)-configuration with high affinity and moderate selectivity for 5-HT_{2A} and 5-HT_{2C} over 5-HT_{2B}Rs (˜20-40-fold). In contrast to analogs (2S,4S)-3a-b′, the (2S,4S)-3c meta-thiophen-2′-yl analog has low affinity at, and no selectivity for, H₁Rs. Meanwhile, (2R,4R)-3c preferentially binds to 5-HT₂A and 5-HT_{2C}Rs, with highest affinity at 5-HT_{2A}Rs and moderate selectivity (˜30-60-fold) over 5-HT_{2B} and H₁Rs. The meta-pyridin-2′-yl (2e, 3e) analogs have moderate to low affinity at 5-HT₂-type receptors, however, (2S,4R)-2e and (2R,4R)-3e have high affinity at H₁Rs, thus, 2e, 3e are not pursued in further investigation.

The reassessment of (2S,4R)-2f indicates that, contrary to a 2015 report where the ligand displayed high affinity and selectivity for 5-HT_{2C}Rs (Sakhuja, et al., 2015), it binds non-selectively to 5-HT_{2A}, 5-HT_{2C}, and H₁Rs, with moderate selectivity over 5-HT_{2B}Rs (70-fold). Substitution effects on phenyl ring D are then investigated using a ‘fluorine-walk’ approach, wherein, fluorine is mono-substituted at each position (2g-i and 3g-i). The 3′-F—C₆H4 substituted diastereomer, (2R,4R)-3h, shows highest affinity at 5-HT_{2A}Rs, with 33-fold selectivity over 5-HT_{2C}Rs, 415-fold selectivity over 5-HT_{2B}Rs, and ˜1,300-fold selectivity over H₁Rs, making it the most 5-HT_{2A}R-selective 4-PAT-type compound reported herein. Its diastereomer, (2S,4R)-2h, exhibits high affinity at 5-HT_{2A} and 5-HT_{2C}Rs, with ˜30-fold selectivity over 5-HT_{2B}Rs, and no selectivity over H₁Rs. Thus, (2R,4R)-3h is chosen as a lead for further characterization in vivo, as well as in vitro and in silico to identify molecular determinants for selective binding to 5-HT_{2A}Rs.

The 4′-F—C₆H₄ substituted analog (2S,4R)-2i has high affinity at 5-HT_{2A} and 5-HT_{2C}Rs, 140-fold selectivity for 5-HT_{2A}Rs over 5-HT_{2B}Rs, though, selectivity over H₁Rs is modest (5-fold). Similarly, the 4′-CI—C₆H₄ substituted analog (2S,4R)-2j has 143-fold selectivity for 5-HT_{2A}Rs over 5-HT_{2B}Rs, and no selectivity over H₁Rs. The diastereomer, (2R,4R)-3j, retains high affinity for 5-HT_{2A} and 5-HT_{2C}Rs, and shows ˜100- and ˜225-fold selectivity for 5-HT_{2A}Rs over 5-HT_{2B} and H₁Rs, respectively.

To explore electronic and steric effects, a 4′-NMe₂—C₆H₄ substituent is introduced on ring C (2k, 3k), yielding stereoisomers with a computationally determined log P˜5.69 (log D˜3.77). Notably, these are slightly lower than that of 2i, 3i (log P˜5.85, log D˜3.86), 2j, 3j (log P˜6.36, log D˜4.41), and the other lead, (2R,4R)-3h (log P˜5.85, log D˜3.79). Like other aryl substituted 4-PATs in the (2R,4R)-configuration (i.e., 3c-3k), compound (2R,4R)-3k shows modest selectivity to bind 5-HT_{2A}Rs over 5-HT_{2C}Rs (˜7-fold) and high selectivity over 5-HT_{2B} and H₁Rs (>350-fold). In contrast, (2S,4R)-2k has similarly high affinity at both 5-HT_{2A} and 5-HT_{2C}Rs, with high selectivity over 5-HT_{2B}Rs (˜180-fold) and moderate selectivity over H₁Rs (˜38-fold). Since (2S,4R)-2k portrays dual 5-HT_{2A}/5-HT_{2C}R activity with selectivity over 5-HT_{2B} and H₁Rs, (2S,4R)-2k is selected, along with the 5-HT_{2A}R selective analog (2R,4R)-3h (above), for further investigation in vitro, in vivo, and in silico.

To compare the affinity of 4-PATs with FDA-approved antipsychotic drugs, an assessment is made of the affinity of PIMA and risperidone at 5-HT₂-type and H₁Rs. In this work, PIMA exhibited high affinity for 5-HT_{2A}Rs, with modest selectivity (12-fold) over 5-HT_{2C}Rs, and high selectivity over 5-HT_{2B} and H₁Rs (>3,000-fold). Risperidone is equipotent to PIMA at 5-HT_{2A}Rs and has high affinity at the other receptors. The results are consistent with those in the literature (Vanover, et al., 2006; Chopko & Lindsley, 2018).

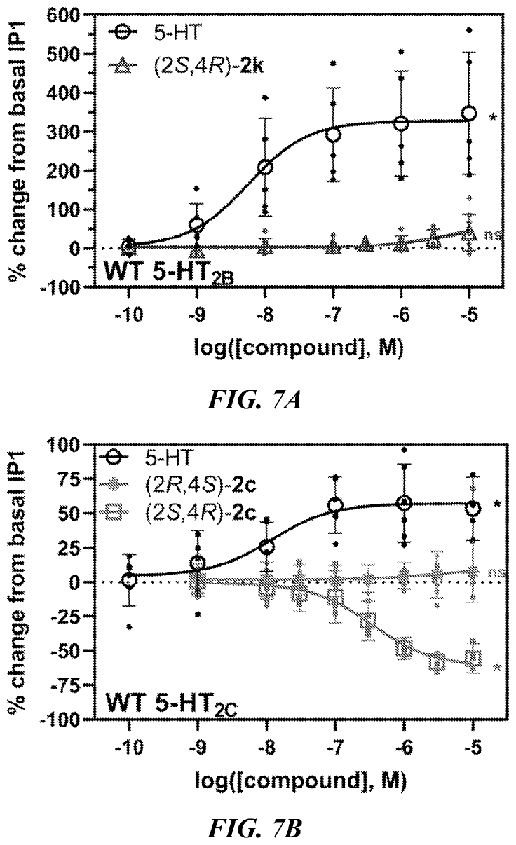

Investigations of the functional activity of 4-PATs and PIMA at 5-HT₂Rs and H₁Rs are conducted with the exploratory functional screening presented in FIG. 2A and continued to develop mechanistic specificity as is now described. To understand the impact of 4-PAT substitution and stereochemistry on the efficacy vector of functional signaling at 5-HT₂-type and H₁Rs, analogs 3a-k and 2c-k are subjected to exploratory screening at 10 μM in inositol monophosphate (IP1) accumulation assays in FIG. 2A. FIG. 2A displays the percent change from basal accumulation of IP1 in clonal cells transiently expressing human wild-type 5-HT_{2A}, 5-HT_{2B}, 5-HT_{2C}, or H₁Rs following incubation with 10 μM of the ligand indicated at the left. Reference ligands (top) include 5-HT (5-hydroxytryptamine), HIS (histamine), DOX (doxepine), RIT (ritanserin) and PIMA. The mean percent change in basal signaling for each compound is normalized to the change for the reference agonist (i.e., 5-HT or histamine), and is shown numerically in each cell as the mean of at least two independent experiments performed using three technical replicates. Crossed spaces indicate no data. In FIG. 2A, no 4-PAT-type compound tested activates 5-HT_{2A}Rs or H₁Rs. At 5-HT_{2B}Rs, however, analogs (2S,4R)-2c, (2S,4S)-3f, and (2S,4R)-2k appear to display partial agonist efficacy (12-14% 5-HT), with lead, (2S,4R)-2k, displaying ˜13% efficacy. Therefore, full concentration-response assays are undertaken for (2S,4R)-2k at 5-HT_{2B}Rs (FIG. 7A), and while a trend toward receptor activation is observed in FIG. 7A, the response did not reach statistical significance due to unequal variance between concentrations. In FIG. 2A at 5-HT_{2C}Rs, only (2R,4S)-2c, (2R,4S)-2d, and (2S,4S)-3f display apparent partial agonist efficacy (19-31% 5-HT). In concentration-response assays, however, one of the most efficacious analogs, (2R,4S)-2c provides no consistent effect on IP1-accumulation (FIG. 7B). In contrast, the (2S,4R)-2c enantiomer demonstrates inverse agonism at 5-HT_{2C}Rs (pIC₅₀=6.60±0.27, Iₘₐₓ=58% below basal; FIG. 7B). Thus, the novel 4-PAT-type ligands reported here have neutral antagonist or inverse agonist activity at 5-HT₂-type and H₁Rs.

The binding and functional screening results indicate that PIMA, (2S,4R)-2k, and (2R,4R)-3h exhibit similar affinity, and inverse agonist efficacy, at therapeutically favorable 5-HT_{2A} and 5-HT_{2C}Rs. Therefore, these compounds are comparatively assessed in concentration-response assays using clonal cells (FIG. 2B) expressing constitutively activated C322K^{6.34} 5-HT_{2A}Rs (Egan, et al., 1998) or WT 5-HT_{2C}Rs (FIGS. 2C-2E). In FIGS. 2B-2C, PIMA, (2S,4R)-2k, and (2R,4R)-3h demonstrate inverse agonist activity at constitutively activated C322K^{6.34} 5-HT_{2A}Rs (FIG. 2B) and WT 5-HT_{2C}Rs (FIG. 2C) by attenuating basal (dotted line) IP1 accumulation. In FIG. 2D, 5-HT_{2B}Rs, PIMA, (2S,4R)-2k, and (2R,4R)-3h competitively antagonize 5-HT-stimulated IP1 accumulation. In FIG. 2E, H₁Rs, (2S,4R)-2k and (2R,4R)-3h competitively antagonize histamine-stimulated IP1 accumulation, with minimal competition by PIMA. Concentration-response curves represent the mean±SD of n=5 independent experiments performed using three (FIG. 2B, FIG. 2C) or two (FIG. 2D, FIG. 2E) technical replicates. It is found that PIMA has 5- and 20-fold higher potency at C322K^{6.34} 5-HT_{2A}Rs than (2S,4R)-2k or (2R,4R)-3h (pIC₅₀=8.12±0.18, 7.43±0.17, and 6.81±0.39, respectively, FIG. 2B), with each compound showing comparable inverse agonist efficacy (˜60% below basal). The pIC₅₀ values are in excellent agreement with the corresponding pK_{b} values at WT 5-HT_{2A}Rs (Table 2). At 5-HT_{2C}Rs, PIMA and (2S,4R)-2k are equipotent (pIC₅₀=6.55±0.42 and 6.64±0.21, respectively) and similarly efficacious (˜75% below basal), whereas an IC₅₀ value of (2R,4R)-cis-3h at 5-HT_{2C}Rs cannot be detected, although it exhibits inverse agonism (FIG. 2C). At off-target 5-HT_{2B}Rs, PIMA, (2S,4R)-2k, and (2R,4R)-3h display low-potency competitive antagonism of 5-HT-mediated IP1 accumulation (pK_{b}=5.86±0.64, 5.59±0.50, and 5.34±0.24, respectively), although only PIMA reduces IP accumulation to basal levels at the concentrations used (FIG. 2D). Low-potency competitive antagonism is also observed with (2S,4R)-2k and (2R,4R)-3h at H₁Rs (pK_{b}=6.33±0.24 and 5.82±0.22, respectively), although the concentration of IP1 is not reduced to basal, and little, if any, antagonism is observed with PIMA (FIG. 2E).

TABLE 2
Functional potencies of reference and 2-aminotetralin antagonists
(pK_{b}), and 5-HT (pEC₅₀), at 5-HT_{2A}R variants^{a, b}.

| | | | | | | |
|---|---|---|---|---|---|---|
| Wild type | I210V^{4.60} | F213K^{4.63} | D231F^{5.35} | V235M^{5.39} | G238S^{5.42} | S242A^{5.46} |

pK_{b} (n)

| | | | | | | | |
|---|---|---|---|---|---|---|---|
| PIMA | 8.24 ± 0.31 | 8.25 ± 0.31 | 8.56 ± 0.35 | NC^{c} | 8.62 ± 0.14* | 4.61 ± 0.50* | 8.22 ± 0.44 |
| | (16) | (5) | (5) | | (5) | (5) | (5) |
| Risperidone | 8.88 ± 0.17 | 8.81 ± 0.31 | 8.83 ± 0.19 | NC | 8.93 ± 0.13 | 8.69 ± 0.09* | 8.95 ± 0.23 |
| | (14) | (5) | (5) | | (5) | (5) | (5) |
| (2S,4R)-2a | 6.97 ± 0.27 | 7.1 ± 0.31 | 6.82 ± 0.25 | NC | 7.25 ± 0.51 | 5.87 ± 0.13* | 6.90 ± 0.38 |
| | (16) | (5) | (5) | | (5) | (5) | (5) |
| (2S,4R)-2k | 7.91 ± 0.26 | 8.06 ± 0.17 | 7.94 ± 0.34 | NC | 8.35 ± 0.11* | 4.92 ± 0.31* | 7.95 ± 0.42 |
| | (18) | (5) | (5) | | (5) | (5) | (5) |
| (2R,4R)-3h | 6.97 ± 0.37 | 7.18 ± 0.22 | 7.04 ± 0.17 | NC | 7.02 ± 0.26 | 4.59 ± 0.48* | 6.85 ± 0.36 |
| | (16) | (5) | (5) | | (5) | (5) | (5) |

pEC₅₀ (n)

| | | | | | | | |
|---|---|---|---|---|---|---|---|
| 5-HT | 7.53 ± 0.16 | 7.44 ± 0.24 | 7.21 ± 0.16* | NC (5) | 7.34 ± 0.13* | 6.37 ± 0.24* | 7.29 ± 0.17* |
| | (19) | (6) | (5) | | (5) | (7) | (6) |

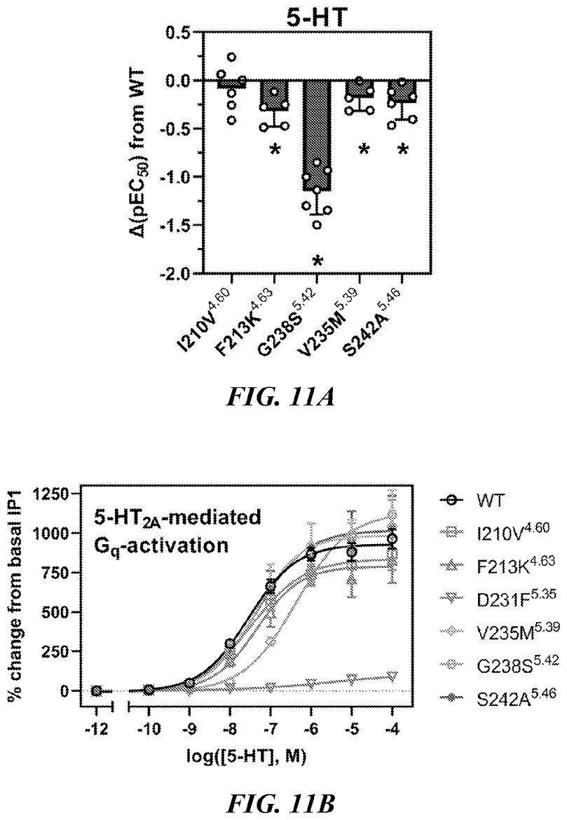

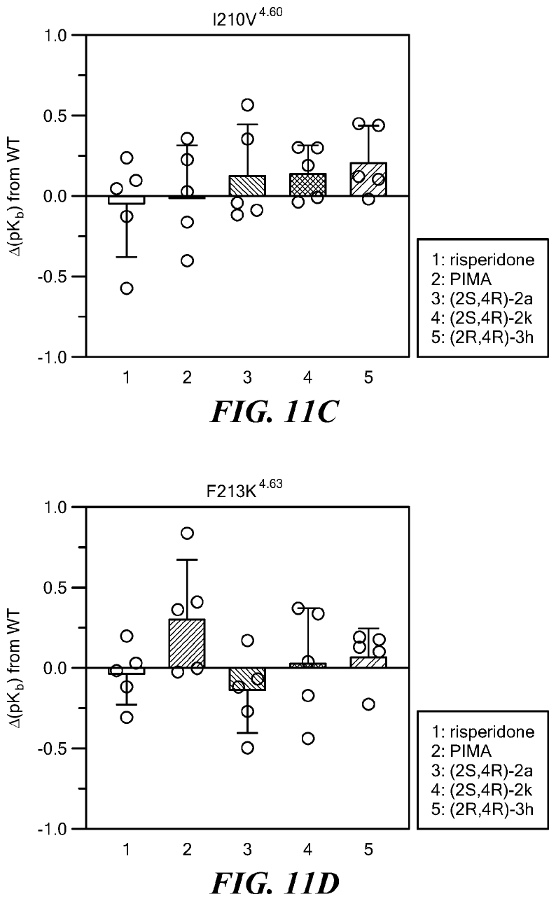

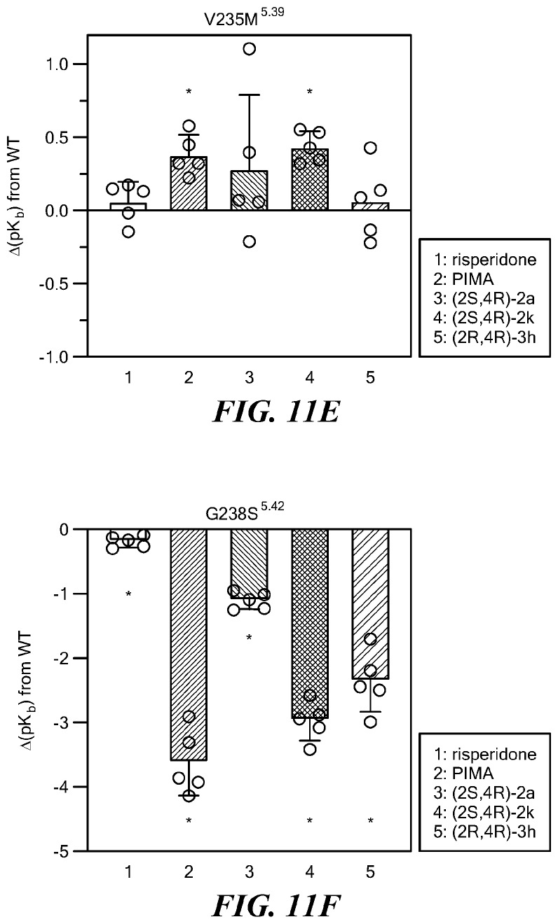

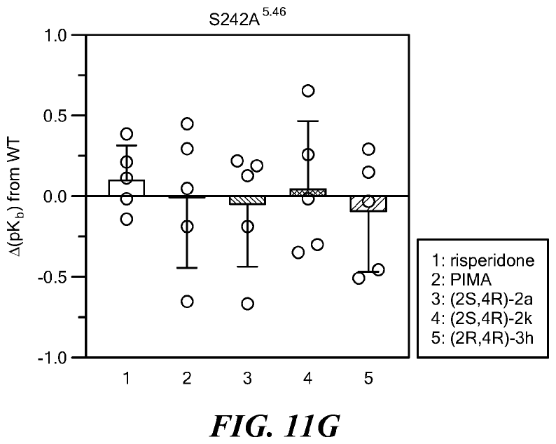

^{a}The equilibrium dissociation constant (pK_{b}) of 5-HT_{2A}R antagonists is determined in the presence of 1 μM 5-HT.
^{b}Data are presented as the pK_{b} ± SD or pEC₅₀ ± SD for the number of independent experiments indicated in parenthesis, see FIGS. 11A-11G for data visualization.
^{c}Not calculable.
Asterisk * indicates statistical significance (p < 0.05) between the wild-type and mutant receptor parameters using an unpaired t-test.

In FIGS. 3A-3C comparative assessments of PIMA, (2S,4R)-2k and (2R,4R)-3h in male C57BL/6J mice are shown. In FIG. 3A, in vivo studies comparing PIMA to (2S,4R)-2k, and (2R,4R)-3h indicate that each ligand attenuates the (±)-DOI-elicited head twitch response, a model sensitive to antipsychotic-like activity, apparently through action at 5-HT_{2A}/5-HT_{2C}Rs and not 5-HT_{1A}, α_{1A}-, D₂, or D₃Rs (Canal & Morgan, 2012). When administered alone, PIMA also suppresses locomotor activity in mice, whereas (2S,4R)-2k and (2R,4R)-3h are behaviorally selective to attenuate the head twitch response while preserving general locomotor ability (FIG. 3B, FIG. 3C). FIG. 3A shows head-twitch response elicited by 1 mg*kg⁻¹ (±)-DOI (s.c.) following pretreatment with vehicle, PIMA, (2S,4R)-2k, or (2R,4R)-3h. FIG. 3B shows locomotor activity of mice administered 1 mg*kg⁻¹ (±)-DOI (s.c.) following pretreatment with vehicle, PIMA, (2S,4R)-2k, or (2R,4R)-3h. FIG. 3C shows, after a 6-week washout period, the locomotor activity of the same mice from (3A) and (3B) is reassessed following administration (s.c.) of vehicle or 3 mg*kg⁻¹ PIMA, (2S,4R)-2k, or (2R,4R)-3h. Data represent the mean±SD of n=6 to 7 treatments, individual values are shown for each condition. Significance is determined using one-way ANOVA, with Tukey's correction for multiple comparisons, *p<0.05. Compared to mice pretreated with saline before injection of 1 mg*kg⁻¹ DOI, locomotor suppression in mice pretreated with 0.3 mg*kg⁻¹ PIMA is observed, but not 0.3 mg*kg⁻¹ (2S,4R)-2k (FIG. 3B). In fact, the distance traveled by mice pretreated with 0.3 mg*kg⁻¹ PIMA is significantly less than that of mice pretreated with 0.3 mg*kg⁻¹ (2S,4R)-2k.

This leads to the question if the apparent greater effect of 0.3 mg*kg⁻¹ PIMA in the DOI-assay might result from behaviorally disruptive effects on locomotor activity. Thus, each compound is administered alone at 3 mg*kg⁻¹, and it is found that PIMA, but not (2S,4R)-2k or (2R,4R)-3h, elicits locomotor suppression (FIG. 3C). These results indicate that (2S,4R)-2k is behaviorally selective in modulating the head twitch response.

Some 2-aminotetralins substituted at the C(5)- or C(8)-position have high affinity at 5-HT_{1A} and 5-HT₇Rs (Perry, et al., 2020), while others target D₂-like receptors (Seiler & Markstein, 1984). Furthermore, affinity at 5-HT_{2B} and α_{1B}-adrenergic receptors may predict ligand promiscuity (Peters, et al., 2012), and high affinity antagonism of central α_{1A/1B}-adrenergic receptors is associated with adverse events such as orthostatic hypotension, dizziness, and sedation (Andersson & Gratzke, 2007). Similarly, antagonism of central H₁Rs is linked to sedation in humans (Nicholson, et al., 1991; Stahl, 2008; Valk & Simons, 2009). The lead 4-PATs from this study, (2S,4R)-2k and (2R,4R)-3h, display high selectivity to bind 5-HT_{2A}/5-HT_{2C}Rs over 5-HT_{1A}, 5HT_{2B}, 5HT₇, D₂, D₃, α_{1A}- and α_{1B}-adrenergic receptors (>100-fold), whereas selectivity over H₁Rs is moderate for (2S,4R)-2k (˜38-fold).

To understand how aryl substituted 4-PATs and PIMA bind to 5-HT_{2A}Rs, molecular modeling studies are performed using a model of the 5-HT_{2A}R (FIGS. 4A-4C). FIGS. 4A-4C show proposed binding modes of PIMA, (2S,4R)-2k, and (2R,4R)-3h in the model of the 5-HT_{2A}R. The mechanism by which (2S,4R)-2k and (2R,4R)-3h selectively bind 5-HT_{2A}/5-HT_{2C}Rs involves aryl ring D occupying a cavity in 5-HT_{2A} and 5-HT_{2C}Rs afforded by the small side chain of G^{5.42}, a residue unique to 5-HT₂-type receptors and key structural determinant of polypharmacology (Peng, et al., 2018). Residue G^{5.42} is depicted in FIGS. 4A-4C in the model of the 5-HT_{2A}R. The SAR results indicate that larger aryl meta-substituents on ring C (e.g., 3a, 3b, 3b, Table 1) can yield moderate to high selectivity to bind 5-HT_{2A}Rs over 5-HT_{2B}Rs in the (2S,4R)-configuration, and over 5-HT_{2B}, 5-HT_{2C} and H₁Rs in the (2R,4R)-configuration. PIMA selectively binds 5-HT_{2A}Rs over 5-HT_{2B} and H₁Rs, with moderate selectivity over 5-HT_{2C}Rs.

FIG. 4A shows the structure of PIMA at top and the proposed binding mode at the 5-HT_{2A}R at bottom. FIG. 4B shows the structure of (2S,4R)-2k at top and the proposed binding at the 5-HT_{2A}R mode at bottom. FIG. 4C shows the structure of (2R,4R)-3h at top and the proposed binding mode at the 5-HT_{2A}R at bottom. Side chains within 4.0 Å of each ligand are shown, as well as F213^{4.63} and D231^{5.35} which are experimentally point mutated herein. For clarity, only the anchoring side chain of D155^{3.32} is shown for residues in TM3.

Site-directed mutagenesis is used to validate the proposed ligand-receptor interactions. Indeed, the mutagenesis studies reveal that (2S,4R)-2k and (2R,4R)-3h have nil affinity (pK_{b}) at G238S^{5.42} 5-HT_{2A}Rs, whereas the affinity of (2S,4R)-2a, which lacks aryl ring D, is less affected. Confounding is that 5-HT_{2B}Rs also present G^{5.42}, yet aryl substituted 4-PATs do not bind with high affinity to 5-HT_{2B}Rs. The molecular modeling results with the 5-HT_{2A}R bound to inverse agonists PIMA, (2S,4R)-2k and (2R,4R)-3h reveal similar binding modes for each ligand (FIG. 4A, FIG. 4B, FIG. 4C). All compounds dock close enough to the side chain of D155^{3.32} (Ballesteros-Weinstein numbering system, Ballesteros, 1995) such that an ionic bond can form with their respective basic amine moieties, a highly conserved interaction critical to the binding of most ligands across aminergic GPCRs (Kristiansen, et al., 2000; Vass, et al., 2019). Each ligand can also interact with conserved residues in TM3, including V156^{3.33}, S169^{3.36}, and T160^{3.37} (Table 3).

Interestingly, aryl substituted 4-PATs in the (2S,4R)-configuration have high affinity for H₁Rs (Table 1), despite the presence of T194^{5.42}, which possesses a bulkier side chain than serine. The molecular modeling results suggest that W158^{4.56}, a residue unique to H₁Rs, might form stereospecific aromatic interactions with 4-PATs to impart high affinity (FIG. 5A, FIG. 5B). For example, ring D of (2S,4R)-2k positions close to TM4, where it could form optimal T-shaped interactions with W158^{4.56}, while ring B of the aminotetralin core could form edge-to-face aromatic interactions with W428^{6.48}. In contrast, ring D of (2R,4R)-3h orients toward TM5, potentially, due to a stereochemical restriction at the C(2)-position. Interactions with residues in TM5 might be disfavored by a negative steric interaction with T194^{5.42}, and cause ring D to position between TM5 and TM6, precluding optimal aromatic interactions between ring B and the side chain of W428^{6.48}. To validate the proposed model, a W1581^{4.56} H₁R is generated. However, W1581^{4.56} H₁Rs are unable to stimulate IP1 accumulation in response to histamine, and specific binding of [³H]mepyramine or [³H]ketanserin cannot be detected.

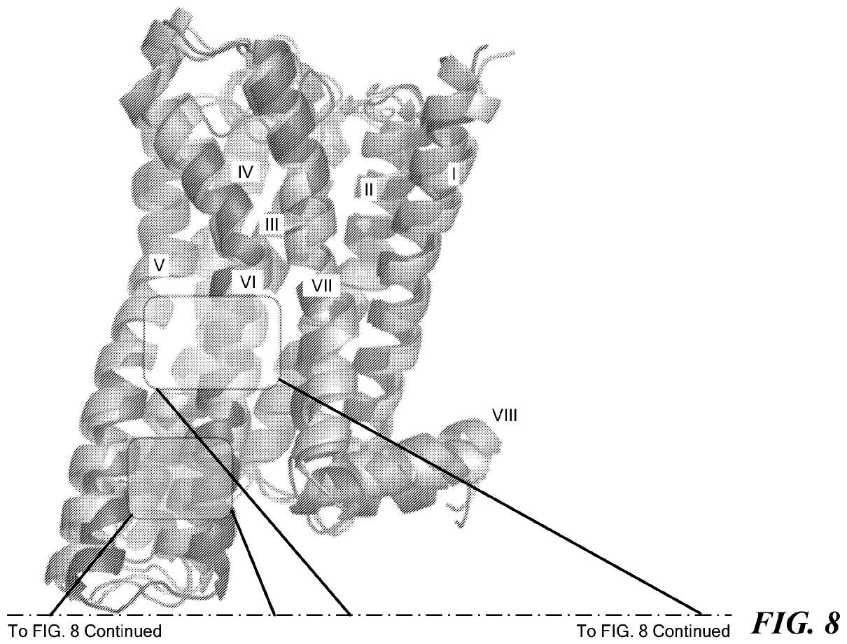

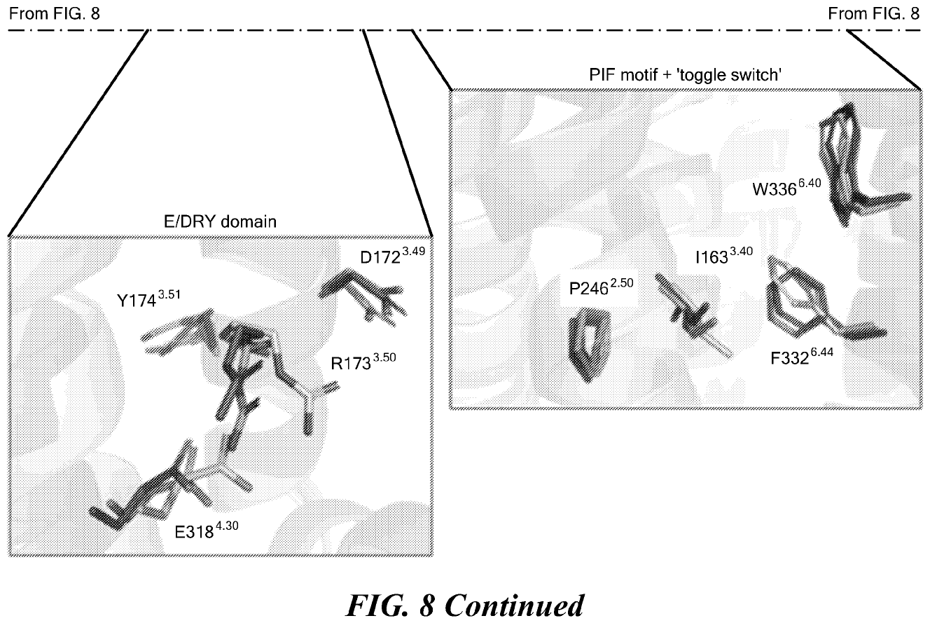

Overall, PIMA, (2S,4R)-2k, and (2R,4R)-3h stabilize an inactive-like conformation of the 5-HT_{2A}R, typified by an ionic lock between R173^{3.50} and E318^{6.30} within the E/DRY domain (FIG. 8). The ionic lock may restrict the intracellular end of TM6 from outward displacement, and thus inhibit productive Ga_{q}-coupling and accumulation of inositol phosphates (Shapiro, et al., 2002). Clues to how the inactive state is stabilized are found at the ligand-receptor interface. For example, the simulations indicate that the fluorobenzyl ring of PIMA, as well as the aminotetralin core of (2S,4R)-2k and (2R,4R)-3h, may situate deep in the hydrophobic cleft of the orthosteric binding pocket. In this way, the fluorobenzyl and aminotetralin moieties can interact directly with I163^{3.40} and F332^{6.44} of the conserved P246^{5.50}-1163^{3.40}-F322^{6.44} motif, thought to be involved in the activation mechanism of 5-HT₂-type GPCRs (Kim, et al., 2020; Kimura, et al., 2019; Peng, et al., 2018). Similar interactions are observed between these moieties and the side chain of W336^{6.48}, a ‘toggle switch’ potentially mediating on/off states of class A GPCRs (Kim, et al., 2020; Peng, et al., 2018; Rasmussen, et al., 2011; Visiers, et al., 2002) (FIG. 4A, FIG. 4B, FIG. 4C, FIG. 8). Moreover, PIMA, (2S,4R)-2k, and (2R,4R)-3h can form edge-to-face aromatic interactions with the side chains of F243^{5.47} and F340^{6.52}, while r-cation interactions can form between the side chain of F339^{6.51} and the basic nitrogen of the PIMA piperidine fragment or the tertiary amine of (2S,4R)-2k and (2R,4R)-3h.

The models also indicate that the isobutoxybenzyl moiety of PIMA and aryl ring D of (2S,4R)-2k and (2R,4R)-3h occupy a side cavity between TM4 and TM5, unimpeded by the small side chain of G238^{5.42}, a residue unique to 5-HT₂-type receptors among aminergic GPCRs. Furthermore, in all models, F234^{5.38} assumes a rotamer conformation oriented away from G238^{5.42}, which is suggested to extend the side cavity (Kimura, et al., 2019). Several amphipathic and hydrophobic side chains in this region of the binding pocket (I210^{4.60}, V235^{5.39}, G238^{5.42}, and S242^{5.46}) are close enough to the isobutoxybenzyl of PIMA and aryl ring D of (2S,4R)-2k and (2R,4R)-3h to facilitate interactions (Table 3), thus providing a potential structural basis for the observed selectivity of these ligands to bind 5-HT_{2A}Rs. In Table 3, conserved residues are highlighted in parenthesis; results for point-mutated 5-HT_{2A}R residues in bold (with quotation marks) are reported herein.

TABLE 3
Residues within 4.0 Å of docked PIMA, (2S,4R)-2k, or (2R,4R)-
3h at 5-HT_{2A}Rs following molecular dynamics simulations,
and equivalent residues at 5-HT_{2B}, 5-HT_{2C}, and H₁ GPCRs.

| 5-HT_{2A} | 5-HT_{2B} | 5-HT_{2C} | H₁ |
|---|---|---|---|
| D155^{3.32} | (D) | (D) | (D) |
| V156^{3.33} | (V) | (V) | (Y) |
| S159^{3.36} | (S) | (S) | (S) |
| T160^{3.37} | (T) | (T) | (T) |
| I163^{3.40} | (I) | (I) | (I) |
| I206^{4.56} | (I) | V | W |
| S207^{4.57} | A | (S) | V |
| P209^{4.59} | (P) | (P) | (P) |
| “I210^{4.60}” | V | (I) | (I) |
| C227^{45.50a} | (C) | (C) | (C) |
| L229^{45.52} | (L) | (L) | T |
| F234^{5.38} | (F) | (F) | (F) |
| “V235^{5.39}” | M | (M) | K |
| I237^{5.41b} | F | (I) | M |
| “G238^{5.42}” | (G) | (G) | T |
| S239^{5.43} | (S) | (S) | A |
| V241^{5.45b} | A | (M) | I |
| “S242^{5.46}” | A | A | N |
| F243^{5.47} | (F) | (F) | (F) |
| F332^{6.44} | (F) | (F) | F) |
| W336^{6.48} | (W) | (W) | (W) |
| F339^{6.51} | (F) | (F) | (Y) |
| F340^{6.52} | (F) | (F) | (F) |
| V366^{7.39} | (V) | (V) | I |
| Y370^{7.43c} | (Y) | (Y) | Y |

^{a}Within 4 Å of only (2S,4R)-2k
^{b}Only within 4 Å of PIMA
^{c}Only within 4 Å of (2R,4R)-3h

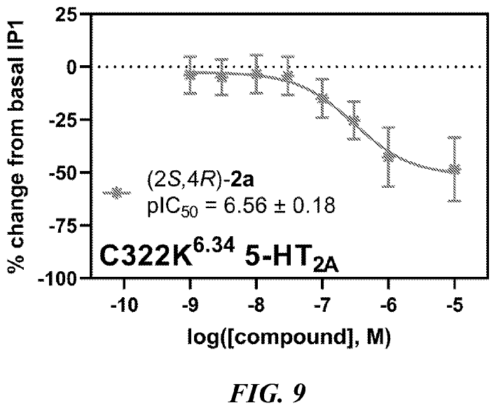

To validate the molecular modeling results, residues are point-mutated in and around the 5-HT_{2A}R side-extended cavity (Kimura, et al., 2019) and quantified the antagonist affinity (pK_{b}) of (2S,4R)-2k and (2R,4R)-3h, as well as (2S,4R)-2a (which lacks 5-HT₂R subtype selectivity) at 5-HT_{2A}R variants to understand how stereochemistry and aryl ring D impact ligand-receptor interactions. Notably, like PIMA, (2S,4R)-2k, and (2R,4R)-3h, key analog (2S,4R)-2a demonstrate inverse agonist activity at C322K^{6.34} 5-HT_{2A}Rs (FIG. 9). An assessment is also made of the antagonist affinity of PIMA and risperidone at point-mutated 5-HT_{2A}Rs, which represent selective and promiscuous 5-HT_{2A}R ligands, respectively.

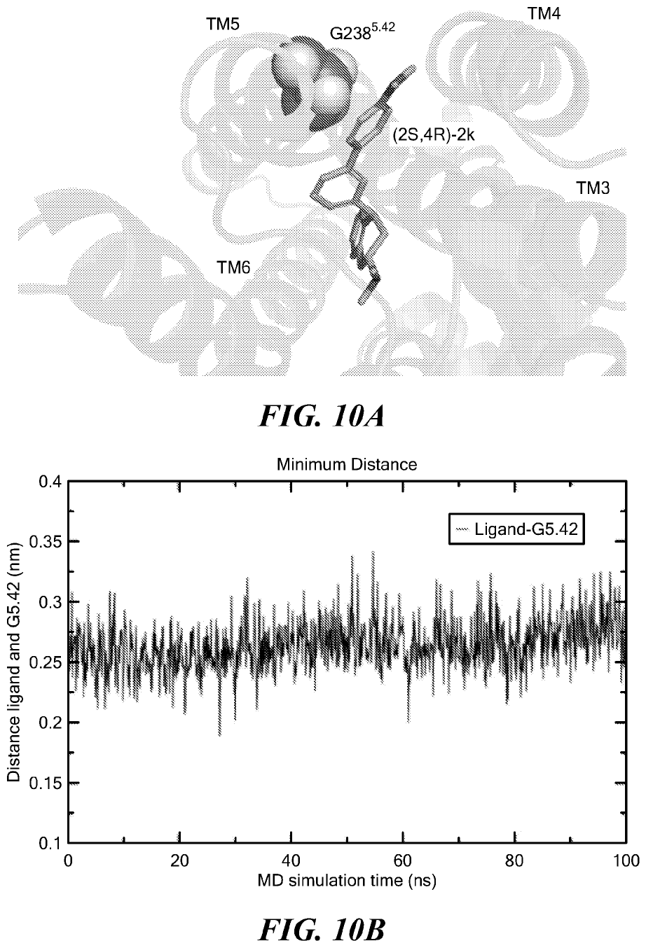

A G238S^{5.42} 5-HT_{2A}R is generated to test the hypothesis that the large side chain of serine precludes ligand access to the side extended cavity, as suggested by the molecular modeling results (FIG. 10A, FIG. 10B) and reported elsewhere for PIMA (Kimura, et al., 2019). Compared to WT 5-HT_{2A}Rs, a modest, but significant, decrease in the pK_{b} of risperidone and (2S,4R)-2a at G238S^{5.42} 5-HT_{2A}Rs is observed. Moreover, the affinity of PIMA, (2S,4R)-2k, and (2R,4R)-3h is nearly abolished at G238S^{5.42} 5-HT_{2A}Rs (Table 2, FIG. 11F). Notably, the Δ(pK_{b}) for (2S,4R)-2a is less than that of (2S,4R)-2k and (2R,4R)-3h suggesting a size-dependent negative steric interaction between the 4-PAT ring C substituent and S^{5.42}. Also, a significant reduction in the potency is observed, but not the efficacy, of 5-HT at G238S^{5.42} compared to WT 5-HT_{2A}Rs (Table 2, FIG. 11A, FIG. 11B), consistent with previous reports (Kimura, et al., 2019).

The experiments are extended by asking if the attenuated affinity of (2S,4R)-2a, (2S,4R)-2k, and (2R,4R)-3h at G238S^{5.42} 5-HT_{2A}Rs translates to aminergic GPCRs natively presenting S^{5.42}. Table 4 shows that (2S,4R)-2k and (2R,4R)-3h have >1,000-fold selectivity for 5-HT_{2A}Rs over 5-HT_{1A}, 5-HT₇, D_{2L}, α_{1A}- and α_{1B}-adrenergic GPCRs. It is noted that (2S,4R)-2k has 270-fold selectivity over D₃Rs, whereas (2R,4R)-3h has >1,000-fold selectivity. In contrast, (2S,4R)-2a exhibits moderate-to-high affinity for 5-HT₇, D_{2L}, D₃, and α_{1A}-adrenergic receptors.

TABLE 4
Affinities (pKᵢ) of (2S,4R)-2a, (2S,4R)-2k, and (2R,4R)-
3h at aminergic GPCRs natively presenting S^{5.42a}

| | 5-HT_{1A} | 5-HT₇ | D_{2L} | D₃ | α_{1A} | α_{1B} |
|---|---|---|---|---|---|---|
| (2S,4R)-2a | <5.0⁽²⁾ | 7.38⁽³⁾ | 7.16⁽³⁾ | 6.92⁽²⁾ | 7.99⁽³⁾ | 5.49⁽³⁾ |
| | | 7.47-7.23 | 7.18-7.14 | 6.90-6.93 | 8.30-7.77 | 5.76-5.22 |
| (2S,4R)-2k | 5.58⁽³⁾ | 6.28⁽³⁾ | 5.70⁽³⁾ | 6.92⁽³⁾ | 6.21 ⁽²⁾ | 6.19⁽²⁾ |
| | 5.81-5.32 | 6.42-6.17 | 6.05-5.60 | 7.21-6.75 | 6.31-6.10 | 6.21-6.17 |
| (2R,4R)-3h | <5.0⁽³⁾ | 6.29⁽⁴⁾ | <5.0⁽³⁾ | 6.21 ⁽³⁾ | 6.13⁽²⁾ | 6.10⁽²⁾ |
| | | 6.66-6.11 | | 6.63-5.94 | 6.20-6.06 | 6.14-6.07 |

^{a}Data are presented as the mean pKᵢ and range for n independent experiments, shown in superscript.

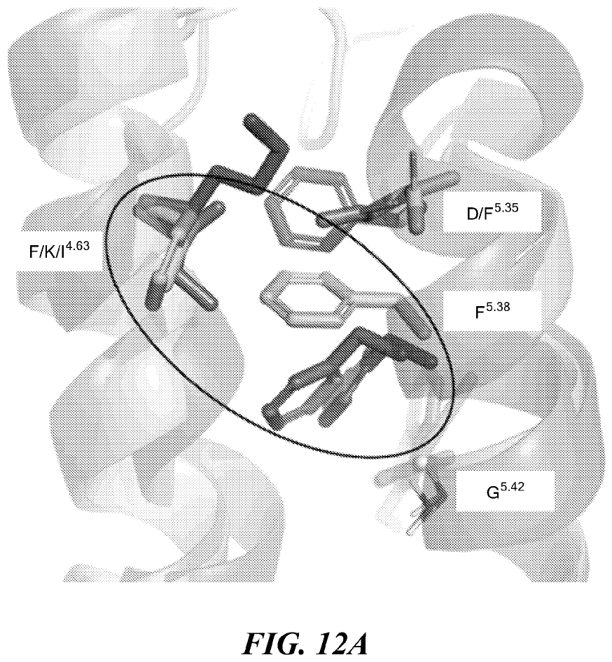

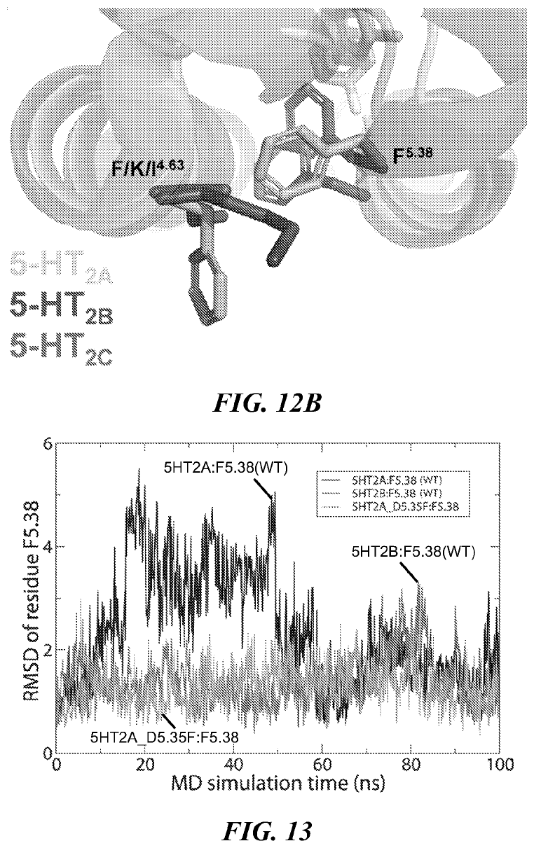

Alignment of 5-HT_{2A}, 5-HT_{2B} and 5-HT_{2C}R crystal structures (FIG. 12A, FIG. 12B) indicates that a side chain rotamer of F234^{5.38}, unique to 5-HT_{2A}Rs (Kimura, et al., 2019), orients toward the extracellular end of TM4 to form a side-extended cavity potentially due to hydrophobic interactions with F213^{4.63}. In contrast, K193^{4.63} and 1192^{4.63} in 5-HT_{2B} and 5-HT_{2C}Rs, respectively, do not form productive interactions with F^{5.38} and may restrict the side cavity (Kimura, et al., 2019). However, no experimental studies have been reported to test this hypothesis. Therefore, a F213K^{4.63} 5-HT_{2A}R is generated to test the hypothesis that the selectivity of PIMA, (2S,4R)-2k, and (2R,4R)-3h to bind 5-HT_{2A}Rs relies on an interaction between F213^{4.63} and F234^{5.38}. Unexpectedly, a change in the pK_{b} of any antagonist tested at F213K^{4.63}5-HT_{2A}Rs is not observed (Table 2, FIG. 11D). However, a decrease in the potency of 5-HT at F213K^{4.63} 5-HT_{2A}Rs is observed, but not its efficacy (Table 2, FIG. 11A, FIG. 11B). These results do not support the hypothesis that F213^{4.63} mediates subtype selective binding of inverse agonists at 5-HT_{2A}Rs, however, F213^{4.63} may be involved in 5-HT binding.

Further inspection of the 5-HT₂-type receptor crystal structures reveals that one helical turn above F^{5.38} in 5-HT_{2A} and 5-HT_{2C}Rs exists a non-conserved residue in 5-HT_{2B}Rs (D2315.35 D211^{5.35}, and F214⁵³⁵, respectively). The root-mean-square deviation (RMSD) of the F^{5.38} side chain in WT 5-HT_{2A} and 5-HT_{2B}Rs is tracked in silico and found that F^{5.38} exhibited large transient variations in RMSD in WT 5-HT_{2A}Rs. Interestingly, the RMSD of F^{5.38} in D231F^{5.35} 5-HT_{2A}Rs recapitulates the restricted pattern observed in silico for WT 5-HT_{2B}Rs, indicating that D231^{5.35} may facilitate flexibility in the side chain of F^{5.38} (FIG. 13).

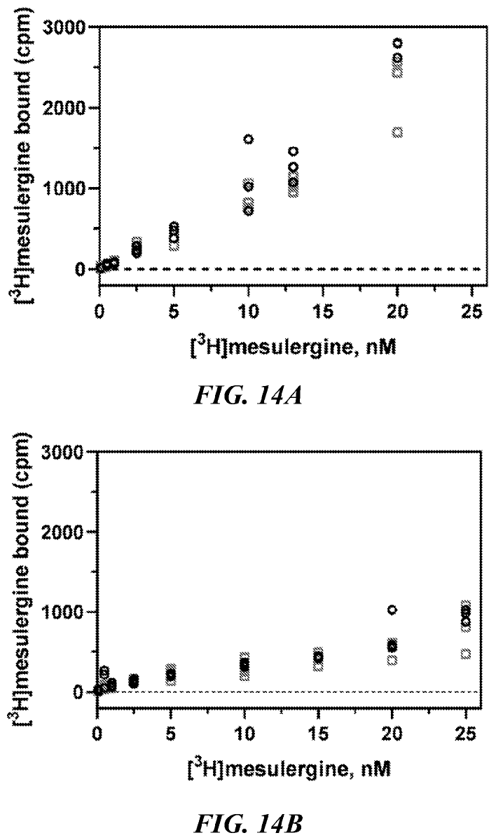

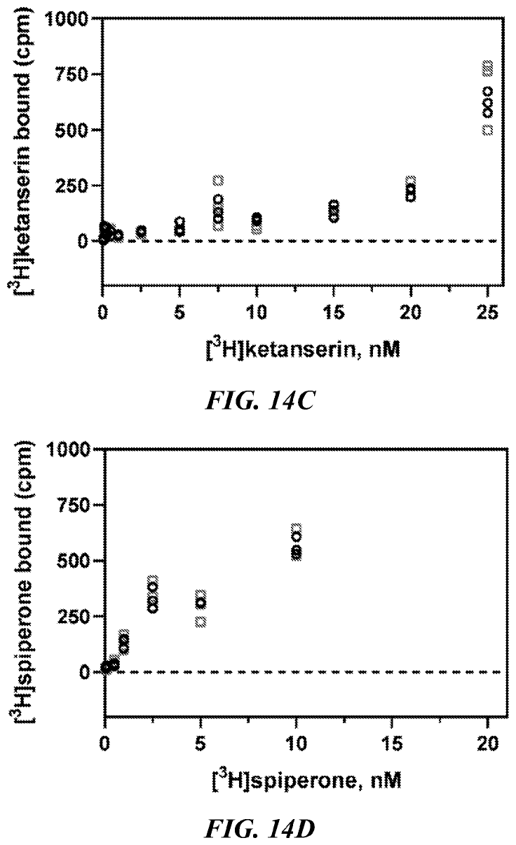

It is therefore hypothesized that D231^{5.35} may modulate the side chain rotamer of F234^{5.38} in 5-HT_{2A}Rs to mediate subtype selective binding. To test this hypothesis, a D231F^{5.35} 5-HT_{2A}R is generated, however, D231F^{5.35} 5-HT_{2A}Rs are insufficiently responsive to 5-HT for competitive antagonism studies (FIG. 11B), thus, antagonist activity cannot be experimentally determined. Furthermore, no specific binding is detected for [³H]ketanserin, [³H]mesulergine, or [³H]spiperone in exploratory studies using membranes from cells transfected with cDNA encoding D231F^{5.35} 5-HT_{2A}Rs (FIGS. 14A-14D).

Residues in TM4 and TM5 lining the side-extended cavity of 5-HT_{2A}Rs and in proximity to PIMA, (2S,4R)-2k, and (2R,4R)-3h (Table 3) are then investigated. Among these are the side chains of I210^{4.60}, V235^{5.39}, and S242^{5.46}. Importantly, the side chains of I210^{4.60} and V235^{5.39} are conserved in 5-HT_{2C}Rs, while S242^{5.46} is unique to 5-HT_{2A}Rs. It is hypothesized that selectivity to bind 5-HT_{2A} and 5-HT_{2C}Rs over 5-HT_{2B}Rs may involve interactions with the side chains of I^{4.60}, V^{5.39}, or the 5-HT_{2A}R-specific residue S242^{5.46}. In fact, a significant increase in the affinity of PIMA and (2S,4R)-2k at V235M^{5.39} 5-HT_{2A}Rs is observed, with no change in affinity for any antagonist at I210V^{4.60} or S242A^{5.46} 5-HT_{2A}Rs (Table 2, FIG. 11C, FIG. 11E, FIG. 11G). Interestingly, the potency of 5-HT (but not the efficacy) is attenuated at V235M^{5.39} and S242A^{5.46} but not 1210V^{4.60} 5-HT_{2A}Rs, consistent with other reports investigating 1210V^{4.60} and S242A^{5.46} 5-HT_{2A}Rs (Table 2, FIG. 11A, FIG. 11B) (Kimura, et al., 2019).

To explain the observed selectivity of PIMA, (2S,4R)-2k, and (2R,4R)-3h to bind 5-HT_{2A}Rs over 5-HT_{2B}Rs, the focus is made on non-conserved residues which line the 5-HT_{2A}R side extended cavity. The results indicate that the affinity and selectivity of inverse agonists to bind 5-HT_{2A}Rs does not individually involve the side chains of I210^{4.60}, F213^{4.63}, V235^{5.39}, or S242^{5.46}, since point mutation to the equivalent residues in 5-HT_{2B}Rs does not attenuate their affinity. Importantly, the finding that selective inverse agonists have similar affinity at F213K^{4.63} and WT 5-HT_{2A}Rs suggests a refinement to the current understanding of subtype selective binding at 5-HT_{2A}Rs (Kimura, et al., 2019). An attempt is made to identify a 5-HT_{2A}R residue other than F213^{4.63} which may impact the side chain rotamer of F^{5.38}, and the computational results directed this work to D231.3⁵. However, D231F^{5.35} 5-HT_{2A}Rs lack sufficient Ga_{q} functionality and cannot bind various radioligands, which, to the best of knowledge of the literature, is reported here for the first time. Each ligand is 9-10 Å from D231^{5.35} in the molecular models herein, thus, direct interactions are unlikely. Instead, it is speculated that D231F^{5.35} may hinder proper protein folding and trafficking of the receptor to the membrane.

In an embodiment, the 4′-NMe₂—C₆H₄ substituent introduced on ring C (2k, 3k) in the stereoisomers is utilized to determine molecular determinants of selective binding to 5-HT_{2A} and 5-HT_{2C}Rs including substituents of a length about that of NMe₂ on ring D (FIG. 1A, FIG. 1B, lower right), for example, —F, —Cl, —Br, —I, —NH₂, —NH(CH₃), —N(CH₃)₂, —N*(CH₃)₃, —N⁺(CH₃)₂(CH₂CH₃), —N*(CH₃)(CH₂CH₃)₂, —N*(CH₂CH₃)₃, —NH(CH₂CH₃), —N(CH₂CH₃)₂, —C═NH, —C═NNH₂, —C═ONH₂, —NO₂, —NO, —CN, —N₃, —N═C═O, —CH₃, —CH₂CH₃, —CH(CH₃)₂, —C═OOH, —CH₂C═OOH, —S═OCH₃, —S(═O)₂CH₃, —S(═O)₂OH, —S(═O)₂NH₂, —S(═O)₂N(CH₃)₂, —OH, —OCN, —OCH₃, —OCH₂CH₃, —CH₂OH, —CH₂CH₂OH, —CHOHCH₂OH, —CHOHCH₃, —SH, —SCN, —SCH₃, —SCH₂CH₃, —CH₂SH, —CH₂CH₂SH, —CHSHCH₂SH, and —CHSHCH₃. In an embodiment, carbon-hydrogen (C—H) bonds have a length of about 1.09 Å, while C—N single bonds have a length of about 1.48 Å. The length of a fluorine atom is about 1.47 Å, and hydrogen is about 1.2 Å. In an embodiment, the substituent introduced on ring D can have a length measured extending from ring D, not including the bond from substituent to ring D, in the range from about 1.0 Å to about 25 Å, or in the range from about 1.0 Å to about 10 Å.

Like the observations with 5-HT_{2B}Rs, the selectivity of some aryl substituted 4-PATs to bind 5-HT_{2A} over H₁Rs cannot be explained by the results with G238S^{5.42} 5-HT_{2A}Rs. For example, H₁Rs present T194^{5.42}, yet aryl substituted 4-PATs in the (2S,4R)-configuration, but not the (2R,4R)-configuration, bind to H₁Rs with high affinity. Molecular dynamics studies revealing the structural basis of 4-PAT stereoselectivity at histamine H₁Rs are shown in FIGS. 5A-5B. FIG. 5A shows the proposed binding mode of (2S,4R)-2k at the H₁R resembles that observed at the 5-HT_{2A}R (FIG. 4B), where the aminotetralin moiety could form aromatic T-stacking interactions with the side chain of W428⁶⁴⁸, while the aryl substituent extends in a cavity between TM4 and TM5 where it could participate in aromatic T-stacking interactions with the side chain of W158^{4.56}. FIG. 5B shows the proposed binding mode of (2R,4R)-3h at the H₁R indicates that a stereochemical restriction at the C(2)-position causes the aryl substituent to position between TM5 and TM6, thus disfavoring productive aromatic interactions between the ligand and the side chains of W158^{4.56} and W428^{6.48}. The modeling studies indicate that high affinity binding of aryl substituted 4-PATs in the (2S,4R)-configuration may be afforded by stereospecific aromatic interactions with W158^{4.56}, a residue unique to H₁Rs among aminergic GPCRs and critical to histamine, mepyramine and (2S,4R)-1 binding (Cordova-Sintjago, et al., 2012). Such interactions may position ring D of aryl substituted 4-PATs in the (2S,4R)-configuration in a cleft between TM4 and TM5, toward TM4 and away from T194^{5.42}. In contrast, T194^{5.42} may preclude high affinity binding of aryl substituted 4-PATs in the (2R,4R)-configuration due to a stereochemical restraint at the C(2)-position, causing ring D to disfavor aromatic interactions with W158^{4.56} It is speculated that these findings can also explain the selectivity of (2S,4S)-3a, 3b, and 3b′ to bind H₁Rs, as the meta-halo substituent on ring C may form van der Waals interactions with W158^{4.56}, while interactions with I/V⁴⁵⁶ in 5-HT₂-type receptors might be too weak to support high affinity binding. Together, the results echo work from other labs showing T194^{5.42} mediates stereoselective binding of H₁R antagonists, such as: (R)- and (S)-hydroxyzine, (R)- and (S)-cetirizine, as well as, (R)-ubc-29992 and (S)-ubc-29993 (Gillard, et al., 2002; Moguilevsky, et al., 1995). These authors speculated that residues such as K191⁵³⁹ may influence stereospecific interactions with T194^{5.42} (Gillard, et al., 2002), while others suggested that W158^{4.56} may impact selective binding to H₁Rs (Shimamura, et al., 2011). Based on knowledge of the literature, this is the first report to suggest W158^{4.56} influences the way stereoisomers interact with T194^{5.42}.

The research disclosed here details the discovery of a novel series of selective 5-HT_{2A}/5-HT_{2C}R inverse agonists (i.e., aryl substituted 4-PATs), which are behaviorally active at comparable doses to PIMA, an FDA-approved drug. Efforts to detail the mechanism of their selectivity over 5-HT_{2B}Rs are challenging, though evidence is provided herein that F213^{4.63} is not a molecular determinant of subtype-selective inverse agonist binding at 5-HT_{2A}Rs. Also G228S^{5.42} 5-HT_{2A}Rs are identified as a point mutated 5-HT_{2A}R which may predict ligand selectivity over several aminergic GPCRs. In most cases, the mutagenesis studies reveal only minor changes in antagonist affinity, i.e., Δ(pK_{b})≤0.5. Therefore, it is surmised that ensembles of non-conserved residues, rather than individual residues, mediate subtype-selective antagonist or inverse agonist binding at 5-HT_{2A}Rs. In line with this, herein is reported a non-conserved, stereochemically sensitive molecular ensemble for H₁R recognition. Taken together, the results warrant further investigation into the ability of aryl substituted 4-PAT diastereomers to unlock the molecular determinants of subtype-selective binding at 5-HT_{2A}Rs, and preclinical characterization of analogs like (2S,4R)-2k in animal models of psychosis.

These data show that antagonist activity at G238S^{5.42} 5-HT_{2A}Rs is predictive of ligand selectivity over several aminergic GPCRs, and non-conserved residues in transmembrane domains 4 and 5 of the H₁R mediate stereoselective ligand binding. The clinical significance is relevant because understanding the molecular determinants of selective binding over H₁Rs may yield non-sedating antipsychotic medications, and aryl substituted 4-PATs demonstrate pharmacology like PIMA yet do not alter locomotor activity in mice.

Examples of psychiatric therapeutic indications

### 1. Schizophrenia.

An example of psychiatric therapeutic indications for 5-HT_{2A} and 5-HT_{2C} inverse agonists or antagonists is schizophrenia. As documented in Casey, et al., 2022 and references therein, reduction of serotonin 5-HT_{2A} receptor signaling via receptor antagonism and/or inverse agonism reported for the compounds in Casey, et al., 2022 is associated with clinical drug efficacy to treat schizophrenia and other conditions involving psychosis characterized by hallucinations and delusions (references in Casey, et al., 2022: Hacksell, et al., 2014; Meltzer, 1999 and Weiner, et al., 2001).

In addition, reduction of serotonin 5-HT_{2C} receptor signaling via receptor antagonism and/or inverse agonism reported for the novel compounds in Casey et al., 2022 also is associated clinically with drug efficacy to treat schizophrenia (references in Casey, et al., 2022: Chagraoui, et al., 2016).

### 2. Psychoses.

Regarding psychoses, as documented in Casey, et al., 2022, inverse agonist activity at the serotonin 5-HT_{2C} receptor reported for the novel compounds in Casey, et al., 2022 is associated clinically with treatment of psychoses (reference in Casey, et al., 2022: Chagraoui, et al., 2016). For example, the selective 5-HT_{2A}/5-HT_{2C} receptor inverse agonist PIMA (Zuplazid®) is FDA approved to treat hallucinations and delusions associated with Parkinson's disease psychosis (reference in Casey, et al., 2022: Cummings, et al., 2014).

In addition, the novel compounds reported in Casey, et al., 2022 likely will be effective to treat psychosis and dementia associated with Alzheimer's disease and other disorders characterized dementia. For example, the selective 5-HT_{2A}/5-HT_{2C} receptor inverse agonist PIMA is undergoing clinical assessment for efficacy to treat psychosis in Alzheimer's disease (Caraci, et al., 2020; Ballard, et al., 2020; Ballard, et al., 2018; Tariot, et al., 2021).

### 3. Depression.

As documented in Casey, et al., 2022, the novel compounds reported demonstrate inverse agonism of 5-HT_{2C} receptors which is thought to be pharmacotherapeutic for major depression (reference in Casey, et al., 2022; Demireva et al., 2018).

### 4. Anxiety.

Regarding anxiety, as documented in Casey, et al., 2022, the novel compounds reported demonstrate inverse agonism of 5-HT_{2C} receptors which is thought to be pharmacotherapeutic for generalized anxiety (reference in Casey, et al., 2022: Demireva, et al., 2018). Note that anxiety behavior is associated with a wide variety of neuropsychiatric (including substance use disorder), neurodegenerative (AD, PD), and neurodevelopmental (autism) disorders, all of which may be therapeutic indications for the novel compounds reported in Casey, et al., 2022 and analogs thereof (Fluyau, et al., 2022; Simonoff, et al., 2008; El Haj, et al., 2020; Wen, et al., 2016).

Examples of non-psychiatric therapeutic indications:

### B. Thrombosis

Thrombosis or the prevention thereof (which can be referred to as ‘blood thinners’) is most often treated with drugs that affect the blood clotting cascade such as heparin (a natural product used intravenously) and warfarin (an orally-active natural product anticoagulant) and the newer synthetic and much more expensive ‘blood thinning’ drugs apixaban (Eliquis), dabigatran (Pradaxa), edoxaban (Savaysa), and rivaroxaban (Xarelto). They are used to prevent blood clots associated with deep vein thrombosis, pulmonary emboli, atrial fibrillation and other heart and cardiovascular system disorders where pathophysiology involves blood clots. Activation of 5HT2_{A} receptors on platelets activates membrane bound phospholipase C to produce the second messengers inositol phosphates and diacylgylcerol which cause the platelets to become ‘sticky’—the platelets adhere to each other and form a clot.

Although thrombosis pathophysiology and potential pharmacotherapy involving 5HT2_{A} receptor antagonism is known (e.g., Lin, et al., 2014), most 5HT2_{A} antagonists also enter the brain to produce psychopharmacological effects that are not necessary and may be counterproductive for cardiovascular disorders. In Lin, the authors note that the 5HT2_{A} antagonists they mention are antidepressants (not commonly used as such because there are much better ones such as the SSRIs that work differently) thus they suggest the therapeutic indication should be depressed patients who have thrombosis disorders. A 4PAT-type 5HT2_{A} antagonist or inverse agonist is claimed to prevent this thrombosis or reverse it in patients where psychotropic actions are not needed.

### C. 5HT2B Inverse Agonists/Antagonists to Treat Cardiac Fibrosis-Related Conditions.

5-HT_{2B} inverse agonists/antagonists can be utilized for treatment of atherosclerosis. Activation of 5HT_{2B} causes atherosclerosis of cardiac valves. In Janssen, et al., 2015, for example, 5-HT_{2B} receptor antagonists have been investigated to inhibit fibrosis and protect from RV (right ventricular) heart failure. The present technology can provide selectivity between the CNS and the periphery with ligand specificity.

D. Hypertension treatment by 5-HT2A antagonists

It is reported in Casey, et al., 2022 that the new chemical entities are potent 5-HT_{2A} antagonists. It is known for several decades that antagonism of 5-HT_{2A} receptor leads to beneficial cardiovascular effects including reduced platelet aggregation and vasodilation. For example, the 5-HT_{2A} antagonist ketanserin is approved to treat hypertension (Hedner & Persson, 1988; Bellos, et al., 2020; Nagatomo, et al., 2004).

E. Migraine treatment by 5-HT2B antagonists.

It is reported in Casey, et al., 2022 that the new chemical entities are potent 5-HT_{2B} antagonists and inverse agonists. Exploiting their expression in the CNS vascular system, 5-HT2B antagonists are proposed to treat migraine headache (Padhariya, et al., 2017).

F. Obesity treatment by 5-HT2B antagonists

It is reported in Casey, et al., 2022 that the new chemical entities are potent 5-HT_{2B} antagonists and inverse agonists. Exploiting their expression in the gastrointestinal system, 5-HT_{2B} antagonists are proposed to obesity (Padhariya, et al., 2017).

G. Irritable bowel syndrome (IBS) treatment by 5-HT2B antagonists

It is reported in Casey, et al., 2022 that the new chemical entities are potent 5-HT_{2B} antagonists and inverse agonists. Exploiting their expression in the gastrointestinal system, 5-HT_{2B} antagonists are proposed to treat irritable bowel syndrome (IBS) (Padhariya, et al., 2017).

H. Safety of the novel chemical entities reported in Casey et al., 2022.

The novel chemical entities reported in Casey, et al., 2022 and analogs thereof are unlikely to demonstrate untoward cardiovascular effects because it is documented in the paper that the compounds are inverse agonists at the serotonin 5-HT_{2B} receptor. Agonists at 5-HT_{2B} receptors have potential to cause valvular heart disease and other untoward cardiovascular effects (references in Casey, et al., 2022: Ayme-Dietrich, et al., 2017; Rothman, et al., 2000).

Also as reported in Casey et al., 2022, the novel compounds did not demonstrate inhibition of locomotor activity (bradykinesia) indicating it is unlikely the compounds and analogs thereof will cause sedation or neurological disorders (Berardelli, et al., 2001).

### EXAMPLES

### Example 1. Chemical Syntheses

All commercially available regents and solvents were purchased and used without purification, unless otherwise specified. Flash column chromatography was performed with the use of Agela Technologies 230-400 mesh silica gel. Analytical thin-layer chromatography (TLC) was carried out on Agela Technologies silica gel 60F254 plates. Final compounds were converted from free base to HCl salt either before or after NMR analysis, as noted. All spectra were recorded by a Varian 500 MHz, 400 MHz NMR in CDCl₃ or CD₃OD as noted and are expressed as chemical shift (6) values in parts per million (ppm). Coupling constants (J) are presented in Hertz. Abbreviations used in the reporting of NMR spectra include s=singlet, bs=broad singlet, d=doublet, t=triplet, q=quartet, dd=doublet of doublets, qd=quartet of doublets, dt=doublet of triplets, m=multiplets. High resolution mass spectrometry (HRMS) was performed with an LTQ Orbitrap XL (Thermo Fisher Scientific) instrument using electron spray ionization (ESI). Sample data were acquired with MS1 scan (m/z 50-500) at 30,000 resolution using the Orbitrap as the MS detector. HPLC separation of both enantiomers was determined by UV Trace 220/254 nm on a Shimadzu™ instrument equipped with a semi-preparative (s-prep)-RegisCell™ (5 μm, 25 cm×10 mm i.d.) chiral (polysaccharide-based) column.

To efficiently produce novel 4-PAT derivatives, designing and implementing an improved synthetic route than reported earlier was accomplished (Sakhuja, et al., 2015). Previously, key compounds like 2a were synthesized using a four-step procedure from commercially available 3-bromostyrene and trifluoroacetyl phenylacetyl anhydride (Canal, et al., 2014; Sakhuja, et al., 2015). The overall yield for this pathway, however, was low, and reproducibility in large scale was unreliable. The modified synthetic pathway utilized here involved Friedel-Crafts cycli-acylalkylation of the commercially available 3-bromostyrene and phenylacetyl chloride to give the intermediate tetralone 6a (Scheme 1). The tetralone was subjected to reductive amination to afford a separable mixture of 3′-Br-4-PAT diastereomers with improved overall yields (2a 32% yield, 3a 25% yield). The reductive amination step was further optimized to obtain racemic cis-4-PATs (e.g., [2S,4S], [2R,4R]) as the major isomer. The modified protocol involved reduction of a preformed enamine with sodium borohydride (Scheme 2).

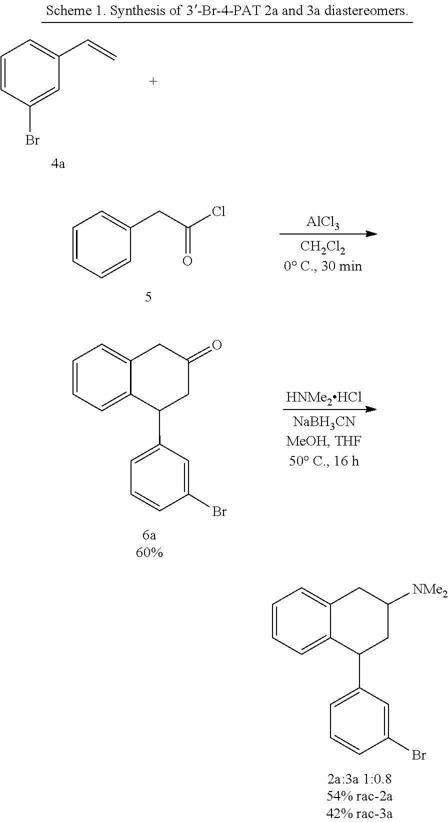

SCHEME 2
Optimized synthesis of 4-PAT analogs (3a, 3b, 3b′).

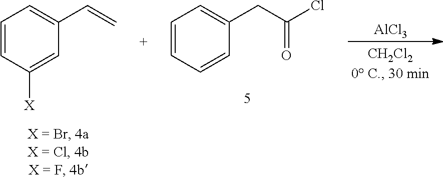

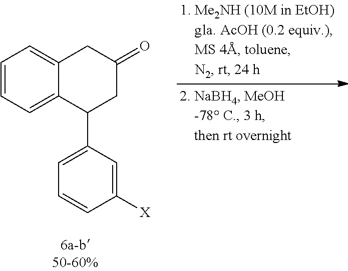

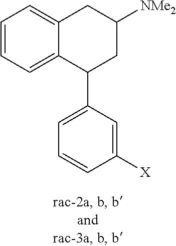

| Analog | X | 3:2 | 3-yield (%) |
|---|---|---|---|
| 3a | Br | 10:1 | 45 |
| 3b | Cl | 7:1 | 40 |
| 3b′ | F | 10:1 | 35 |

Diastereomeric 3′-Br-4-PATs 2a and 3a (Scheme 1, FIG. 1A) served as key intermediates to generate 4-PAT analogs substituted at the meta-position of ring C with a variety of aryl substituents (2c-k, 3c-k, Table 1) via coupling with commercially-available boronic acids (7e-k) or esters (8c, d). Substituted phenyl rings were introduced (rac-2f-k and rac-3f-k, 82-98%, Scheme 3) with excellent yields via Suzuki-Miyaura coupling of respective 3′-Br-4-PAT diastereomers with the corresponding boronic acids (7e-k), in the presence of Pd(II)acetate and SPhos (Scheme 3) (Altman & Buchwald, 2007). Coupling performed with the slow reacting pyridine boronic acid in the presence of Pd₂(dba)₃ and PCy₃ gave analogs 2e and 3e, with moderate yields (55-60%, Scheme 3) (Kudo, et al., 2006). Conventional Suzuki-Miyaura coupling failed to give thiophen-2′-yl or furan-2′-yl analogs (2c, d and 3c, d) due to in situ decomposition of the unstable heterocyclic boronic acids. Pioneering cross-coupling by Burke, with the less nucleophilic ‘bench-top stable’ MIDA ester was used successfully to introduce thiophen-2′-yl and furan-2′-yl fragments into the 3′-Br-4-PATs with ˜60% yield (Scheme 3) (Knapp, et al., 2009).

SCHEME 3
Synthesis of aryl substituted analogs 2c-k and 3c-k.

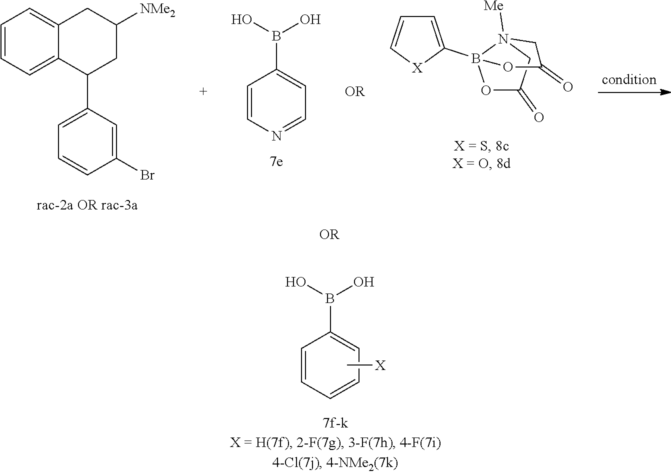

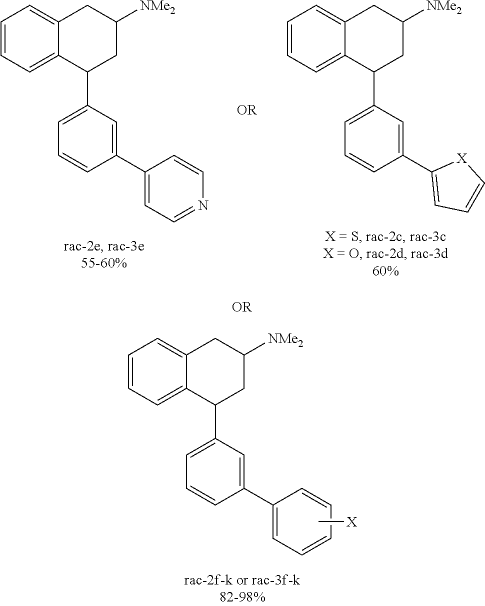

| Analog | condition |
|---|---|
| rac-2e, rac-3e | Pd₂(dba)₃ (5 mol %), PCy₃ (12 mol %) |
| | K₃PO₄ (5 equiv.), dioxane:H₂O(5:1) |
| | 95° C., 24h |
| rac-2c, rac-3c | Pd(OAc)₂ (5 mol %), SPhos (10 mol %) |
| rac-2d, rac-3d | K₃PO₄ (7.5 equiv.), dioxane:H₂O(5:1) |
| | 60° C., 20h |
| rac-2f-k, rac-3f-k | Pd(OAc)₂ (5 mol %), SPhos (15 mol %) |
| | K₃PO₄ (2.0 equiv.), 110°C., toluene, 4h |

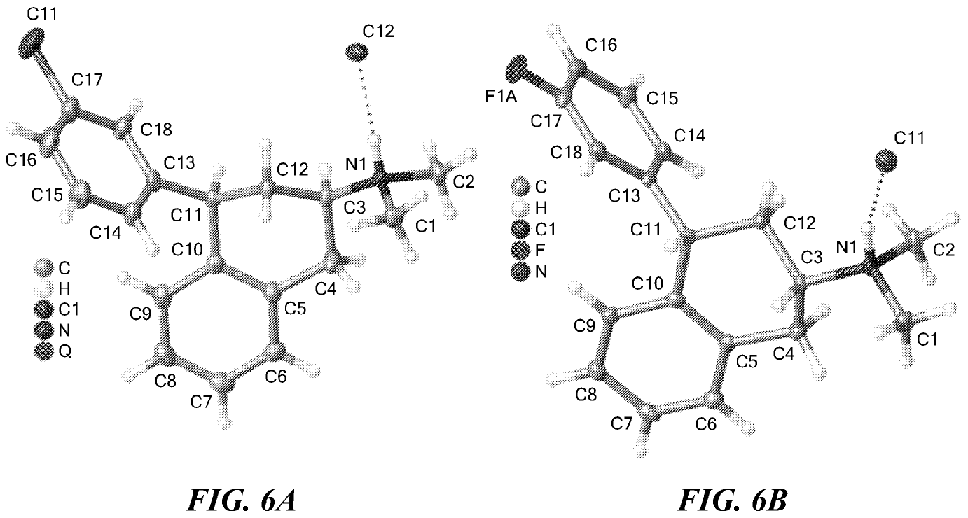

The absolute stereochemistry of optically pure cis-4-PAT isomers (separated by chiral HPLC) was determined using X-ray crystallography on crystals of (2R,4R)-3b and (2S,4S)-3b′ (FIG. 6A, FIG. 6B). The absolute stereochemistry of trans-4-PAT stereoisomers (i.e., [2S,4R], [2R,4S]) has been reported (Booth, Fang, et al., 2009; Sakhuja, et al., 2015).

4-(3-bromophenyl)-3,4-dihydronaphthalen-2(1H)-one 6a: To an oven dried 100 mL round bottom flask with stir bar was added anhydrous AlCl₃ (800 mg, 6.0 mmol) and CH₂Cl₂ (20 ml) under nitrogen atmosphere. The resulting suspension was transferred to an ice bath and cooled for 10 minutes. To the reaction flask was slowly added phenylacetyl chloride 5 (661 μL, 5.0 mmol). The resulting mixture was stirred for 10 minutes under nitrogen atmosphere in an ice bath. To the reaction mixture was then added 3-bromostyrene 4 (664 μL, 5.1 mmol) and the resulting solution was stirred for 30 minutes on ice bath. Water (50 mL) was added to the flask and the organic layer was separated. The aqueous layer was extracted with CH₂Cl₂ (20 mL×3). The combined organic layer was washed with saturated aq NaHCO₃ (30 mL×2) followed by brine (30 mL) and dried over Na₂SO₄. After evaporation of solvent the crude reaction mixture was purified by silica gel column chromatography (97:3 hexanes: ethyl acetate) to afford 4-(3-bromophenyl)-3,4-dihydronaphthalen-2(1H)-one 6a as colorless solid with 60% yield.

Trans-4-(3-bromophenyl)-N, N-dimethyl-1,2,3,4-tetrahydronaphthalen-2-amine 2a and cis-4-(3-bromophenyl)-N, N-dimethyl-1,2,3,4-tetrahydronaphthalen-2-amine 3a: To an oven dried 100 mL round bottom flask with stir bar was added ketone 6 (1.12 g, 3.72 mmol), dimethylamine hydrochloride (3.0 g, 37.2 mmol), Tetrahydrofuran (28 mL) and methanol (37 mL). The resulting mixture was stirred at room temperature under nitrogen atmosphere until all solid dissolved. To the reaction mixture was added sodium cyanoborohydride (1.18 g, 18.8 mmol) and the flask was transferred to an oil bath. The resulting reaction mixture was stirred for 16 h under nitrogen atmosphere at 50° C. The solvent was evaporated and to the residue was added saturated aq NaHCO₃ (50 mL) and ethyl acetate (25 mL). The organic layer was separated, and the aqueous layer was extracted with ethyl acetate (25 mL×4). The combined organic layer was washed with brine (30 mL) and dried over Na₂SO₄. After evaporation of solvent the crude reaction mixture was purified by silica gel column chromatography (4:2:0.1 hexanes:ethyl acetate:triethylamine) to afford cis-3a as colorless oil with 42% yield and trans-2a as colorless oil with 54% isolated yield.

General procedure to synthesize amine 3a, 3b and 3b′: To an oven dried 10 mL round bottom flask with stir bar was added ketone 6 (1.0 mmol), 10M solution of dimethylamine in ethanol (130 μL, 1.3 mmol), 4 Å molecular sieves and toluene (1.0 mL). To the resulting mixture was added glacial acetic acid (11 μL, 0.2 mmol). The reaction mixture was stirred at room temperature for 24 h under nitrogen atmosphere (Scheme 4). In an another over dried flask with stir bar was added sodium borohydride (95 mg, 2.5 mmol) and methanol (3 mL). The flask was cooled to −78° C. under nitrogen atmosphere. To the cooled mixture was added previously formed imine reaction mixture through a filter syringe. Resulting reaction mixture was stirred at −78° C. for 3 h, then the reaction flask was slowly warmed to rt. The reaction mixture was stirred overnight at rt under nitrogen atmosphere. The solvent was evaporated and to the residue was added saturated aq NaHCO₃ (15 mL) and ethyl acetate (10 mL). The organic layer was separated, and the aqueous layer was extracted with ethyl acetate (10 mL×4). The combined organic layer was washed with brine (15 mL) and dried over Na₂SO₄. After evaporation of solvent the crude reaction mixture was purified by silica gel column chromatography to afford cis-amines as colorless oil.

SCHEME 4
Optimized synthesis of Cis-analogues (3a, 3b, 3b′).

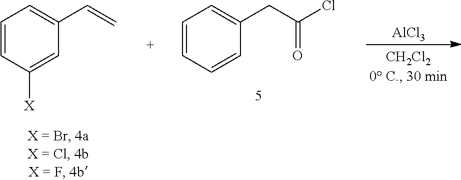

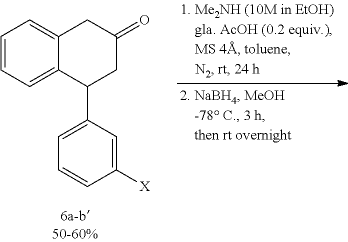

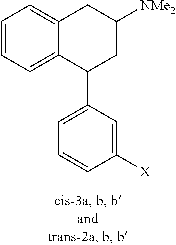

| cis-Analogue | X | cis:trans | cis-yield (%) |
|---|---|---|---|
| 3a | Br | 10:1 | 45 |
| 3b | Cl | 7:1 | 40 |
| 3b′ | F | 10:1 | 35 |

The racemic mixtures of cis-analogs were separated by semi-preparative chiral HPLC Regiscell™ column using conditions and solvents specific to each analog to elute the cis-(2S,4S) and -(2R,4R) enantiomers at retention time t₁ at t₂, respectively. The absolute stereochemistry was assigned by correlating retention time to the x-ray crystal structure of (2R,4R)-cis-3′-CI-4-PAT, 3b and (2S,4S)-cis-3′-F-4-PAT, 3b′ analogs (FIG. 6A, FIG. 6B). Both enantiomers were converted to HCl salts for use in pharmacological assays by adding 2M HCl solution in ether to the solution of free amine in ether.

Cis-4-(3-bromophenyl)-N, N-dimethyl-1,2,3,4-tetrahydronaphthalen-2-amine, 3a: amine 3a was synthesized from ketone 6a following the general procedure as described above. The crude reaction mixture (cis:trans 10:1) was purified by silica gel column chromatography (4:2:0.1 hexanes: ethyl acetate: triethylamine) to afford racemic cis-4-(3-bromophenyl)-N, N-dimethyl-1,2,3,4-tetrahydronaphthalen-2-amine 3a as colorless oil with 45% isolated yield. ¹H-NMR (500 MHz; CDCl₃): δ 7.38 (d, J=7.9 Hz, 1H), 7.33 (s, 1H), 7.19 (t, J=7.8 Hz, 1H), 7.15-7.10 (m, 3H), 7.03 (t, J=7.3 Hz, 1H), 6.73 (d, J=7.8 Hz, 1H), 4.07 (dd, J=12.2, 5.2 Hz, 1H), 3.04-3.00 (m, 1H), 2.95-2.90 (m, 1H), 2.80 (tdd, J=11.5, 4.7, 2.3 Hz, 1H), 2.37 (s, 6H), 2.33 (ddd, J=9.9, 5.2, 2.5 Hz, 1H), 1.68 (q, J=12.1 Hz, 1H). ¹³C-NMR (100 MHz, CDCl3): δ 149.14, 138.64, 136.49, 131.86, 130.29, 129.68, 129.63, 129.28, 127.57, 126.46, 126.17, 122.74, 60.56, 47.10, 41.60, 36.93, 33.08.

HCl salt ¹H-NMR (500 MHz; CDCl₃): δ 13.01 (s, 1H), 7.42 (d, J=8.0 Hz, 1H), 7.31 (s, 1H), 7.23-7.17 (m, 3H), 7.13-7.10 (m, 2H), 6.77 (d, J=7.8 Hz, 1H), 4.19 (dd, J=12.1, 5.2 Hz, 1H), 3.65-3.59 (m, 1H), 3.43-3.39 (m, 1H), 3.31-3.26 (m, J=13.6 Hz, 1H), 2.85-2.84 (m, 6H), 2.69 (dt, J=12.2, 2.6 Hz, 1H), 2.00 (q, J=12.2 Hz, 1H). ¹³C-NMR (100 MHz, CDCl₃): δ 146.64, 137.05, 132.01, 131.67, 130.58, 130.47, 129.52, 129.38, 127.57, 127.42, 127.33, 122.95, 61.76, 45.82, 39.76, 39.44, 34.24, 30.19. Calculated C1₈H₂₁BrN for [M+H]⁺: 330.0858. Found: 330.0859. HPLC (s-prep): Solvent System: hexanes:MeOH:ⁱPrOH (85:10:5) 0.1% TEA (modifier), 0.1% TFA (modifier), flow rate=1.5 mL/min; t₁=14.41 min, t₂=19.71 min.

4-(3-chlorophenyl)-3,4-dihydronaphthalen-2(1H)-one 6b: Ketone 6b was synthesized from 3-chlorostyrene 4b and phenylacetyl chloride 5 in presence of AlCl₃ following the procedure as described above for 6a. The crude reaction mixture was purified by silica gel column chromatography (95:5 hexanes: ethyl acetate) to afford 4-(3-chlorophenyl)-3,4-dihydronaphthalen-2(1)-one 6b as colorless oil with 60% isolated yield. ¹H and ¹³C NMRs are in agreement with previously published data (Vincek & Booth, 2009).

Cis-4-(3-chlorophenyl)-N, N-dimethyl-1,2,3,4-tetrahydronaphthalen-2-amine, 3b: amine 3b was synthesized from ketone 6b following the general procedure as described above. The crude reaction mixture (cis:trans 7:1) was purified by silica gel column chromatography (4:2:0.1 hexanes:ethyl acetate:triethylamine) to afford racemic cis-4-(3-chlorophenyl)-N, N-dimethyl-1,2,3,4-tetrahydronaphthalen-2-amine 6b as colorless oil with 40% isolated yield. ¹H-NMR (500 MHz; CDCl₃): δ 7.25-7.21 (m, 2H), 7.17-7.12 (m, 3H), 7.07 (d, J=7.1 Hz, 1H), 7.03 (t, J=7.2 Hz, 1H), 6.73 (d, J=7.8 Hz, 1H), 4.08 (dd, J=12.2, 5.1 Hz, 1H), 3.03 (ddd, J=15.6, 4.5, 1.9 Hz, 1H), 2.93 (dd, J=12.4, 11.4 Hz, 1H), 2.81 (tdd, J=11.5, 4.6, 2.3 Hz, 1H), 2.38 (s, 6H), 2.35-2.31 (m, J=2.6 Hz, 1H), 1.69 (q, J=12.1 Hz, 1H). ¹³C-NMR (100 MHz, CDCl₃): δ 148.80, 138.62, 136.43, 134.42, 129.93, 129.60, 129.24, 128.92, 127.07, 126.72, 126.43, 126.13, 60.54, 47.08, 41.55, 36.87, 33.02.

HCl salt ¹H-NMR (500 MHz; CDCl₃): δ 12.88 (s, 1H), 7.29-7.27 (m, 2H), 7.21-7.15 (m, 3H), 7.12-7.07 (m, 2H), 6.77 (d, J=7.7 Hz, 1H), 4.21 (dd, J=12.0, 5.1 Hz, 1H), 3.64 (td, J=11.0, 1.9 Hz, 1H), 3.42-3.39 (m, 1H), 3.31-3.26 (m, 1H), 2.85 (s, 6H), 2.70-2.67 (m, 1H), 2.00 (q, J=12.2 Hz, 1H). ¹³C-NMR (100 MHz, CDCl₃): δ 146.36, 137.06, 134.67, 132.03, 130.24, 129.48, 129.33, 128.76, 127.48, 127.34, 127.26, 127.08, 61.72, 45.79, 39.75, 39.43, 34.16, 30.19. Calculated C₁₈H₂₁ClN for [M+H]⁺: 286.1363. Found: 286.1359. HPLC (s-prep): Solvent System: hexanes:MeOH:ⁱPrOH:ⁿPrOH (80:10:5:5) 0.1% TEA (modifier) 0.1% TFA (modifier); flow rate=1.5 mL/min; t₁=11.0 min, t₂=13.0 min.

4-(3-fluorophenyl)-3,4-dihydronaphthalen-2(1H)-one 6b′: Ketone 6b′ was synthesized from 3-fluorostyrene 4b′ and phenylacetyl chloride 5 in presence of AlCl₃ following the procedure as described above for 6a. The crude reaction mixture was purified by silica gel column chromatography (95:5 hexanes: ethyl acetate) to afford 4-(3-fluorophenyl)-3,4-dihydronaphthalen-2(1)-one 6b′ as colorless oil with 50% isolated yield. ¹H and ¹³C NMRs are in agreement with previously published data (Vincek & Booth, 2009).

Cis-4-(3-fluorophenyl)-N, N-dimethyl-1,2,3,4-tetrahydronaphthalen-2-amine, 3b′: amine 3b′ was synthesized from ketone 6b′ following the general procedure as described above. The crude reaction mixture (cis:trans 10:1) was purified by silica gel column chromatography (4:2:0.1 hexanes:ethyl acetate:triethylamine) to afford racemic cis-4-(3-fluorophenyl)-N, N-dimethyl-1,2,3,4-tetrahydronaphthalen-2-amine 6b′ as colorless oil with 35% isolated yield. ¹H-NMR (500 MHz; CDCl₃): 7.26-7.23 (m, 1H), 7.15-7.12 (m, 2H), 7.08-7.04 (m, 1H), 6.95-6.91 (m, 2H), 6.80 (d, J=9.3 Hz, 1H), 6.71 (d, J=7.7 Hz, 1H), 4.09 (dd, J=12.2, 5.2 Hz, 1H), 3.06-3.02 (m, 1H), 2.94 (dd, J=12.4, 11.2 Hz, 1H), 2.79-2.74 (m, 1H), 2.39 (s, 6H), 2.34-2.31 (m, 1H), 1.69 (q, J=12.2 Hz, 1H). ¹³C-NMR (100 MHz, CDCl₃): δ 164.18, 166.66, 148.95, 148.88, 138.71, 133.94, 130.47, 130.39, 129.35, 129.04, 126.71, 126.95, 124.07, 114.75, 114.53, 113.42, 113.21, 60.75, 47.92, 41.53, 36.53, 33.19.

HCl salt ¹H-NMR (500 MHz; CDCl₃): δ 12.70 (brs, 1H), 7.31-7.26 (m, 1H), 7.18-7.15 (m, 2H), 7.10-7.06 (m, 1H), 6.98-6.94 (m, 2H), 6.84 (d, J=9.4 Hz, 1H), 6.75 (d, J=7.7 Hz, 1H), 4.22 (br d, J=9.1 Hz, 1H), 3.68-3.60 (m, 1H), 3.38 (d, J=14.0 Hz, 1H), 3.29-3.24 (m, 1H), 2.84 (s, 6H), 2.67 (d, J=9.5 Hz, 1H), 2.03-1.96 (m, 1H). ¹³C-NMR (100 MHz, CDCl₃): δ 164.39, 161.93, 146.82, 146.75, 137.16, 131.95, 130.58, 130.50, 129.57, 129.42, 127.41, 127.35, 124.67, 115.77, 115.55, 114.44, 114.23, 61.98, 45.99, 39.81, 39.47, 34.33, 30.37. Calculated C1₈H₂₁FN for [M+H]⁺: 270.1659. Found: 270.1655. HPLC (s-prep): Solvent System: hexanes:MeOH:ⁱPrOH:ⁿPrOH (80:10:5:5) 0.1% TEA (modifier) flow rate=1.5 mL/min; t₁=11.77 min, t₂=13.14 min.

Racemic 2f-k were prepared. To an oven dried 25 mL round bottom flask with stir bar was added racemic trans-3′Br-4-PAT 2a (66 mg, 0.2 mmol), Aryl boronic acid (0.3 mmol) and toluene (1.5 mL). The resulting mixture was degassed by sparging with N₂ gas for 45 minutes. To the reaction mixture was added potassium phosphate (85 mg, 0.4 mmol), Palladium(II) acetate (2 mg, 0.01 mmol) and SPhos (12 mg, 0.03 mmol). The flask was fitted with a reflux condenser and the reaction mixture was heated to 110° C. for 4 h under nitrogen atmosphere (Scheme 5). The reaction was quenched with 1N NaOH aq (3 mL) and ethyl acetate (4 mL). The organic layer was separated, and the aqueous layer was extracted with ethyl acetate (5 mL×4). The combined organic layer was washed with brine (10 mL) and dried over Na₂SO₄. After evaporation of solvent the crude reaction mixture was purified by silica gel column chromatography (4:2:0.1 hexanes:ethyl acetate:triethylamine) to afford racemic 2f-k.

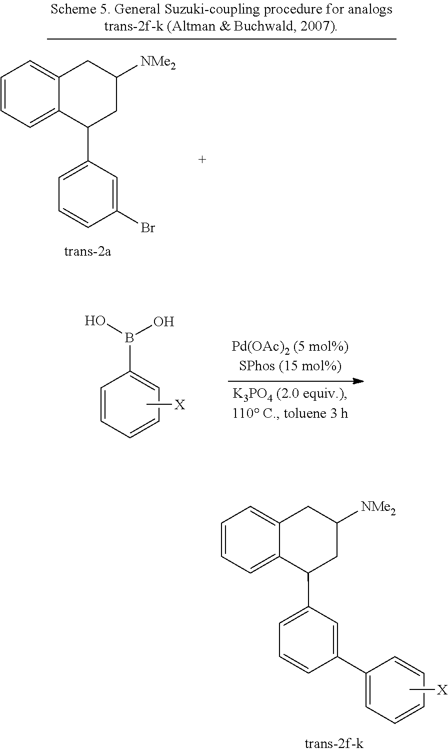

The racemic mixtures of trans-analogs were separated by semi-preparative chiral HPLC Regiscell column using conditions and solvents specific to each analog to elute the trans-(2R,4S) and -(2S,4R) enantiomers at retention time t₁ at t₂, respectively, with absolute stereochemistry assigned according to retention time of the previously published trans-3′-CI-4-PAT analog (Sakhuja, et al., 2015). Both enantiomers were converted to HCl salts for use in pharmacological assays by adding 2M HCl solution in ether to the solution of free amine in ether.

Trans-4-([1,1′-biphenyl]-3-yl)-N,N-dimethyl-1,2,3,4-tetrahydronaphthalen-2-amine, 2f: trans-amine 2f was synthesized from trans-3′Br-4-PAT 2a (66 mg, 0.2 mmol), phenylboronic acid (37 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic trans-amine 2f as colorless oil with 82% isolated yield.

¹H-NMR (500 MHz; CDCl₃): δ 7.52 (d, J=7.3 Hz, 2H), 7.41 (dd, J=7.5, 7.5 Hz, 3H), 7.32 (dd, J=7.7, 7.7 Hz, 2H), 7.27 (s, 1H), 7.20-7.16 (m, 2H), 7.11 (t, J=6.9 Hz, 1H), 6.98 (dd, J=14.5, 7.6 Hz, 2H), 4.44 (t, J=5.1 Hz, 1H), 3.07 (dd, J=16.2, 4.7 Hz, 1H), 2.90 (dd, J=16.1, 9.5 Hz, 1H), 2.75 (tt, J=9.1, 4.5 Hz, 1H), 2.32 (s, 6H), 2.23-2.15 (m, 2H). ¹³C-NMR (100 MHz, CDCl₃): δ 147.27, 141.37, 141.25, 137.79, 136.14, 130.15, 129.49, 128.83, 128.72, 127.81, 127.62, 127.34, 127.32, 126.57, 126.29, 125.12, 56.64, 44.20, 41.83, 34.83, 32.25. ¹H and ¹³C NMRs are in agreement with previously published data (Sakhuja, et al., 2015).

Trans-4-(2′-fluoro-[1,1′-biphenyl]-3-yl)-N, N-dimethyl-1,2,3,4-tetrahydronaphtha-len-2-amine, 2g: trans-amine 2g was synthesized from trans-3′Br-4-PAT 2a (66 mg, 0.2 mmol), 2-fluorophenylboronic acid (42 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic trans-amine 2g as colorless oil with 98% isolated yield.

¹H-NMR (500 MHz; CDCl₃): δ 7.39-7.36 (m, 2H), 7.32 (t, J=7.7 Hz, 1H), 7.29-7.24 (m, 2H), 7.18-7.15 (m, 3H), 7.13-7.09 (m, 2H), 6.99 (d, J=7.5 Hz, 2H), 4.43 (t, J=5.2 Hz, 1H), 3.05 (dd, J=16.2, 4.8 Hz, 1H), 2.88 (dd, J=16.2, 9.5 Hz, 1H), 2.71 (tt, J=9.1, 4.5 Hz, 1H), 2.28 (s, 6H), 2.21-2.13 (m, 2H). ¹³C-NMR (100 MHz, CDCl₃): δ 161.10, 158.63, 147.08, 137.91, 136.45, 135.75, 130.91, 130.87, 130.15, 129.54, 129.51, 129.48, 129.35, 129.21, 129.01, 128.93, 128.35, 128.19, 126.88, 126.85, 126.48, 126.18, 124.40, 124.37, 116.29, 116.06, 56.44, 44.22, 41.97, 35.04, 32.40.

HCl salt ¹H-NMR (500 MHz; CDCl₃): δ 12.65 (br s, 1H), 7.39-7.30 (m, 4H), 7.26-7.25 (m, 2H), 7.22-7.18 (m, 3H), 7.15-7.10 (m, 1H), 7.07 (d, J=7.5 Hz, 1H), 6.88 (d, J=6.9 Hz, 1H), 4.59 (br s, 1H), 3.53 (br d, J=13.7 Hz, 1H), 3.42 (br s, 1H), 3.29-3.24 (m, 1H), 2.73 (s, 6H), 2.48 (br s, 2H). ¹³C-NMR (100 MHz, CDCl₃): δ 160.90, 158.44, 144.62, 136.15, 135.39, 132.76, 130.73, 130.70, 130.14, 129.47, 129.30, 129.22, 128.97, 128.94, 128.88, 128.66, 128.53, 127.69, 127.57, 127.55, 127.45, 127.25, 124.55, 124.51, 116.23, 116.00, 58.75, 43.61, 40.99, 38.93, 31.57, 30.73. Calculated C₂₄H₂₅FN for [M+H]⁺: 346.1971. Found: 346.1969. HPLC (s-prep): Solvent System: hexanes:MeOH:ⁱPrOH (85:10:5) 0.1% TEA (modifier), 0.1% TFA (modifier) flow rate=3.0 mL/min; t, =8.0 min, t₂=17.65 min.

Trans-4-(3′-fluoro-[1,1′-biphenyl]-3-yl)-N,N-dimethyl-1,2,3,4-tetrahydronaphtha-len-2-amine, 2h: trans-amine 2h was synthesized from trans-3′Br-4-PAT 2a (66 mg, 0.2 mmol), 3-fluorophenylboronic acid (42 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic trans-amine 2h as colorless oil with 98% isolated yield.

¹H-NMR (500 MHz; CDCl₃): δ 7.39-7.37 (m, 1H), 7.35-7.29 (m, 3H), 7.27 (s, 1H), 7.23-7.16 (m, 3H), 7.12-7.09 (m, 1H), 7.03-6.96 (m, 3H), 4.43 (t, J=5.3 Hz, 1H), 3.04 (dd, J=16.3, 4.8 Hz, 1H), 2.88 (dd, J=16.2, 9.3 Hz, 1H), 2.70-2.64 (m, 1H), 2.28 (s, 6H), 2.17 (t, J=6.1 Hz, 2H). ¹³C-NMR (100 MHz, CDCl₃): δ 164.46, 162.01, 147.75, 143.75, 143.67, 139.95, 139.93, 137.92, 136.54, 130.28, 130.19, 130.06, 129.52, 128.79, 128.45, 127.55, 126.51, 126.18, 124.97, 122.91, 122.89, 114.25, 114.16, 114.03, 113.95, 56.45, 44.23, 42.06, 35.28, 32.39. HCl salt ¹H-NMR (500 MHz; CDCl₃): δ 12.75 (s, 1H), 7.43 (d, J=7.6 Hz, 1H), 7.43-7.34 (m, 2H), 7.29-7.26 (m, 3H), 7.23-7.17 (m, 3H), 7.06-7.02 (m, 2H), 6.89 (d, J=7.6 Hz, 1H), 4.60 (br s, 1H), 3.53 (dd, J=15.5, 4.2 Hz, 1H), 3.41-3.40 (m, 1H), 3.24 (dd, J=14.9, 11.7 Hz, 1H), 2.72 (br s, 6H), 2.49-2.44 (m, 2H). ¹³C-NMR (100 MHz, CDCl₃): δ 164.32, 161.88, 145.18, 143.00, 142.92, 140.35, 140.33, 135.40, 132.70, 130.37, 130.29, 130.06, 129.45, 129.24, 127.87, 127.48, 127.27, 126.97, 125.73, 122.86, 122.83, 114.36, 114.15, 114.11, 113.89, 58.63, 43.60, 40.67, 38.98, 31.53, 30.45. Calculated C₂₄H₂₅FN for [M+H]⁺: 346.1971. Found: 346.1969. HPLC (s-prep): Solvent System: hexanes:MeOH:ⁱPrOH (85:10:5) 0.1% TEA (modifier), 0.1% TFA (modifier) flow rate=3.0 mL/min; t₁=8.23 min, t₂=13.42 min.

Trans-4-(4′-fluoro-[1,1′-biphenyl]-3-yl)-N,N-dimethyl-1,2,3,4-tetrahydronaphtha-len-2-amine, 2i: trans-amine 2i was synthesized from trans-3′Br-4-PAT 2a (66 mg, 0.2 mmol), 4-fluorophenylboronic acid (42 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic trans-amine 2i as colorless oil with 92% isolated yield.

¹H-NMR (500 MHz; CDCl₃): δ 7.47 (td, J=6.0, 2.7 Hz, 2H), 7.35 (d, J=7.8 Hz, 1H), 7.31 (dd, J=7.6, 7.6 Hz, 1H), 7.23 (br s, 1H), 7.20-7.16 (m, 2H), 7.12-7.07 (m, 3H), 6.97 (t, J=7.6 Hz, 2H), 4.43 (t, J=5.3 Hz, 1H), 3.06 (dd, J=16.2, 4.8 Hz, 1H), 2.89 (dd, J=16.2, 9.4 Hz, 1H), 2.74-2.69 (m, 1H), 2.31 (s, 6H), 2.20-2.17 (m, 2H). ¹³C-NMR (100 MHz, CDCl₃): δ 163.76, 161.30, 147.66, 140.24, 138.02, 137.55, 137.52, 136.55, 130.09, 129.51, 128.86, 128.79, 128.73, 127.88, 127.50, 126.49, 126.17, 124.89, 115.76, 115.55, 56.47, 44.26, 42.05, 35.27, 32.38.

HCl salt ¹H-NMR (500 MHz; CDCl₃): δ 12.76 (s, 1H), 7.46 (dd, J=8.6, 5.3 Hz, 2H), 7.40 (d, J=7.7 Hz, 1H), 7.34 (t, J=7.6 Hz, 1H), 7.29-7.20 (m, 3H), 7.14-7.09 (m, 3H), 7.06 (d, J=7.6 Hz, 1H), 6.86 (d, J=7.5 Hz, 1H), 4.60 (br s, 1H), 3.52 (dd, J=15.5, 3.1 Hz, 1H), 3.45-3.38 (m, 1H), 3.24 (dd, J=14.4, 12.0, 1H), 2.72 (br s, 6H), 2.52-2.44 (m, 2H). ¹³C-NMR (100 MHz, CDCl₃): δ 163.84, 161.38, 145.12, 140.75, 136.89, 136.85, 135.50, 132.72, 130.17, 129.52, 129.23, 128.87, 128.79, 127.54, 127.33, 126.98, 125.72, 115.85, 115.64, 58.70, 43.70, 40.75, 39.11, 31.63, 30.49. Calculated C₂₄H₂₅FN for [M+H]⁺: 346.1971. Found: 346.1968. HPLC (s-prep): Solvent System: hexanes:MeOH:ⁱPrOH (85:10:5) 0.1% TEA (modifier), 0.1% TFA (modifier) flow rate=3.0 mL/min; t, =9.96 min, t₂=16.64 min.

Trans-4-(4′-chloro-[1,1′-biphenyl]-3-yl)-N,N-dimethyl-1,2,3,4-tetrahydronaphtha-len-2-amine, 2j: trans-amine 2j was synthesized from trans-3′Br-4-PAT 2a (66 mg, 0.2 mmol), 4-chlorophenylboronic acid (47 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic trans-amine 2j as colorless oil with 97% isolated yield.

¹H-NMR (500 MHz; CDCl₃): δ 7.45 (d, J=8.5 Hz, 2H), 7.37 (d, J=8.6 Hz, 3H), 7.31 (t, J=7.6 Hz, 1H), 7.24 (s, 1H), 7.26-7.20 (m, 2H), 7.10 (t, J=7.1 Hz, 1H), 6.97 (t, J=7.9 Hz, 2H), 4.42 (t, J=5.3 Hz, 1H), 3.04 (dd, J=16.3, 4.8 Hz, 1H), 2.87 (dd, J=16.2, 9.3 Hz, 1H), 2.68-2.65 (m, 1H), 2.28 (s, 6H), 2.17 (t, J=6.0 Hz, 2H).

¹³C-NMR (100 MHz, CDCl₃): δ 147.74, 139.98, 139.86, 137.95, 136.54, 133.40, 130.08, 129.52, 128.96, 128.80, 128.54, 128.20, 127.46, 126.51, 126.18, 124.86, 56.44, 44.24, 42.06, 35.29, 32.34.

HCl salt ¹H-NMR (500 MHz; CDCl₃): δ 12.77 (br s, 1H), 7.44-7.38 (m, 5H), 7.34 (t, J=7.6 Hz, 1H), 7.29-7.26 (m, 2H), 7.25-7.20 (m, 1H), 7.15 (s, 1H), 7.05 (d, J=7.5 Hz, 1H), 6.87 (d, J=7.5 Hz, 1H), 4.60 (br s, 1H), 3.51 (dd, J=15.4, 4.5 Hz, 1H), 3.47-3.43 (m, 1H), 3.23 (dd, J=15.0, 11.7 Hz, 1H), 2.71 (d, J=4.6 Hz, 6H), 2.52-2.43 (m, 2H). ¹³C-NMR (100 MHz, CDCl₃): δ 145.19, 140.48, 139.19, 135.45, 133.67, 132.70, 130.15, 129.51, 129.28, 129.00, 128.49, 127.63, 127.54, 127.33, 126.93, 125.67, 58.65, 43.66, 40.57, 39.04, 31.59, 30.40. Calculated C₂₄H₂₅ClN for [M+H]⁺: 362.1676. Found: 362.1673. HPLC (s-prep): Solvent System: hexanes: ⁱPrOH (98:2) 0.1% TEA (modifier), flow rate=2.0 mL/min; t, =12.42 min, t₂=14.52 min.

Trans-4-(4′-(dimethylamino)-[1,1′-biphenyl]-3-yl)-N,N-dimethyl-1,2,3,4-tetra-hydronaph-thalen-2-amine, 2k: trans-amine 2k was synthesized from trans-3′Br-4-PAT 2a (66 mg, 0.2 mmol), 4-(dimethylamino)phenylboronic acid (50 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic trans-amine 2k as colorless oil with 90% isolated yield.

¹H-NMR (500 MHz; CDCl₃): δ 7.43 (d, J=8.7 Hz, 2H), 7.37 (d, J=7.7 Hz, 1H), 7.28-7.23 (m, 2H), 7.18-7.14 (m, 2H), 7.09 (t, J=7.1 Hz, 1H), 6.99 (d, J=7.6 Hz, 1H), 6.88 (d, J=7.6 Hz, 1H), 6.77 (d, J=8.7 Hz, 2H), 4.40 (t, J=5.1 Hz, 1H), 3.04 (dd, J=16.2, 4.7 Hz, 1H), 2.97 (s, 6H), 2.86 (dd, J=16.2, 9.5 Hz, 1H), 2.71-2.66 (m, 1H), 2.28 (s, 6H), 2.20-2.11 (m, 2H). ¹³C-NMR (100 MHz, CDCl₃): δ 150.07, 147.28, 141.14, 138.26, 136.51, 130.16, 129.48, 129.39, 128.53, 127.85, 126.89, 126.61, 126.36, 126.11, 124.14, 112.87, 56.51, 44.30, 42.07, 40.72, 35.06, 32.60.

HCl salt ¹H-NMR (500 MHz; CDCl₃): δ 12.67 (s, 1H), 7.84 (d, J=8.2 Hz, 2H), 7.66 (d, J=8.1 Hz, 2H), 7.39 (dt, J=15.2, 7.6 Hz, 2H), 7.31-7.22 (m, 3H), 7.15 (s, 1H), 7.07 (d, J=7.5 Hz, 1H), 6.94 (d, J=7.3 Hz, 1H), 4.62 (br s, 1H), 3.50-3.43 (m, 2H), 3.21-3.11 (m, 7H), 2.73 (dd, J=8.0, 4.8 Hz, 6H), 2.62 (br d, J=12.0 Hz, 1H), 2.44 (td, J=11.9, 5.3 Hz, 1H). ¹³C-NMR (100 MHz, CDCl₃): δ 164.93, 145.28, 139.70, 135.38, 132.64, 130.14, 129.54, 129.36, 129.36, 129.11, 128.11, 127.57, 127.31, 127.17, 125.73, 120.66, 58.51, 46.19, 45.97, 43.54, 40.14, 39.29, 31.60, 30.00. Calculated C2₆H3₁N₂ for [M+H]*: 371.2487. Found: 371.2483. HPLC (s-prep): Solvent System: hexanes:MeOH:ⁱPrOH:ⁿPrOH (80:10:5:5) 0.1% TEA (modifier), flow rate=1.7 mL/min; t, =11.55 min, t₂=12.93 min.

The cis analogues 3f-3k (Scheme 6) were synthesized from racemic cis-3′Br-4-PAT 3a and corresponding Aryl boronic acid following the general procedure as described above for trans 2f-k. The racemic mixtures of cis-analogs were separated by semi-preparative chiral HPLC Regiscell column using conditions and solvents specific to each analog to elute the cis-(2S,4S) and -(2R,4R) enantiomers at retention time t₁ at t₂, respectively. The absolute stereochemistry was assigned by correlating retention time to the x-ray crystal structure of (2R,4R)-cis-3′CI-4-PAT, 3b and (2S,4S)-cis-3′F-4-PAT, 3b′ analogs. Both enantiomers were converted to HCl salts for use in pharmacological assays by adding 2M HCl solution in ether to the solution of free amine in ether.

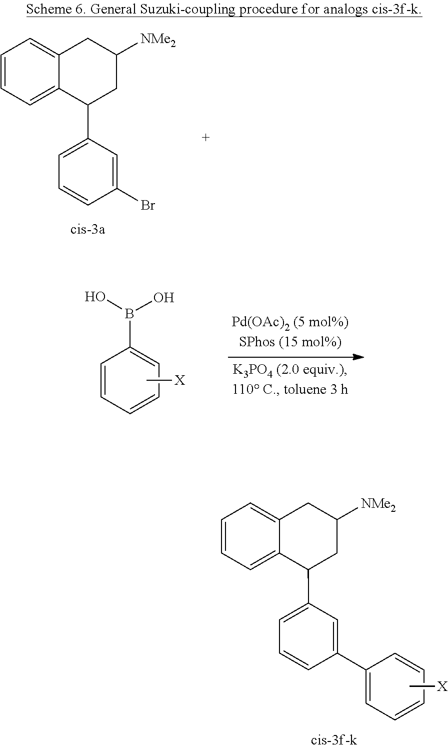

Cis-4-([1,1′-biphenyl]-3-yl)-N,N-dimethyl-1,2,3,4-tetrahydronaphthalen-2-amine, 3f: cis-amine 3f was synthesized from cis-3′Br-4-PAT 3a (66 mg, 0.2 mmol), phenylboronic acid (37 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic cis-amine 3f as colorless oil with 85% isolated yield.

¹H-NMR (500 MHz; CDCl₃): δ 7.58-7.56 (m, 2H), 7.48 (d, J=7.7 Hz, 1H), 7.47-7.37 (m, 4H), 7.37-7.30 (m, 1H), 7.16 (d, J=8.2 Hz, 2H), 7.12 (t, J=7.3 Hz, 1H), 7.02 (t, J=7.4 Hz, 1H), 6.82 (d, J=7.8 Hz, 1H), 4.16 (dd, J=12.2, 5.1 Hz, 1H), 3.05 (dd, J=15.6, 2.9 Hz, 1H), 2.96 (dd, J=15.6, 11.3 Hz, 1H), 2.84 (tdd, J=11.4, 4.8, 2.3 Hz, 1H), 2.42-2.38 (m, 7H), 1.79 (q, J=12.1 Hz, 1H). ¹³C-NMR (100 MHz, CDCl₃): δ 147.21, 141.63, 141.29, 139.35, 136.39, 129.53, 129.40, 129.12, 128.83, 127.84, 127.77, 127.37, 127.33, 126.26, 126.07, 125.41, 60.72, 47.45, 41.60, 36.99, 33.18.

HCl salt ¹H-NMR (500 MHz; CDCl₃): δ 12.93 (s, 1H), 7.55 (d, J=7.4 Hz, 2H), 7.52 (d, J=7.6 Hz, 1H), 7.44-7.35 (m, 4H), 7.36-7.33 (m, 1H), 7.20-7.19 (m, 2H), 7.16-7.10 (m, 2H), 6.86 (d, J=7.8 Hz, 1H), 4.28 (d, J=9.2 Hz, 1H), 3.70-3.63 (m, 1H), 3.47-3.44 (m, 1H), 3.34-3.29 (m, 1H), 2.86 (s, 6H), 2.73-2.71 (m, 1H), 2.08 (q, J=11.8 Hz, 1H). ¹³C-NMR (100 MHz, CDCl₃): δ 144.83, 142.00, 140.90, 137.77, 132.03, 129.52, 129.44, 128.89, 127.71, 127.60, 127.58, 127.30, 127.14, 126.15, 62.02, 46.31, 39.86, 39.67, 34.29, 30.45. Calculated C₂₄H₂₆N for [M+H]*: 328.2066. Found: 328.2059. HPLC (s-prep): Solvent System: hexanes:MeOH:ⁱPrOH (90:5:5) 0.1% TEA (modifier), 0.1% TFA (modifier), flow rate=3.0 mL/min; t, =10.17 min, t2=24.59 min.

Cis-4-(2′-fluoro-[1,1′-biphenyl]-3-yl)-N,N-dimethyl-1,2,3,4-tetrahydronaphthalen-2-amine, 3g: cis-amine 3g was synthesized from cis-3′Br-4-PAT 3a (66 mg, 0.2 mmol), 2-fluorophenylboronic acid (42 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic cis-amine 3g as colorless oil with 96% isolated yield.

¹H-NMR (500 MHz; CDCl₃): δ 7.45-7.38 (m, 4H), 7.31-7.27 (m, 1H), 7.19-7.11 (m, 5H), 7.03 (t, J=7.3 Hz, 1H), 6.83 (d, J=7.8 Hz, 1H), 4.16 (dd, J=12.2, 5.0 Hz, 1H), 3.04 (dd, J=15.5, 2.6 Hz, 1H), 2.95 (dd, J=15.5, 11.4 Hz, 1H), 2.83 (tdd, J=11.5, 4.7, 2.2 Hz, 1H), 2.42-2.38 (m, 7H), 1.78 (q, J=12.1 Hz, 1H). ¹³C NMR (100 MHz; CDCl₃): δ 161.10, 158.60, 146.9, 139.30, 136.40, 136.10, 130.95, 130.91, 129.65, 129.62, 129.52, 129.42, 129.09, 129.00, 128.80, 128.20, 127.28, 127.25, 126.30, 126.10, 124.43, 124.39, 116.30, 116.10, 60.70, 47.40, 41.60, 37.00, 33.20.

HCl salt: ¹H-NMR (500 MHz; CDCl₃): δ 12.69 (s, 1H), 7.46 (d, J=7.5 Hz, 1H), 7.41 (t, J=7.1 Hz, 2H), 7.35 (s, 1H), 7.30 (dd, J=14.9, 9.0 Hz, 1H), 7.21-7.08 (m, 6H), 6.85 (d, J=7.7 Hz, 1H), 4.27 (br d, J=8.3 Hz, 1H), 3.70-3.65 (m, 1H), 3.43 (br d, J=14.3 Hz, 1H), 3.32-3.27 (m, 1H), 2.85 (d, J=11.5 Hz, 7H), 2.70 (d, J=9.8 Hz, 1H), 2.07 (q, J=11.8 Hz, 1H). ¹³C-NMR (100 MHz, CDCl₃): δ 160.92, 158.45, 144.49, 137.65, 136.42, 132.03, 130.84, 130.81, 129.46, 129.42, 129.39, 129.27, 129.19, 129.04, 128.04, 127.95, 127.92, 127.21, 127.08, 124.49, 124.46, 116.24, 116.02, 61.89, 46.12, 39.78, 39.48, 34.15, 30.40. Calculated C₂₄H₂₅FN for [M+H]*: 346.1971. Found: 346.1968. HPLC (s-prep): Solvent System: hexanes:ⁱPrOH (94:6) 0.2% TEA (modifier), 0.1% TFA (modifier), flow rate=3.0 mL/min; t, =13.11 min, t₂=29.15 min.

Cis-4-(3′-fluoro-[1,1′-biphenyl]-3-yl)-N,N-dimethyl-1,2,3,4-tetrahydronaphthalen-2-amine, 3h: cis-amine 3h was synthesized from cis-3′Br-4-PAT 3a (66 mg, 0.2 mmol), 3-fluorophenylboronic acid (42 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic cis-amine 3h as colorless oil with 97% isolated yield.

¹H-NMR (500 MHz; CDCl₃): δ 7.46 (d, J=7.7 Hz, 1H), 7.41-7.34 (m, 4H), 7.28-7.26 (m, 1H), 7.20-7.12 (m, 3H), 7.04-7.01 (m, 2H), 6.80 (d, J=7.8 Hz, 1H), 4.17 (dd, J=12.2, 5.1 Hz, 1H), 3.06 (dd, J=15.6, 2.6 Hz, 1H), 2.97 (dd, J=15.6, 11.4 Hz, 1H), 2.88-2.83 (m, 1H), 2.41-2.39 (m, 7H), 1.78 (q, J=12.1 Hz, 1H). ¹³C-NMR (100 MHz, CDCl3): δ 164.49, 162.04, 147.45, 143.62, 143.55, 140.37, 140.35, 139.22, 136.46, 130.31, 130.22, 129.58, 129.34, 129.25, 128.45, 127.65, 126.32, 126.08, 125.35, 122.95, 122.92, 114.29, 114.24, 114.07, 114.03, 60.71, 47.45, 41.63, 37.08, 33.20.

HCl salt ¹H-NMR (500 MHz; CDCl₃): δ 13.00 (s, 1H), 7.49 (d, J=7.7 Hz, 1H), 7.44-7.32 (m, J=8.0 Hz, 4H), 7.23 (s, 1H), 7.20-7.19 (m, 3H), 7.13-7.09 (m, 1H), 7.04 (td, J=8.2, 1.1 Hz, 1H), 6.84 (d, J=7.8 Hz, 1H), 4.28 (dd, J=11.8, 4.5 Hz, 1H), 3.69-3.65 (m, 1H), 3.44 (d, J=14.0 Hz, 1H), 3.34-3.29 (m, 1H), 2.85 (dd, J=4.9, 4.9 Hz, 6H), 2.74-2.72 (m, 1H), 2.08 (q, J=12.2 Hz, 1H). ¹³C-NMR (100 MHz, CDCl₃): δ 164.33, 161.88, 145.02, 143.10, 143.02, 140.52, 140.50, 137.60, 132.05, 130.34, 130.26, 129.46, 129.39, 129.32, 128.26, 127.43, 127.15, 127.04, 125.94, 122.87, 122.84, 114.32, 114.12, 114.11, 113.91, 61.80, 46.11, 39.58, 34.20, 30.23. Calculated C₂₄H₂₅FN for [M+H]⁺: 346.1971. Found: 346.1967. HPLC (s-prep): Solvent System: hexanes:MeOH:ⁱPrOH (90:5:5) 0.1% TEA (modifier), 0.1% TFA (modifier), flow rate=3.0 mL/min; t₁=10.07 min, t₂=16.80 min.

Cis-4-(4′-fluoro-[1,1′-biphenyl]-3-yl)-N,N-dimethyl-1,2,3,4-tetrahydronaphthalen-2-amine, 3i: cis-amine 3i was synthesized from cis-3′Br-4-PAT 3a (66 mg, 0.2 mmol), 4-fluorophenylboronic acid (42 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic cis-amine 3i as colorless oil with 98% isolated yield. ¹H-NMR (500 MHz; CDCl₃): δ 7.52 (dd, J=8.6, 5.4 Hz, 2H), 7.43-7.36 (m, 3H), 7.17-7.07 (m, 5H), 7.02 (t, J=7.4 Hz, 1H), 6.81 (d, J=7.8 Hz, 1H), 4.16 (dd, J=12.2, 5.1 Hz, 1H), 3.05 (dd, J=15.7, 3.0 Hz, 1H), 2.98-2.93 (m, 1H), 2.83 (tdd, J=11.4, 4.6, 2.2 Hz, 1H), 2.40-2.38 (m, 7H), 1.78 (q, J=12.1 Hz, 1H). ¹³C-NMR (100 MHz, CDCl₃): δ 163.78, 161.32, 147.35, 140.64, 139.29, 137.40, 136.46, 129.56, 129.35, 129.18, 128.89, 128.81, 127.87, 127.57, 126.28, 126.06, 125.26, 115.78, 115.56, 60.70, 47.45, 41.65, 37.08, 33.21.

HCl salt ¹H-NMR (500 MHz; CDCl₃): δ 12.77 (s, 1H), 7.51 (dd, J=7.6, 5.8 Hz, 2H), 7.45 (d, J=7.6 Hz, 1H), 7.39 (t, J=7.6 Hz, 1H), 7.34 (s, 1H), 7.18-7.08 (m, 6H), 6.83 (d, J=7.7 Hz, 1H), 4.27 (dd, J=11.3, 3.6 Hz, 1H), 3.69-3.65 (m, 1H), 3.42 (br d, J=13.9 Hz, 1H), 3.34-3.28 (m, 1H), 2.85 (dd, J=6.8, 4.8 Hz, 6H), 2.72 (br d, J=9.9 Hz, 1H), 2.08 (q, J=12.1 Hz, 1H). ¹³C-NMR (100 MHz, CDCl₃): δ 163.81, 161.35, 144.91, 140.94, 137.67, 136.98, 132.00, 129.47, 129.45, 129.44, 128.88, 128.80, 127.68, 127.41, 127.27, 127.14, 125.98, 115.83, 115.62, 61.91, 46.23, 39.68, 39.53, 34.31, 30.29. Calculated C₂₄H₂₅FN for [M+H]*: 346.1971. Found: 346.1967. HPLC (s-prep): Solvent System: hexanes:MeOH:ⁱPrOH (90:5:5) 0.1% TEA (modifier), 0.1% TFA (modifier), flow rate=2.0 mL/min; t₁=16.53 min, t₂=23.22 min.

Cis-4-(4′-chloro-[1,1′-biphenyl]-3-yl)-N,N-dimethyl-1,2,3,4-tetrahydronaphthalen-2-amine, 3j: cis-amine 3j was synthesized from cis-3′Br-4-PAT 3a (66 mg, 0.2 mmol), 4-chlorophenylboronic acid (47 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic cis-amine 3j as colorless oil with 98% isolated yield.

¹H-NMR (500 MHz; CDCl₃): δ 7.49 (d, J=8.5 Hz, 2H), 7.45-7.37 (m, 5H), 7.18-7.12 (m, 3H), 7.03 (t, J=7.3 Hz, 1H), 6.80 (d, J=7.8 Hz, 1H), 4.17 (dd, J=12.1, 4.9 Hz, 1H), 3.06 (dd, J=15.5, 2.5 Hz, 1H), 3.00-2.94 (m, 1H), 2.90-2.86 (m, 1H), 2.41-2.38 (m, 7H), 1.79 (q, J=12.1 Hz, 1H). ¹³C-NMR (100 MHz, CDCl₃): δ 147.32, 140.43, 139.71, 139.16, 136.22, 133.49, 129.59, 129.36, 129.29, 128.99, 128.59, 128.20, 127.54, 126.38, 126.16, 125.30, 60.73, 47.39, 41.52, 36.93, 33.02.

HCl salt ¹H-NMR (500 MHz; CDCl₃): δ 12.88 (s, 1H), 7.49-7.46 (m, 3H), 7.42-7.38 (m, 3H), 7.35 (s, 1H), 7.19-7.15 (m, 3H), 7.12-7.10 (m, 1H), 6.84 (d, J=7.8 Hz, 1H), 4.28 (br d, J=8.2 Hz, 1H), 3.69-3.64 (m, 1H), 3.43 (br d, J=14.2 Hz, 1H), 3.34-3.29 (m, 1H), 2.86 (s, 6H), 2.73 (br d, J=10.4 Hz, 1H), 2.08 (q, J=11.7 Hz, 1H). ¹³C-NMR (100 MHz, CDCl₃): δ 145.01, 140.70, 139.30, 137.64, 133.64, 132.00, 129.56, 129.46, 129.01, 128.53, 128.02, 127.39, 127.30, 127.17, 125.96, 61.92, 46.24, 39.72, 39.52, 34.34, 30.28. Calculated C₂₄H₂₅CIN for [M+H]*: 362.1676. Found: 362.1673. HPLC (s-prep): Solvent System: hexanes:MeOH:ⁱPrOH (85:10:5) 0.1% TEA (modifier), 0.1% TFA (modifier), flow rate=1.5 mL/min; t, =23.00 min, t₂=30.59 min.

Cis-4-(4′-(dimethylamino)-[1,1′-biphenyl]-3-yl)-N,N-dimethyl-1,2,3,4-tetrahydro-naphthalen-2-amine, 3k: cis-amine 3k was synthesized from cis-3′Br-4-PAT 3a (66 mg, 0.2 mmol), 4-(dimethylamino)phenylboronic acid (50 mg, 0.3 mmol) in presence of potassium phosphate (85 mg, 0.4 mmol), Palladium(II) acetate (2 mg, 0.01 mmol) and SPhos (12 mg, 0.03 mmol) following general procedure described above. Purification of crude reaction mixture by silica gel column chromatography (4:2:0.1 hexanes:ethyl acetate:triethylamine) afforded racemic cis-amine 3k as colorless oil with 92% isolated yield.

¹H-NMR (500 MHz; CDCl₃): δ 7.48 (d, J=8.7 Hz, 2H), 7.44 (d, J=7.7 Hz, 1H), 7.37 (s, 1H), 7.34 (t, J=7.6 Hz, 1H), 7.13 (dt, J=14.7, 7.4 Hz, 2H), 7.06 (d, J=7.5 Hz, 1H), 7.01 (t, J=7.4 Hz, 1H), 6.83 (d, J=7.8 Hz, 1H), 6.79 (d, J=8.7 Hz, 2H), 4.14 (dd, J=12.3, 4.9 Hz, 1H), 3.07-3.03 (m, 1H), 2.98-2.94 (m, 7H), 2.88-2.85 (m, 1H), 2.42-2.38 (m, 7H), 1.79 (q, J=12.1 Hz, 1H). ¹³C-NMR (100 MHz, CDCl₃): δ 150.07, 146.95, 141.57, 139.50, 136.26, 129.46, 129.28, 128.99, 127.85, 126.96, 126.55, 126.17, 126.05, 124.52, 112.83, 60.70, 47.45, 41.51, 40.71, 36.83, 33.13.

HCl salt ¹H-NMR (500 MHz; CD30D): δ 7.85-7.80 (m, 2H), 7.75-7.70 (m, 2H), 7.59-7.55 (m, 2H), 7.47 (t, J=6.8 Hz, 1H), 7.30-7.24 (m, 2H), 7.17 (t, J=7.1 Hz, 1H), 7.07 (t, J=7.3 Hz, 1H), 6.77 (d, J=7.6 Hz, 1H), 4.41-4.40 (m, 1H), 3.87-3.86 (m, 1H), 3.21 (q, J=7.0 Hz, 2H), 2.98 (s, 6H), 2.60-2.59 (m, 1H), 2.17-2.15 (m, 1H). ¹³C-NMR (100 MHz, CDCl₃): 164.94, 144.93, 140.15, 136.62, 132.41, 129.43, 129.34, 128.96, 128.45, 128.11, 127.50, 127.23, 127.02, 125.72, 120.63, 62.69, 48.27, 44.74, 43.91, 40.25, 32.12, 30.42. Calculated C2₆H3₁N₂ for [M+H]⁺: 371.2487. Found: 371.2486. HPLC (s-prep): Solvent System: hexanes:MeOH:ⁱPrOH (85:10:5), 0.1% TEA (modifier), 0.1% TFA (modifier), flow rate=3.0 mL/min; t₁=10.76 min, t2=21.76 min.

Racemic compounds 2c-d were synthesized. To an oven dried 25 mL round bottom flask with stir bar was added trans-3′Br-4-PAT 2a (66 mg, 0.2 mmol), Aryl MIDA boronate (0.3 mmol) and dioxane (2.4 mL). The resulting mixture was sparged with N₂ for 30 min. To the flask was added palladium(II) acetate (2 mg, 0.01 mmol), SPhos (8 mg, 0.02 mmol) and aq K₃PO₄ (3.0 M, 0.5 mL, degassed by sparging with N₂ for 30 min). The resulting reaction mixture was stirred for 20 h under nitrogen atmosphere at 60° C. (Scheme 7). The reaction was quenched with 1 N NaOH aq (3 mL) and ethyl acetate (4 mL). The organic layer was separated, and the aqueous layer was extracted with ethyl acetate (5 mL×4). The combined organic layer was washed with brine (10 mL) and dried over Na₂SO₄. After evaporation of solvent the crude reaction mixture was purified by silica gel column chromatography (4:1:0.1 hexanes: dichloromethane:triethylamine) to afford racemic 2c-d.

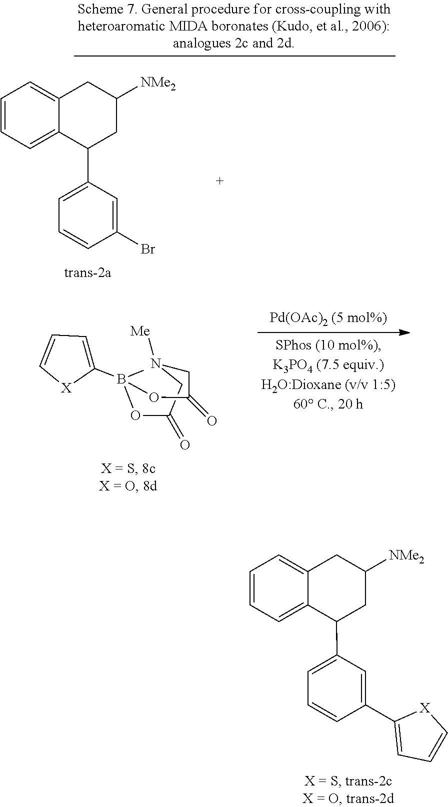

The racemic mixtures of trans-analogs were separated by semi-preparative chiral HPLC Regiscell column using conditions and solvents specific to each analog to elute the trans-(2R,4S) and -(2S,4R) enantiomers at retention time t₁ at t₂, respectively, with absolute stereochemistry assigned according to retention time of the previously published trans-3′CI-4-PAT analog (Sakhuja, et al., 2015). Both enantiomers were converted to HCl salts for use in pharmacological assays by adding 2M HCl solution in ether to the solution of free amine in ether.

Trans-4-(3-(thiophen-2-yl)phenyl)-N,N-dimethyl-1,2,3,4-tetrahydronaphthalen-2-amine, 2c: trans-amine 2c was synthesized from trans-3′Br-4-PAT 2a (66 mg, 0.2 mmol) and 2-thiopheneboronic acid MIDA ester (72 mg, 0.3 mmol) in presence of aq. potassium phosphate Palladium(II) acetate (2 mg, 0.01 mmol) and SPhos (8 mg, 0.02 mmol) following general procedure described above. Purification of crude reaction mixture by silica gel column chromatography (4:1:0.1 hexanes:dichloromethane:triethylamine) afforded racemic trans-amine 2c as colorless oil with 60% isolated yield.

¹H-NMR (500 MHz; CDCl₃): δ 7.43 (d, J=7.6 Hz, 1H), 7.32 (s, 1H), 7.26-7.23 (m, 3H), 7.20-7.16 (m, 2H), 7.10 (t, J=6.9 Hz, 1H), 7.05 (dd, J=4.7, 3.9 Hz, 1H), 6.96 (d, J=7.6 Hz, 1H), 6.89 (d, J=7.7 Hz, 1H), 4.39 (t, J=5.2 Hz, 1H), 3.04 (dd, J=16.2, 4.7 Hz, 1H), 2.87 (dd, J=16.2, 9.4 Hz, 1H), 2.66 (tt, J=9.0, 4.5 Hz, 1H), 2.28 (s, 6H), 2.17-2.14 (m, 2H). ¹³C-NMR (100 MHz, CDCl₃): δ 147.66, 144.67, 137.85, 136.49, 134.40, 130.10, 129.49, 128.83, 128.08, 128.06, 126.53, 126.44, 126.21, 124.84, 123.92, 123.20, 56.50, 44.15, 42.04, 34.98, 32.48. HCl salt ¹H-NMR (500 MHz; CDCl₃): δ 12.71 (s, 1H), 7.47 (d, J=7.7 Hz, 1H), 7.29-7.21 (m, 7H), 7.07-7.03 (m, 2H), 6.77 (d, J=7.7 Hz, 1H), 4.57-4.55 (m, 1H), 3.53 (dd, J=15.5, 4.8 Hz, 1H), 3.40-3.36 (m, 1H), 3.25 (dd, J=15.3, 11.5 Hz, 1H), 2.71 (t, J=5.1 Hz, 6H), 2.48-2.45 (m, 2H). ¹³C-NMR (100 MHz, CDCl₃): δ 145.33, 143.88, 135.30, 134.95, 132.78, 130.21, 129.54, 129.39, 128.22, 127.61, 127.52, 127.39, 125.78, 125.25, 124.69, 123.57, 58.77, 43.64, 41.08, 38.82, 31.51, 30.83. Calculated C₂₂H₂₄NS for [M+H]⁺: 334.1630. Found: 334.1628. HPLC (s-prep): Solvent System: hexanes:ⁱPrOH (98:2) 0.1% TEA (modifier), flow rate=2.0 mL/min; t, =10.55 min, t₂=12.20 min.

Trans-4-(3-(furan-2-yl)phenyl)-N,N-dimethyl-1,2,3,4-tetrahydronaphthalen-2-amine, 2d: trans-amine 2d was synthesized from trans-3′Br-4-PAT 2a (66 mg, 0.2 mmol) and 2-furanboronic acid MIDA ester (67 mg, 0.3 mmol) in presence of aq. potassium phosphate Palladium(II) acetate (2 mg, 0.01 mmol) and SPhos (8 mg, 0.02 mmol) following general procedure described above. Purification of crude reaction mixture by silica gel column chromatography (4:1:0.1 hexanes:dichloromethane:triethylamine) afforded racemic trans-amine 2d as colorless oil with 60% isolated yield.

¹H-NMR (500 MHz; CDCl₃): δ 7.49 (d, J=7.7 Hz, 1H), 7.44 (s, 1H), 7.39 (s, 1H), 7.27-7.24 (m, 2H), 7.21-7.17 (m, 2H), 7.11 (t, J=6.9 Hz, 1H), 6.96 (d, J=7.6 Hz, 1H), 6.86 (d, J=7.5 Hz, 1H), 6.59 (d, J=3.2 Hz, 1H), 6.45-6.44 (m, 1H), 4.41 (t, J=5.0 Hz, 1H), 3.09 (d, J=15.1 Hz, 1H), 2.91 (dd, J=15.9, 9.6 Hz, 1H), 2.72 (br s, 1H), 2.32 (s, 6H), 2.22-2.17 (m, 2H). ¹³C-NMR (126 MHz, CDCl₃): δ 154.17, 147.46, 142.07, 137.97, 136.52, 130.87, 130.07, 129.45, 128.62, 127.98, 126.45, 126.16, 124.18, 121.73, 111.70, 105.07, 56.49, 44.13, 42.12, 35.02, 32.64.

HCl-salt ¹H-NMR (500 MHz; CDCl₃): δ 12.71 (br s, 1H), 7.52 (d, J=7.7 Hz, 1H), 7.45 (s, 1H), 7.31-7.28 (m, 4H), 7.22-7.19 (m, 1H), 7.03 (d, J=7.7 Hz, 1H), 6.75 (d, J=7.7 Hz, 1H), 6.61 (d, J=3.2 Hz, 1H), 6.47-6.46 (m, 1H), 4.58-4.56 (br m, 1H), 3.56 (dd, J=15.6, 4.6 Hz, 1H), 3.43-3.36 (m, 1H), 3.25 (dd, J=15.2, 11.7 Hz, 1H), 2.71 (s, 6H), 2.47-2.44 (m, 2H). ¹³C-NMR (126 MHz, CDCl₃): δ 153.39, 145.08, 142.30, 135.32, 132.76, 131.29, 130.10, 129.45, 129.10, 127.45, 127.34, 127.28, 123.45, 122.44, 111.78, 105.60, 58.68, 43.62, 41.05, 38.67, 31.37, 30.78. Calculated C₂₂H₂₄NO for [M+H]⁺: 318.1858. Found: 318.1858. HPLC (s-prep): Solvent System: hexanes:ⁱPrOH (98:2) 0.1% TEA (modifier), flow rate=2.0 mL/min; t₁=12.96 min, t2=15.57 min.

The cis analogues 3c and 3d (Scheme 8) were synthesized from cis-3′Br-4-PAT 3a and corresponding Aryl MIDA boronates following the general procedure as described above for trans 2c and 2d. The racemic mixtures of cis-analogs were separated by semi-preparative chiral HPLC Regiscell column using conditions and solvents specific to each analog to elute the cis-(2S,4S) and -(2R,4R) enantiomers at retention time t₁ at t₂, respectively. The absolute stereochemistry was assigned by correlating retention time to the x-ray crystal structure of (2R,4R)-cis-3′CI-4-PAT, 3b and (2S,4S)-cis-3′F-4-PAT, 3b′ analogs. Both enantiomers were converted to HCl salts for use in pharmacological assays by adding 2M HCl solution in ether to the solution of free amine in ether.

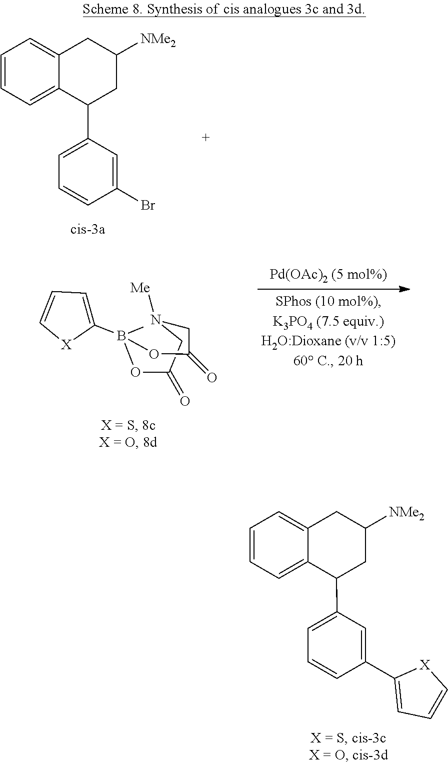

Cis-4-(3-(thiophen-2-yl) phenyl)-N,N-dimethyl-1,2,3,4-tetrahydronaphthalen-2-amine, 3c: cis-amine 3c was synthesized from cis-3′Br-4-PAT 3a (66 mg, 0.2 mmol) and 2-thiopheneboronic acid MIDA ester (72 mg, 0.3 mmol) in presence of aq. potassium phosphate Palladium(II) acetate (2 mg, 0.01 mmol) and SPhos (8 mg, 0.02 mmol) following general procedure described above. Purification of crude reaction mixture by silica gel column chromatography (4:1:0.1 hexanes:dichloromethane:triethylamine) afforded racemic cis-amine 3c as colorless oil with 60% isolated yield.

¹H-NMR (500 MHz; CDCl3): δ 7.51 (d, J=7.8 Hz, 1H), 7.45 (s, 1H), 7.33 (t, J=7.7 Hz, 1H), 7.30-7.27 (m, 2H), 7.17-7.12 (m, 2H), 7.09-7.05 (m, 2H), 7.05-7.02 (m, 1H), 6.81 (d, J=7.8 Hz, 1H), 4.15 (dd, J=12.6, 5.4 Hz, 1H), 3.10-2.98 (m, 3H), 2.46-2.39 (m, 7H), 1.80 (q, J=11.8 Hz, 1H). ¹³C-NMR (100 MHz, CDCl₃): δ 147.39, 144.52, 139.17, 136.43, 134.76, 129.56, 129.36, 129.31, 128.08, 128.03, 126.55, 126.31, 126.10, 124.91, 124.29, 123.31, 60.72, 47.35, 41.68, 36.89, 33.27.

HCl salt ¹H-NMR (500 MHz; CDCl₃): δ 13.00 (br s, 1H), 7.54 (d, J=7.8 Hz, 1H), 7.42 (s, 1H), 7.35 (t, J=7.7 Hz, 1H), 7.30 (m, J=4.7 Hz, 2H), 7.19 (d, J=4.0 Hz, 2H), 7.12-7.05 (m, 3H), 6.84 (d, J=7.8 Hz, 1H), 4.24 (dd, J=12.1, 5.4 Hz, 1H), 3.68-3.64 (m, 1H), 3.48-3.44 (m, 1H), 3.34-3.28 (m, 1H), 2.87-2.84 (m, 6H), 2.72-2.69 (m, 1H), 2.06 (q, J=12.5 Hz, 1H). ¹³C-NMR (100 MHz, CDCl₃): δ 145.02, 143.97, 137.55, 135.08, 132.03, 129.63, 129.46, 128.16, 127.81, 127.31, 127.18, 126.36, 125.16, 124.97, 123.56, 61.92, 46.17, 39.68, 39.49, 34.08, 30.38. Calculated C₂₂H₂₄NS for [M+H]⁺: 334.1630. Found: 334.1629. HPLC (s-prep): Solvent System: hexanes:MeOH:ⁱPrOH (85:10:5) 0.1% TEA (modifier), 0.1% TFA (modifier), flow rate=3.0 mL/min; t₁=8.58 min, t₂=25.12 min.

Cis-4-(3-(furan-2-yl)phenyl)-N,N-dimethyl-1,2,3,4-tetrahydronaphthalen-2-amine, 3d: cis-amine 3d was synthesized from cis-3′Br-4-PAT 3a (66 mg, 0.2 mmol) and 2-furanboronic acid MIDA ester (67 mg, 0.3 mmol) in presence of aq. potassium phosphate Palladium(II) acetate (2 mg, 0.01 mmol) and SPhos (8 mg, 0.02 mmol) following general procedure described above. Purification of crude reaction mixture by silica gel column chromatography (4:1:0.1 hexanes:dichloromethane:triethylamine) afforded racemic cis-amine 3d as colorless oil with 60% isolated yield.

¹H-NMR (500 MHz; CDCl₃): δ 7.57 (d, J=7.8 Hz, 1H), 7.54 (s, 1H), 7.46 (s, 1H), 7.35 (t, J=7.7 Hz, 1H), 7.15 (dt, J=15.4, 7.6 Hz, 2H), 7.08 (d, J=7.6 Hz, 1H), 7.03 (t, J=7.4 Hz, 1H), 6.81 (d, J=7.8 Hz, 1H), 6.65 (d, J=3.2 Hz, 1H), 6.48-6.45 (m, 1H), 4.15 (dd, J=12.1, 4.8 Hz, 1H), 3.07 (dd, J=15.5, 2.8 Hz, 1H), 2.98 (dd, J=15.8, 11.4 Hz, 1H), 2.88-2.83 (m, 1H), 2.40-2.37 (m, 7H), 1.79 (q, J=12.2 Hz, 1H). ¹³C-NMR (126 MHz, CDCl₃): δ 154.05, 142.10, 139.22, 136.37, 131.22, 129.52, 129.34, 129.09, 127.93, 126.26, 126.08, 124.26, 122.12, 111.74, 105.20, 60.69, 47.38, 41.62, 36.73, 33.21.

HCl salt ¹H-NMR (500 MHz; CDCl₃): δ 12.66 (s, 1H), 7.55 (d, J=7.7 Hz, 1H), 7.47 (s, 1H), 7.41 (s, 1H), 7.31 (t, J=7.7 Hz, 1H), 7.17-7.12 (m, 2H), 7.05-7.01 (m, 2H), 6.77 (d, J=7.8 Hz, 1H), 6.62 (d, J=3.0 Hz, 1H), 6.44-6.42 (m, 1H), 4.21 (d, J=8.1 Hz, 1H), 3.67-3.60 (br m, 1H), 3.39 (d, J=14.4 Hz, 1H), 3.30-3.25 (m, 1H), 2.81 (d, J=9.8 Hz, 6H), 2.65-2.63 (m, 1H), 2.07-2.01 (m, 1H). ¹³C-NMR (126 MHz, CDCl₃): δ 153.50, 144.75, 142.26, 132.01, 131.49, 129.42, 129.38, 129.37, 127.75, 127.12, 127.01, 124.02, 122.77, 112.57, 111.82, 105.55, 61.91, 46.20, 39.75, 39.50, 33.99, 30.39. C₂₂H₂₄NO for [M+H]⁺: 318.1858. Found: 318.1859. HPLC (s-prep): Solvent System: hexanes:MeOH:ⁱPrOH (85:10:5) 0.1% TEA (modifier), 0.1% TFA (modifier), flow rate=3.0 mL/min; t₁=9.27 min, t₂=27.19 min.

Trans-4-(3-(pyridin-4-yl)phenyl)-N,N-dimethyl-1,2,3,4-tetrahydronaphthalen-2-amine 2e (Knapp, et al., 2009): To an oven dried 25 mL round bottom flask with stir bar was added trans-3′Br-4-PAT 2a (66 mg, 0.2 mmol), 4-pyridinylboronic acid (30 mg, 0.24 mmol) and dioxane (2.4 mL). The resulting mixture was sparged with N₂ for 30 min. To the flask was added Tris(dibenzylideneacetone)dipalladium(0) (9 mg, 0.01 mmol), PCy₃ (7 mg, 0.024 mmol) and aq K₃PO₄ (3.0 M, 0.5 mL, degassed by sparging with N₂ for 30 min). The resulting reaction mixture was stirred for 12 h under nitrogen atmosphere at 95° C. (Scheme 9). The reaction was quenched with 1N NaOH aq (3 mL) and ethyl acetate (4 mL). The organic layer was separated, and the aqueous layer was extracted with ethyl acetate (5 mL×4). The combined organic layer was washed with brine (10 mL) and dried over Na₂SO₄.

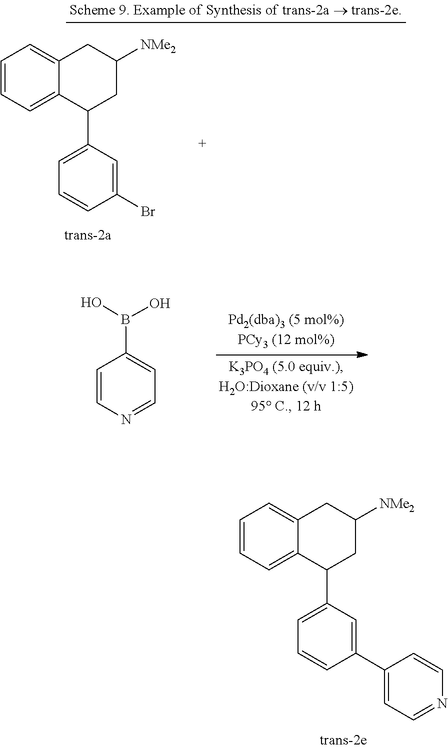 [SMILES: CN(C)C1Cc2ccccc2C(c2cccc(Br)c2)C1 ; OB(O)c1ccncc1 ; CN(C)C1Cc2ccccc2C(c2cccc(-c3ccncc3)c2)C1]

After evaporation of solvent the crude reaction mixture was purified by silica gel column chromatography (1:1:0.2 hexanes: ethyl acetate:triethylamine) to afford racemic 2e as colorless oil with 60% isolated yield.

¹H-NMR (500 MHz; CDCl₃): δ 8.63 (dd, J=4.6, 1.4 Hz, 2H), 7.47-7.43 (m, 3H), 7.37 (t, J=7.7 Hz, 1H), 7.32 (br s, 1H), 7.21-7.17 (m, 2H), 7.13-7.10 (m, 1H), 7.07 (d, J=7.7 Hz, 1H), 6.96 (d, J=7.6 Hz, 1H), 4.46 (t, J=5.2 Hz, 1H), 3.05 (dd, J=16.3, 4.8 Hz, 1H), 2.89 (dd, J=16.2, 9.3 Hz, 1H), 2.67 (tt, J=8.8, 4.4 Hz, 1H), 2.29 (s, 7H), 2.20-2.16 (m, 1H). ¹³C-NMR (100 MHz, CDCl₃): δ 150.32, 148.55, 148.06, 138.21, 136.52, 130.04, 129.65, 129.61, 129.08, 127.42, 126.65, 126.27, 124.91, 121.81, 56.42, 44.24, 41.97, 35.23, 32.23.

HCl salt ¹H-NMR (500 MHz; CD₃OD): δ 8.87 (d, J=6.7 Hz, 2H), 8.39 (d, J=6.7 Hz, 2H), 7.87 (d, J=7.8 Hz, 1H), 7.75 (s, 1H), 7.57 (t, J=7.8 Hz, 1H), 7.36 (d, J=7.7 Hz, 1H), 7.28 (dd, J=16.5, 7.9 Hz, 2H), 7.22 (t, J=7.4 Hz, 1H), 7.04 (d, J=7.6 Hz, 1H), 4.75 (t, J=4.1 Hz, 1H), 3.64-3.60 (m, 1H), 3.43 (dd, J=15.8, 4.8 Hz, 1H), 3.35 (s, 1H), 3.19 (dd, J=15.8, 10.9 Hz, 1H), 2.89 (d, J=11.2 Hz, 6H), 2.59-2.48 (m, 2H). ¹³C-NMR (100 MHz, CDCl₃): 148.83, 148.02, 147.86, 136.95, 134.92, 129.60, 129.51, 129.26, 128.02, 126.85, 126.93, 126.00, 124.28, 121.58, 57.62, 45.35, 39.75, 39.54, 32.87, 30.01. C₂₃H₂₅N₂ for [M+H]⁺: 329.2018. Found: 329.2018. HPLC (s-prep): Solvent System: hexanes:MeOH:ⁱPrOH:EtOH (70:10:10:10) 0.1% TEA (modifier), flow rate=3.0 mL/min; t₁=9.27 min, t₂=14.68 min.

Cis-4-(3-(pyridin-4-yl)phenyl)-N,N-dimethyl-1,2,3,4-tetrahydronaphthalen-2-amine 3e: cis-amine 3e was synthesized from cis-3′Br-4-PAT 3a (66 mg, 0.2 mmol) and 4-pyridinylboronic acid (30 mg, 0.24 mmol) in presence of aq. potassium phosphate Tris(dibenzylideneacetone)dipalladium(0) (9 mg, 0.01 mmol) and PCy₃ (7 mg, 0,024 mmol) following procedure described above for trans-2e (Scheme 10). Purification of crude reaction mixture by silica gel column chromatography (1:1:0.2 hexanes: ethyl acetate:triethylamine) afforded racemic cis-amine 3e as colorless oil with 55% isolated yield.

¹H-NMR (500 MHz; CDCl₃): δ 8.64-8.62 (m, 2H), 7.52 (d, J=8.1 Hz, 1H), 7.48 (dd, J=4.6, 1.4 Hz, 2H), 7.45-7.42 (m, 2H), 7.26-7.24 (m, 1H), 7.15 (dt, J=13.7, 7.0 Hz, 2H), 7.03 (t, J=7.4 Hz, 1H), 6.77 (d, J=7.8 Hz, 1H), 4.19 (dd, J=12.2, 4.8 Hz, 1H), 3.08 (d, J=14.6 Hz, 1H), 3.06-2.87 (m, 2H), 2.38-2.37 (m, 7H), 1.87-1.75 (m, 1H). ¹³C-NMR (126 MHz, CDCl₃): δ 150.33, 148.28, 147.07, 138.67, 138.54, 129.60, 129.30, 127.43, 126.64, 126.45, 125.48, 121.80, 60.98, 47.05, 41.06, 36.39, 32.29.

HCl salt ¹H-NMR (500 MHz; CDCl₃): δ 8.64-8.62 (m, 2H), 7.52 (d, J=8.1 Hz, 1H), 7.48 (dd, J=4.6, 1.4 Hz, 2H), 7.45-7.42 (m, 2H), 7.26-7.24 (m, 1H), 7.15 (dt, J=13.7, 7.0 Hz, 2H), 7.03 (t, J=7.4 Hz, 1H), 6.77 (d, J=7.8 Hz, 1H), 4.19 (dd, J=12.2, 4.8 Hz, 1H), 3.08 (d, J=14.6 Hz, 1H), 3.06-2.87 (m, 2H), 2.38-2.37 (m, 7H), 1.87-1.75 (m, 1H). ¹³C-NMR (100 MHz, CDCl₃): 149.05, 148.88, 147.95, 140.69, 134.84, 129.88, 129.15, 127.55, 126.98, 126.32, 125.59, 121.92, 62.12, 45.98, 39.01, 39.26, 34.65, 30.15. C₂₃H₂₅N₂ for [M+H]*: 329.2018. Found: 329.2018. HPLC (s-prep): Solvent System: hexanes:EtOH (85:15) 0.1% TEA (modifier), 0.1% TFA (modifier), flow rate=3.0 mL/min; t₁=18.60 min, t₂=23.79 min.

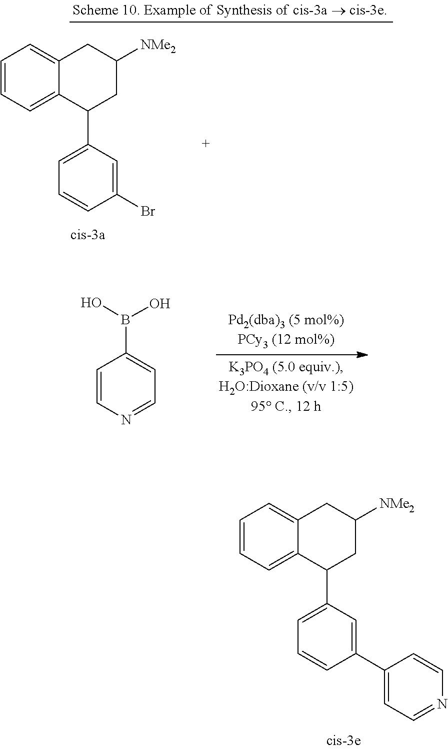 [SMILES: CN(C)C1Cc2ccccc2C(c2cccc(Br)c2)C1 ; OB(O)c1ccncc1 ; CN(C)C1Cc2ccccc2C(c2cccc(-c3ccncc3)c2)C1]

Examples of synthesized and purified chemical structures are shown below:

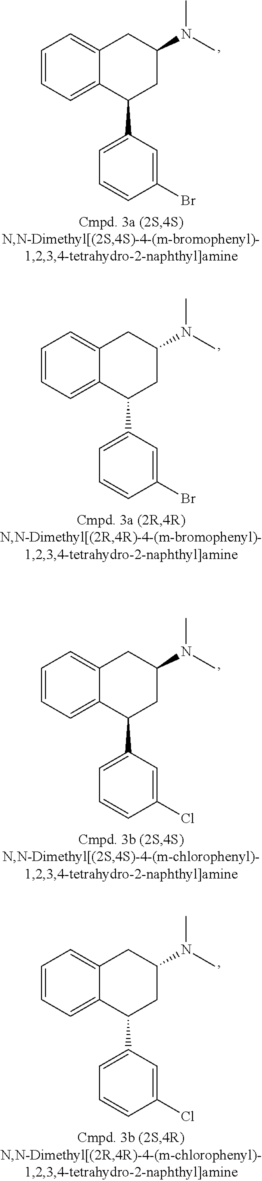 [SMILES: CN(C)[C@@H]1Cc2ccccc2[C@H](c2cccc(Br)c2)C1 ; CN(C)[C@H]1Cc2ccccc2[C@@H](c2cccc(Br)c2)C1 ; CN(C)[C@@H]1Cc2ccccc2[C@H](c2cccc(Cl)c2)C1 ; CN(C)[C@H]1Cc2ccccc2[C@@H](c2cccc(Cl)c2)C1]

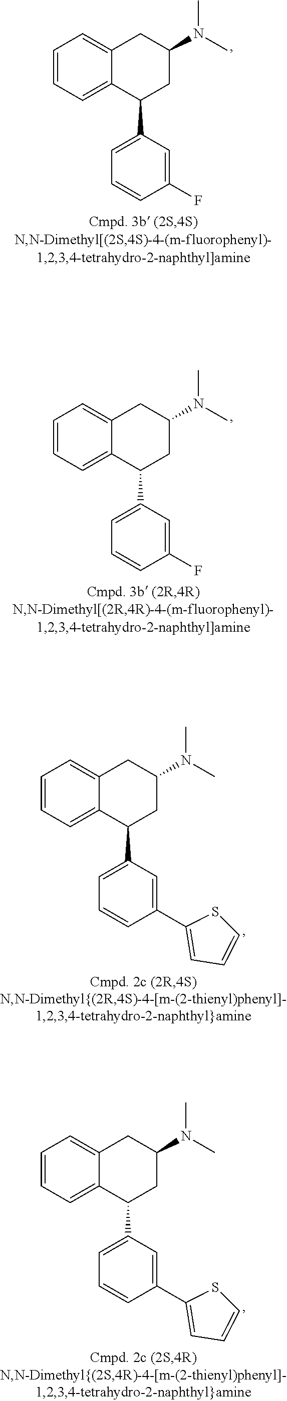 [SMILES: CN(C)[C@@H]1Cc2ccccc2[C@H](c2cccc(F)c2)C1 ; CN(C)[C@H]1Cc2ccccc2[C@@H](c2cccc(F)c2)C1 ; CN(C)[C@H]1Cc2ccccc2[C@H](c2cccc(-c3cccs3)c2)C1 ; CN(C)[C@@H]1Cc2ccccc2[C@@H](c2cccc(-c3cccs3)c2)C1]

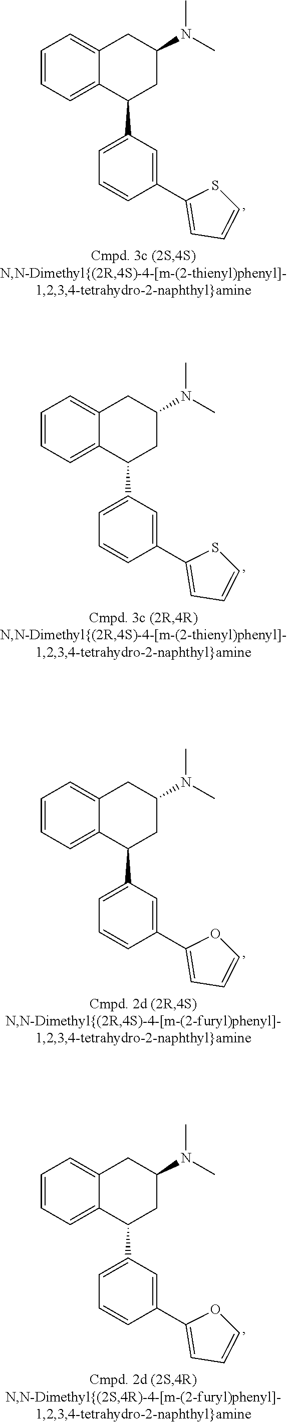 [SMILES: CN(C)[C@@H]1Cc2ccccc2[C@H](c2cccc(-c3cccs3)c2)C1 ; CN(C)[C@H]1Cc2ccccc2[C@@H](c2cccc(-c3cccs3)c2)C1 ; CN(C)[C@H]1Cc2ccccc2[C@H](c2cccc(-c3ccco3)c2)C1 ; CN(C)[C@@H]1Cc2ccccc2[C@@H](c2cccc(-c3ccco3)c2)C1]

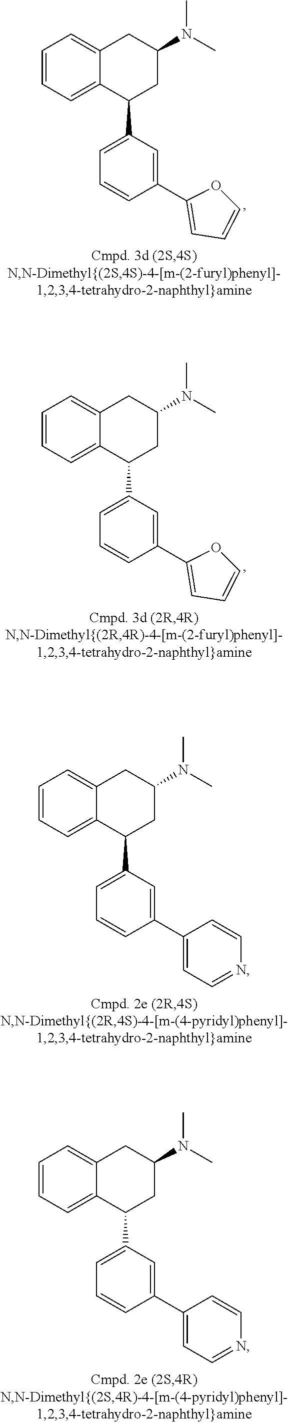 [SMILES: CN(C)[C@@H]1Cc2ccccc2[C@H](c2cccc(-c3ccco3)c2)C1 ; CN(C)[C@H]1Cc2ccccc2[C@@H](c2cccc(-c3ccco3)c2)C1 ; CN(C)[C@H]1Cc2ccccc2[C@H](c2cccc(-c3ccncc3)c2)C1 ; CN(C)[C@@H]1Cc2ccccc2[C@@H](c2cccc(-c3ccncc3)c2)C1]

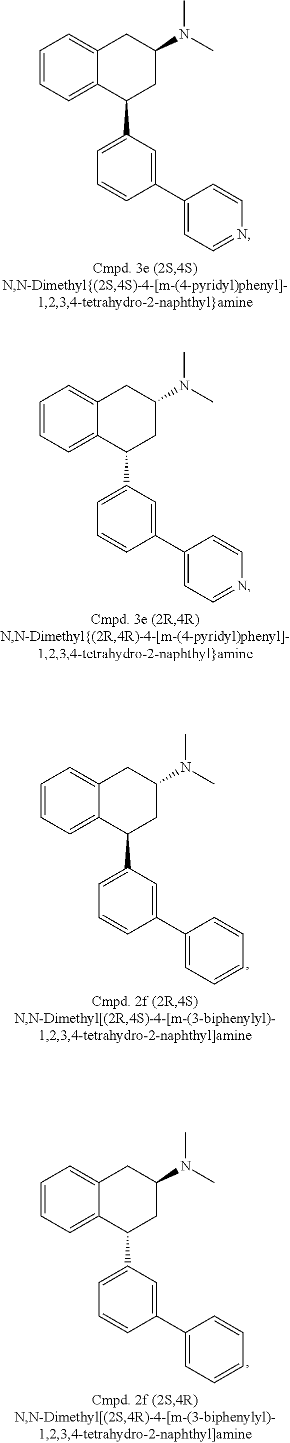 [SMILES: CN(C)[C@@H]1Cc2ccccc2[C@H](c2cccc(-c3ccncc3)c2)C1 ; CN(C)[C@H]1Cc2ccccc2[C@@H](c2cccc(-c3ccncc3)c2)C1 ; CN(C)[C@H]1Cc2ccccc2[C@H](c2cccc(-c3ccccc3)c2)C1 ; CN(C)[C@@H]1Cc2ccccc2[C@@H](c2cccc(-c3ccccc3)c2)C1]

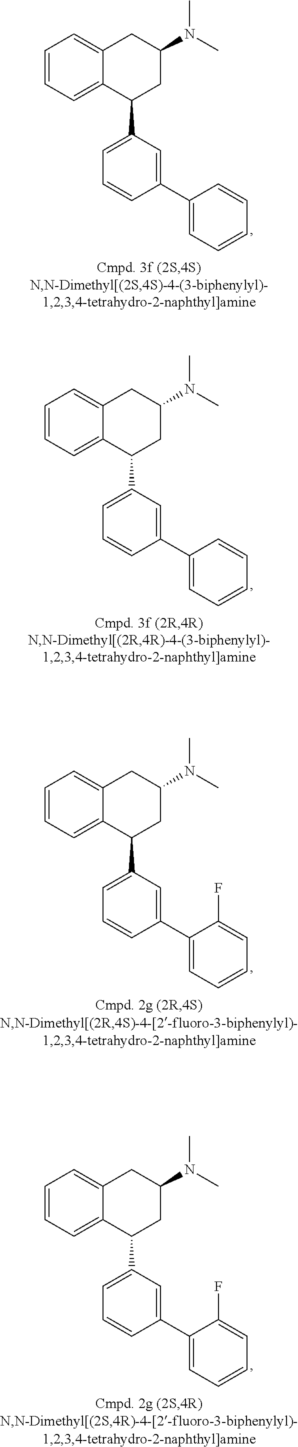 [SMILES: CN(C)[C@@H]1Cc2ccccc2[C@H](c2cccc(-c3ccccc3)c2)C1 ; CN(C)[C@H]1Cc2ccccc2[C@@H](c2cccc(-c3ccccc3)c2)C1 ; CN(C)[C@H]1Cc2ccccc2[C@H](c2cccc(-c3ccccc3F)c2)C1 ; CN(C)[C@@H]1Cc2ccccc2[C@@H](c2cccc(-c3ccccc3F)c2)C1]

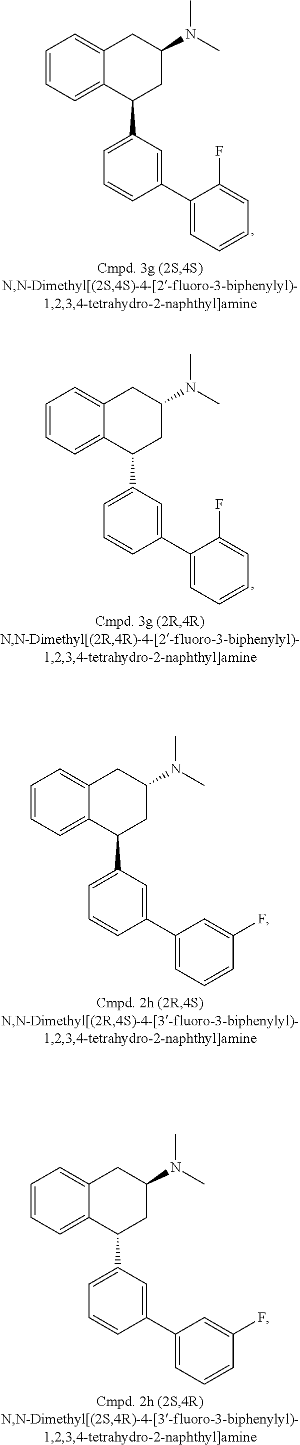 [SMILES: CN(C)[C@@H]1Cc2ccccc2[C@H](c2cccc(-c3ccccc3F)c2)C1 ; CN(C)[C@H]1Cc2ccccc2[C@@H](c2cccc(-c3ccccc3F)c2)C1 ; CN(C)[C@H]1Cc2ccccc2[C@H](c2cccc(-c3cccc(F)c3)c2)C1 ; CN(C)[C@@H]1Cc2ccccc2[C@@H](c2cccc(-c3cccc(F)c3)c2)C1]

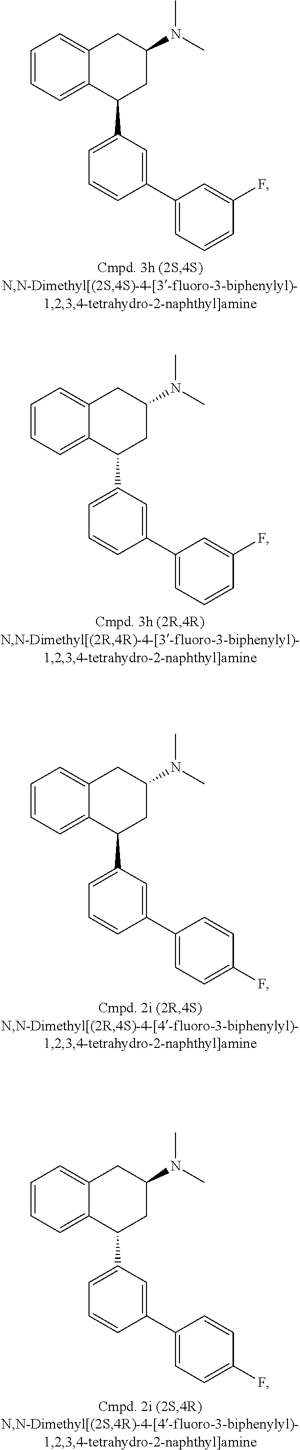 [SMILES: CN(C)[C@@H]1Cc2ccccc2[C@H](c2cccc(-c3cccc(F)c3)c2)C1 ; CN(C)[C@H]1Cc2ccccc2[C@@H](c2cccc(-c3cccc(F)c3)c2)C1 ; CN(C)[C@H]1Cc2ccccc2[C@H](c2cccc(-c3ccc(F)cc3)c2)C1 ; CN(C)[C@@H]1Cc2ccccc2[C@@H](c2cccc(-c3ccc(F)cc3)c2)C1]

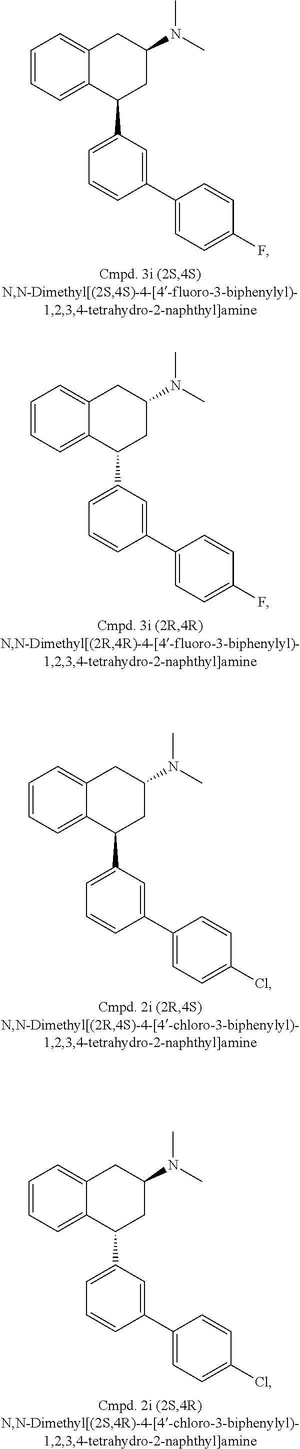 [SMILES: CN(C)[C@@H]1Cc2ccccc2[C@H](c2cccc(-c3ccc(F)cc3)c2)C1 ; CN(C)[C@H]1Cc2ccccc2[C@@H](c2cccc(-c3ccc(F)cc3)c2)C1 ; CN(C)[C@H]1Cc2ccccc2[C@H](c2cccc(-c3ccc(Cl)cc3)c2)C1 ; CN(C)[C@@H]1Cc2ccccc2[C@@H](c2cccc(-c3ccc(Cl)cc3)c2)C1]

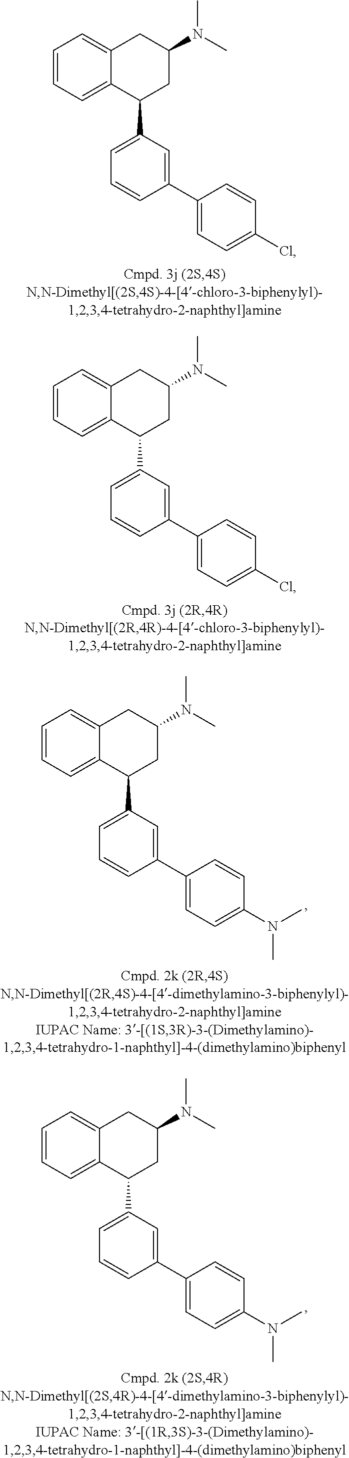 [SMILES: CN(C)[C@@H]1Cc2ccccc2[C@H](c2cccc(-c3ccc(Cl)cc3)c2)C1 ; CN(C)[C@H]1Cc2ccccc2[C@@H](c2cccc(-c3ccc(Cl)cc3)c2)C1 ; CN(C)c1ccc(-c2cccc([C@@H]3C[C@@H](N(C)C)Cc4ccccc43)c2)cc1 ; CN(C)c1ccc(-c2cccc([C@H]3C[C@H](N(C)C)Cc4ccccc43)c2)cc1]

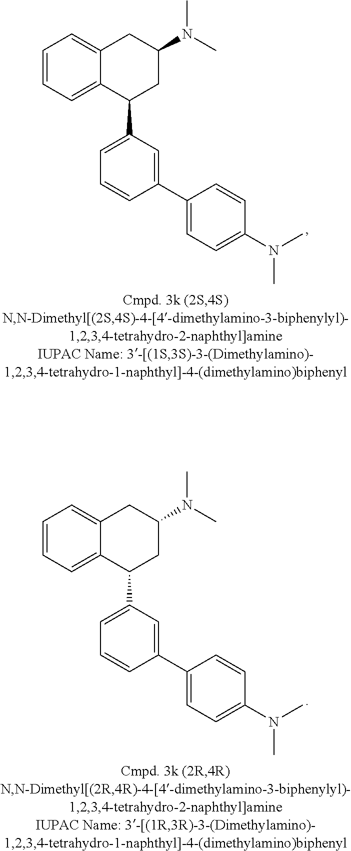 [SMILES: CN(C)c1ccc(-c2cccc([C@@H]3C[C@H](N(C)C)Cc4ccccc43)c2)cc1 ; CN(C)c1ccc(-c2cccc([C@H]3C[C@@H](N(C)C)Cc4ccccc43)c2)cc1]

### Example 2. Functional Assays

For cell culture and transfection, HEK293 (ATCC no. CRL-1573) and HEK293T cells (ATCC no. CRL-3216) were maintained in MEM and DMEM (Corning), respectively, supplemented with 10% regular FBS and 1% penicillin/streptomycin; CHO cells (ATCC no. CRL-61) were maintained in Kaighn's modification of Ham's F-12K (Gibco) using the same supplementation. All cells were grown adherently in 10 cm plates in a humidified incubator kept at 37° C. and 5% carbon dioxide. All human wild type aminergic GPCR clones, encoded in a pcDNA3.1(+) vector, were obtained from the cDNA Resource Center (cdna.orq). Transient transfections were used to express 5-HT_{2A}, 5-HT_{2C}, α_{1A}- or α_{1B}-adrenergic receptors, whereas 5-HT₇₍ₐ₎Rs were stably expressed in HEK293 cells. H₁Rs were transiently expressed, and D₂Rs were stably expressed in CHO cells. 5-HT_{2B} and 5-HT_{1A}Rs in HEK293T cells were transiently expressed because HEK293 cells failed to provide sufficient expression. D₃Rs stably expressed in HEK293 cells were generously provided by Dr. David Sibley's laboratory.

Transient transfections were performed on cells in the log growth phase (70-90% confluence). First, a transfection cocktail was prepared by adding 10 μg of cDNA and 40 μg of linear polyethylenimine (˜40,000 g/mol; Polysciences Inc.) separately to two 2.5 mL aliquots of Opti-MEM (Gibco, Ref. 31985-070). Each solution was mixed by inversion before combining, mixed by inversion again, and incubated at 37° C. for 30 minutes. Cells were then washed with 1 mL 1×PBS, followed by the gentle addition of 5 mL of transfection cocktail and 5 mL of cell culture medium with 5% (final) dialyzed FBS. Transfections were performed for 48 hours. This represents an economic modification of the previous transfection methods using lipofectamine or Turbofect (Invitrogen, #11668027 and Thermo Scientific, #R0532, respectively), which gave similar levels of expression for the receptors studied here. Cell membranes were homogenized as previously described (Perry, et al., 2020).

The affinity of ligands was determined via radioligand binding techniques using human recombinant receptors expressed in mammalian clonal cells. Details on assay conditions, radioligand, nonspecific binding and receptor expression are shown in Table 5. Number of independent radioligand binding experiments is shown in Table 6. In Table 6, all independent experiments listed were performed using 3 technical replicates.

Ligand affinity was assessed using established methodology and 2-5 μg of protein per well, determined by the Pierce bicinchoninic acid protein assay according to the manufacturers protocol (Thermo Scientific), (Perry, et al., 2020; Roth, 2013). Saturation binding experiments were performed on membranes expressing 5-HT_{2A}, 5-HT_{2B}, 5-HT_{2C}, or H₁Rs in triplicate across 8 concentrations. Competition binding assays were performed in at least triplicate with approximate K_{d} concentrations of radioligand. Total and nonspecific binding were determined in octet. Each compound was assessed in at least two independent experiments across 10-14 concentrations in half-log units (1 μM-100 μM) where the center of the concentration range was the approximate pKᵢ. Serial dilutions of unlabeled compound were made in assay buffer at 2.5× the final concentration using 10 mM DMSO stocks (final [DMSO]≤1%). Assays were terminated using rapid filtration via Whatman GF/B Fired Filters (Brandel Inc., Gaithersburg, MD) soaked in 0.3% (w/v) PEI, using an automated Tomtec Harvester 96 (Hamden, CT).

Filters were washed with 50 mM Tri-HCl (pH=7.4, 4° C.) before being oven dried and placed into scintillation vials containing 1 mL SX18-4 ScintiVerse^{T}M BD Cocktail (Fisher Chemical, Fair Lawn, NJ). Scintillation was detected using a Tri-Carb 2910 TR Liquid Scintillation Analyzer (Perkin Elmer, Boston, MA).

TABLE 5
Radioligand binding assay conditions for membranes used in this study, including the binding
parameters used to derive Kᵢ values and the results of saturation binding assays.

| | | | | | | | Bₘₐₓ | |
|---|---|---|---|---|---|---|---|---|
| | | | | | | K_{d} | (pmol/mg | |
| | | | Nonspecific | Incubation | | (nM ± | protein ± | Hill Slope |
| Receptor | n | Radioligand | Binding (10 μM) | time (min) | Buffer | SEM) | SEM) | (n_{H}) |
| 5-HT_{1A}^{d} | — | [³H]5-CT | 5-HT | 90 | a | 2.5 | — | — |
| 5-HT_{2A} | 3 | [³H]Ketanserin | mianserin | 90 | a | 0.57 ± 0.24 | 3.48 ± 0.42 | 1.1 ± 0.2 |
| 5-HT_{2B} | 2 | [³H]Mesulergine | mianserin | 90 | a | 2.82 ± 0.35 | 2.03 ± 0.28 | 1.0 ± 0.1 |
| 5-HT_{2C} | 3 | [³H]Mesulergine | mianserin | 90 | a | 1.00 ± 0.17 | 7.65 ± 0.84 | 1.3 ± 0.2 |
| 5-HT₇^{d} | — | [³H]5-CT | 5-HT | 180 | a | 0.3 | — | — |
| H₁ | 3 | [³H]Mepyramine | triprolidine | 90 | a | 2.59 ± 0.22 | 8.48 ± 0.20 | 0.90 ± 0.36 |
| D_{2L} | 3 | [³H]Spiperone | chlorpromazine | 180 | b | 0.28 ± 0.031 | 2.470 ± 110 | — |
| D₃ | 3 | [³H]Spiperone | chlorpromazine | 180 | b | 0.14 ± 0.016 | 500 ± 90 | — |
| α_{1A}^{e} | — | [³H]Prazosin | chlorpromazine | 90 | c | 0.64 | — | — |
| α_{1B}^{e} | — | [³H]Prazosin | chlorpromazine | 90 | c | 0.75 | — | — |

Original data are represented as the mean ± SEM from the indicated number of individual experiments performed in triplicate. Buffer:
^{a}50 mM Tris-HCl, 10 mM MgCl₂, 0.1 mM EDTA, pH = 7.4,
^{b}50 mM HEPES, 50 mM NaCl, 5 mM MgCl₂, 0.5 mM EDTA, 0.1% BSA, pH = 7.4,
^{c}20 mM Tris-HCl, 145 mM NaCl, pH = 7.4
^{d}Values obtained from (Armstrong, Casey et al., 2020).
^{e}Values obtained from (Roth, 2013).

TABLE 6
Number of independent radioligand binding experiments performed
for each ligand at 5-HT_{2A}, 5-HT_{2B}, 5-HT2c, and H₁Rs listed in Table 1.

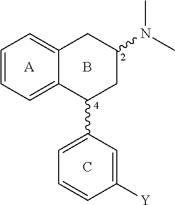

| | | | Affinity (pKᵢ ± SD) | | | |
|---|---|---|---|---|---|---|
| Cmpd | Y | Chirality | 5-HT_{2A}R 5-HT_{2B}R 5-HT_{2C}R H₁R | | | |
| 3a | 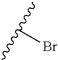 [SMILES: *Br] | (2S,4S) (2R,4R) | 2 3 | 2 2 | 3 4 | 3 2 |
| 3b | 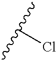 [SMILES: *Cl] | (2S,4S) (2R,4R) | 2 2 | 3 3 | 4 2 | 4 4 |
| 3b′ | 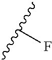 [SMILES: *F] | (2S,4S) (2R,4R) | 4 3 | 2 3 | 2 4 | 2 2 |
| 2c 3c | 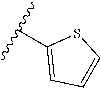 [SMILES: *c1cccs1] | (2R,4S) (28,4R) (2S,4S) (2R,4R) | 2 2 3 5 | 5 3 3 4 | 3 3 2 2 | 2 4 2 2 |
| 2d 3d | 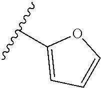 [SMILES: *c1ccco1] | (2R,4S) (2S,4R) (2S,4S) (2R,4R) | 2 3 3 3 | 3 3 3 3 | 4 5 2 2 | 0 2 0 2 |
| 2e 3e | 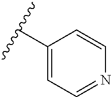 [SMILES: *c1ccncc1] | (2R,4S) (2S,4R) (2S,4S) (2R,4R) | 2 3 2 2 | 2 2 2 2 | 2 3 2 2 | 0 2 0 3 |
| 2f 3f | 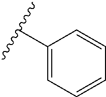 [SMILES: *c1ccccc1] | (2R,4S) (2S,4R) (2S,4S) (2R,4R) | 2 3 3 4 | 2 2 3 4 | 2 2 2 2 | 2 3 2 2 |
| 2g 3g | 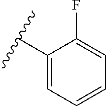 [SMILES: *c1ccccc1F] | (2R,4S) (2S,4R) (2S,4S) (2R,4R) | 2 2 3 4 | 2 3 3 2 | 3 3 2 2 | 2 4 0 2 |
| 2h 3h | 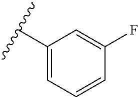 [SMILES: *c1cccc(F)c1] | (2R,4S) (2S,4R) (2S,4S) (2R,4R) | 4 3 3 4 | 2 5 2 4 | 2 3 2 3 | 2 3 2 2 |
| 2i 3i | 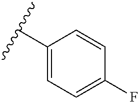 [SMILES: *c1ccc(F)cc1] | (2R,4S) (2S,4R) (2S,4S) (2R,4R) | 2 4 2 3 | 3 2 2 2 | 2 3 3 3 | 2 3 0 3 |
| 2j 3j | 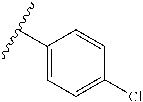 [SMILES: *c1ccc(Cl)cc1] | (2R,4S) (2S,4R) (2S,4S) (2S,4S) | 4 6 4 2 | 4 4 2 2 | 2 4 3 2 | 2 3 0 3 |
| 2k 3k | 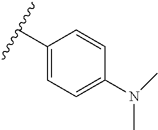 [SMILES: *c1ccc(N(C)C)cc1] | (2R,4S) (25,4R) (2S,4S) (2R,4R) | 2 3 2 5 | 2 3 2 2 | 2 5 2 4 | 2 2 0 2 |
| Pimavanserin | | — | 2 | 5 | 2 | 2 |
| Risperidone | | — | 2 | 2 | 2 | 2 |

The pharmacological parameters (e.g., pEC₅₀, pK_{b}, pIC₅₀, Iₘₐₓ) of agonist and antagonist-mediated signal transduction through Ga_{q}-coupled 5-HT₂A, 5-HT_{2B}, 5-HT_{2C}, and H₁Rs was quantified using the Cisbio (Bedford, MA) IP-One homogeneous time resolved fluorescence (HTRF) immunoassay. The protocol used was consistent with that recommended by the manufacturer for suspension cells in 384-well plate format, with minor modifications. Immediately following transfection, cells were washed twice with 10 mL of prewarmed 1×PBS, dissociated in 10 mL of 1×PBS, and centrifuged at 270 g for 5 minutes at room temperature. Cells (˜2,000 cells/μL, >90% viability, determined by the Corning® Cell Counter) were resuspended in the manufacturer's stimulation buffer (pH=7.4, 37° C.) modified to include 0.1% bovine serum albumin stabilizer (PerkinElmer, part #: CR84-100) and added to a white 384-well OptiPlate (PerkinElmer). Next, stimulation buffer, or 2× compound diluted in stimulation buffer, was added to each well. For competitive antagonism (pK_{b}) experiments, 2× antagonist was diluted in stimulation buffer containing 2× reference agonist (e.g., 2 μM 5-HT for WT and point mutated 5-HT_{2A}Rs, 20 nM 5-HT for 5-HT_{2B}Rs, and 20 μM histamine for H₁Rs) such that agonist and antagonist were added to the cells simultaneously. Cells were then incubated in the dark for two hours at 37° C. to ensure equilibrium was obtained. Plates were covered with an aluminum foil seal to prevent evaporation.

Following incubation, the manufacturer's lysis solution (detection buffer) containing inositol monophosphate (IP1) covalently bound to a fluorescent acceptor dye (d2) was added to each well. This process generated a homogenous mixture of cellular and d2-labeled IP1, in a ratio dependent on the concentration- and activity-dependent efficacy of test ligands to modulate cellular IP1 levels. Next, detection buffer containing an anti-IP1 antibody covalently bound to a terbium-cryptate, which serves as a fluorescent acceptor, was added to each well. A one-hour incubation at room temperature was used to allow competitive interactions between cellular and d2-labeled IP1 to equilibrate with anti-IP1-cryptate. Time-resolved fluorescent resonance energy transfer (TR-FRET) was then quantified by a Synergy H1 plate reader equipped with a HTRF filter cube (BioTek). Light pulse at 320 or 340 nm was used to excite the fluorescent donor, and emission at 615 and 665 nm was detected. The relative levels of TR-FRET were used to obtain an emission ratio at 665/620 nm, which was then used to interpolate the concentration of IP1 in each well.

Site directed mutagenesis experiments were performed using 5′-phosphorylated, PAGE-purified custom primers (Life Technologies, Carlsbad CA) and the Quikchange Site-Directed Mutagenesis kit (Agilent, Santa Clara CA) according to the manufacturer's protocol. Reactions were performed in thin-walled PCR tubes using a TOne Gradient 96 Thermal Cycler (Biometra). Primer sequences and optimized reaction conditions are provided in Table 7A, and the primer sequences are provided in Table 7B. Following Dpn1 digestion, 2 μL of the PCR product mixture was transformed into 50 μL XL1-Blue competent cells using a 45 second pulse of heat in a 42° C. water bath. The transformed bacteria were then incubated for 1 hour at 37° C. in 0.5 mL of LB broth before being plated on LB-agar plates and incubated overnight at 37° C. The next day, two colonies for each mutated receptor were grown overnight in LB broth at 37° C. The mutated cDNA was extracted the following day using the PureYield™ Plasmid Maxiprep System (Promega Corp., Madison WI). Purified DNA was sequence validated by Psomagen Inc. (Cambridge, MA).

TABLE 7A
Experimental conditions used to perform site-directed mutagenesis.

| Receptor | | 5-HT_{2A} | | | | | | | H₁ |
|---|---|---|---|---|---|---|---|---|---|
| Mutation | G238S^{5.42} | I210V^{4.60} | S242A^{5.46} | C322K^{6.34} | V235M^{5.39} | F213K^{4.63} | D231F^{5.35} | | W158I^{4.56} |
| Forward | 5′- | 5′- | 5′- | 5′- | 5′- | 5′- | 5′ | | 5′- |
| | CCTGATCTC | GGTATATCCA | CCTGATCGGC | GAGCAAAAGG | CGATGATA | CCAATACCA | GCTTACTCGCC | | CCTGGTTTC |
| | CTCTTTTGTG | TGCCAGTACC | TCTTTTGTGG | CAAAGAAGGT | ACTTTATG | GTCAAAGGG | TTTGATAACTTT | | TCTCTTTTC |
| | TCATTTTTC- | AGTCTTTG-3′ | CATTTTTCATT | GCTGGG-3′ | CTGATCGG | CTACAGGAT | GTCCTG-3′ | | TGATTGTTA |
| | 3′ | (SEQ ID NO: 2) | CCCTTAAC-3′ | (SEQ ID NO: 4) | CTC-3′ | TCG-3′ | (SEQ ID NO: 7) | | TTCCC-3′ |
| | (SEQ ID NO: 1) | | (SEQ ID NO: 3) | | (SEQ ID NO: 5) | (SEQ ID NO: 6) | | | (SEQ ID NO: 8) |
| Reverse | 5′- | 5′- | 5′- | 5′- | 5′- | 5′ | 5′- | | 5′- |
| | CAAAAGAGG | CCAAGACTGG | GTTAAGGGAA | CCCAGCACCT | GAGCCGAT | CCTGTAGCC | GACAAAGTTAT | | GCCTAGAAT |
| | AGATCAGGA | TACTGGCATG | TGAAAAATGC | TCTTTGCCTTT | CAGCATAA | CTTTGACTG | CAAAGGCGAG | | GGGAATAA |
| | CAAAGTTATC- | GATATACC-3′ | CACAAAAGAG | TGCTC-3′ | AGTTATCA | GTATTGGCA | TAAGCAAC-3′ | | CAATCAGAA |
| | 3′ | (SEQ ID NO: 10) | CCGATCAGG-3′ | (SEQ ID NO: 12) | TCG-3′ | TGG-3′ | (SEQ ID NO: 15) | | AAGAG-3′ |
| | (SEQ ID NO: 9) | | (SEQ ID NO: 11) | | (SEQ ID NO: 13) | (SEQ ID NO: 14) | | | (SEQ ID NO: 16) |
| Initial | 95 (60 sec) | | | | | | | | |
| Denaturation | | | | | | | | | |
| (° C.) | | | | | | | | | |
| Denaturation | 95 (60 sec) | | | | | | | | |
| (° C.) | | | | | | | | | |
| Annealing | 55 (60 sec) | 58 (60 sec) | 72 (60 sec) | 66 (60 sec) | 72 (60 sec) | 58 (60 sec) | 64 C | 62 | 58 (60 sec) |
| (° C.) | | | | | | | (60 sec) | (60 sec) | |
| Extension | 72 (7 min) | | | | | | | | |
| Final | 72 (10 min) | | | | | | | | |
| Extension | | | | | | | | | |
| (° C.) | | | | | | | | | |
| Hold | 4 (O/N) | | | | | | | | |
| (° C.) | | | | | | | | | |
| Cycles (n) | 25 | | | | | | | | |
| ng DNA | 10 | | | | | | | | |
| DMSO | 0 | | | 5% | 0 | | | | |
| (% v/v) | | | | | | | | | |

TABLE 7B
Primer Sequences from Table 7A.

| Sequence | | |
|---|---|---|
| name: | Sequence: | Desc.: |
| SEQ ID | CCTGATCTCCTCTTTTGTGTCATTTTTC | Primer |
| NO: 1 | | sequence |
| SEQ ID | GGTATATCCATGCCAGTACCAGTCTTTG | Primer |
| NO: 2 | | sequence |
| SEQ ID | CCTGATCGGCTCTTTTGTGGCATTTTTCATTCCCTTAAC | Primer |
| NO: 3 | | sequence |
| SEQ ID | GAGCAAAAGGCAAAGAAGGTGCTGGG | Primer |
| NO: 4 | | sequence |
| SEQ ID | CGATGATAACTTTATGCTGATCGGCTC | Primer |
| NO: 5 | | sequence |
| SEQ ID | CCAATACCAGTCAAAGGGCTACAGGATTCG | Primer |
| NO: 6 | | sequence |
| SEQ ID | GCTTACTCGCCTTTGATAACTTTGTCCTG | Primer |
| NO: 7 | | sequence |
| SEQ ID | CCTGGTTTCTCTCTTTTCTGATTGTTATTCCC | Primer |
| NO: 8 | | sequence |
| SEQ ID | CAAAAGAGGAGATCAGGACAAAGTTATC | Primer |
| NO: 9 | | sequence |
| SEQ ID | CCAAGACTGGTACTGGCATGGATATACC | Primer |
| NO: 10 | | sequence |
| SEQ ID | GTTAAGGGAATGAAAAATGCCACAAAAGAGCCGATCAGG | Primer |
| NO: 11 | | sequence |
| SEQ ID | CCCAGCACCTTCTTTGCCTTTTGCTC | Primer |
| NO: 12 | | sequence |
| SEQ ID | GAGCCGATCAGCATAAAGTTATCATCG | Primer |
| NO: 13 | | sequence |
| SEQ ID | CCTGTAGCCCTTTGACTGGTATTGGCATGG | Primer |
| NO: 14 | | sequence |
| SEQ ID | GACAAAGTTATCAAAGGCGAGTAAGCAAC | Primer |
| NO: 15 | | sequence |
| SEQ ID | GCCTAGAATGGGAATAACAATCAGAAAAGAG | Primer |
| NO: 16 | | sequence |

GraphPad Prism (La Jolla, CA) version 9.1.1 was used to analyze all experimental data in this work. To analyze data from saturation binding studies, the radioligand counts per minute (cpm) were normalized to fmol/mg protein bound and then fit the data to a ‘specific binding with hill slope’ model. For competitive radioligand displacement studies, a baseline correction was performed by subtracting the mean nonspecific binding value from the radioligand binding values with and without competitor to obtain specific binding values. Specific binding values were then normalized such that total binding in the absence of competitor represented 100% radioligand binding, and the radioactivity associated with each concentration of competitor was a percentage of total binding. The normalized data was then fit to the ‘one-site Fit Kᵢ’ nonlinear regression model. Ligand selectivity is reported as the fold change between mean affinity (Kᵢ) values.

The functional activity of each compound was determined by incubating cells expressing recombinant receptors with or without a compound of interest in parallel with well containing only buffer and 8 known concentrations of IP1 to generate a standard curve for each experiment. The IP1 concentration in cell-containing wells was then interpolated using nonlinear regression and the ‘log(inhibitor) vs. response (three parameters)’ model in Prism. To control for variation between assays, the resulting concentrations were transformed into molar units and change from basal was calculated using the following equation (Equation 1), where B is the basal concentration of IP, and Y is the concentration of IP generated by cells incubated with compound.

$$\% change from basal = \frac{Y - B}{B} \cdot 100 \qquad Equation 1$$

The antagonist equilibrium dissociation constant (K_{b}), determined in parallel with the EC₅₀ of reference agonists, was calculated using the following Equation 2 (Cheng, 2001):

$${K}_{b} = \frac{{IC}_{50}}{1 + {( \frac{A}{{EC}_{50}} )}^{K}} \qquad Equation 2$$

where IC₅₀ is the concentration of antagonist which inhibited 50% of the IP1 elicited by a constant concentration (A) of reference agonist (i.e., 1 μM 5-HT, 10 nM 5-HT, or 10 μM histamine for 5-HT_{2A}, 5-HT_{2B}, and H₁Rs, respectively), EC₅₀ is the concentration of the reference agonist which elicited a half maximal response, and K is the Hill slope of the reference agonist. The IC₅₀ of antagonists was determined using the ‘log([inhibitor]) vs. response (three parameter)’ model, where cells stimulated only by A represented the lowest X value (−12). The same model was used to calculate the IC₅₀ of inverse agonists in the absence of A. In contrast, the EC₅₀ was derived from a log([agonist]) vs. response-variable slope (four parameters)’ model to simultaneously obtain K. Each K_{b}, IC₅₀, and EC₅₀ value was logarithmically transformed into the pK_{b}, pIC₅₀, and pEC₅₀, respectively for ease of presentation and statistical analyses. Since standard deviations tend to be symmetrically distributed around log normal pharmacological parameters (e.g., pK_{b}, pIC₅₀, and pEC₅₀), but not equilibrium constants (e.g., K_{b}, IC₅₀, and EC₅₀), statistical comparisons of in vitro functional data were only performed using log normal values (Neubig, et al., 2003).

No blinding or randomization methods were used for in vitro studies since these measurements are insensitive to time of day or experimenter interpretation.

Data exclusion, sample size and statistical analyses were utilized. For purposes of efficient lead identification, each competitive radioligand displacement assay condition was performed using three or four technical replicates in at least two independent experiments. Some results from radioligand binding assays were not included in the reported data, for example, when excessive or incomplete radioligand displacement (>30%) occurred at the lowest or highest concentration of unlabeled ligand, respectively. Among these experiments were three of 130 pK values at the 5-HT_{2A}R (pK=6.99 for [2S,4S]-3h, pKᵢ=9.84 and 9.65 for [2S,4R]-2i), one of 125 pKᵢ values at the 5-HT_{2B}R (pKᵢ=7.32 for (2S,4R)-2k), and two of 119 pKᵢ values at the 5-HT_{2C}R (pKᵢ=9.08 for [2S,4R)-2c, and pKᵢ=9.17 for [2S,4R]-2d). These results were attributed to experimenter error during assay optimization or chemical contamination.

The number of concentrations (7-9) used between receptors varied in functional assays. Three technical replicates were used in all functional assays except for antagonism (K_{b}) assays, which utilized two technical replicates. Five independent experiments were performed to define the functional concentration-response relationships for ligands acting at point mutated 5-HT_{2A}Rs, WT 5-HT_{2B}Rs, WT 5-HT_{2C}Rs, and WT H₁Rs. The number of independent experiments performed at WT 5-HT_{2A}Rs to determine the EC₅₀ of 5-HT, and the K_{b} of antagonists, however, varied (n=14-19) since these experiments were performed in parallel with each other and often in parallel with analogous experiments at point mutated 5-HT_{2A}Rs (see Table 2 for exact n). For all in vivo conditions (Example 3), n=6 treatments were administered, except mice treated with vehicle+(±)-DOI (n=7) due to an additional mouse being procured in case of error.

The data and statistical analyses in this study comply with the recommendations on experimental design and analysis in pharmacology (Curtis, et al., 2018). Where n ? 5, all pharmacological parameters are presented according to Hopkin's two-digit rule (Hopkins, et al., 2011). Parametric one-way ANOVA and unpaired t-tests were used to analyze the data in this work. The p<0.05 was considered as statistically significant, and only performed statistical analyses on groups with n>5 independent samples. The potential agonist activity of (2S,4R)-2k or (2R,4S)-2c at 5-HT_{2B} or 5-HT_{2C}Rs, respectively, indicated by an exploratory screening effort (FIG. 2A), was assessed using a one-way ANOVA comparison of the mean percent change in basal response for n≥5 independent experiments performed in triplicate. One-way ANOVA was also used to compare the impact of compound treatment on the DOI-elicited head twitch response and locomotor activity, in mice relative to 1 mg*kg⁻¹ (±)-DOI or vehicle control, respectively. Tukey's or Dunnett's multiple comparison tests were used to complement these comparisons of in vivo data. The impact of individual point mutations within the 5-HT_{2A}R on antagonist affinity (pK_{b}) was assessed using unpaired parametric t-tests between the pK_{b(WT)} and pK_{b(mutant)} values for each antagonist, as well as between the pEC_{50(WT)} and pEC₍ₘᵤₜₐₙₜ₎ of 5-HT.

To ensure data adhered to assumptions of ordinary one-way ANOVA and unpaired parametric t-tests, diagnostic statistics were performed using Brown-Forsythe and F-tests to measure variance distribution, and Shapiro-Wilk tests for normality. Data abided by these assumptions in the majority of cases with few deviations, and where deviations occurred, nonparametric tests (Mann-Whitney U or Kruskal-Wallis) were performed to assess the robustness of a result. No deviations were endemic to a particular compound, receptor variant, or experimental technique, and no comparison violated both assumptions.

All tritiated radioligands were purchased from PerkinElmer (Boston, MA) and are shown in Table 5. 5-hydroxytryptamine hydrochloride and doxepine hydrochloride were purchased from Alfa Aesar (Ward Hill, MA). (±)-2,5-Dimethoxy-4-iodoamphetamine hydrochloride, chlorpromazine hydrochloride, histamine dihydrochloride, and tripolidine hydrochloride were purchased from Sigma Aldrich (St. Louis, MO). Mianserin hydrochloride, and risperidone (free base), were purchased from Tocris Biosciences (Bristol BS11 OQL, UK). PIMA tartrate was purchased from Selleck Chemical LLC (Houston, TX).

For nomenclature of targets and ligands, key protein targets and ligands are hyperlinked to corresponding entries in (guidetopharmacology.org), the common portal for data from the IUPHAR/BPS Guide to Pharmacology (Harding, et al., 2018), and are permanently archived in the Concise Guide to Pharmacology 2017/18 (Alexander, et al., 2017).

### Example 3. Comparative In Vivo Assessment of PIMA, (2S,4R)-2k and (2R,4R)-3h

Adult male C57BL/6J mice were procured from Jackson Laboratories (Bar Harbor, ME) at 8 weeks of age, and housed 4/cage inside sterile ventilated caging by Innovive (San Diego, CA) on irradiated corn cob. All cages were changed in an animal transfer station using forceps soaked in Clidox-S® solution (Genestil). Animals were maintained in an SPF facility on a 12-hr light:dark cycle with ad libitum access to pre-filled acidified water (Innovive) and irradiated rodent diet (Prolab Isopro). After at least one week at Northeastern University, mice were transported two floors above their vivarium to a testing facility kept at approximately 22° C. with a constant background noise level of 62 dB and fluorescent lighting. All animals were habituated to the novel environment for a minimum of one hour before handling. To eliminate bias during in vivo studies, mice were marked on the tail with a permanent marker and placed in an alphanumeric home cage. A random number generator was used to select the order of mice undergoing treatment, and treatments were blinded to the administrator and observers.

Compounds were prepared in vehicle (5% (v/v) DMSO in MilliQ water) and filtered through a 0.22 μm syringe filter. All injections were performed s.c. on the neck at 0.1 mL/10 g body weight. PIMA and (2S,4R)-2k were administered at 0.3 or 3 mg kg⁻¹, whereas (2R,4R)-3h was administered at elevated doses (3.0 or 5.6 mg kg⁻¹) as pilot studies indicated analogs in the (2R,4R)-configuration were less active in vivo. For all testing procedures, mice were pretreated with compound, placed in their home cage for the time indicated below, and then in an open field arena (43 cm×43 cm, Med Associates, St. Albans, VT). Trials were video-taped using a ceiling-mounted video tracking system connected to Noldus Ethovision XT9 software (Noldus Information Technology, Leesburg, VA) allowing for locomotor activity tracking (distance traveled, cm). Animals were sacrificed by cervical dislocation after being anesthetized with isoflurane.

All behavioral procedures comply with the Guide for the Care and Use of Laboratory Animals (Council, 2011) and were approved by the Northeastern University Institutional Animal Care and Use Committee. The animal care and use program is fully accredited by AAALAC, International and holds an Assurance with OLAW. Moreover, these studies are in accordance with the ARRIVE 2.0 guidelines (Percie du Sert, et al., 2020) and recommendations made by the British Journal of Pharmacology.

DOI-elicited head twitch response assays were performed (FIG. 3A). Similar to previous reports (Canal, et al., 2014; Canal, et al., 2015), treatment naive subjects at 9 weeks of age were administered either vehicle or compound and returned to their home cage for 15 minutes. Subjects were then injected once more, this time with 1 mg kg⁻¹ (±)-DOI and returned to their home cage for 10 minutes before being placed in the open field arena for the 10-minute test session. Two trained observers (A.B.C and R.P.M) counted the head twitch response, defined as rapid, discrete back-and-forth twisting of the head. Among 43 trials, 34.9% of the scores were entirely consistent, whereas scores differed by 1, 2, 3, 4, 5, 6 or 7 head twitches in 23.3%, 18.6%, 14%, 4.7%, 2.3%, 0%, and 2.3% of trials. All scores by the two observers were averaged for each subject.

Locomotor activity assays were performed (FIG. 3B, FIG. 3C). In accordance with the principles of reduction, replacement and refinement of animal welfare, the subjects used in the head twitch response assay were also used to assess compound-induced alterations in spontaneous locomotion following a 6-week washout period (15 weeks of age). Subjects were randomized, pretreated with vehicle or 3 mg*kg⁻¹ compound, and placed in their home cage for 15 minutes before vehicle administration. Ten minutes later, subjects were placed in an open field for a 10-minute test session while the experimenter stepped out of view. This format was intended to mirror the conditions used in the (±)-DOI-elicited head twitch response assay while allowing for the measurement of ligand-induced locomotor activity in isolation. It was aimed to mirror the conditions in 2.6.1 since the associated locomotor results indicated that PIMA had a higher propensity to induce locomotor suppression compared to 4-PATs when PIMA was co-administered with (±)-DOI.

To compare the in vivo activity of PIMA, (2S,4R)-2k, and (2R,4R)-3h the DOI-elicited head twitch response assay in mice was used as a model of central 5-HT_{2A}R engagement sensitive to antipsychotic-like activity (Canal & Morgan, 2012). Acute administration of each compound significantly attenuated the head twitch response at every dose tested (FIG. 3A). The lower dose of PIMA (i.e., 0.3 mg*kg⁻¹) was significantly more effective at attenuating the head twitch response than the same dose of (2S,4R)-2k. In contrast, no differences were observed between 3 mg*kg⁻¹ PIMA and 3 mg*kg⁻¹ (2S,4R)-2k, 3 mg*kg⁻¹ PIMA and 5.6 mg*kg⁻¹ (2R,4R)-3h, or 3 mg*kg⁻¹ (2S,4R)-2k and 5.6 mg*kg⁻¹ (2R,4R)-3h.

Notably, only male mice were used in this study, and it is unclear if the behavioral findings generalize to female mice, although sex differences in the sensitivity of mice to DOI are nil (Canal & Morgan, 2012). Future studies investigating the efficacy and safety of novel 4-PATs in more comprehensive animal models of psychosis should consider sex as an experimental variable.

### Example 4. Molecular Modeling and Site-Directed Mutagenesis

For computational chemistry and molecular modeling, physicochemical parameters (log P and log D) were determined computationally using StarDrop™ (Optibrium) version 6.5 and the freebase form of 4-PAT-type compounds (Optibrium). All molecular modeling images were generated using the PyMOL Molecular Graphics System, Version 2.0 Schrödinger, LLC (Schrodinger, 2015).

In molecular docking work, the 3D PAT analogs were built using Maestro (Schrodinger, LLC) and were optimized using an ab initio quantum chemistry method at the HF/6-31G* level, followed by single point energy calculations of molecular electrostatic potential for charge fitting using Gaussian 16 (Gaussian, Inc.) (Bayly, et al., 1993). The atomic charges derived from ab initio calculations were used for molecular docking simulations. The crystal structures for 5-HT_{2A}R (PDB: 6A94) and H₁R (PDB: 3RZE) were processed to add missing sidechains and loops with Discovery Studio software (BIOVIA). AutoDock 4.2 (Morris, et al., 1998) was used to dock molecules into the receptors with selected sidechain flexible residues in the binding pocket. A grid map was generated for the receptor using C, H, N, O, S, F, Cl, Br, I (i.e., carbon, hydrogen, nitrogen, oxygen, sulfur, fluorine, chlorine, bromine, and iodine) elements sampled on a uniform grid containing 80×80×80 points, 0.375 Å apart. The Lamarckian genetic algorithm was selected to identify ligand binding conformations. For each ligand, 100 docking simulations were performed. The final docked ligand conformations were selected based on binding energies and cluster analysis.

The molecular dynamics simulations were performed was follows: the protonation states of the titratable residues of 5-HT_{2A}, 5-HT_{2B}, 5-HT_{2C} and H₁ structures were calculated at pH=7.4 using the H++ server (biophysics.cs.vt.edu/). The PAT-bound receptor complexes obtained from molecular docking studies were inserted into a simulated lipid bilayer composed of POPC:POPE:cholesterol (2:2:1), (Grossfield, et al., 2008) and a water box using CHARMM-GUI Membrane Builder webserver (charmm-gui.org). Sodium chloride (150 mM) and extra neutralizing counter ions were added into the systems. The PMEMD.CUDA program of AMBER 16 was used to conduct MD simulations. The Amber ff14SB, lipid17, and TIP3P force field was used for the receptors, lipids, and water. The parameters of PAT analogs were generated using general AMBER force field by the Antechamber module of AmberTools 17. The partial charges for the compounds were calculated using a restrained electrostatic potential charge-fitting scheme by ab initio quantum chemistry at the HF/6-31G* level 15 (Gaussian 16) (Bayly, et al., 1993). System topology and coordinate files were generated by using the tleap module of Amber. The systems were energy minimized by 500 steps (with position restraint of 500 kcal/mol/A²) followed by 2000 steps (without position restraint) using the steepest descent algorithm. Subsequently, the systems were heated from 0-303 K using Langevin dynamics with a collision frequency of 1 ps⁻¹. Receptor complexes were position-restrained using an initial constant force of 500 kcal/mol/Å² during the heating process, and weakened to 10 kcal/mol/Å², allowing lipid and water molecules free movement. Next, systems went through 5 ns equilibrium MD simulations. Finally, a total of 100-1000 ns MD simulations were conducted, and coordinates were saved every 100 ps for analysis. The MD simulations were conducted under NPT (constant temperature and pressure). Pressure was regulated using an isotropic position scaling algorithm with the pressure relaxation time fixed at 2.0 ps. Long range electrostatics was calculated by a particle mesh Ewald method (Darden, 1993) with a 10 Å cutoff.

The SAR results reported above and elsewhere (Canal, et al., 2014; Sakhuja, et al., 2015) indicated that selectivity to bind 5-HT_{2A} and/or 5-HT_{2C}Rs is negligible for 4-PATs with small meta-substituents on ring C (e.g., 3a, 3b, 3b, Table 1). In contrast, larger aryl substituents at this position can yield moderate to high selectivity to bind 5-HT_{2A}Rs over 5-HT_{2B}Rs in the (2S,4R)-configuration, and over 5-HT_{2B}, 5-HT_{2C} and H₁Rs in the (2R,4R)-configuration. Meanwhile, PIMA selectively bound 5-HT_{2A}Rs over 5-HT_{2B} and H₁Rs, with moderate selectivity over 5-HT_{2C}Rs. To understand how aryl substituted 4-PATs and PIMA bind to 5-HT_{2A}Rs, molecular modeling studies were performed using a model of the 5-HT_{2A}R (FIGS. 4A-4C). Site-directed mutagenesis was used to validate the proposed ligand-receptor interactions. The molecular modeling results with the 5-HT_{2A}R bound to inverse agonists PIMA, (2S,4R)-2k and (2R,4R)-3h revealed similar binding modes for each ligand (FIG. 4A, FIG. 4B, FIG. 4C). All compounds docked close enough to the side chain of D155^{3.32} (Ballesteros-Weinstein numbering system, Ballesteros, 1995) such that an ionic bond could form with their respective basic amine moieties, a highly conserved interaction critical to the binding of most ligands across aminergic GPCRs (Kristiansen, et al., 2000; Vass, et al., 2019). Each ligand could also interact with conserved residues in TM3, including V156^{3.33} S169^{3.36} and T160^{3.37} (Table 3).

Overall, PIMA, (2S,4R)-2k, and (2R,4R)-3h stabilized an inactive-like conformation of the 5-HT_{2A}R, typified by an ionic lock between R173^{3.50} and E318^{6.30} within the E/DRY domain (FIG. 8). The ionic lock may restrict the intracellular end of TM6 from outward displacement, and thus inhibit productive Ga_{q}-coupling and accumulation of inositol phosphates (Shapiro, et al., 2002). Clues to how the inactive state was stabilized are found at the ligand-receptor interface. For example, the simulations indicated that the fluorobenzyl ring of PIMA, as well as the aminotetralin core of (2S,4R)-2k and (2R,4R)-3h, may situate deep in the hydrophobic cleft of the orthosteric binding pocket. In this way, the fluorobenzyl and aminotetralin moieties could interact directly with I163^{3.40} and F332^{6.44} of the conserved P246^{5.50}-I163^{3.40}-F322^{6.44} motif, thought to be involved in the activation mechanism of 5-HT₂-type GPCRs (Kim, et al., 2020; Kimura, et al., 2019; Peng, et al., 2018). Similar interactions were observed between these moieties and the side chain of W336^{6.48}, a ‘toggle switch’ potentially mediating on/off states of class A GPCRs (Kim, et al., 2020; Peng, et al., 2018; Rasmussen, et al., 2011; Visiers, et al., 2002) (FIG. 4A, FIG. 4B, FIG. 4C, FIG. 8). Moreover, PIMA, (2S,4R)-2k, and (2R,4R)-3h could form edge-to-face aromatic interactions with the side chains of F243^{5.47} and F340^{6.52}, while π-cation interactions could form between the side chain of F339^{6.51} and the basic nitrogen of the PIMA piperidine fragment or the tertiary amine of (2S,4R)-2k and (2R,4R)-3h.

The models also indicated that the isobutoxybenzyl moiety of PIMA and aryl ring D of (2S,4R)-2k and (2R,4R)-3h occupied a side cavity between TM4 and TM5, unimpeded by the small side chain of G238^{5.42}, a residue unique to 5-HT₂-type receptors among aminergic GPCRs. Furthermore, in all models, F234^{5.38} assumed a rotamer conformation oriented away from G238^{5.42}, which has been suggested to extend the side cavity (Kimura, et al., 2019). Several amphipathic and hydrophobic side chains in this region of the binding pocket (I210^{4.60}, V235^{5.39}, G238^{5.42}, and S242^{5.46}) were close enough to the isobutoxybenzyl of PIMA and aryl ring D of (2S,4R)-2k and (2R,4R)-3h to facilitate interactions (Table 3), thus providing a potential structural basis for the observed selectivity of these ligands to bind 5-HT_{2A}Rs.

To validate the molecular modeling results, residues were point-mutated in and around the 5-HT_{2A}R side-extended cavity (Kimura, et al., 2019) and quantified the antagonist affinity (pK_{b}) of (2S,4R)-2k and (2R,4R)-3h, as well as (2S,4R)-2a (which lacks 5-HT₂R subtype selectivity) at 5-HT_{2A}R variants to understand how stereochemistry and aryl ring D impact ligand-receptor interactions. Notably, like PIMA, (2S,4R)-2k, and (2R,4R)-3h, key analog (2S,4R)-2a demonstrated inverse agonist activity at C322K^{6.34} 5-HT_{2A}Rs (FIG. 9). An assessment was also made of the antagonist affinity of PIMA and risperidone at point-mutated 5-HT_{2A}Rs, which represent selective and promiscuous 5-HT_{2A}R ligands, respectively.

A G238S^{5.42} 5-HT_{2A}R was generated to test the hypothesis that the large side chain of serine precludes ligand access to the side extended cavity, as suggested by the molecular modeling results (FIG. 10A, FIG. 10B) and reported elsewhere for PIMA (Kimura, et al., 2019). Compared to WT 5-HT_{2A}Rs, a modest, but significant, decrease in the pK_{b} of risperidone and (2S,4R)-2a at G238S^{5.42} 5-HT_{2A}Rs was observed. Moreover, the affinity of PIMA, (2S,4R)-2k, and (2R,4R)-3h was nearly abolished at G238S^{5.42} 5-HT_{2A}Rs (Table 2, FIG. 11F). Notably, the Δ(pK_{b}) for (2S,4R)-2a was less than that of (2S,4R)-2k and (2R,4R)-3h suggesting a size-dependent negative steric interaction between the 4-PAT ring C substituent and S^{5.42}. Also, a significant reduction in the potency was observed, but not the efficacy, of 5-HT at G238S^{5.42} compared to WT 5-HT_{2A}Rs (Table 2, FIG. 11A, FIG. 11B), consistent with previous reports (Kimura, et al., 2019).

The above experiments were extended by asking if the attenuated affinity of (2S,4R)-2a, (2S,4R)-2k, and (2R,4R)-3h at G238S^{5.42} 5-HT_{2A}Rs translates to aminergic GPCRs natively presenting S^{5.42}. Table 4 shows that (2S,4R)-2k and (2R,4R)-3h had >1,000-fold selectivity for 5-HT_{2A}Rs over 5-HT₁A, 5-HT₇, D_{2L}, α₁A- and α_{1B}-adrenergic GPCRs. It was noted that (2S,4R)-2k had 270-fold selectivity over D₃Rs, whereas (2R,4R)-3h had >1,000-fold selectivity. In contrast, (2S,4R)-2a exhibited moderate-to-high affinity for 5-HT₇, D_{2L}, D₃, and α₁A-adrenergic receptors.

Interestingly, aryl substituted 4-PATs in the (2S,4R)-configuration had high affinity for H₁Rs (Table 1), despite the presence of T194^{5.42}, which possesses a bulkier side chain than serine. The molecular modeling results suggested that W158^{4.56}, a residue unique to H₁Rs, might form stereospecific aromatic interactions with 4-PATs to impart high affinity (FIG. 5A, FIG. 5B). For example, ring D of (2S,4R)-2k positioned close to TM4, where it could form optimal T-shaped interactions with W158^{4.56} while ring B of the aminotetralin core could form edge-to-face aromatic interactions with W428⁶⁴⁸. In contrast, ring D of (2R,4R)-3h oriented toward TM5, potentially, due to a stereochemical restriction at the C(2)-position. Interactions with residues in TM5 might be disfavored by a negative steric interaction with T194^{5.42}, and cause ring D to position between TM5 and TM6, precluding optimal aromatic interactions between ring B and the side chain of W428^{6.48}. To validate the proposed model, a W1581^{4.56} H₁R was generated. However, W1581^{4.56} H₁Rs were unable to stimulate IP1 accumulation in response to histamine, and specific binding of [³H]mepyramine or [³H]ketanserin could not be detected.

Alignment of 5-HT_{2A}, 5-HT_{2B} and 5-HT_{2C}R crystal structures (FIG. 12A, FIG. 12B) indicated that a side chain rotamer of F234^{5.38}, unique to 5-HT_{2A}Rs (Kimura, et al., 2019), orients toward the extracellular end of TM4 to form a side-extended cavity potentially due to hydrophobic interactions with F213^{4.63}. In contrast, K193^{4.63} and 1192^{4.63} in 5-HT_{2B} and 5-HT_{2C}Rs, respectively, do not form productive interactions with F^{5.38} and may restrict the side cavity (Kimura, et al., 2019). However, no experimental studies have been reported to test this hypothesis. Therefore, a F213K^{4.63} 5-HT_{2A}R was generated to test the hypothesis that the selectivity of PIMA, (2S,4R)-2k, and (2R,4R)-3h to bind 5-HT_{2A}Rs relies on an interaction between F213^{4.63} and F234^{5.38}. Unexpectedly, a change in the pK_{b} of any antagonist tested at F213K^{4.63}5-HT₂ARs was not observed (Table 2, FIG. 11D). However, a decrease in the potency of 5-HT at F213K^{4.63} 5-HT_{2A}Rs was observed, but not its efficacy (Table 2, FIG. 11A, FIG. 11B). These results do not support the hypothesis that F213^{4.63} mediates subtype selective binding of inverse agonists at 5-HT_{2A}Rs, however, F213^{4.63} may be involved in 5-HT binding.

Further inspection of the 5-HT₂-type receptor crystal structures revealed that one helical turn above F^{5.38} in 5-HT_{2A} and 5-HT_{2C}Rs exists a non-conserved residue in 5-HT_{2B}Rs (D231^{5.35}, D211^{5.35}, and F214^{5.35}, respectively). The root-mean-square deviation (RMSD) of the F^{5.38} side chain in WT 5-HT_{2A} and 5-HT_{2B}Rs was tracked in silico and found that F^{5.38} exhibited large transient variations in RMSD in WT 5-HT₂ARs. Interestingly, the RMSD of F^{5.38} in D231F^{5.35} 5-HT₂ARs recapitulated the restricted pattern observed in silico for WT 5-HT_{2B}Rs, indicating that D231^{5.35} may facilitate flexibility in the side chain of F^{5.38} (FIG. 13).

It was therefore hypothesized that D231^{5.35} may modulate the side chain rotamer of F234^{5.38} in 5-HT_{2A}Rs to mediate subtype selective binding. To test this hypothesis, a D231F^{5.35} 5-HT_{2A}R was generated, however, D231F^{5.35} 5-HT_{2A}Rs were insufficiently responsive to 5-HT for competitive antagonism studies (FIG. 11B), thus, antagonist activity could not be experimentally determined. Furthermore, no specific binding was detected for [³H]ketanserin, [³H]mesulergine, or [³H]spiperone in exploratory studies using membranes from cells transfected with cDNA encoding D231F^{5.35} 5-HT_{2A}Rs (FIGS. 14A-14D).

Residues in TM4 and TM5 lining the side-extended cavity of 5-HT_{2A}Rs and in proximity to PIMA, (2S,4R)-2k, and (2R,4R)-3h (Table 3) were then investigated. Among these were the side chains of 1210⁴-6, V235^{5.39}, and S242^{5.46}. Importantly, the side chains of 1210^{4.60} and V235^{5.39} are conserved in 5-HT_{2C}Rs, while S242^{5.46} is unique to 5-HT_{2A}Rs. It was hypothesized that selectivity to bind 5-HT_{2A} and 5-HT_{2C}Rs over 5-HT_{2B}Rs may involve interactions with the side chains of 1^{4.60}, V^{5.39}, or the 5-HT_{2A}R-specific residue S242^{5.46}. In fact, a significant increase in the affinity of PIMA and (2S,4R)-2k at V235M^{5.39} 5-HT_{2A}Rs was observed, with no change in affinity for any antagonist at 1210V^{4.60} or S242A^{5.46} 5-HT_{2A}Rs (Table 2, FIG. 11C, FIG. 11E, FIG. 11G). Interestingly, the potency of 5-HT (but not the efficacy) was attenuated at V235M^{5.39} and S242A^{5.46} but not 1210V^{4.60} 5-HT_{2A}Rs, consistent with other reports investigating 1210V^{4.60} and S242A^{5.46} 5-HT_{2A}Rs (Table 2, FIG. 11A, FIG. 11B) (Kimura, et al., 2019).

### Example 5. X-Ray Crystal Structures

X-ray crystal data for compound 3b was acquired. The computing details were as follows: data collection: APEX3 (Bruker, 2016); cell refinement: SAINT V8.40A (Bruker, 2016); data reduction: SAINT V8.40A (Bruker, 2016); program(s) used to solve structure: ShelXT (Sheldrick, 2015); program(s) used to refine structure: SHELXL (Sheldrick, 2015); molecular graphics: Olex2 (Dolomanov et al., 2009); software used to prepare material for publication: Olex2 (Dolomanov et al., 2009). The identification code was: mukherjee_neu2_Om.

TABLE 8
Crystal data for compound 3b.

| | |
|---|---|
| C₁₈H₂₁ClN•Cl | Dₓ = 1.272 Mg m⁻³ |
| Mᵣ = 322.26 | Cu Ka radiation, λ = 1.54178 Å |
| Orthorhombic, P2₁2₁2₁ | Cell parameters from 9860 reflections |
| a = 5.9420 (3) Å | θ = 4.3-66.9° |
| b = 11.1529 (5) Å | μ = 3.40 mm-1 |
| C = 25.3900 (12) Å | T = 100 K |
| V = 1682.61 (14) Å³ | Bar, clear colourless |
| Z = 4 | 0.18 × 0.07 × 0.04 mm |
| F(000) = 680 | |

TABLE 9
Data collection for compound 3b.

| | |
|---|---|
| Bruker X8 Proteum-R | 2968 independent |
| diffractometer | reflections |
| Radiation source: rotating anode | 2884 reflections with I > 2σ(I) |
| Montel monochromator | Rᵢₙₜ = 0.060 |
| ϕ and ω scans | θₘₐₓ = 66.9°, θₘᵢₙ = 4.3° |
| Absorption correction: multi-scan | h = −7→7 |
| SADABS2016/2 (Bruker, 2016/2) was | |
| used for absorption correction. | |
| wR2(int) was 0.1166 before and | |
| 0.0912 after correction. The | |
| Ratio of minimum to maximum | |
| transmission is 0.8256. The λ/2 | |
| correction factor is not present. | |
| Tₘᵢₙ = 0.615, Tₘₐₓ = 0.744 | k = −13→13 |
| 45402 measured reflections | l = −29→30 |

TABLE 10
Refinement for compound 3b.

| | |
|---|---|
| Refinement on F² | Hydrogen site location: mixed |
| Least-squares matrix: full | H atoms treated by a mixture of |
| | independent and constrained refinement |
| R[F² > 2σ(F²)] = 0.031 | w = 1/[σ²(Fₒ²) + (0.0349P)² + 1.1454P] |
| | where P = (Fₒ² + 2F_{c}²)/3 |
| wR(F²) = 0.078 | (Δ/σ)ₘₐₓ < 0.001 |
| S = 1.02 | Δₘₐₓ = 0.24 e Å⁻³ |
| 2968 reflections | Δₘᵢₙ = −0.36 e Å⁻³ |
| 196 parameters | Absolute structure: Refined as an |
| | inversion twin. |
| 0 restraints | Absolute structure parameter: 0.069 (19) |
| Primary atom site location: | |
| dual | |

Special details were as follows: Geometry. All esds (except the esd in the dihedral angle between two I.s. planes) are estimated using the full covariance matrix. The cell esds are taken into account individually in the estimation of esds in distances, angles and torsion angles; correlations between esds in cell parameters are only used when they are defined by crystal symmetry. An approximate (isotropic) treatment of cell esds is used for estimating esds involving I.s. planes. Refinement. Refined as a 2-component inversion twin.

TABLE 11
Fractional atomic coordinates and isotropic or equivalent
isotropic displacement parameters (Å²) for (2R,4R)-3b.

| | x | | y | | z | | Uᵢₛₒ*/U_{eq} |
|---|---|---|---|---|---|---|---|
| Cl2 | 0.88258 | (11) | 0.55367 | (6) | 0.24390 | (3) | 0.02293 (17) |
| Cl1 | 0.58356 | (17) | 0.15348 | (8) | 0.46069 | (4) | 0.0499 (3) |
| N1 | 0.5574 | (4) | 0.7492 | (2) | 0.27226 | (9) | 0.0171 (5) |
| H1 | 0.660 | (5) | 0.693 | (3) | 0.2646 | (12) | 0.020* |
| C12 | 0.3887 | (5) | 0.5955 | (2) | 0.33127 | (10) | 0.0176 (5) |
| H12A | 0.509165 | | 0.540350 | | 0.319457 | | 0.021* |
| H12B | 0.255240 | | 0.582857 | | 0.308608 | | 0.021* |
| C5 | 0.1331 | (5) | 0.7709 | (2) | 0.38592 | (10) | 0.0198 (6) |
| C3 | 0.4679 | (5) | 0.7240 | (2) | 0.32688 | (11) | 0.0186 (6) |
| H3 | 0.597167 | | 0.733302 | | 0.351783 | | 0.022* |
| C9 | −0.0116 | (5) | 0.6228 | (3) | 0.44571 | (11) | 0.0214 (6) |
| H9 | −0.006632 | | 0.544144 | | 0.460043 | | 0.026* |
| C10 | 0.1436 | (5) | 0.6548 | (2) | 0.40696 | (10) | 0.0177 (6) |
| C11 | 0.3285 | (4) | 0.5698 | (2) | 0.38904 | (10) | 0.0180 (6) |
| H11 | 0.465101 | | 0.588122 | | 0.410584 | | 0.022* |
| C13 | 0.2790 | (5) | 0.4371 | (3) | 0.39580 | (10) | 0.0197 (6) |
| C8 | −0.1720 | (5) | 0.7020 | (3) | 0.46377 | (11) | 0.0244 (7) |
| H8 | −0.275419 | | 0.678034 | | 0.490290 | | 0.029* |
| C6 | −0.0307 | (5) | 0.8499 | (3) | 0.40423 | (11) | 0.0213 (6) |
| H6 | −0.038832 | | 0.928285 | | 0.389719 | | 0.026* |
| C7 | −0.1816 | (5) | 0.8169 | (3) | 0.44304 | (11) | 0.0244 (7) |
| H7 | −0.290824 | | 0.872409 | | 0.455378 | | 0.029* |
| C1 | 0.3876 | (5) | 0.7457 | (3) | 0.22867 | (11) | 0.0225 (6) |
| H1A | 0.290992 | | 0.675139 | | 0.232978 | | 0.034* |
| H1B | 0.465557 | | 0.741106 | | 0.194713 | | 0.034* |
| H1C | 0.295337 | | 0.818430 | | 0.229816 | | 0.034* |
| C14 | 0.0908 | (5) | 0.3851 | (3) | 0.37229 | (11) | 0.0242 (6) |
| H14 | −0.014184 | | 0.433647 | | 0.353817 | | 0.029* |
| C2 | 0.6871 | (5) | 0.8633 | (3) | 0.27007 | (12) | 0.0240 (6) |
| H2A | 0.584391 | | 0.931203 | | 0.274793 | | 0.036* |
| H2B | 0.762154 | | 0.869971 | | 0.235823 | | 0.036* |
| H2C | 0.800244 | | 0.863869 | | 0.298162 | | 0.036* |
| C18 | 0.4296 | (5) | 0.3655 | (3) | 0.42272 | (11) | 0.0244 (6) |
| H18 | 0.558587 | | 0.399882 | | 0.438880 | | 0.029* |
| C15 | 0.0567 | (6) | 0.2616 | (3) | 0.37591 | (13) | 0.0331 (8) |
| H15 | −0.071234 | | 0.226364 | | 0.359695 | | 0.040* |
| C17 | 0.3915 | (6) | 0.2427 | (3) | 0.42604 | (12) | 0.0313 (7) |
| C4 | 0.2915 | (6) | 0.8136 | (3) | 0.34327 | (13) | 0.0325 (8) |
| H4A | 0.201480 | | 0.835611 | | 0.311909 | | 0.039* |
| H4B | 0.368078 | | 0.887146 | | 0.355773 | | 0.039* |
| C16 | 0.2082 | (6) | 0.1901 | (3) | 0.40297 | (14) | 0.0359 (8) |

| | | | | |
|---|---|---|---|---|
| H16 | 0.185177 | 0.105960 | 0.405476 | 0.043* |

TABLE 12
Atomic displacement parameters (Å²) for (mukherjee_neu2_0m).

| | U¹¹ | | U²² | | U³³ | | U¹² | | U¹³ | | U²³ | |
|---|---|---|---|---|---|---|---|---|---|---|---|---|
| Cl2 | 0.0159 | (3) | 0.0189 | (3) | 0.0340 | (4) | 0.0016 | (3) | −0.0004 | (3) | −0.0022 | (3) |
| Cl1 | 0.0519 | (6) | 0.0334 | (4) | 0.0644 | (6) | 0.0042 | (4) | −0.0121 | (5) | 0.0231 | (4) |
| N1 | 0.0149 | (11) | 0.0157 | (11) | 0.0205 | (12) | −0.0015 | (9) | 0.0009 | (9) | 0.0025 | (9) |
| C12 | 0.0180 | (13) | 0.0160 | (12) | 0.0189 | (12) | −0.0010 | (12) | 0.0013 | (11) | 0.0015 | (10) |
| C5 | 0.0225 | (14) | 0.0202 | (13) | 0.0167 | (13) | −0.0011 | (12) | 0.0009 | (12) | 0.0001 | (10) |
| C3 | 0.0206 | (14) | 0.0167 | (14) | 0.0186 | (13) | −0.0023 | (11) | 0.0014 | (11) | 0.0024 | (11) |
| C9 | 0.0250 | (14) | 0.0232 | (15) | 0.0158 | (13) | −0.0074 | (12) | −0.0038 | (11) | 0.0006 | (11) |
| C10 | 0.0190 | (13) | 0.0185 | (13) | 0.0156 | (12) | −0.0044 | (11) | −0.0033 | (11) | −0.0010 | (10) |
| C11 | 0.0167 | (13) | 0.0177 | (14) | 0.0195 | (13) | −0.0022 | (11) | −0.0026 | (10) | 0.0027 | (11) |
| C13 | 0.0235 | (14) | 0.0194 | (14) | 0.0163 | (12) | −0.0042 | (12) | 0.0024 | (11) | 0.0046 | (11) |
| C8 | 0.0233 | (16) | 0.0328 | (17) | 0.0172 | (13) | −0.0049 | (12) | 0.0023 | (11) | −0.0023 | (12) |
| C6 | 0.0239 | (14) | 0.0193 | (14) | 0.0206 | (14) | 0.0010 | (12) | −0.0027 | (12) | −0.0019 | (12) |
| C7 | 0.0224 | (15) | 0.0307 | (17) | 0.0202 | (14) | 0.0030 | (12) | −0.0001 | (11) | −0.0083 | (12) |
| C1 | 0.0204 | (14) | 0.0253 | (14) | 0.0218 | (14) | −0.0009 | (14) | −0.0033 | (12) | 0.0025 | (11) |
| C14 | 0.0269 | (16) | 0.0227 | (14) | 0.0229 | (14) | −0.0069 | (14) | −0.0045 | (13) | 0.0045 | (11) |
| C2 | 0.0234 | (14) | 0.0228 | (15) | 0.0260 | (15) | −0.0079 | (12) | 0.0017 | (12) | 0.0031 | (12) |
| C18 | 0.0259 | (15) | 0.0204 | (14) | 0.0269 | (14) | −0.0022 | (12) | −0.0021 | (12) | 0.0052 | (11) |
| C15 | 0.039 | (2) | 0.0259 | (16) | 0.0345 | (17) | −0.0118 | (15) | −0.0056 | (15) | 0.0007 | (14) |
| C17 | 0.0376 | (17) | 0.0256 | (15) | 0.0308 | (16) | −0.0033 | (16) | 0.0005 | (16) | 0.0106 | (13) |
| C4 | 0.0421 | (19) | 0.0206 | (16) | 0.0348 | (17) | 0.0058 | (14) | 0.0188 | (15) | 0.0069 | (13) |
| C16 | 0.050 | (2) | 0.0199 | (16) | 0.0379 | (18) | −0.0063 | (15) | −0.0004 | (16) | 0.0062 | (14) |

TABLE 13
Geometric parameters (Å, °) for (mukherjee_neu2_0m).

| | | | |
|---|---|---|---|
| Cl1—C17 | 1.751 (3) | C9—C8 | 1.378 (4) |
| N1—C3 | 1.512 (3) | C10—C11 | 1.521 (4) |
| N1—C1 | 1.498 (3) | C11—C13 | 1.518 (4) |
| N1—C2 | 1.489 (4) | C13—C14 | 1.395 (4) |
| C12—C3 | 1.513 (4) | C13—C18 | 1.381 (4) |
| C12—C11 | 1.537 (4) | C8—C7 | 1.387 (4) |
| C5—C10 | 1.401 (4) | C6—C7 | 1.382 (4) |
| C5—C6 | 1.393 (4) | C14—C15 | 1.394 (4) |
| C5—C4 | 1.512 (4) | C18—C17 | 1.391 (4) |
| C3—C4 | 1.507 (4) | C15—C16 | 1.385 (5) |
| C9—C10 | 1.395 (4) | C17—C16 | 1.369 (5) |
| C1—N1—C3 | 115.9 (2) | C13—C11—C10 | 115.7 (2) |
| C2—N1—C3 | 112.0 (2) | C14—C13—C11 | 120.8 (2) |
| C2—N1—C1 | 110.1 (2) | C18—C13—C11 | 119.6 (2) |
| C3—C12—C11 | 108.6 (2) | C18—C13—C14 | 119.4 (3) |
| C10—C5—C4 | 122.4 (3) | C9—C8—C7 | 119.6 (3) |
| C6—C5—C10 | 119.2 (3) | C7—C6—C5 | 121.5 (3) |
| C6—C5—C4 | 118.3 (3) | C6—C7—C8 | 119.3 (3) |
| N1—C3—C12 | 110.7 (2) | C15—C14—C13 | 119.9 (3) |
| C4—C3—N1 | 112.0 (2) | C13—C18—C17 | 119.6 (3) |
| C4—C3—C12 | 113.1 (2) | C16—C15—C14 | 120.5 (3) |
| C8—C9—C10 | 121.8 (3) | C18—C17—Cl1 | 118.9 (3) |
| C5—C10—C11 | 119.6 (2) | C16—C17—Cl1 | 119.4 (2) |
| C9—C10—C5 | 118.4 (3) | C16—C17—C18 | 121.7 (3) |
| C9—C10—C11 | 121.9 (2) | C3—C4—C5 | 114.9 (2) |
| C10—C11—C12 | 109.7 (2) | C17—C16—C15 | 118.8 (3) |
| C13—C11—C12 | 109.6 (2) | | |

X-ray crystal data for compound 3b′ was acquired. The identification code used below is: mukherjee_neu1_0m.

TABLE 14
Crystal data for compound 3b′.

| | |
|---|---|
| Cl•C₁₈H₂₁FN | Dₓ = 1.311 Mg m⁻³ |
| Mᵣ = 305.81 | Cu Kα radiation, λ = 1.54178 Å |
| Orthorhombic, P2₁2₁2₁ | Cell parameters from 9957 reflections |
| a = 8.6282 (5) Å | θ = 4.9-66.7° |
| b = 10.8425 (6) Å | μ = 2.21 mm⁻¹ |
| c = 16.5630 (8) Å | T = 100 K |
| V = 1549.49 (14) Å³ | Block, colorless |
| Z = 4 | 0.18 × 0.17 × 0.16 mm |
| F(000) = 648 | |

TABLE 15
Data collection for compound 3b′.

| | |
|---|---|
| Bruker X8 Proteum-R diffractometer | 2723 reflections with I > 2θ(I) |
| Radiation source: rotating anode | Rᵢₙₜ = 0.043 |
| Montel monochromator | θₘₐₓ = 66.7°, θₘᵢₙ = 4.9° |
| ϕ and ω scans | h = −10→9 |
| 63173 measured reflections | k = −12→12 |
| 2725 independent reflections | l = −19→19 |

TABLE 16
Refinement for compound 3b′.

| | |
|---|---|
| Refinement on F² | Secondary atom site location: difference |
| | Fourier map |
| Least-squares matrix: full | Hydrogen site location: mixed |
| R[F² > 2σ(F²)] = 0.025 | H atoms treated by a mixture of |
| | independent and constrained refinement |
| wR(F²) = 0.062 | w= 1/[σ²(Fₒ²) + (0.0176P)² + 0.690P] |
| | where P = (Fₒ² + 2F_{c}²)/3 |
| S = 1.16 | (Δ/σ)ₘₐₓ = 0.001 |
| 2725 reflections | Δₘₐₓ = 0.17 e Å⁻³ |
| 206 parameters | Δₘᵢₙ = −0.20 e Å⁻³ |
| 2 restraints | Absolute structure: Refined as an |
| | inversion twin. |
| Primary atom site location: | Absolute structure parameter: 0.079 (17) |
| dual | |

Special details were as follows: geometry. All esds (except the esd in the dihedral angle between two I.s. planes) are estimated using the full covariance matrix. The cell esds are taken into account individually in the estimation of esds in distances, angles and torsion angles; correlations between esds in cell parameters are only used when they are defined by crystal symmetry. An approximate (isotropic) treatment of cell esds is used for estimating esds involving I.s. planes. Refinement. Refined as a 2-component inversion twin.

TABLE 17
Fractional atomic coordinates and isotropic or equivalent isotropic
displacement parameters (Å²) for (2S,4S)-3b′.

| | | | | | | | | Occ. |
|---|---|---|---|---|---|---|---|---|
| | x | | y | | z | | Uᵢₛₒ*/U_{eq} | (<1) |
| Cl1 | 0.69912 | (6) | 0.82202 | (5) | 0.75000 | (3) | 0.02173 (14) | |
| F1A | 0.6256 | (2) | 0.49037 | (16) | 0.31538 | (10) | 0.0277 (5) | 0.823 (4) |
| N003 | 0.3699 | (2) | 0.73623 | (16) | 0.76393 | (10) | 0.0160 (4) | |
| H003 | 0.470 | (3) | 0.754 | (2) | 0.7501 | (17) | 0.019* | |
| C13 | 0.3977 | (2) | 0.5182 | (2) | 0.49729 | (12) | 0.0146 (4) | |
| C12 | 0.3875 | (2) | 0.6236 | (2) | 0.63301 | (12) | 0.0159 (4) | |
| H12A | 0.391418 | | 0.539805 | | 0.656723 | | 0.019* | |
| H12B | 0.494473 | | 0.656288 | | 0.630547 | | 0.019* | |
| C17 | 0.5477 | (3) | 0.4594 | (2) | 0.38254 | (13) | 0.0215 (5) | |
| H17 | 0.599455 | | 0.482388 | | 0.334211 | | 0.026* | 0.177 (4) |
| C6 | −0.1049 | (3) | 0.5955 | (2) | 0.61602 | (13) | 0.0178 (5) | |
| H6 | −0.164619 | | 0.605955 | | 0.663701 | | 0.021* | |
| C11 | 0.3185 | (2) | 0.61731 | (19) | 0.54739 | (12) | 0.0142 (4) | |
| H11 | 0.339830 | | 0.698303 | | 0.520734 | | 0.017* | |
| C14 | 0.3964 | (3) | 0.3950 | (2) | 0.52103 | (13) | 0.0176 (5) | |
| H14 | 0.343216 | | 0.371583 | | 0.568858 | | 0.021* | |
| C18 | 0.4734 | (3) | 0.5504 | (2) | 0.42596 | (13) | 0.0174 (4) | |
| H18 | 0.473744 | | 0.633460 | | 0.407632 | | 0.021* | |
| C10 | 0.1431 | (2) | 0.59977 | (19) | 0.54831 | (13) | 0.0146 (4) | |
| C8 | −0.0915 | (3) | 0.5515 | (2) | 0.47410 | (13) | 0.0193 (5) | |
| H8 | −0.141018 | | 0.529928 | | 0.424866 | | 0.023* | |
| C5 | 0.0556 | (2) | 0.6136 | (2) | 0.61874 | (12) | 0.0142 (4) | |
| C7 | −0.1776 | (3) | 0.5625 | (2) | 0.54483 | (14) | 0.0198 (5) | |
| H7 | −0.286034 | | 0.547475 | | 0.544175 | | 0.024* | |
| C4 | 0.1295 | (3) | 0.6481 | (2) | 0.69864 | (13) | 0.0178 (5) | |
| H4A | 0.141059 | | 0.573369 | | 0.732423 | | 0.021* | |
| H4B | 0.061427 | | 0.706704 | | 0.727712 | | 0.021* | |
| C9 | 0.0670 | (3) | 0.5721 | (2) | 0.47598 | (13) | 0.0175 (5) | |
| H9 | 0.124898 | | 0.567368 | | 0.427302 | | 0.021* | |
| C16 | 0.5512 | (3) | 0.3376 | (2) | 0.40501 | (13) | 0.0226 (5) | |
| H16 | 0.605009 | | 0.277501 | | 0.374053 | | 0.027* | |
| C3 | 0.2877 | (3) | 0.70688 | (19) | 0.68536 | (12) | 0.0144 (4) | |
| H3 | 0.271798 | | 0.786112 | | 0.655588 | | 0.017* | |
| C15 | 0.4721 | (3) | 0.3063 | (2) | 0.47541 | (13) | 0.0235 (5) | |
| H15 | 0.469921 | | 0.222628 | | 0.492467 | | 0.028* | 0.823 (4) |
| C2 | 0.3824 | (3) | 0.6312 | (2) | 0.82160 | (13) | 0.0212 (5) | |
| H2A | 0.283091 | | 0.619989 | | 0.849382 | | 0.032* | |
| H2B | 0.463527 | | 0.648859 | | 0.861356 | | 0.032* | |
| H2C | 0.408661 | | 0.555840 | | 0.791995 | | 0.032* | |
| C1 | 0.3031 | (3) | 0.8460 | (2) | 0.80562 | (14) | 0.0229 (5) | |
| H1A | 0.311371 | | 0.918207 | | 0.770334 | | 0.034* | |
| H1B | 0.360182 | | 0.861285 | | 0.855777 | | 0.034* | |
| H1C | 0.193805 | | 0.830461 | | 0.818139 | | 0.034* | |
| F1B | 0.4922 | (12) | 0.2008 | (6) | 0.4961 | (5) | 0.035 (3) | 0.177 (4) |

TABLE 18
Atomic displacement parameters (Å²) for (mukherjee_neu1_0m).

| | U¹¹ | | U²² | | U³³ | | U¹² | | U¹³ | | U²³ | |
|---|---|---|---|---|---|---|---|---|---|---|---|---|
| Cl1 | 0.0214 | (3) | 0.0216 | (2) | 0.0222 | (2) | −0.0002 | (2) | −0.0012 | (2) | −0.0008 | (2) |
| F1A | 0.0280 | (10) | 0.0383 | (10) | 0.0168 | (8) | −0.0075 | (8) | 0.0095 | (7) | −0.0028 | (7) |
| N003 | 0.0192 | (9) | 0.0151 | (8) | 0.0137 | (9) | 0.0006 | (7) | −0.0023 | (8) | −0.0032 | (7) |
| C13 | 0.0106 | (10) | 0.0211 | (11) | 0.0122 | (10) | 0.0010 | (8) | −0.0023 | (8) | −0.0028 | (8) |
| C12 | 0.0149 | (11) | 0.0185 | (11) | 0.0142 | (10) | 0.0004 | (9) | −0.0006 | (9) | −0.0031 | (8) |
| C17 | 0.0146 | (11) | 0.0364 | (14) | 0.0136 | (11) | −0.0046 | (10) | 0.0032 | (9) | −0.0060 | (10) |
| C6 | 0.0172 | (11) | 0.0182 | (11) | 0.0180 | (10) | 0.0009 | (9) | 0.0023 | (9) | 0.0029 | (9) |
| C11 | 0.0142 | (10) | 0.0148 | (9) | 0.0135 | (10) | 0.0007 | (9) | 0.0009 | (8) | −0.0018 | (8) |
| C14 | 0.0202 | (11) | 0.0203 | (11) | 0.0122 | (10) | 0.0024 | (10) | 0.0000 | (9) | 0.0003 | (8) |
| C18 | 0.0157 | (10) | 0.0220 | (11) | 0.0144 | (10) | −0.0043 | (9) | −0.0013 | (8) | −0.0009 | (9) |
| C10 | 0.0150 | (11) | 0.0122 | (9) | 0.0168 | (10) | 0.0020 | (8) | −0.0006 | (8) | 0.0010 | (8) |
| C8 | 0.0193 | (12) | 0.0201 | (11) | 0.0184 | (11) | 0.0015 | (10) | −0.0060 | (9) | −0.0003 | (9) |
| C5 | 0.0160 | (11) | 0.0129 | (10) | 0.0137 | (10) | 0.0012 | (8) | −0.0003 | (8) | 0.0012 | (8) |
| C7 | 0.0137 | (11) | 0.0200 | (10) | 0.0258 | (11) | 0.0006 | (9) | −0.0024 | (9) | 0.0029 | (9) |
| C4 | 0.0172 | (11) | 0.0223 | (11) | 0.0139 | (10) | −0.0010 | (9) | 0.0010 | (8) | −0.0022 | (8) |
| C9 | 0.0164 | (11) | 0.0213 | (11) | 0.0149 | (11) | 0.0034 | (9) | 0.0000 | (8) | −0.0028 | (9) |
| C16 | 0.0185 | (11) | 0.0309 | (13) | 0.0184 | (11) | 0.0056 | (10) | −0.0027 | (8) | −0.0095 | (10) |
| C3 | 0.0166 | (11) | 0.0163 | (10) | 0.0104 | (9) | −0.0009 | (8) | −0.0019 | (8) | −0.0010 | (8) |
| C15 | 0.0288 | (12) | 0.0228 | (12) | 0.0189 | (11) | 0.0089 | (10) | −0.0058 | (9) | −0.0029 | (9) |
| C2 | 0.0286 | (13) | 0.0200 | (11) | 0.0148 | (10) | 0.0002 | (10) | −0.0043 | (10) | 0.0021 | (9) |
| C1 | 0.0275 | (12) | 0.0213 | (11) | 0.0199 | (10) | 0.0045 | (10) | −0.0014 | (10) | −0.0064 | (9) |
| F1B | 0.066 | (6) | 0.014 | (4) | 0.024 | (4) | 0.013 | (4) | 0.003 | (4) | −0.001 | (3) |

TABLE 19
Geometric parameters (Å, °) for (mukherjee_neu1_0m).

| | | | |
|---|---|---|---|
| F1A—C17 | 1.343 (3) | C6—C7 | 1.383 (3) |
| N003—C3 | 1.516 (3) | C11—C10 | 1.525 (3) |
| N003—C2 | 1.490 (3) | C14—C15 | 1.387 (3) |
| N003—C1 | 1.492 (3) | C10—C5 | 1.397 (3) |
| C13—C11 | 1.520 (3) | C10—C9 | 1.399 (3) |
| C13—C14 | 1.393 (3) | C8—C7 | 1.393 (3) |
| C13—C18 | 1.394 (3) | C8—C9 | 1.385 (3) |
| C12—C11 | 1.539 (3) | C5—C4 | 1.516 (3) |
| C12—C3 | 1.519 (3) | C4—C3 | 1.523 (3) |
| C17—C18 | 1.379 (3) | C16—C15 | 1.393 (3) |
| C17—C16 | 1.373 (4) | C15—F1B | 1.206 (7) |
| C6—C5 | 1.399 (3) | | |
| C2—N003—C3 | 115.07 (16) | C5—C10—C9 | 119.00 (19) |
| C2—N003—C1 | 109.90 (17) | C9—C10—C11 | 118.94 (18) |
| C1—N003—C3 | 112.58 (17) | C9—C8—C7 | 119.6 (2) |
| C14—C13—C11 | 121.35 (19) | C6—C5—C4 | 118.61 (19) |
| C14—C13—C18 | 118.9 (2) | C10—C5—C6 | 119.48 (19) |
| C18—C13—C11 | 119.75 (19) | C10—C5—C4 | 121.91 (19) |
| C3—C12—C11 | 109.44 (17) | C6—C7—C8 | 119.8 (2) |
| F1A—C17—C18 | 119.1 (2) | C5—C4—C3 | 110.74 (17) |
| F1A—C17—C16 | 117.0 (2) | C8—C9—C10 | 121.1 (2) |
| C16—C17—C18 | 123.9 (2) | C17—C16—C15 | 116.8 (2) |
| C7—C6—C5 | 120.9 (2) | N003—C3—C12 | 110.47 (17) |
| C13—C11—C12 | 111.11 (17) | N003—C3—C4 | 112.54 (16) |
| C13—C11—C10 | 111.31 (17) | C12—C3—C4 | 109.97 (17) |
| C10—C11—C12 | 112.33 (17) | C14—C15—C16 | 121.2 (2) |
| C15—C14—C13 | 120.5 (2) | F1B—C15—C14 | 124.8 (4) |
| C17—C18—C13 | 118.7 (2) | F1B—C15—C16 | 113.5 (5) |
| C5—C10—C11 | 122.04 (19) | | |

Document origin: publCIF [Westrip, S. P. (2010). J. Apply. Cryst., 43, 920-925].

### Example 6. Synthesis of Charged Substituent on Tetralin Core

Each of the compounds or formulas disclosed herein can be derivatized to a corresponding positively charged quaternary amine, for example, by the addition of a third alkyl group to the amine to render it impermeable to the blood-brain barrier and specific for peripheral 5-HT receptors. For example, any of the compounds or formulas described herein may be derivatized at an amino group at the 2 position of the tetralin core via Scheme 11 shown below, wherein R⁴ can represent ring ‘C’ including substituents described above or shown in FIG. 1B.

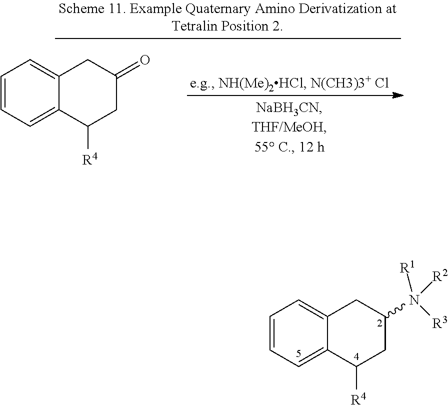

Examples compounds that can be synthesized, with ‘E’ representing a charged group or charged amine, are shown below:

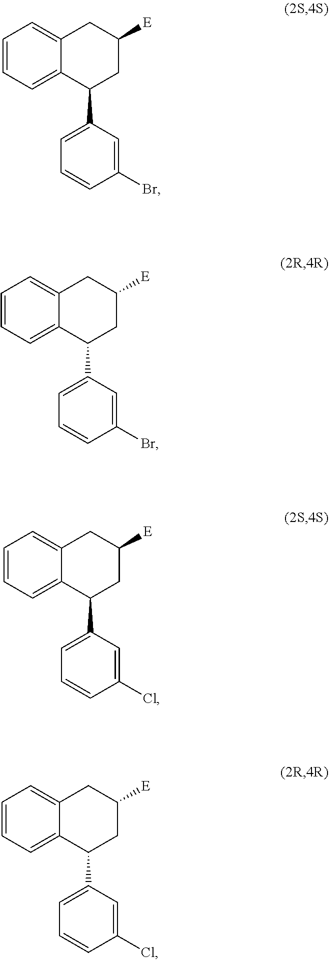

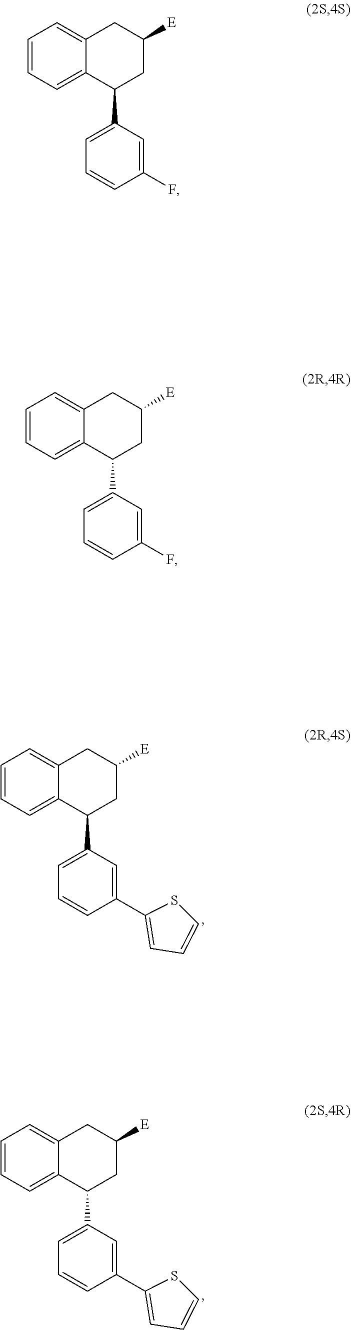

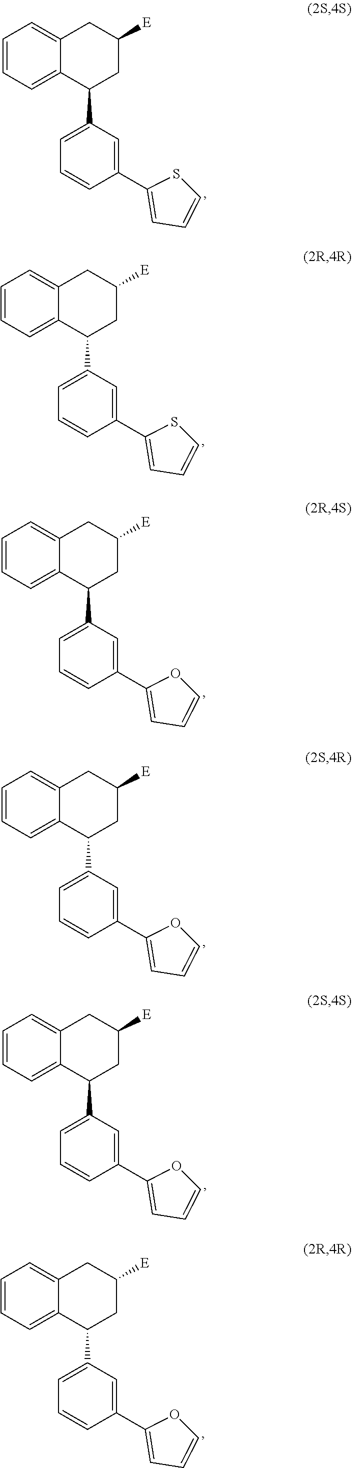

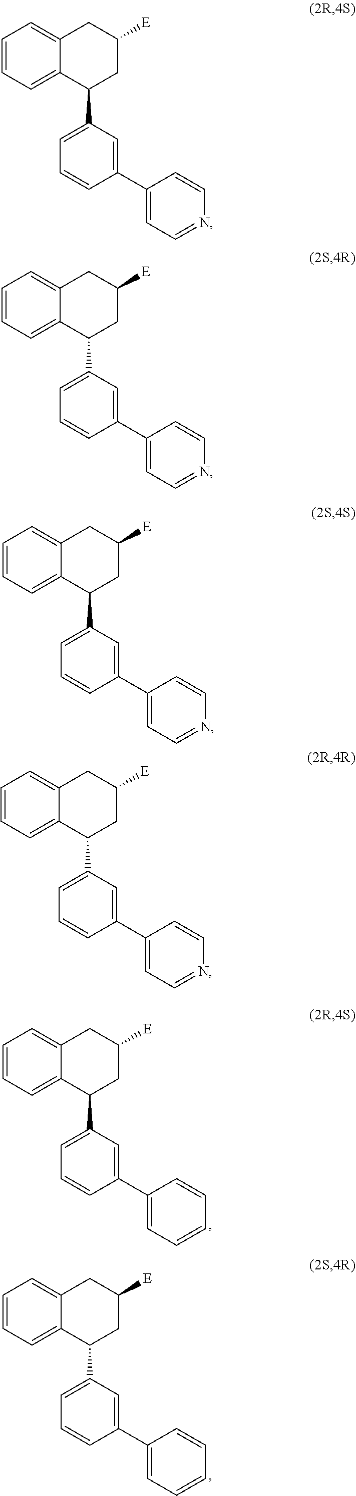

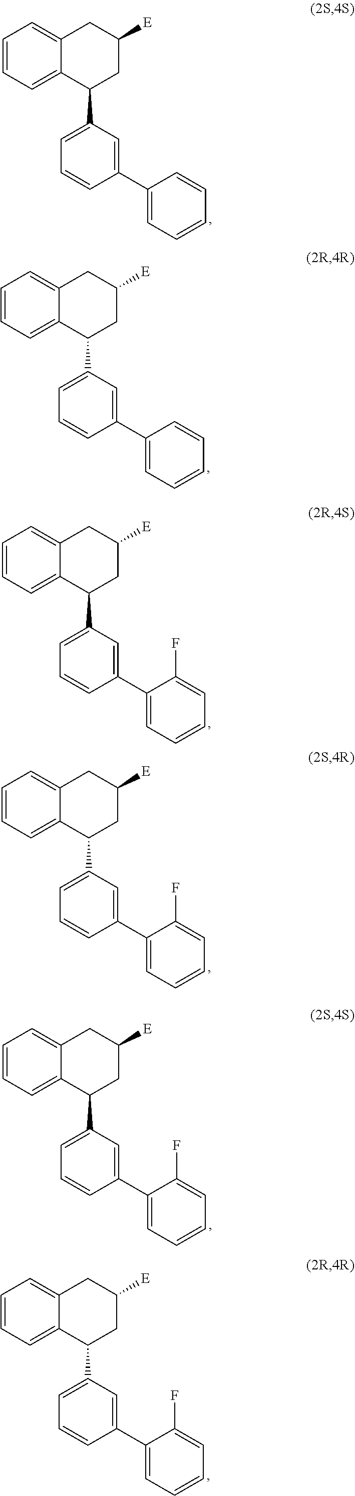

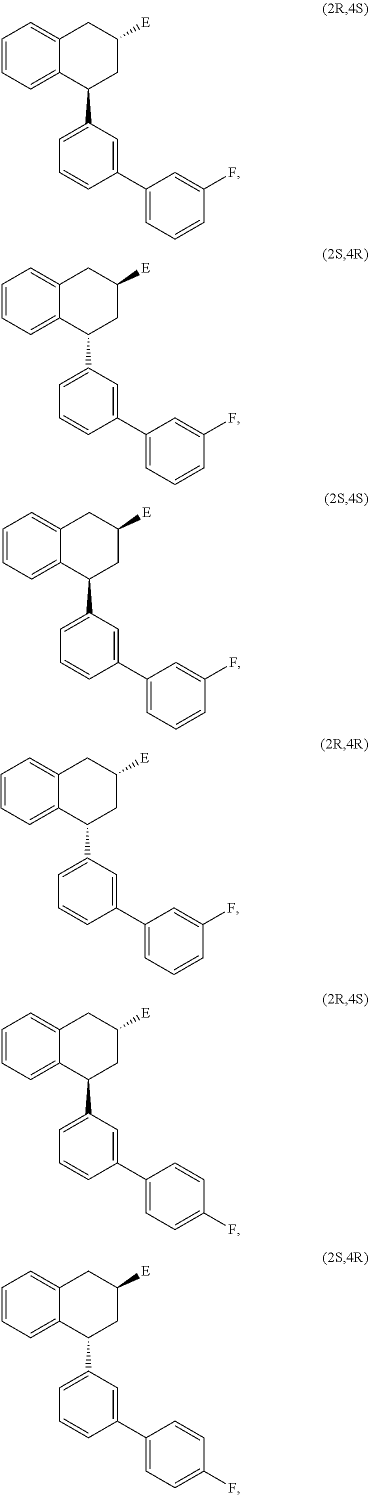

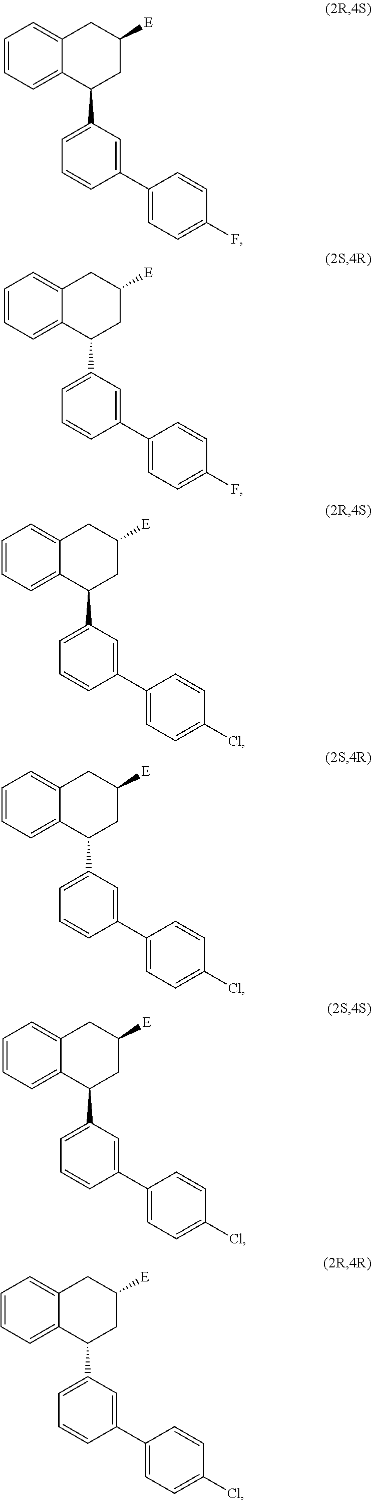

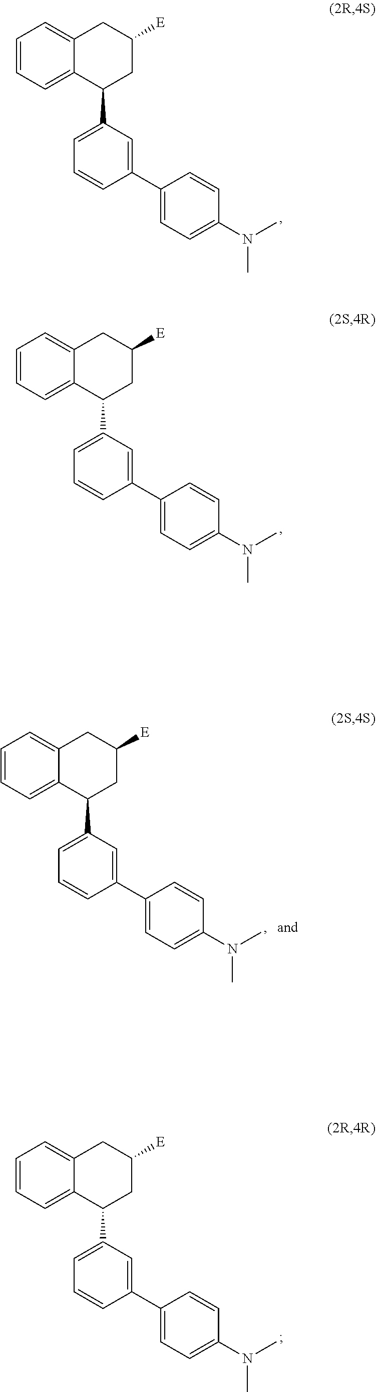

or a pharmaceutically acceptable salt, hydrate, or solvate thereof.

### Example 7. Compounds Bearing a Positively Charged Amino Group at the 2 Position of the Tetralin Core Do Not Readily Cross the Blood-Brain Barrier

In an example to demonstrate the compound or composition does not substantially accumulate in the human brain, adult, male, C57Bl/6J mice, approximately six months old, and treatment-naïve for at least six weeks prior to testing, can be injected sc with any of the compounds described herein, bearing a positively charged amino group at the 2 position of the tetralin core (the “test compound”) at a dose of about 3.0 mg/kg and returned to their home cages. At 30, 60, or 90 min later, mice are euthanized by rapid cervical dislocation and decapitation. Trunk blood is collected in pre-chilled, heparin-coated tubes. Brains are quickly excised and frozen in liquid nitrogen. Plasma is collected from blood after centrifugation for 5 min at 13,000 g. Whole brain samples are wrapped in foil, and brain and plasma samples are labeled and stored at −80° C. until liquid chromatography-mass spectrometry/mass spectrometry (LC-MS/MS) assays are performed. Frozen brain samples are weighed and homogenized in phosphate buffered saline (PBS), pH 7.4. After the first analysis, the extra brain homogenate is stored at −80° C. until they are thawed for a second, more dilute, analysis. Plasma samples are used directly upon arrival. The proteins from each plasma sample and a portion of each brain homogenate are immediately precipitated with 1:1 methanol:acetonitrile (4× starting volume) and internal standard (e.g. (−)-MBP⁶⁸) followed by centrifugation at 14,000 g for 5 minutes at 4° C. The resulting supernatants from each sample are dried under nitrogen. Each sample is reconstituted in methanol, vortexed, sonicated briefly, and centrifuged prior to LC-MS/MS analysis. Calibration curves are constructed from the ratios of the peak areas of test compound versus internal standard in extracted standards made in mouse plasma or homogenized mouse brain.

LC-MS/MS analysis can be performed using an Agilent 1100 series HPLC and a Thermo Finnigan Quantum Ultra triple quad mass spectrometer. Example mobile phases used are 0.1% formic acid in water (A) and 0.1% formic acid in methanol (B) in a 5 minute gradient. Samples of 10 μL each are injected onto a Phenomenex Gemini C18 column (2×50 mm, 5μ) with a C18 guard column. The test compound and its internal standard ((−)-MBP) were ionized in ESI+ and detected in SRM mode. Internal standards are used for quantification of the test compound level per g tissue or per μL plasma.

The test compounds are not expected to accumulate in the brain and, rather, are expected to be more prevalent in the plasma.

Each of the compounds disclosed herein can be derivatized to a corresponding positively charged quaternary amine, for example, by the addition of a third alkyl group, to render it impermeable to the blood-brain barrier and specific for peripheral 5-HT receptors.

### REFERENCES

Alexander S P, Christopoulos A, Davenport A P, Kelly E, Marrion N V, Peters J A, . . . Collaborators C (2017). THE CONCISE GUIDE TO PHARMACOLOGY 2017/18: G protein-coupled receptors. Br J Pharmacol 174 Suppl 1: S17-S129.
Altman R A, & Buchwald S L (2007). Pd-catalyzed Suzuki-Miyaura reactions of aryl halides using bulky biarylmonophosphine ligands. Nat Protoc 2: 3115-3121.
Ancoli-Israel S, Vanover K E, Weiner D M, Davis R E, & van Kammen D P (2011). Pimavanserin tartrate, a 5-HT(2A) receptor inverse agonist, increases slow wave sleep as measured by polysomnography in healthy adult volunteers. Sleep Med 12: 134-141.
Andersson K E, & Gratzke C (2007). Pharmacology of alpha1-adrenoceptor antagonists in the lower urinary tract and central nervous system. Nat Clin Pract Urol 4: 368-378.
Armstrong J L, Casey A B, Saraf T S, Mukherjee M, Booth R G, & Canal C E (2020). (S)-5-(2′-Fluorophenyl)-N,N-dimethyl-1,2,3,4-tetrahydronaphthalen-2-amine, a Serotonin Receptor Modulator, Possesses Anticonvulsant, Prosocial, and Anxiolytic-like Properties in an Fmr1 Knockout Mouse Model of Fragile X Syndrome and Autism Spectrum Disorder. ACS Pharmacol Transl Sci 3: 509-523.
Ayme-Dietrich E, Lawson R, Cote F, de Tapia C, Da Silva S, Ebel C, . . . Monassier L (2017). The role of 5-HT2B receptors in mitral valvulopathy: bone marrow mobilization of endothelial progenitors. Br J Pharmacol 174: 4123-4139.
Ballard C, Kales H C, Lyketsos C, Aarsland D, Creese B, Mills R, Williams H, Sweet R A. Psychosis inAlzheimer's Disease. Curr Neurol Neurosci Rep. 2020; 20(12):57. doi: 10.1007/s11910-020-01074-y. PMID: 33048274.
Ballard C, Banister C, Khan Z, Cummings J, Demos G, Coate B, Youakim J M, Owen R, Stankovic S. Evaluation of the safety, tolerability, and efficacy of pimavanserin versus placebo in patients with Alzheimer's disease psychosis: a phase 2, randomised, placebo-controlled, double-blind study. Lancet Neurol. 2018; 17(3):213-222. doi: 10.1016/S1474-4422(18)₃₀₀₃₉-5.PMID: 29452684).
Ballesteros J A, Weinstein, H. (1995). Integrated Methods for the Construction of Three-Dimensional Models and Computational Probing of Structure-Function Relations in G Protein-Coupled Receptors Methods in Neuroscience 25: 366-428.
Bayly C I, Cieplak P, Cornell W D, & Kollman P A (1993). A Well-Behaved Electrostatic Potential Based Method Using Charge Restraints for Deriving Atomic Charges—the Resp Model. J Phys Chem-Us 97: 10269-10280.
Bellos I, Pergialiotis V, Papapanagiotou A, Loutradis D, Daskalakis G. Comparative efficacy and safety of oral antihypertensive agents in pregnant women with chronic hypertension: a network metaanalysis. Am J Obstet Gynecol. 2020 October; 223(4):525-537. doi: 10.1016/j.ajog.2020.03.016. PMID: 32199925.
Berardelli A, Rothwell J C, Thompson P D, Hallett M. Pathophysiology of bradykinesia in Parkinson's disease.Brain. 2001; 124(Pt 11):2131-46. doi: 10.1093/brain/124.11.2131. PMID: 11673316.
Bevilacqua L, Doly S, Kaprio J, Yuan Q, Tikkanen R, Paunio T, . . . Goldman D (2010). A population-specific HTR2B stop codon predisposes to severe impulsivity. Nature 468: 1061-1066.
Booth R G, Fang L, Huang Y, Wilczynski A, & Sivendran S (2009). (1R, 3S)-(−)-trans-PAT: a novel full-efficacy serotonin 5-HT2C receptor agonist with 5-HT_{2A} and 5-HT2B receptor inverse agonist/antagonist activity. Eur J Pharmacol 615: 1-9.
Canal C E, & Morgan D (2012). Head-twitch response in rodents induced by the hallucinogen 2,5-dimethoxy-4-iodoamphetamine: a comprehensive history, a re-evaluation of mechanisms, and its utility as a model. Drug Test Anal 4: 556-576.
Canal C E, Morgan D, Felsing D, Kondabolu K, Rowland N E, Robertson K L, . . . Booth R G (2014). A novel aminotetralin-type serotonin (5-HT) 2C receptor-specific agonist and 5-HT2A competitive antagonist/5-HT2B inverse agonist with preclinical efficacy for psychoses. J Pharmacol Exp Ther 349: 310-318.
Caraci F, Santagati M, Caruso G, Cannavb D, Leggio G M, Salomone S, Drago F. New antipsychotic drugs for the treatment of agitation and psychosis in Alzheimer's disease: focus on brexpiprazole and pimavanserin. F1000Res. 2020; 9:F1000. doi: 10.12688/f1000research.22662.1. PMID:32695312.
Casey A B, Mukherjee M, McGlynn R P, Cui M, Kohut S J, Booth R G. A new class of 5-HT2A/5-HT2C receptor inverse agonists: Synthesis, molecular modeling, in vitro and in vivo pharmacology of novel 2-aminotetralins. Br J Pharmacol. 2022 June; 179(11):2610-2630. doi: 10.1111/bph.15756. Epub 2022 Mar. 7. PMID: 34837227.
Chagraoui A, Thibaut F, Skiba M, Thuillez C, & Bourin M (2016). 5-HT2C receptors in psychiatric disorders: A review. Prog Neuropsychopharmacol Biol Psychiatry 66: 120-135.
Cheng H C (2001). The power issue: determination of KB or Ki from IC50. A closer look at the Cheng-Prusoff equation, the Schild plot and related power equations. J Pharmacol Toxicol Methods 46: 61-71.
Chopko T C, & Lindsley C W (2018). Classics in Chemical Neuroscience: Risperidone. ACS Chem Neurosci 9: 1520-1529.
Cordova-Sintjago T C, Fang L, Bruysters M, Leurs R, & Booth R G (2012). Molecular determinants of ligand binding at the human histamine H1 receptor: Site-directed mutagenesis results analyzed with ligand docking and molecular dynamics studies at H1 homology and crystal structure models. J Chem Pharm Res 4: 2937-2951.
Council N R (2011) Guide for the care and use of laboratory animals. National Academy Press. Washington, D.C. Available from http://ebookcentral.proquest.com/lib/northeastern-ebooks/detail.action?doclD=3378734.
Cummings J, Isaacson S, Mills R, Williams H, Chi-Burris K, Corbett A, . . . Ballard C (2014). Pimavanserin for patients with Parkinson's disease psychosis: a randomised, placebo-controlled phase 3 trial. Lancet 383: 533-540.
Curtis M J, Alexander S, Cirino G, Docherty J R, George C H, Giembycz M A, . . . Ahluwalia A (2018). Experimental design and analysis and their reporting II: updated and simplified guidance for authors and peer reviewers. Br J Pharmacol 175: 987-993.
Darden T. YD, Pedersen L. (1993). Particle Mesh Ewald-an N.Log(n) Method for Ewald Sums in Large Systems. J Chem Phys 98: 10089-10092.
Demireva E Y, Suri D, Morelli E, Mahadevia D, Chuhma N, Teixeira C M, . . . Ansorge M S (2018). 5-HT2C receptor blockade reverses SSRI-associated basal ganglia dysfunction and potentiates therapeutic efficacy. Mol Psychiatry.
Egan C T, Herrick-Davis K, & Teitler M (1998). Creation of a constitutively activated state of the 5-hydroxytryptamine2A receptor by site-directed mutagenesis: inverse agonist activity of antipsychotic drugs. J Pharmacol Exp Ther 286: 85-90.
El Haj M, Altintas E, Chapelet G, Kapogiannis D, Gallouj K. High depression and anxiety in people with Alzheimer's disease living in retirement homes during the covid-19 crisis. Psychiatry Res. 2020; 291:113294. doi: 10.1016/j.psychres.2020.113294. PMID: 32763552.
Fava M, Dirks B, Freeman M P, Papakostas G I, Shelton R C, Thase M E, . . . Stankovic S (2019). A Phase 2, Randomized, Double-Blind, Placebo-Controlled Study of Adjunctive Pimavanserin in Patients With Major Depressive Disorder and an Inadequate Response to Therapy (CLARITY). J Clin Psychiatry 80.
Fluyau D, Mitra P, Jain A, Kailasam V K, Pierre C G. Selective serotonin reuptake inhibitors in the treatment of depression, anxiety, and post-traumatic stress disorder in substance use disorders: a Bayesian meta-analysis. Eur J Clin Pharmacol. 2022; 78(6):931-942. doi: 10.1007/s00228-022-03303-4. PMID: 35246699.
Gillard M, Van Der Perren C, Moguilevsky N, Massingham R, & Chatelain P (2002). Binding characteristics of cetirizine and levocetirizine to human H(1) histamine receptors: contribution of Lys(191) and Thr(194). Mol Pharmacol 61: 391-399.
Grossfield A, Pitman M C, Feller S E, Soubias 0, & Gawrisch K (2008). Internal hydration increases during activation of the G-protein-coupled receptor rhodopsin. J Mol Biol 381: 478-486.
Gutierrez M G, Mansfield K S, & Malmstadt N (2016). The Functional Activity of the Human Serotonin 5-HT1A Receptor Is Controlled by Lipid Bilayer Composition. Biophys J 110: 2486-2495.
Hacksell U, Burstein E S, McFarland K, Mills R G, & Williams H (2014). On the discovery and development of pimavanserin: a novel drug candidate for Parkinson's psychosis. Neurochem Res 39: 2008-2017.
Harding S D, Sharman J L, Faccenda E, Southan C, Pawson A J, Ireland S, . . . Nc I (2018). The IUPHAR/BPS Guide to PHARMACOLOGY in 2018: updates and expansion to encompass the new guide to IMMUNOPHARMACOLOGY. Nucleic Acids Res 46: D1091-D1106.
Hauser A S, Attwood M M, Rask-Andersen M, Schioth H B, & Gloriam D E (2017). Trends in GPCR drug discovery: new agents, targets and indications. Nat Rev Drug Discov 16: 829-842.
Hedner T, Persson B. Effects of a new serotonin antagonist, ketanserin, in experimental and clinical hypertension. Am J Hypertens. 1988; 1(3 Pt 3):317S-323S. doi: 10.1093/ajh/1.3.317s. PMID: 3046635.
Hopkins W G, Batterham A M, Pyne D B, & Impellizzeri F M (2011). Misplaced decimal places. Scand J Med Sci Sports 21: 867-868.
Janssen W, Schymura Y, Novoyatleva T, Kojonazarov B, Boehm M, Wietelmann A, Luitel H, Murmann K, Krompiec D R, Tretyn A, Pullamsetti S S, Weissmann N, Seeger W, Ghofrani H A, Schermuly R T. Biomed Res Int. 2015; 2015:438403. doi: 10.1155/2015/438403. Epub 2015 Feb. 1. PMID: 25667920.
Kim K, Che T, Panova O, DiBerto J F, Lyu J, Krumm B E, . . . Roth B L (2020). Structure of a Hallucinogen-Activated Gq-Coupled 5-HT_{2A} Serotonin Receptor. Cell 182: 1574-1588 e1519.
Kimura K T, Asada H, Inoue A, Kadji F M N, Im D, Mori C, . . . Shimamura T (2019). Structures of the 5-HT_{2A} receptor in complex with the antipsychotics risperidone and zotepine. Nat Struct Mol Biol 26: 121-128.
Knapp D M, Gillis E P, & Burke M D (2009). A general solution for unstable boronic acids: slow-release cross-coupling from air-stable MIDA boronates. J Am Chem Soc 131: 6961-6963.
Kristiansen K, Kroeze W K, Willins D L, Gelber El, Savage J E, Glennon R A, & Roth B L (2000). A highly conserved aspartic acid (Asp-155) anchors the terminal amine moiety of tryptamines and is involved in membrane targeting of the 5-HT(2A) serotonin receptor but does not participate in activation via a “salt-bridge disruption” mechanism. J Pharmacol Exp Ther 293: 735-746.
Kudo N, Perseghini M, & Fu G C (2006). A versatile method for Suzuki cross-coupling reactions of nitrogen heterocycles. Angew Chem Int Ed Engl 45: 1282-1284.
Lee J S, Kildegaard H F, Lewis N E, & Lee G M (2019). Mitigating Clonal Variation in Recombinant Mammalian Cell Lines. Trends Biotechnol 37: 931-942.
Lefkowitz R J, Pierce K L, & Luttrell L M (2002). Dancing with different partners: protein kinase a phosphorylation of seven membrane-spanning receptors regulates their G protein-coupling specificity. Mol Pharmacol 62: 971-974.
Meltzer H Y (1999). The role of serotonin in antipsychotic drug action. Neuropsychopharmacology 21: 106S-115S.
Meltzer H Y, Mills R, Revell S, Williams H, Johnson A, Bahr D, & Friedman J H (2010). Pimavanserin, a serotonin(2A) receptor inverse agonist, for the treatment of parkinson's disease psychosis. Neuropsychopharmacology 35: 881-892.
Moguilevsky N, Varsalona F, Guillaume J P, Noyer M, Gillard M, Daliers J, . . . Bollen A (1995). Pharmacological and functional characterisation of the wild-type and site-directed mutants of the human H1 histamine receptor stably expressed in CHO cells. J Recept Signal Transduct Res 15: 91-102.
Moniri N H, Covington-Strachan D, & Booth R G (2004). Ligand-directed functional heterogeneity of histamine H1 receptors: novel dual-function ligands selectively activate and block H1-mediated phospholipase C and adenylyl cyclase signaling. J Pharmacol Exp Ther 311: 274-281.
Morris G M, Goodsell D S, Halliday R S, Huey R, Hart W E, Belew R K, & Olson A J (1998). Automated docking using a Lamarckian genetic algorithm and an empirical binding free energy function. J Comput Chem 19: 1639-1662.
Nagatomo T, Rashid M, Abul Muntasir H, Komiyama T. Functions of 5-HT_{2A} receptor and its antagonists in the cardiovascular system. Pharmacol Ther. 2004; 104(1):59-81. doi: 10.1016/j.pharmthera.2004.08.005. PMID: 15500909.
Neubig R R, Spedding M, Kenakin T, Christopoulos A, International Union of Pharmacology Committee on Receptor N, & Drug C (2003). International Union of Pharmacology Committee on Receptor Nomenclature and Drug Classification. XXXVIII. Update on terms and symbols in quantitative pharmacology. Pharmacol Rev 55: 597-606.
Nicholson A N, Pascoe P A, Turner C, Ganellin C R, Greengrass P M, Casy A F, & Mercer A D (1991). Sedation and histamine H1-receptor antagonism: studies in man with the enantiomers of chlorpheniramine and dimethindene. Br J Pharmacol 104: 270-276. Optibrium StarDrop v. 6.5 (optibrium.com).
Padhariya K, Bhandare R, Canney D, Velingkar V. Cardiovascular Concern of 5-HT2B Receptor and Recent Vistas in the Development of Its Antagonists. Cardiovasc Hematol Disord Drug Targets. 2017; 17(2):86-104. doi: 10.2174/1871529X17666170703115111. PMID: 28676029.
Pandy-Szekeres G, Munk C, Tsonkov T M, Mordalski S, Harpsoe K, Hauser A S, . . . Gloriam D E (2018). GPCRdb in 2018: adding GPCR structure models and ligands. Nucleic Acids Res 46: D440-D446.
Peng Y, McCorvy J D, Harpsoe K, Lansu K, Yuan S, Popov P, . . . Liu Z J (2018). 5-HT2C Receptor Structures Reveal the Structural Basis of GPCR Polypharmacology. Cell 172: 719-730 e714.
Percie du Sert N, Hurst V, Ahluwalia A, Alam S, Avey M T, Baker M, . . . Wurbel H (2020). The ARRIVE guidelines 2.0: Updated guidelines for reporting animal research. PLoS Biol 18: e3000410.
Perry C K, Casey A B, Felsing D E, Vemula R, Zaka M, Herrington N B, . . . Booth R G (2020). Synthesis of novel 5-substituted-2-aminotetralin analogs: 5-HT1A and 5-HT7 G protein-coupled receptor affinity, 3D-QSAR and molecular modeling. Bioorg Med Chem 28: 115262.
Peters J U, Hert J, Bissantz C, Hillebrecht A, Gerebtzoff G, Bendels S, . . . Kansy M (2012). Can we discover pharmacological promiscuity early in the drug discovery process?Drug Discov Today 17: 325-335.
Pitychoutis P M, Belmer A, Moutkine I, Adrien J, & Maroteaux L (2015). Mice Lacking the Serotonin Htr2B Receptor Gene Present an Antipsychotic-Sensitive Schizophrenic-Like Phenotype. Neuropsychopharmacology 40: 2764-2773.
Rasmussen S G, Choi H J, Fung J J, Pardon E, Casarosa P, Chae P S, . . . Kobilka B K (2011). Structure of a nanobody-stabilized active state of the beta(2) adrenoceptor. Nature 469: 175-180.
Roth B L (2013). Assay Protocol Book Version II. University of North Carolina at Chapel Hill, Chapel Hill, NC.
Rothman R B, Baumann M H, Savage J E, Rauser L, McBride A, Hufeisen S J, & Roth B L (2000). Evidence for possible involvement of 5-HT(2B) receptors in the cardiac valvulopathy associated with fenfluramine and other serotonergic medications. Circulation 102: 2836-2841.
Sakhuja R, Kondabolu K, Cordova-Sintjago T, Travers S, Vincek A S, Kim M S, . . . Booth R G (2015). Novel 4-substituted-N,N-dimethyltetrahydronaphthalen-2-amines: synthesis, affinity, and in silico docking studies at serotonin 5-HT2-type and histamine H1 G protein-coupled receptors. Bioorg Med Chem 23: 1588-1600.
Schrodinger, LLC (2015). The PyMOL Molecular Graphics System, Version 1.8.
Seiler M P, & Markstein R (1984). Further characterization of structural requirements for agonists at the striatal dopamine D2 receptor and a comparison with those at the striatal dopamine D1 receptor. Studies with a series of monohydroxyaminotetralins on acetylcholine release from rat striatum. Mol Pharmacol 26: 452-457.
Shapiro D A, Kristiansen K, Weiner D M, Kroeze W K, & Roth B L (2002). Evidence for a model of agonist-induced activation of 5-hydroxytryptamine 2A serotonin receptors that involves the disruption of a strong ionic interaction between helices 3 and 6. J Biol Chem 277: 11441-11449.
Shimamura T, Shiroishi M, Weyand S, Tsujimoto H, Winter G, Katritch V, . . . Iwata S (2011). Structure of the human histamine H1 receptor complex with doxepin. Nature 475: 65-70.
Simonoff E, Pickles A, Charman T, Chandler S, Loucas T, Baird G. Psychiatric disorders in children with autism spectrum disorders: prevalence, comorbidity, and associated factors in a population-derived sample J Am Acad Child Adolesc Psychiatry. 2008; 47(8):921-9. doi: 10.1097/CHI.0b013e318179964f. PMID: 18645422.
Stahl S M (2008). Selective histamine H1 antagonism: novel hypnotic and pharmacologic actions challenge classical notions of antihistamines. CNS Spectr 13: 1027-1038.
Stahl S M (2016). Mechanism of action of pimavanserin in Parkinson's disease psychosis: targeting serotonin 5HT_{2A} and 5HT2C receptors. CNS Spectr 21: 271-275.
Symons J L, Cho K J, Chang J T, Du G, Waxham M N, Hancock J F, . . . Levental K R (2021). Lipidomic atlas of mammalian cell membranes reveals hierarchical variation induced by culture conditions, subcellular membranes, and cell lineages. Soft Matter 17: 288-297.
Tariot P N, Cummings J L, Soto-Martin M E, Ballard C, Erten-Lyons D, Sultzer D L, Devanand D P, Weintraub D, McEvoy B, Youakim J M, Stankovic S, Foff E P. Trial of Pimavanserin in Dementia-Related Psychosis. N Engl J Med. 2021; 385(4):309-319. doi: 10.1056/NEJMoa2034634.PMID: 34289275.
Tran K T, Pallesen J S, Solbak S M O, Narayanan D, Baig A, Zang J, . . . Bach A (2019). A Comparative Assessment Study of Known Small-Molecule Keap1-Nrf2 Protein-Protein Interaction Inhibitors: Chemical Synthesis, Binding Properties, and Cellular Activity. J Med Chem 62: 8028-8052.
Unett D J, Gatlin J, Anthony T L, Buzard D J, Chang S, Chen C, . . . Gaidarov I (2013). Kinetics of 5-HT2B receptor signaling: profound agonist-dependent effects on signaling onset and duration. J Pharmacol Exp Ther 347: 645-659.
Valk P J, & Simons M (2009). Effects of loratadine/montelukast on vigilance and alertness task performance in a simulated cabin environment. Adv Ther 26: 89-98.
Vanover K E, Weiner D M, Makhay M, Veinbergs I, Gardell L R, Lameh J, . . . Davis R E (2006). Pharmacological and behavioral profile of N-(4-fluorophenylmethyl)-N-(1-methylpiperidin-4-yl)-N′-(4-(2-methylpropyloxy)phen ylmethyl) carbamide (2R,3R)-dihydroxybutanedioate (2:1) (ACP-103), a novel 5-hydroxytryptamine(2A) receptor inverse agonist. J Pharmacol Exp Ther 317: 910-918.
Vass M, Podlewska S, de Esch I J P, Bojarski A J, Leurs R, Kooistra A J, & de Graaf C (2019). Aminergic GPCR-Ligand Interactions: A Chemical and Structural Map of Receptor Mutation Data. J Med Chem 62: 3784-3839.
Vincek A S, & Booth R G (2009). New Approach to 4-Phenyl-beta-aminotetralin from 4-(3-Halophenyl)tetralen-2-ol Phenylacetate. Tetrahedron Lett 50: 5107-5109.
Visiers I, Ballesteros J A, & Weinstein H (2002). Three-dimensional representations of G protein-coupled receptor structures and mechanisms. Methods Enzymol 343: 329-371.
Weiner D M, Burstein E S, Nash N, Croston G E, Currier E A, Vanover K E, . . . Brann M R (2001). 5-hydroxytryptamine2A receptor inverse agonists as antipsychotics. J Pharmacol Exp Ther 299: 268-276.
Wen M C, Chan L L, Tan L C, Tan E K. Depression, anxiety, and apathy in Parkinson's disease: insights from neuroimaging studies. Eur J Neurol. 2016; 23(6):1001-19. doi: 10.1111/ene.13002. PMID: 27141858.
Zhang Y, Baycin-Hizal D, Kumar A, Priola J, Bahri M, Heffner K M, . . . Betenbaugh M J (2017). High-Throughput Lipidomic and Transcriptomic Analysis To Compare SP2/0, CHO, and HEK-293 Mammalian Cell Lines. Anal Chem 89: 1477-1485.

1. A compound for selective modulation of one or more of serotonin 5-HT2A and 5-HT2C receptors, the compound having a structure according to Formula I: 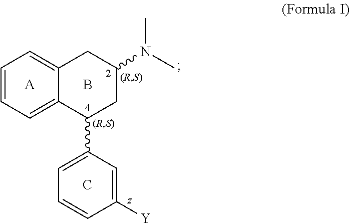
wherein Y is selected from the group consisting of 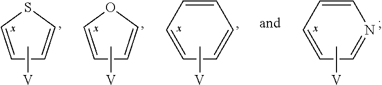
wherein covalent bond z is attached at any carbon atom of Y;
wherein Y is unsubstituted or is substituted with one or more moieties V, each of the one or more moieties V independently selected from the group consisting —F, —Cl, —Br, —I, —NH₂, —NH(CH₃), —N(CH₃)₂, —NH(CH₂CH₃), —N(CH₂CH₃)₂, —C═NH, —C═NNH₂, —C═ONH₂, —NO₂, —NO, —CN, —N₃, —N═C═O, —CH₃, —CH₂CH₃, —CH(CH₃)₂, —C═OOH, —CH₂C═OOH, —S═OCH₃, —S(═O)₂CH₃, —S(═O)₂OH, —S(═O)₂NH₂, —S(═O)₂N(CH₃)₂, —OH, —OCN, —OCH₃, —OCH₂CH₃, —CH₂OH, —CH₂CH₂OH, —CHOHCH₂OH, —CHOHCH₃, —SH, —SCN, —SCH₃, —SCH₂CH₃, —CH₂SH, —CH₂CH₂SH, —CHSHCH₂SH, —CHSHCH₃, and substituted or unsubstituted thiophene, furanyl, phenyl and pyridyl; and
wherein the compound comprises at least 50% of a single stereoisomer selected from the group of stereoisomers consisting of 2R4R, 2S4S, 2R4S, and 2S4R;
or a pharmaceutically acceptable salt, hydrate, or solvate thereof.

2. The compound of claim 1, wherein one or more moieties V are independently selected from the group consisting of 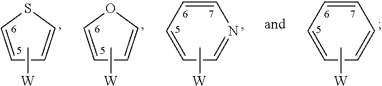
wherein V is attached to Y via a covalent bond to any one of carbons 5-7 of V; and
wherein V is substituted with one or more substituents W, each of the one or more substituents W independently selected from the group consisting of —F, —Cl, —Br, —I, —NH₂, —NH(CH₃), —N(CH₃)₂, —NH(CH₂CH₃), —N(CH₂CH₃)₂, —C═NH, —C═NNH₂, —C═ONH₂, —NO₂, —NO, —CN, —N₃, —N═C═O, —CH₃, —CH₂CH₃, —CH(CH₃)₂, —C═OOH, —CH₂C═OOH, —S═OCH₃, —S(═O)₂CH₃, —S(═O)₂OH, —S(═O)₂NH₂, —S(═O)₂N(CH₃)₂, —OH, —OCN, —OCH₃, —OCH₂CH₃, —CH₂OH, —CH₂CH₂OH, —CHOHCH₂OH, —CHOHCH₃, —SH, —SCN, —SCH₃, —SCH₂CH₃, —CH₂SH, —CH₂CH₂SH, —CHSHCH₂SH and —CHSHCH₃.

3. The compound of claim 1, wherein the compound comprises at least 60%, at least 70%, at least 80%, at least 90%, at least 95%, or at least 99% of said single stereoisomer.

4. The compound of claim 1, wherein Y is bound to C through the bond z attached at carbon atom x of Y.

5. The compound of claim 1, wherein the compound is selected from the group consisting of the following compounds: 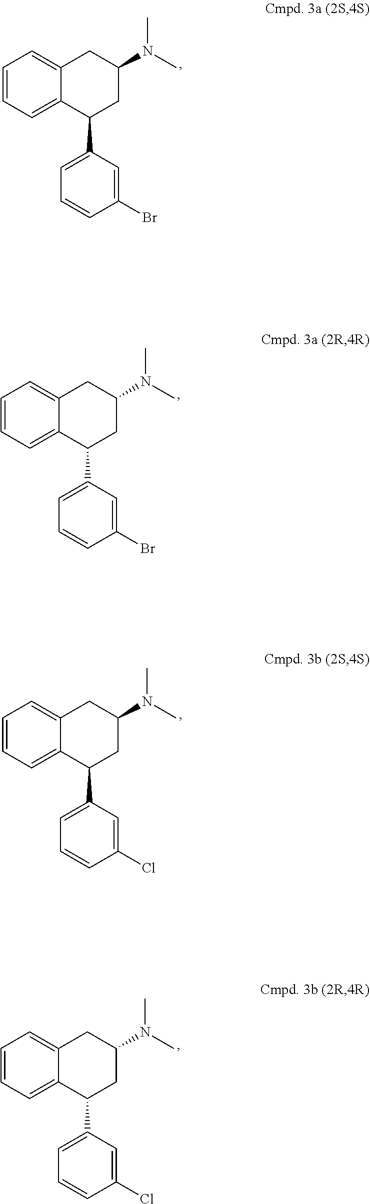 [SMILES: CN(C)[C@@H]1Cc2ccccc2[C@H](c2cccc(Br)c2)C1 ; CN(C)[C@H]1Cc2ccccc2[C@@H](c2cccc(Br)c2)C1 ; CN(C)[C@@H]1Cc2ccccc2[C@H](c2cccc(Cl)c2)C1 ; CN(C)[C@H]1Cc2ccccc2[C@@H](c2cccc(Cl)c2)C1] 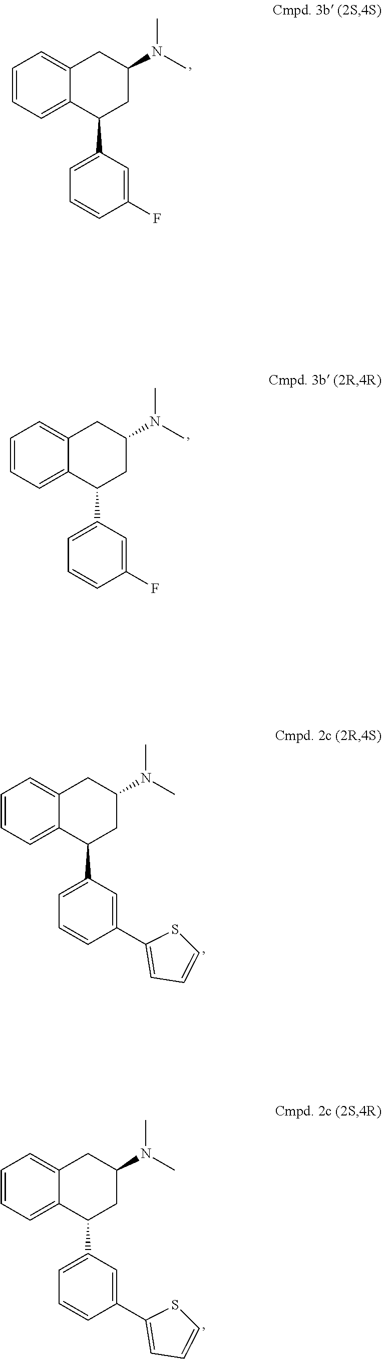 [SMILES: CN(C)[C@@H]1Cc2ccccc2[C@H](c2cccc(F)c2)C1 ; CN(C)[C@H]1Cc2ccccc2[C@@H](c2cccc(F)c2)C1 ; CN(C)[C@H]1Cc2ccccc2[C@H](c2cccc(-c3cccs3)c2)C1 ; CN(C)[C@@H]1Cc2ccccc2[C@@H](c2cccc(-c3cccs3)c2)C1] 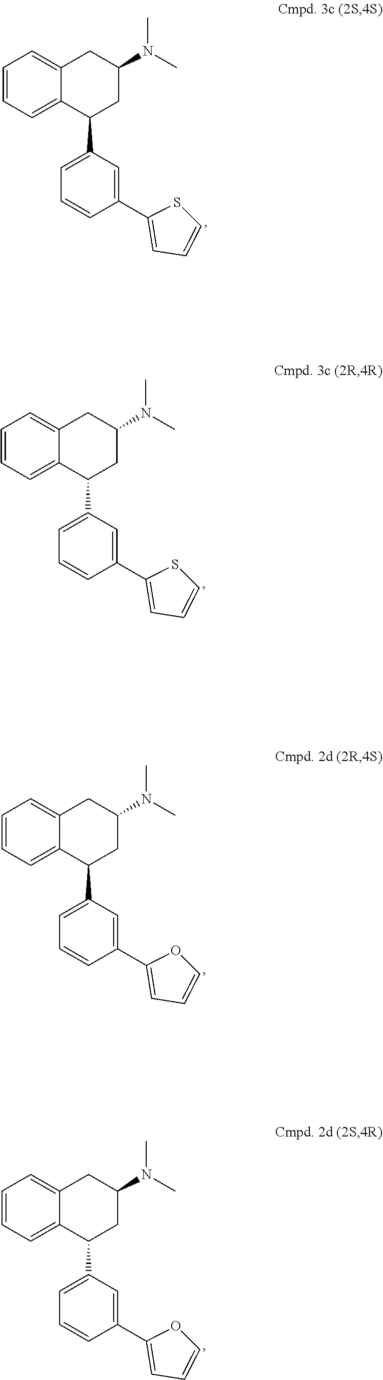 [SMILES: CN(C)[C@@H]1Cc2ccccc2[C@H](c2cccc(-c3cccs3)c2)C1 ; CN(C)[C@H]1Cc2ccccc2[C@@H](c2cccc(-c3cccs3)c2)C1 ; CN(C)[C@H]1Cc2ccccc2[C@H](c2cccc(-c3ccco3)c2)C1 ; CN(C)[C@@H]1Cc2ccccc2[C@@H](c2cccc(-c3ccco3)c2)C1] 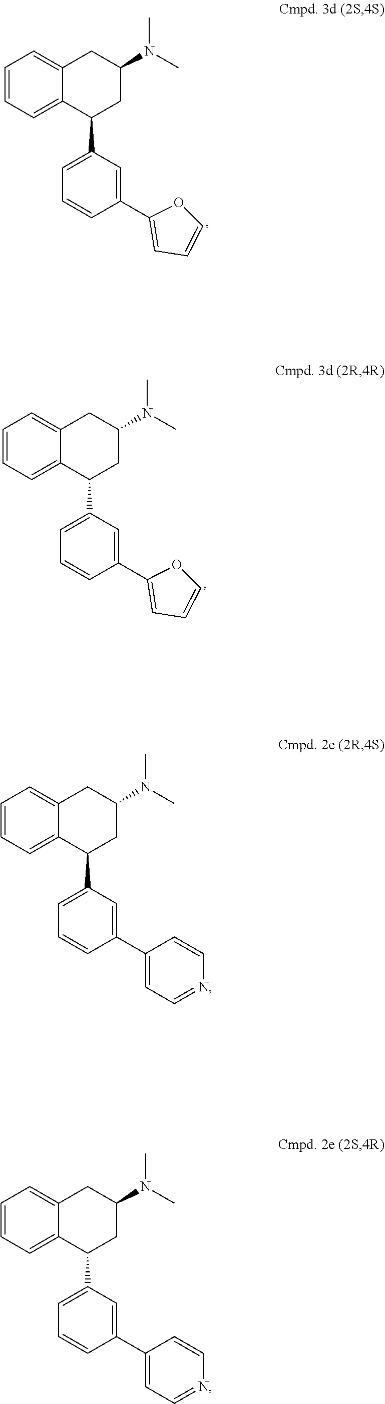 [SMILES: CN(C)[C@@H]1Cc2ccccc2[C@H](c2cccc(-c3ccco3)c2)C1 ; CN(C)[C@H]1Cc2ccccc2[C@@H](c2cccc(-c3ccco3)c2)C1 ; CN(C)[C@H]1Cc2ccccc2[C@H](c2cccc(-c3ccncc3)c2)C1 ; CN(C)[C@@H]1Cc2ccccc2[C@@H](c2cccc(-c3ccncc3)c2)C1] 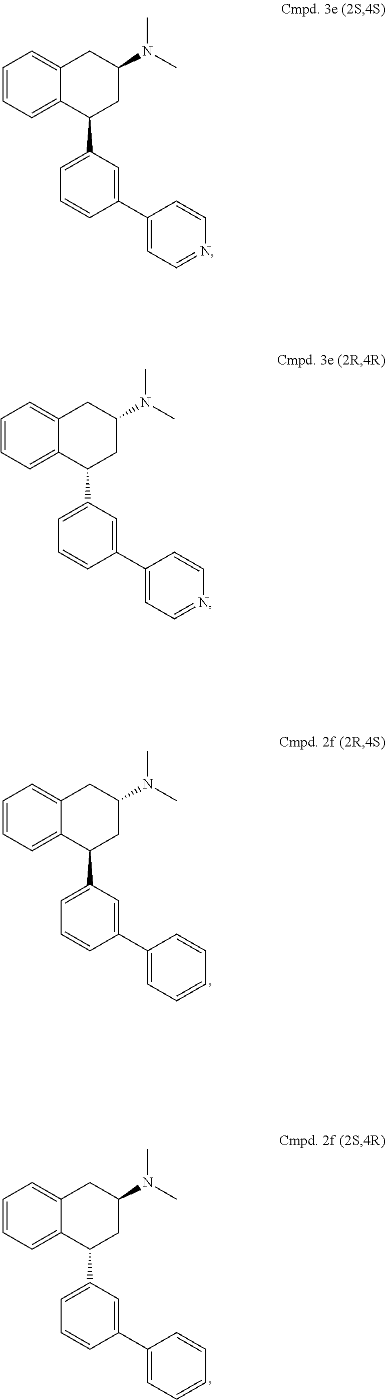 [SMILES: CN(C)[C@@H]1Cc2ccccc2[C@H](c2cccc(-c3ccncc3)c2)C1 ; CN(C)[C@H]1Cc2ccccc2[C@@H](c2cccc(-c3ccncc3)c2)C1 ; CN(C)[C@H]1Cc2ccccc2[C@H](c2cccc(-c3ccccc3)c2)C1 ; CN(C)[C@@H]1Cc2ccccc2[C@@H](c2cccc(-c3ccccc3)c2)C1] 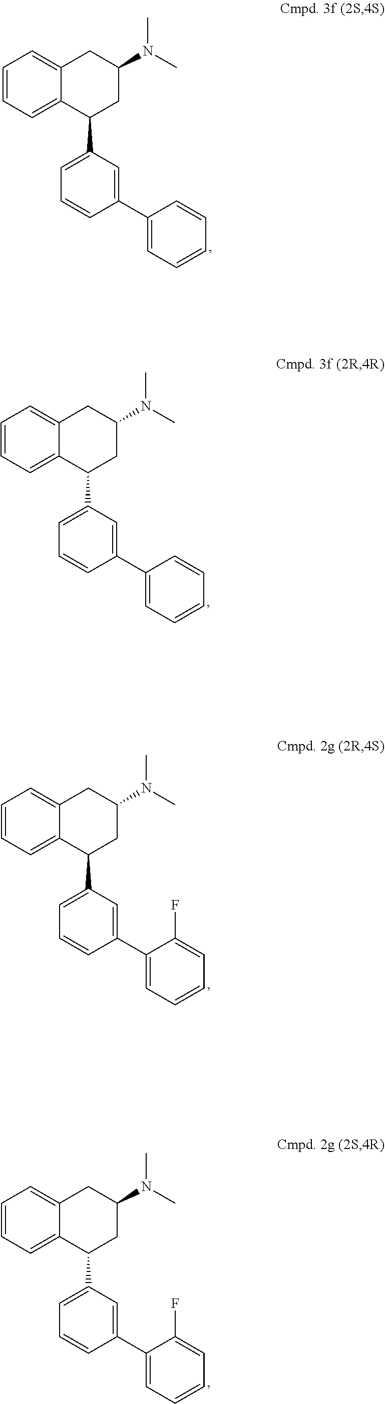 [SMILES: CN(C)[C@@H]1Cc2ccccc2[C@H](c2cccc(-c3ccccc3)c2)C1 ; CN(C)[C@H]1Cc2ccccc2[C@@H](c2cccc(-c3ccccc3)c2)C1 ; CN(C)[C@H]1Cc2ccccc2[C@H](c2cccc(-c3ccccc3F)c2)C1 ; CN(C)[C@@H]1Cc2ccccc2[C@@H](c2cccc(-c3ccccc3F)c2)C1] 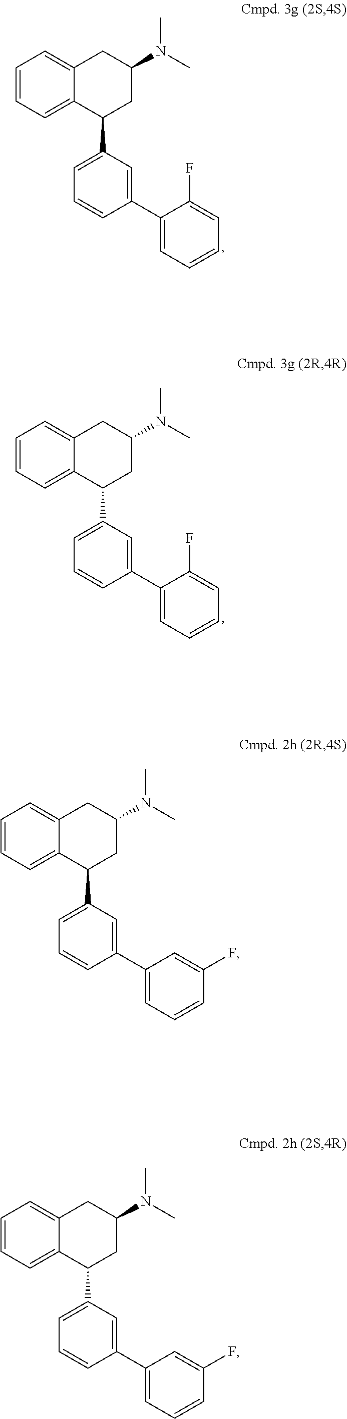 [SMILES: CN(C)[C@@H]1Cc2ccccc2[C@H](c2cccc(-c3ccccc3F)c2)C1 ; CN(C)[C@H]1Cc2ccccc2[C@@H](c2cccc(-c3ccccc3F)c2)C1 ; CN(C)[C@H]1Cc2ccccc2[C@H](c2cccc(-c3cccc(F)c3)c2)C1 ; CN(C)[C@@H]1Cc2ccccc2[C@@H](c2cccc(-c3cccc(F)c3)c2)C1] 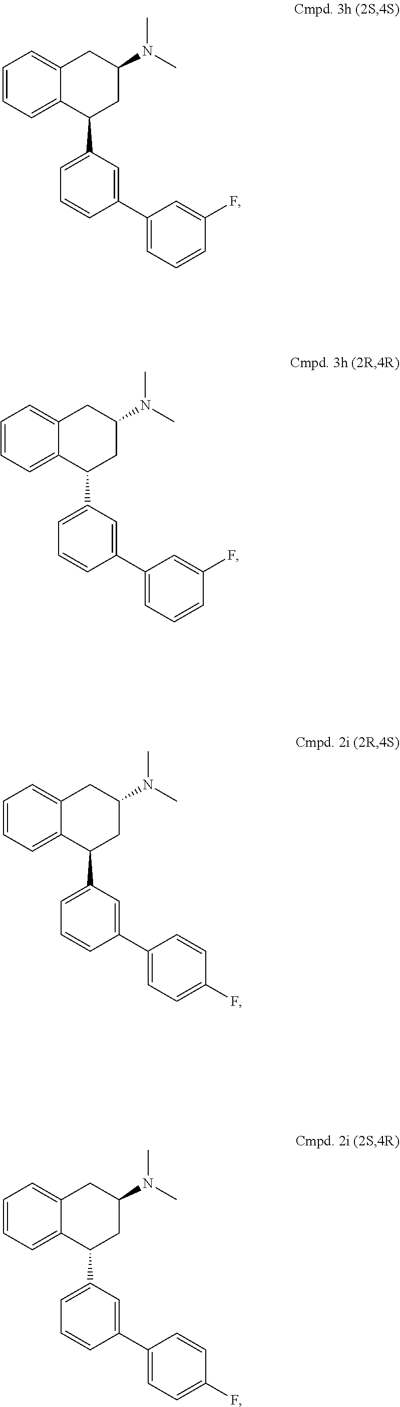 [SMILES: CN(C)[C@@H]1Cc2ccccc2[C@H](c2cccc(-c3cccc(F)c3)c2)C1 ; CN(C)[C@H]1Cc2ccccc2[C@@H](c2cccc(-c3cccc(F)c3)c2)C1 ; CN(C)[C@H]1Cc2ccccc2[C@H](c2cccc(-c3ccc(F)cc3)c2)C1 ; CN(C)[C@@H]1Cc2ccccc2[C@@H](c2cccc(-c3ccc(F)cc3)c2)C1] 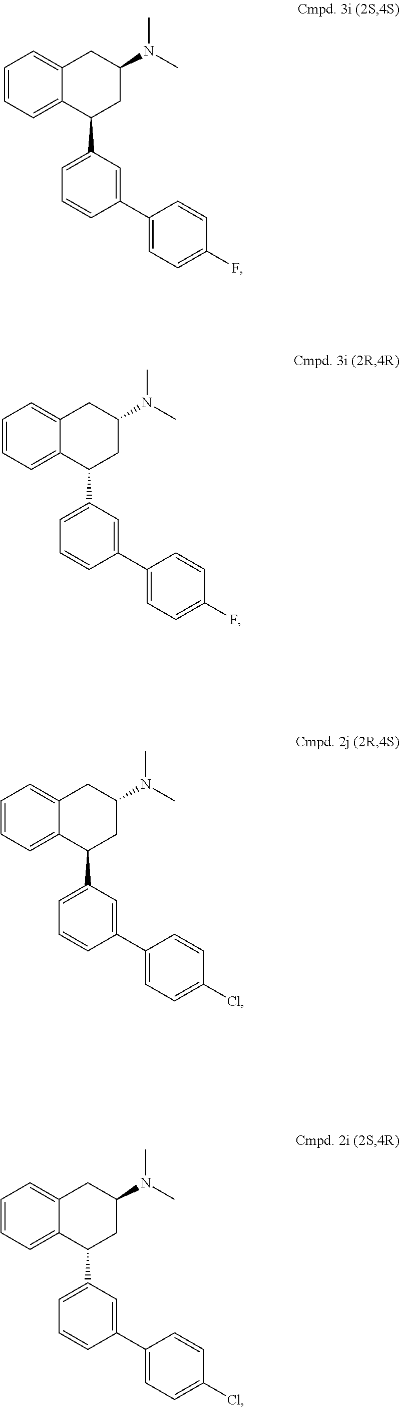 [SMILES: CN(C)[C@@H]1Cc2ccccc2[C@H](c2cccc(-c3ccc(F)cc3)c2)C1 ; CN(C)[C@H]1Cc2ccccc2[C@@H](c2cccc(-c3ccc(F)cc3)c2)C1 ; CN(C)[C@H]1Cc2ccccc2[C@H](c2cccc(-c3ccc(Cl)cc3)c2)C1 ; CN(C)[C@@H]1Cc2ccccc2[C@@H](c2cccc(-c3ccc(Cl)cc3)c2)C1] 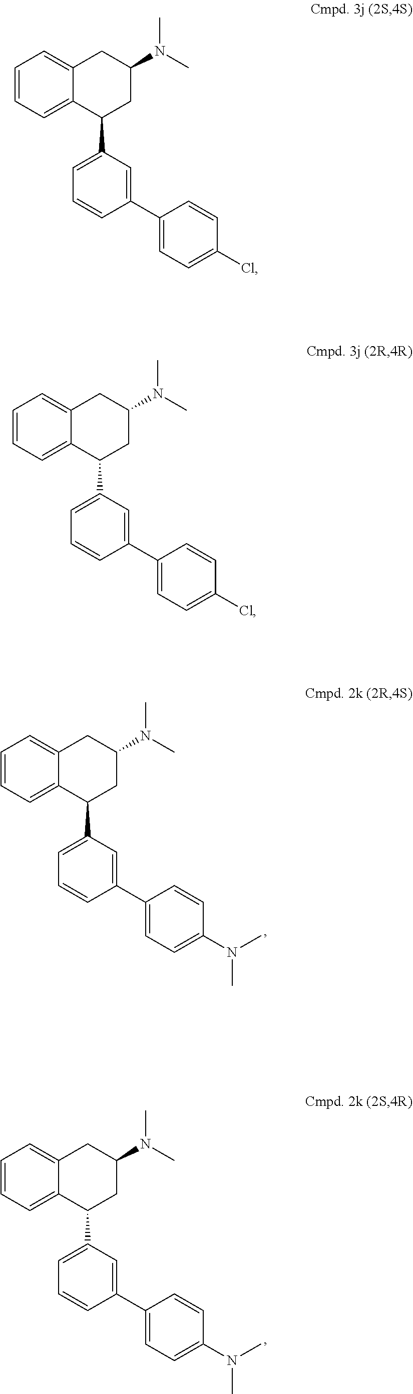 [SMILES: CN(C)[C@@H]1Cc2ccccc2[C@H](c2cccc(-c3ccc(Cl)cc3)c2)C1 ; CN(C)[C@H]1Cc2ccccc2[C@@H](c2cccc(-c3ccc(Cl)cc3)c2)C1 ; CN(C)c1ccc(-c2cccc([C@@H]3C[C@@H](N(C)C)Cc4ccccc43)c2)cc1 ; CN(C)c1ccc(-c2cccc([C@H]3C[C@H](N(C)C)Cc4ccccc43)c2)cc1] 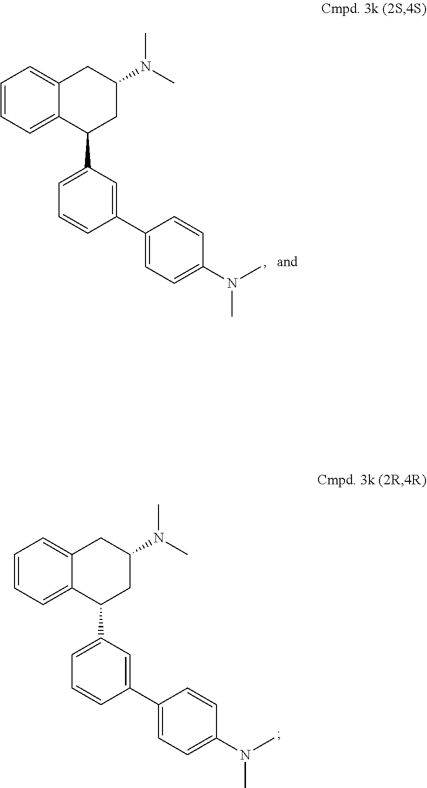 [SMILES: CN(C)c1ccc(-c2cccc([C@@H]3C[C@@H](N(C)C)Cc4ccccc43)c2)cc1 ; CN(C)c1ccc(-c2cccc([C@H]3C[C@@H](N(C)C)Cc4ccccc43)c2)cc1]
or a pharmaceutically acceptable salt, hydrate, or solvate thereof.

6. The compound of claim 1, wherein the compound is a neutral antagonist or an inverse agonist at one or more of the 5-HT2A and 5-HT2C receptors.

7. The compound of claim 1, wherein the compound does not cause sedation when administered to a subject at physiologically relevant levels.

8. The compound of claim 1, wherein the compound comprises a greater binding affinity at 5-HT2A receptor and/or 5-HT2C receptor than at 5-HT2B receptor.

9. The compound of claim 1, wherein the compound has a greater binding affinity for 5-HT2A receptor and 5-HT2C receptor than for 5-HT1A, 5-HT2B, 5-HT7, D2, D3, alpha1A, and/or alpha1B receptors.

10. The compound of claim 1, wherein the compound is a neutral antagonist or an inverse agonist at a histamine (H1) receptor at physiologically relevant levels.

11. The compound of claim 1, wherein the compound comprises a greater binding affinity at 5-HT2A receptors and/or 5-HT2C receptors than at the H1 receptor.

12. The compound of claim 1, wherein the one or more moieties V and/or W comprise a positive and/or a negative charge at a physiological pH.

13. The compound of claim 12, comprising a pharmaceutically acceptable anion comprising acetate, adipate, aspartate, benzenesulfonate, benzoate, besylate, bicarbonate, bitartrate, bromide, camsylate, caprate, caproate, caprylate, carbonate, chloride, citrate, decanoate, dodecylsulfate, edetate, esylate, formate, fumarate, gluceptate, gluconate, glutamate, glycolate, hexanoate, hydroxynaphthoate, iodide, isethionate, lactate, lactobionate, laurate, malate, maleate, mandelate, mesylate, methylsulfate, mucate, napsylate, nitrate, octanoate, oleate, oxalate, palmitate, pamoate, pantothenate, phosphate, dihydrogen phosphate dodecahydrate, dihydrogen phosphate dihydrate, polygalacturonate, propionate, sabacate, salicylate, stearate, acetate, succinate, sulfate, tartrate, teoclate, thiocyanate, tosylate, or undecylenate.

14. The compound of claim 12, comprising a pharmaceutically acceptable cation comprising aluminum, arginine, benzathine, calcium, chloroprocaine, choline, diethanolamine, ethanolamine, ethylenediamine, lysine, magnesium, histidine, lithium, meglumine, potassium, procaine, sodium, triethylamine, or zinc.

15. The compound of claim 1, wherein the compound comprises a hydrate or a solvate comprising one or more water molecules and/or one or more solvent molecules associated via hydrogen bonding and/or ionic bonding to the compound and/or to an anion or cation associated with the compound.

16. The compound of claim 1, wherein the compound comprises one or more of ¹⁸F, ¹⁹F, ⁷⁵Br, ⁷⁶Br, ¹²³I, ¹²⁴I, ¹²⁵I, ¹³¹I, ¹¹C, ¹³C, ¹³N, ¹⁵O, or ³H.

17. The compound of claim 1, wherein the compound selectively modulates a physiological activity of 5-HT2A and/or 5-HT2C receptors over a physiological activity of one or more of 5-HT1A, 5-HT2B, 5HT7, D2, D3, α1A, and α1B receptors.

18. The compound of claim 17, wherein said selective modulation is associated with a difference in binding affinity, inverse agonism, agonism, partial agonism, allosteric agonism, antagonism, partial antagonism, or allosteric antagonism.

19. A pharmaceutical composition comprising a therapeutically effective amount of a compound of claim 1 and an excipient.

20. The pharmaceutical composition of claim 19 comprising an amount of said compound that aids in treating psychosis, fragile X syndrome, autism, substance use disorder, or an impulsive behavior.

21. The pharmaceutical composition of claim 19 comprising an amount of said compound that aids in treating hypertension, migraine, obesity, irritable bowel syndrome, Parkinson's disease, attention deficit hyperactivity disorder, anxiety or generalized anxiety, depression, schizophrenia, binge eating, opioid use disorder, amphetamine use disorder, panic disorder, social anxiety disorder, obsessive-compulsive disorder, pain, Alzheimer's disease, or Huntington's disease.

22. The pharmaceutical composition of claim 19, wherein the compound comprises (2R,4S)-(trans)-4-(3-(thiophen-2-yl)phenyl)-N,N-dimethyl-1,2,3,4-tetrahydronaphthalen-2-amine, (2R,4S)-(trans)-4-(3-(furan-2-yl)phenyl)-N,N-dimethyl-1,2,3,4-tetrahydronaphthalen-2-amine, or (2S,4S)-(cis)-4-([1,1′-biphenyl]-3-yl)-N,N-dimethyl-1,2,3,4-tetrahydronaphthalen-2-amine.

23. A method to aid in treating a disease or disorder, the method comprising administering an effective amount of a compound of any of claims 1-18 to a mammalian subject in need thereof.

24. The method of claim 23, wherein the compound is administered as the pharmaceutical composition of claim 19.

25. The method of claim 23, wherein said administering does not cause sedation, dizziness, and/or orthostatic hypotension.

26. The method of claim 23, wherein the disease or disorder is a neuropsychiatric disorder is selected from the group consisting of psychosis, fragile X syndrome, autism, substance use disorder, and impulsive behaviors.

27. The method of claim 23, wherein the disease or disorder is selected from the group consisting of hypertension, migraine, obesity, irritable bowel syndrome, Parkinson's disease, attention deficit hyperactivity disorder, anxiety or generalized anxiety, depression, schizophrenia, binge eating, opioid use disorder, amphetamine use disorder, panic disorder, social anxiety disorder, obsessive-compulsive disorder, pain, Alzheimer's disease, or Huntington's disease.

28. The method of claim 23, wherein said administering results in selective modulation of a serotonin 5-HT2A or 5-HT2C receptor in the subject.

29. The method of claim 18, wherein the selective modulation comprises inverse agonism, agonism, partial agonism, allosteric agonism, antagonism, partial antagonism, allosteric antagonism, or a difference in binding affinity compared to a different receptor type.

30. Use of the compound of claim 1 to treat or prevent psychosis, fragile X syndrome, autism, substance use disorder, impulsive behaviors, hypertension, migraine, obesity, irritable bowel syndrome, Parkinson's disease, attention deficit hyperactivity disorder, anxiety or generalized anxiety, depression, schizophrenia, binge eating, opioid use disorder, amphetamine use disorder, panic disorder, social anxiety disorder, obsessive-compulsive disorder, pain, Alzheimer's disease, and/or Huntington's disease in a mammalian subject.

31. The use of claim 30, wherein said use does not cause sedation in the subject.

32. The use of claim 30, wherein said use does not agonize 5-HT2B receptors and/or does not antagonize H1 receptors in the subject.

33. A compound for selective modulation of one or more of peripheral serotonin 5-HT2A, 5-HT2B, and 5-HT2C receptors, the compound having a structure according to Formula I: 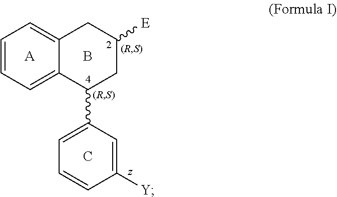
wherein E is a quaternary amine selected from the group consisting of —N⁺(CH₃)₃, —N⁺(CH₃)₂(CH₂CH₃), —N⁺(CH₃)(CH₂CH₃)₂, and —N⁺(CH₂CH₃)₃;
wherein Y is selected from the group consisting of: 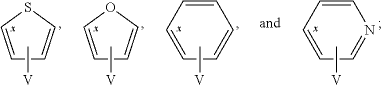
wherein covalent bond z is attached at any carbon atom of Y;
wherein Y is unsubstituted or is substituted with one or more moieties V, each of the one or more moieties V independently selected from the group consisting —F, —Cl, —Br, —I, —NH₂, —NH(CH₃), —N(CH₃)₂, —NH(CH₂CH₃), —N(CH₂CH₃)₂, —C═NH, —C═NNH₂, —C═ONH₂, —NO₂, —NO, —CN, —N₃, —N═C═O, —CH₃, —CH₂CH₃, —CH(CH₃)₂, —C═OOH, —CH₂C═OOH, —S═OCH₃, —S(═O)₂CH₃, —S(═O)₂OH, —S(═O)₂NH₂, —S(═O)₂N(CH₃)₂, —OH, —OCN, —OCH₃, —OCH₂CH₃, —CH₂OH, —CH₂CH₂OH, —CHOHCH₂OH, —CHOHCH₃, —SH, —SCN, —SCH₃, —SCH₂CH₃, —CH₂SH, —CH₂CH₂SH, —CHSHCH₂SH, —CHSHCH₃, and substituted or unsubstituted thiophene, furanyl, phenyl and pyridyl; and
wherein the compound comprises at least 50% of a single stereoisomer selected from the group of stereoisomers consisting of 2R4R, 2S4S, 2R4S, and 2S4R;
or a pharmaceutically acceptable salt, hydrate, or solvate thereof.

34. The compound of claim 33, wherein one or more moieties V are independently selected from the group consisting of: 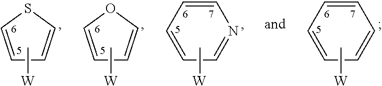
wherein V is attached to Y via a covalent bond to any one of carbons 5-7 of V; and
wherein V is substituted with one or more substituents W, each of the one or more substituents W independently selected from the group consisting of —F, —Cl, —Br, —I, —NH₂, —NH(CH₃), —N(CH₃)₂, —NH(CH₂CH₃), —N(CH₂CH₃)₂, —C═NH, —C═NNH₂, —C═ONH₂, —NO₂, —NO, —CN, —N₃, —N═C═O, —CH₃, —CH₂CH₃, —CH(CH₃)₂, —C═OOH, —CH₂C═OOH, —S═OCH₃, —S(═O)₂CH₃, —S(═O)₂OH, —S(═O)₂NH₂, —S(═O)₂N(CH₃)₂, —OH, —OCN, —OCH₃, —OCH₂CH₃, —CH₂OH, —CH₂CH₂OH, —CHOHCH₂OH, —CHOHCH₃, —SH, —SCN, —SCH₃, —SCH₂CH₃, —CH₂SH, —CH₂CH₂SH, —CHSHCH₂SH and —CHSHCH₃.

35. The compound of claim 33, wherein the compound comprises at least 60%, at least 70%, at least 80%, at least 90%, at least 95%, or at least 99% of said single stereoisomer.

36. The compound of claim 33, wherein Y is bound to C through the bond z attached at the carbon atom x of Y.

37. The compound of claim 33, wherein the compound is selected from the group consisting of: 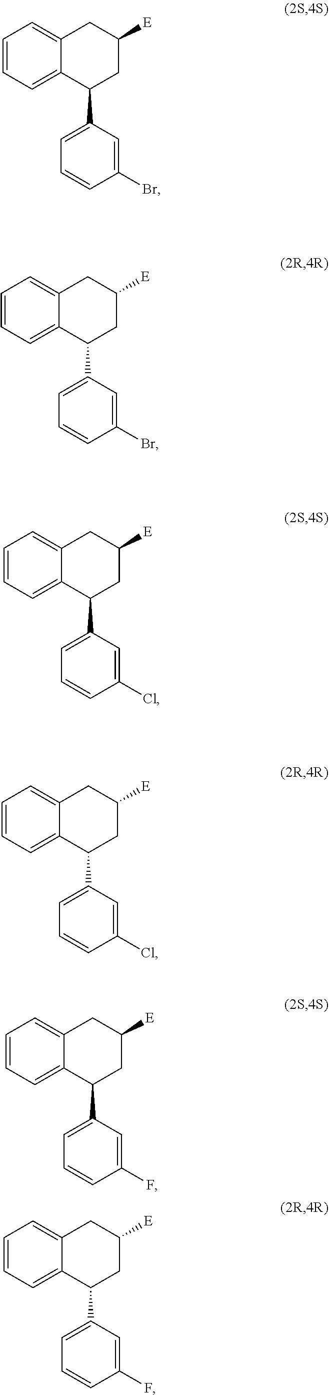 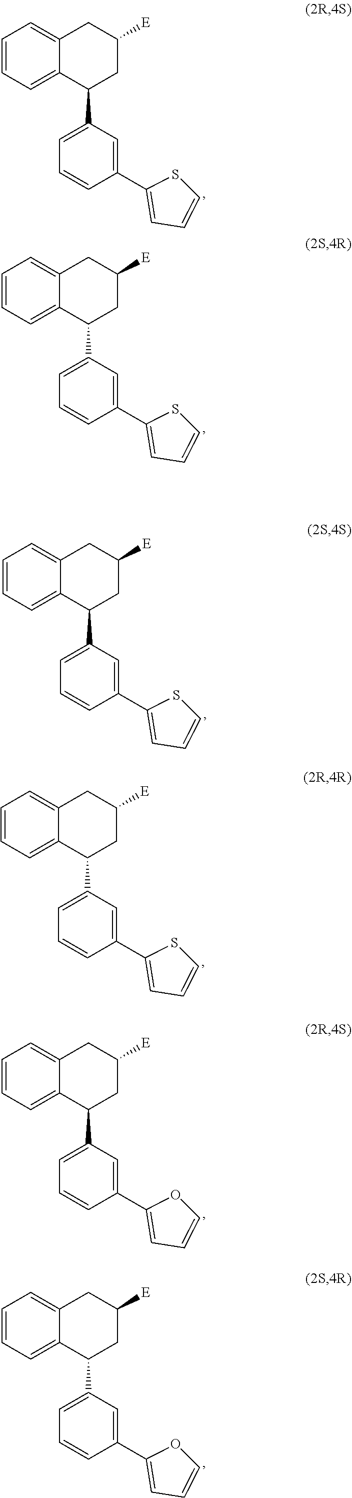 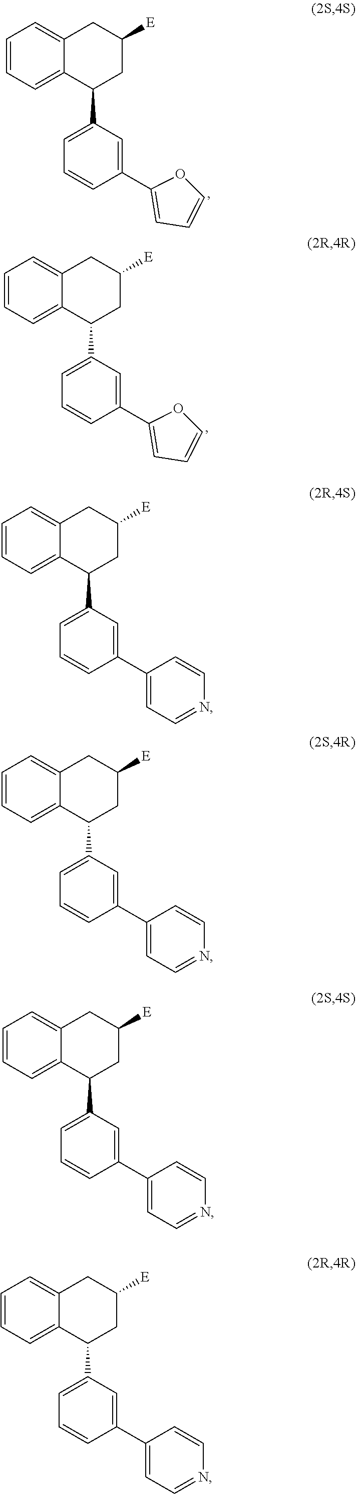 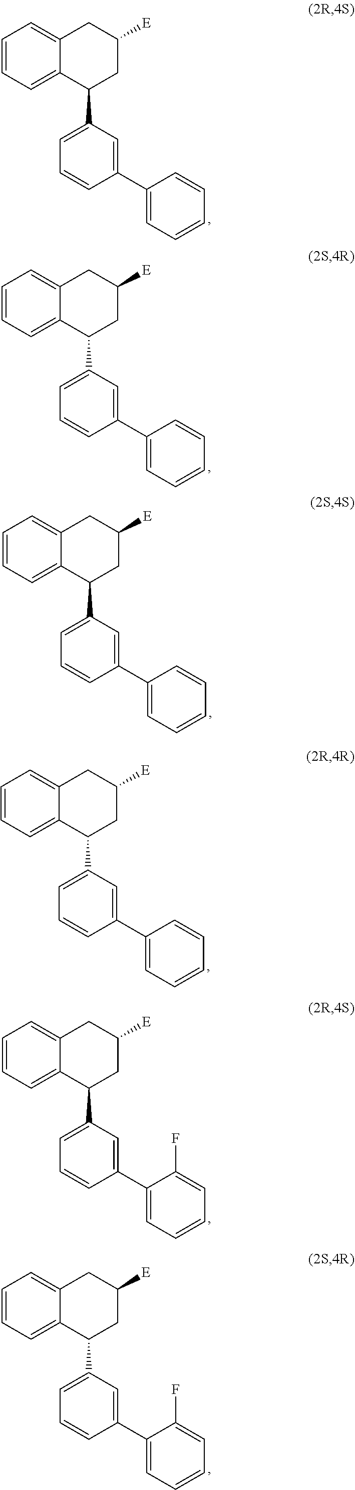 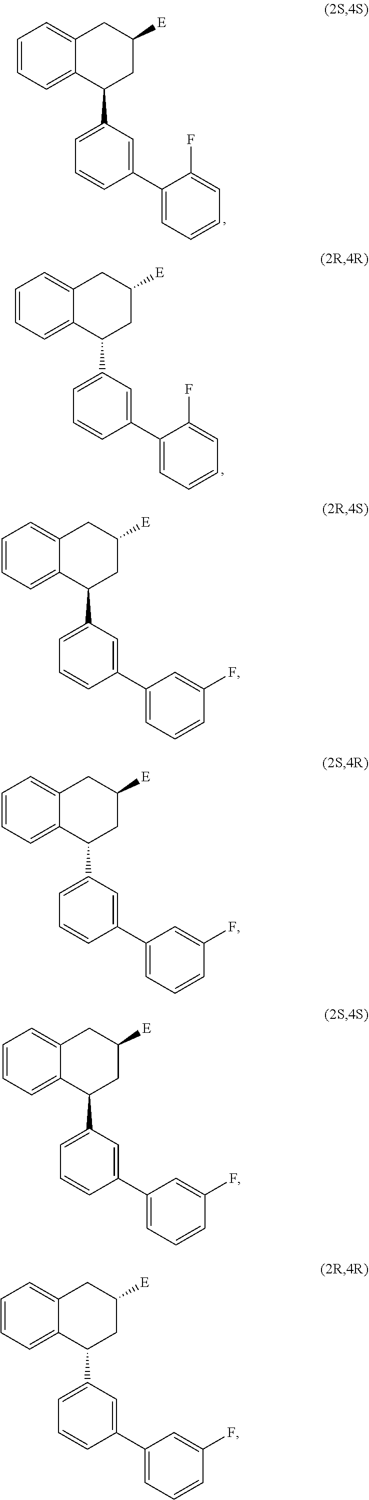 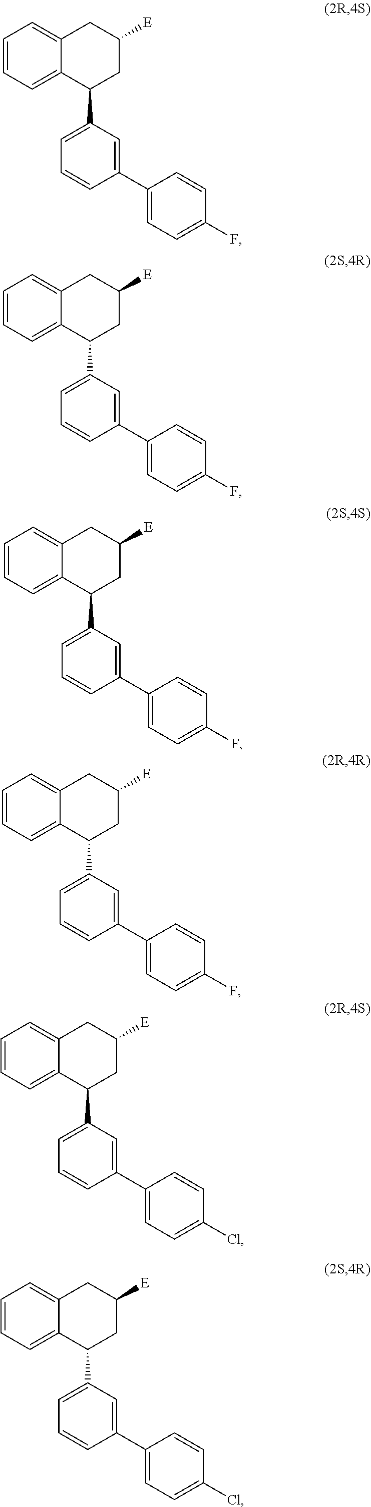 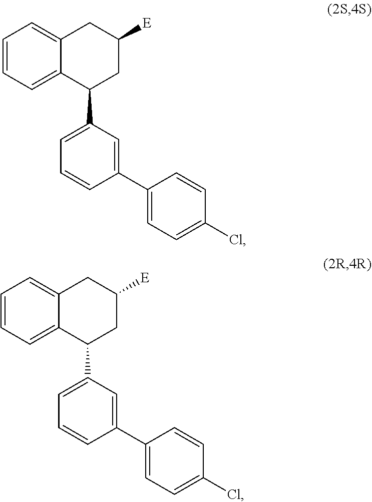 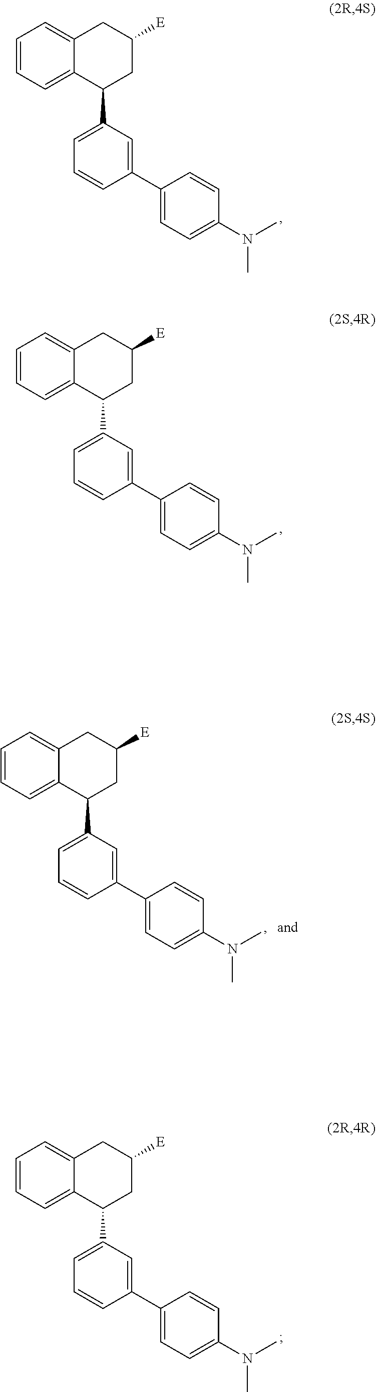
or a pharmaceutically acceptable salt, hydrate, or solvate thereof.

38. The compound of claim 33, wherein the compound is an antagonist, a neutral antagonist or an inverse agonist at one or more of the 5-HT2A, 5-HT2B, and 5-HT2C receptors.

39. The compound of claim 33, wherein the compound has a greater binding affinity for 5-HT2A, 5-HT2B, and/or 5-HT2C receptors than for 5-HT1A, 5-HT7, D2, D3, alpha1A, and/or alpha1B receptors.

40. The compound of claim 33, comprising a pharmaceutically acceptable anion selected from the group consisting of acetate, adipate, aspartate, benzenesulfonate, benzoate, besylate, bicarbonate, bitartrate, bromide, camsylate, caprate, caproate, caprylate, carbonate, chloride, citrate, decanoate, dodecylsulfate, edetate, esylate, formate, fumarate, gluceptate, gluconate, glutamate, glycolate, hexanoate, hydroxynaphthoate, iodide, isethionate, lactate, lactobionate, laurate, malate, maleate, mandelate, mesylate, methylsulfate, mucate, napsylate, nitrate, octanoate, oleate, oxalate, palmitate, pamoate, pantothenate, phosphate, dihydrogen phosphate dodecahydrate, dihydrogen phosphate dihydrate, polygalacturonate, propionate, sabacate, salicylate, stearate, acetate, succinate, sulfate, tartrate, teoclate, thiocyanate, tosylate, and undecylenate.

41. The compound of claim 33, wherein the compound comprises one or more of ¹⁸F, ¹⁹F, ⁷⁵Br, ⁷⁶Br, ¹²³I, ¹²⁴I, ¹²⁵I, ¹³¹I, ¹¹C, ¹³C, ¹³N, ¹⁵O, or ³H.

42. The compound of claim 33, wherein the compound selectively modulates a physiological activity of 5-HT2A, 5-HT2B, and/or 5-HT2C receptors over a physiological activity of one or more of 5-HT1A, 5HT7, D2, D3, α1A, and α1B receptors.

43. The compound of claim 42, wherein said selective modulation is associated with a difference in binding affinity, inverse agonism, agonism, partial agonism, allosteric agonism, antagonism, partial antagonism, or allosteric antagonism.

44. A pharmaceutical composition comprising a compound of claim 33 and an excipient.

45. The pharmaceutical composition of claim 44, wherein the compound comprises (2R,4S)-(trans)-4-(3-(thiophen-2-yl)phenyl)-N,N,N-trimethyl-1,2,3,4-tetrahydronaphthalen-2-amine, (2R,4S)-(trans)-4-(3-(furan-2-yl)phenyl)-N,N,N-trimethyl-1,2,3,4-tetrahydronaphthalen-2-amine, or (2S,4S)-(cis)-4-([1,1′-biphenyl]-3-yl)-N,N,N-trimethyl-1,2,3,4-tetrahydronaphthalen-2-amine.

46. A method to aid in treating a disease or disorder, the method comprising administering an effective amount of a compound of 33 to a mammalian subject in need thereof.

47. The method of claim 46, wherein the disease or disorder is selected from the group consisting of hypertension, thrombosis, deep vein thrombosis, pulmonary embolus, atrial fibrillation, atherosclerosis, valvular atherosclerosis, cardiac fibrosis, obesity, irritable bowel syndrome, and lack of bladder control.

48. The method of claim 47, wherein the subject further suffers from a neuropsychiatric disease or disorder, such as depression.

49. The method of claim 46, wherein the method results in inverse agonism, antagonism, partial antagonism, or allosteric antagonism at a peripheral 5-HT-2A, 5-HT2B, and/or 5-HT2C receptor.
